(19) United States
(12) Patent Application Publication
(10) Pub. No.: US 2021/0221832 A1
(43) Pub. Date: Jul. 22, 2021

(54) SUBSTITUTED PYRIDOINDOLES FOR THE TREATMENT AND PROPHYLAXIS OF BACTERIAL INFECTION

(71) Applicant: Hoffmann-La Roche Inc., Little Falls, NJ (US)

(72) Inventors: Xiao DING, Shanghai (CN); Yongqiang LIU, Shanghai (CN); Hong SHEN, Shanghai (CN); Houguang SHI, Shanghai (CN); Xuefei TAN, Shanghai (CN); Chengang ZHOU, Shanghai (CN); Mingwei ZHOU, Shanghai (CN)

(73) Assignee: Hoffmann-La Roche Inc., Little Falls, NJ (US)

(21) Appl. No.: 17/213,125

(22) Filed: Mar. 25, 2021

Related U.S. Application Data
(63) Continuation of application No. PCT/EP2019/075583, filed on Sep. 24, 2019.

(30) Foreign Application Priority Data
Sep. 26, 2018 (CN) PCT/CN2018/107584

(51) Int. Cl. C07F 9/6561; C07D 519/00; A61P 31/04; B01J 23/44

(52) U.S. Cl. CPC C07F 9/6561; B01J 23/44; A61P 31/04; C07D 519/00

(57) ABSTRACT

The present invention relates to novel compounds of formula (I) or (II),

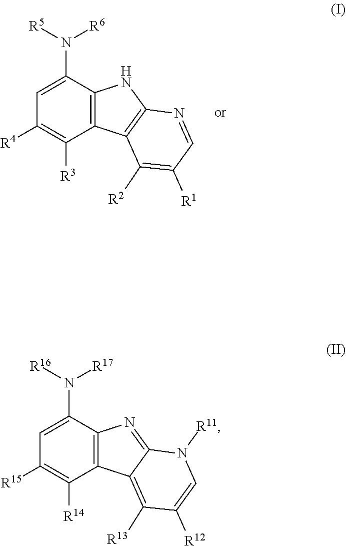

wherein R¹ to R⁶, R¹¹ to R¹⁷ are as described herein, and their pharmaceutically acceptable salt thereof, and compositions including the compounds and methods of using the compounds.

### CROSS-REFERENCE TO RELATED APPLICATIONS

This application is a continuation of International Application No. PCT/EP2019/075583 having an International filing date of Sep. 24, 2019, which claims priority to PCT/CN2018/107584 filed on Sep. 26, 2018, both of which are incorporated herein in their entirety.

### BACKGROUND OF THE INVENTION

The present invention relates to organic compounds useful for therapy and/or prophylaxis in a mammal, and in particular to inhibitors of DNA gyrase and/or topoisomerase IV useful for treatment and/or prophylaxis of bacterial infection.

### FIELD OF THE INVENTION

Bacterial infections pose a continuing medical problem because anti-bacterial drugs eventually engender resistance in the bacteria on which they are used. Bacterial resistance against virtually all current antibiotic drugs are increasing. Many forms of antibiotic resistance can even cross international boundaries and spread with remarkable speed. Thus novel classes of antibacterial compounds are urgently needed.

One target for development of anti-bacterial drugs has been DNA gyrase and topoisomerase IV (bacterial type IIA topoisomerases), which are essential to cell life, that solve DNA topological problems resulting from the replication, transcription, and recombination of DNA. DNA Gyrase controls DNA supercoiling and relieves topological stress that occurs when the DNA strands are untwisted such as during replication. Topoisomerase IV primarily resolves linked chromosome dimers at the conclusion of DNA replication. Both enzymes can introduce double stranded DNA breaks; pass a second DNA strand through the break and rejoining the broken strands. The activity of both enzymes is driven by the binding and hydrolysis of ATP. Bacterial DNA gyrase consists of two A (GyrA) and two B (GyrB) subunits. Binding and cleavage of the DNA is associated with GyrA, whereas ATP is bound and hydrolyzed by GyrB. Bacterial Topoisomerase IV is also a hetero-tetramer that consists of two C (ParC) and two E (ParE) subunits. The latter subunits bind ATP like GyrB in order to supply energy necessary for catalytic turnover of the enzymes.

Inhibition of DNA gyrase and topoisomerase IV has potential for the development of broad-spectrum antibiotics. The enzymes are highly conserved across a broad range of gram-positive and gram-negative pathogens. There are two classes of antibiotics that demonstrated such mechanism of action. The first, well-represented by the quinolones, inhibits GyrA and ParC subunits by stabilizing the cleaved DNA-enzyme complex, thus inhibiting overall gyrase function, leading to cell death. Novobiocin, the only marketed drug in the second class, exerts its effect by blocking the ATPase activity of the enzymes. Novobiocin was identified in 1950s. But its use declined rapidly and it was eventually withdrawn from the market, mainly due to its low permeability in many bacteria strains, rise of spontaneous resistance development, and the development of more effective drugs, such as penicillinase-stable penicillins and the first cephalosporins in 1960s and 1970s.

Recently, strong inhibition of DNA gyrase and/or topoisomerase IV has been recognized to be important for low resistance development in bacterial strains treated by inhibitors of the enzymes. Inhibitors of bacterial DNA gyrase and/or topoisomerase IV with different mechanism of action compare to the widely used quinolones will exhibit minimal cross resistance, and will be potentially useful in combating quinolone resistance that has increased significantly in the past few years.

On the other hand, during both drug discovery and development, about 5˜7% of drugs approved worldwide can be classified as prodrugs. Prodrugs are bioreversible derivatives of drug molecules that undergo an enzymatic and/or chemical transformation in vivo to release the active parent drug, which can then exert the desired pharmacological effect. For antibiotic research, most of the time, due to the high dose needed for clinical effect, the parent antibiotic drug often does not possess the good aqueous solubility needed for formulation delivery, the prodrug that can provide improved aqueous solubility is then pursued. “Prodrug strategies to overcome poor water solubility”, Advanced Drug Delivery Reviews 59 (2007) 677-694. In the present invention, new types of prodrugs were disclosed with greatly improved aqueous solubility, which can be suitable for different routes of administration.

### SUMMARY OF THE INVENTION

The present invention relates to novel compounds of formula (I),

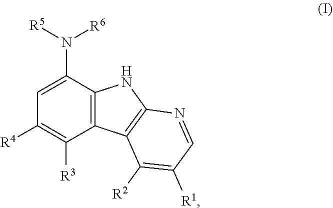

wherein

### R¹ is

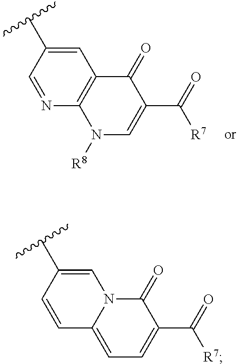 [0011] wherein R⁷ is selected from the group consisting of OH; (C₁₋₆alkyl-2-oxo-1,3-dioxol-4-yl)C₁₋₆alkoxy; aminoC₁₋₆alkylcarbonyloxyC₁₋₆alkoxy; C₁₋₆alkoxy; C₁₋₆alkoxycarbonyloxyC₁₋₆alkoxy; C₁₋₆alkylcarbonyloxyC₁₋₆alkoxy; C₁₋₆alkylsulfonylamino; carboxyC₁₋₆alkylaminocarbonylC₁₋₆alkylamino and phenylC₁₋₆alkoxy;
   R⁸ is C₁₋₆alkyl;
      R² is selected from

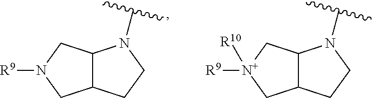

and —N(R⁹)₂;
wherein R⁹ is C₁₋₆alkyl;
   R¹⁰ is selected from the group consisting of C₁₋₆alkyl-2-oxo-1,3-dioxol-4-yl; C₁₋₆alkoxy(hydroxy)phosphoryloxyC₁₋₆alkyl and phosphonooxyC₁₋₆alkyl;
      R³ is halogen or cyano;
      R⁴ is halogen;
      R⁵ is selected from the group consisting of H; ((aminoC₁₋₆alkylcarbonyl)aminoC₁₋₆alkylcarbonyl; ((aminoC₁₋₆alkylcarbonyl)aminoC₁₋₆alkylcarbonyl)aminoC₁₋₆alkylcarbonyl; (C₁₋₆alkoxy(hydroxy)phosphoryl)C₁₋₆alkoxycarbonyl; (C₁₋₆alkoxy)₂phosphoryl; aminoC₁₋₆alkylcarbonyloxyC₁₋₆alkoxycarbonyl; C₁₋₆alkylcarbonyloxyC₁₋₆alkoxycarbonyl; carboxyC₂₋₆alkenylcarbonyloxyC₁₋₆alkoxycarbonyl; formyl; phenylC₁₋₆alkoxy(hydroxy)phosphoryl; phosphonooxyC₁₋₆alkoxycarbonyl and sulfo;
      R⁶ is C₁₋₆alkyl;
      with the proviso that when R⁵ is H and R⁷ is OH, R² is not

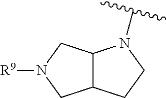

or —N(R⁹)₂;
or a pharmaceutically acceptable salt thereof.

The present invention also relates to novel compounds of formula (II),

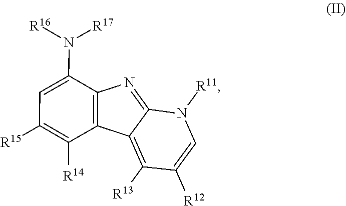

wherein
   R¹¹ is selected from the group consisting of C₁₋₆alkyl-2-oxo-1,3-dioxol-4-yl; C₁₋₆alkoxy(hydroxy)phosphoryloxyC₁₋₆alkyl and phosphonooxyC₁₋₆alkyl;

### R¹² is

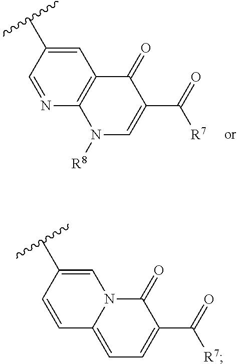 [0018] wherein R⁷ is OH;
   R⁸ is C₁₋₆alkyl;

### R¹³ is

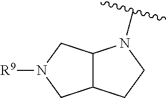

or —N(R⁹)₂; wherein R⁹ is C₁₋₆alkyl;
R¹⁴ is halogen or cyano;
R¹⁵ is halogen;

### R¹⁶ is H;

R¹⁷ is C₁₋₆alkyl;
or a pharmaceutically acceptable salt thereof.

Further objects of the present invention are novel compounds of formula (I) or (II), their manufacture, medicaments based on a compound in accordance with the invention and their production as well as the use of compounds of formula (I) or (II) for the treatment or prophylaxis of bacterial infection. The use of compounds of formula (I) or (II) as DNA gyrase and/or topoisomerase IV inhibitors is also one of the objections of present invention. The compounds of formula (I) or (II) showed superior solubility compared to parent compounds, good CC₅₀ profiles, microsomal stability and/or PK profile.

### DETAILED DESCRIPTION OF THE INVENTION

Unless otherwise defined, all technical and scientific terms used herein have the same meaning as commonly understood by one of ordinary skill in the art to which this invention belongs. Furthermore, the following definitions are set forth to illustrate and define the meaning and scope of the various terms used to describe the invention.

### Definitions

The term “C₁₋₆alkyl” denotes a saturated, linear or branched chain alkyl group containing 1 to 6, particularly 1 to 4 carbon atoms, for example methyl, ethyl, propyl, isopropyl, w-butyl, isobutyl, tert-butyl and the like. Particular “C₁₋₆alkyl” groups are methyl, ethyl and propyl.

The term “C₁₋₆alkoxy” denotes C₁₋₆alkyl-O—, wherein the “C₁₋₆alkyl” is as defined above; for example methoxy, ethoxy, propoxy, iso-propoxy, n-butoxy, iso-butoxy, 2-butoxy, tert-butoxy, pentoxy, hexyloxy and the like. Particular “C₁₋₆alkoxy” groups are methoxy, ethoxy and propoxy.

The term “C₂₋₆alkenyl” denotes an unsaturated, linear or branched chain alkenyl group containing 2 to 6, particularly 2 to 4 carbon atoms, for example vinyl, propenyl, allyl, butenyl and the like. Particular “C₂₋₆alkenyl” group is propenyl.

The term “halogen” and “halo” are used interchangeably herein and denote fluoro, chloro, bromo, or iodo.

The term “carbonyloxy” denotes —C(O)—O—.

The term “cis-isomers” and “trans-isomers” denote the relative stereochemistry of the molecule or moiety. For example: Intermediate C1

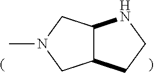

as the “cis-isomers” refers to a mixture of

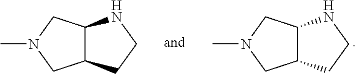

The way of showing relative stereochemistry also applies to the final compounds.

The term “enantiomer” denotes two stereoisomers of a compound which are non-superimposable mirror images of one another.

The term “diastereomer” denotes a stereoisomer with two or more centers of chirality and whose molecules are not mirror images of one another. Diastereomers have different physical properties, e.g. melting points, boiling points, spectral properties, and reactivities.

The term “pharmaceutically acceptable salts” denotes salts which are not biologically or otherwise undesirable. Pharmaceutically acceptable salts include both acid and base addition salts.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, tri ethyl amine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “pharmaceutical composition” denotes a mixture or solution comprising a therapeutically effective amount of an active pharmaceutical ingredient together with pharmaceutically acceptable excipients to be administered to a mammal, e.g., a human in need thereof.

Inhibitors of DNA Gyrase and/or Topoisomerase IV

The present invention relates to a compound of formula (I),

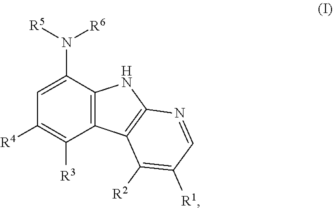

wherein

### R¹ is

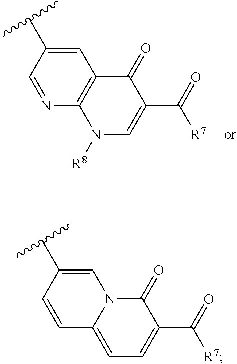 [0040] wherein R⁷ is selected from the group consisting of OH; (C₁₋₆alkyl-2-oxo-1,3-dioxol-4-yl)C₁₋₆alkoxy; aminoC₁₋₆alkylcarbonyloxyC₁₋₆alkoxy; C₁₋₆alkoxy; C₁₋₆alkoxycarbonyloxyC₁₋₆alkoxy; C₁₋₆alkylcarbonyloxyC₁₋₆alkoxy; C₁₋₆alkylsulfonylamino; carboxyC₁₋₆alkylaminocarbonylC₁₋₆alkylamino and phenylC₁₋₆alkoxy;
   R⁸ is C₁₋₆alkyl;
      R² is selected from

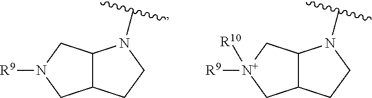

and —N(R⁹)₂;
wherein R⁹ is C₁₋₆alkyl;
   R¹⁰ is selected from the group consisting of C₁₋₆alkyl-2-oxo-1,3-dioxol-4-yl; C₁₋₆alkoxy(hydroxy)phosphoryloxyC₁₋₆alkyl and phosphonooxyC₁₋₆alkyl;
      R³ is halogen or cyano;
      R⁴ is halogen;
      R⁵ is selected from the group consisting of H; ((aminoC₁₋₆alkylcarbonyl)aminoC₁₋₆alkylcarbonyl; ((aminoC₁₋₆alkylcarbonyl)aminoC₁₋₆alkylcarbonyl)aminoC₁₋₆alkylcarbonyl; (C₁₋₆alkoxy(hydroxy)phosphoryl)C₁₋₆alkoxycarbonyl; (C₁₋₆alkoxy)₂phosphoryl; aminoC₁₋₆alkylcarbonyloxyC₁₋₆alkoxycarbonyl; C₁₋₆alkylcarbonyloxyC₁₋₆alkoxycarbonyl; carboxyC₂₋₆alkenylcarbonyloxyC₁₋₆alkoxycarbonyl; formyl; phenylC₁₋₆alkoxy(hydroxy)phosphoryl; phosphonooxyC₁₋₆alkoxycarbonyl and sulfo;
      R⁶ is C₁₋₆alkyl;
      with the proviso that when R⁵ is H and R⁷ is OH, R² is not

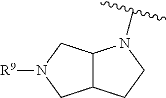

or —N(R⁹)₂;
or a pharmaceutically acceptable salt thereof.

A further embodiment of present invention is (ii) a compound of formula (I) according to (i), wherein

### R¹ is

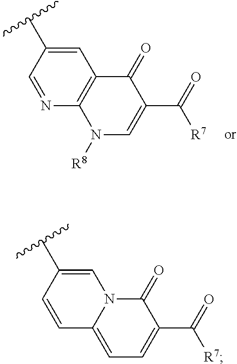 [0046] wherein R⁷ is selected from the group consisting of OH; (5-methyl-2-oxo-1,3-dioxol-4-yl)methoxy; acetoxyethoxy; amino(methyl)butanoyloxyethoxy; benzyloxy; carboxymethylaminocarbonylmethylamino; ethoxy; isopropoxycarbonyloxyethoxy and methylsulfonylamino;
   R⁸ is methyl;
      R² is selected from

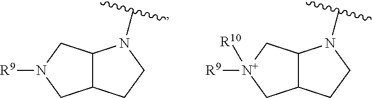

and —N(R⁹)₂;
wherein R⁹ is methyl;
   R¹⁰ is selected from the group consisting of 5-methyl-2-oxo-1,3-dioxol-4-ylmethyl; tert-butoxy(hydroxy)phosphoryloxymethyl and phosphonooxymethyl;
      R³ is fluoro or cyano;
      R⁴ is fluoro;
      R⁵ is selected from the group consisting of H; ((aminoacetyl)aminoacetyl)aminoacetyl; (aminoacetyl)aminoacetyl; (tert-butoxy(hydroxy)phosphoryloxy)methoxycarbonyl; acetoxyethoxy carbonyl; aminoacetyloxymethoxy carbonyl; amino(methyl)butanoyloxyethoxycarbony; benzyloxy(hydroxy)phosphoryl; carboxypropenoyloxymethoxycarbonyl; diethoxyphosphoryl; formyl; phosphonooxymethoxycarbonyl and sulfo;
      R⁶ is methyl;
      with the proviso that when R⁵ is H and R⁷ is OH, R² is not

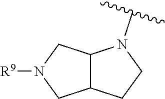

or —N(R⁹)₂;
or a pharmaceutically acceptable salt thereof.

A further embodiment of present invention is (iii) a compound of formula (I) according to (i), wherein

### R¹ is

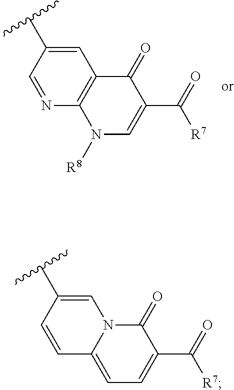 [0052] wherein R⁷ is selected from the group consisting of (C₁₋₆alkyl-2-oxo-1,3-dioxol-4-yl)C₁₋₆alkoxy; aminoC₁₋₆alkylcarbonyloxyC₁₋₆alkoxy; C₁₋₆alkoxy; C₁₋₆alkoxycarbonyloxyC₁₋₆alkoxy; C₁₋₆alkylcarbonylC₁₋₆alkoxy; C₁₋₆alkylsulfonylamino; carboxyC₁₋₆alkylaminocarbonylC₁₋₆alkylamino and phenylC₁₋₆alkoxy;
   R⁸ is C₁₋₆alkyl;

### R² is

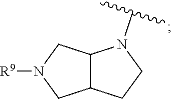

wherein R⁹ is C₁₋₆alkyl;
R³ is halogen or cyano;
R⁴ is halogen;

### R⁵ is H;

R⁶ is C₁₋₆alkyl;
or a pharmaceutically acceptable salt thereof.

A further embodiment of present invention is (iv) a compound of formula (I) according to (iii), or a pharmaceutically acceptable salt thereof, wherein R⁷ is selected from the group consisting of (C₁₋₆alkyl-2-oxo-1,3-dioxol-4-yl)C₁₋₆alkoxy; aminoC₁₋₆alkylcarbonyloxyC₁₋₆alkoxy; C₁₋₆alkoxycarbonyloxyC₁₋₆alkoxy and C₁₋₆alkylcarbonyloxyC₁₋₆alkoxy.

A further embodiment of present invention is (v) a compound of formula (I) according to (iii) or (iv), or a pharmaceutically acceptable salt thereof, wherein R⁷ is selected from the group consisting of (5-methyl-2-oxo-1,3-dioxol-4-yl)methoxy; acetoxyethoxy; amino(methyl)butanoyloxyethoxy and isopropoxycarbonyloxyethoxy.

A further embodiment of present invention is (vi) a compound of formula (I) according to any one of (iii) to (v), or a pharmaceutically acceptable salt thereof, wherein R³ is fluoro or cyano; R⁴ is fluoro; R⁶ is methyl; R⁸ is methyl; R⁹ is methyl.

A further embodiment of present invention is (vii) a compound of formula (I), or a pharmaceutically acceptable salt thereof, according to (i), wherein

### R¹ is

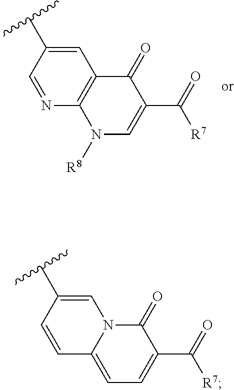 [0061] wherein R⁷ is OH;
   R⁸ is C₁₋₆alkyl;

### R² is

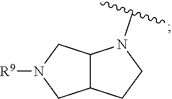

wherein R⁹ is C₁₋₆alkyl;
R³ is halogen or cyano;
R⁴ is halogen;
R⁵ is selected from the group consisting of ((aminoC₁₋₆alkylcarbonyl)aminoC₁₋₆alkylcarbonyl; ((aminoC₁₋₆alkylcarbonyl)aminoC₁₋₆alkylcarbonyl)aminoC₁₋₆alkylcarbonyl; (C₁₋₆alkoxy(hydroxy)phosphoryl)C₁₋₆alkoxycarbonyl; (C₁₋₆alkoxy)₂phosphoryl; aminoC₁₋₆alkylcarbonyloxyC₁₋₆alkoxycarbonyl; C₁₋₆alkylcarbonyloxyC₁₋₆alkoxycarbonyl; carboxyC₂₋₆alkenylcarbonyloxyC₁₋₆alkoxycarbonyl; formyl; phenylC₁₋₆alkoxy(hydroxy)phosphoryl; phosphonooxyC₁₋₆alkoxycarbonyl and sulfo;
R⁶ is C₁₋₆alkyl;
or a pharmaceutically acceptable salt thereof.

A further embodiment of present invention is (viii) a compound of formula (I) according to (vii), or a pharmaceutically acceptable salt thereof, wherein R⁵ is selected from the group consisting of C₁₋₆alkylcarbonyloxyC₁₋₆alkoxycarbonyl; carboxyC₂₋₆alkenylcarbonyloxyC₁₋₆alkoxycarbonyl and phosphonooxyC₁₋₆alkoxycarbonyl.

A further embodiment of present invention is (ix) a compound of formula (I) according to (vii) or (viii), or a pharmaceutically acceptable salt thereof, wherein R⁵ is selected from the group consisting of acetoxy ethoxy carbonyl; carboxypropenoyloxymethoxycarbonyl and phosphonooxymethoxycarbonyl.

A further embodiment of present invention is (x) a compound of formula (I) according to any one of (vii) to (ix), or a pharmaceutically acceptable salt thereof, wherein R³ is fluoro or cyano; R⁴ is fluoro; R⁶ is methyl; R⁸ is methyl; R⁹ is methyl.

An another embodiment of present invention is (xi) a compound of formula (II),

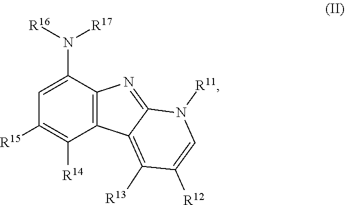

wherein
   R¹¹ is selected from the group consisting of C₁₋₆alkyl-2-oxo-1,3-dioxol-4-yl; C₁₋₆alkoxy(hydroxy)phosphoryloxyC₁₋₆alkyl and phosphonooxyC₁₋₆alkyl;

### R¹² is

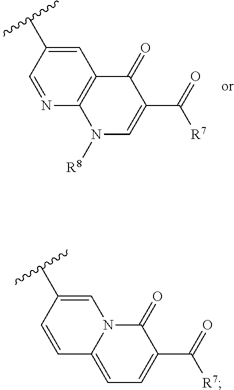 [0070] wherein R⁷ is OH;
   R⁸ is C₁₋₆alkyl;

### R¹³ is

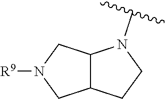

or —N(R⁹)₂; wherein R⁹ is C₁₋₆alkyl;
R¹⁴ is halogen or cyano;
R¹⁵ is halogen;

### R¹⁶ is H;

R¹⁷ is C₁₋₆alkyl;
or a pharmaceutically acceptable salt thereof.

A further embodiment of present invention is (xii) a compound of formula (II) according to (xii), or a pharmaceutically acceptable salt thereof, wherein

R¹¹ is selected from the group consisting of 5-methyl-2-oxo-1,3-dioxol-4-ylmethyl; tert-butoxy(hydroxy)phosphoryloxymethyl and phosphonooxymethyl;

### R¹² is

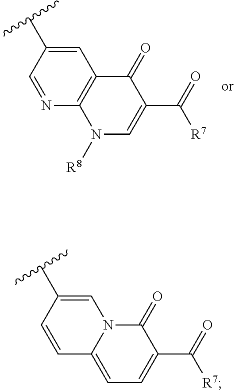 [0076] wherein R⁷ is OH; R⁸ is methyl;

### R¹³ is

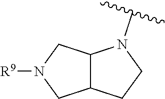

or —N(R⁹)₂; wherein R⁹ is methyl;
R¹⁴ is fluoro or cyano;
R¹⁵ is fluoro;

### R¹⁶ is H;

R¹⁷ is methyl;

or a pharmaceutically acceptable salt thereof.

Any of the above embodiments may be combined.

Another embodiment of present invention is that compounds of formula (I) or (II) are selected from:
6-[5-Cyano-6-fluoro-8-[methyl(phosphonooxymethoxycarbonyl)amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
6-[5-Cyano-6-fluoro-8-[methyl-[[(E)-3-carboxyprop-2-enoyl]oxymethoxycarbonyl]amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
6-[5-Cyano-6-fluoro-8-[formyl(methyl)amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
6-[5-Cyano-6-fluoro-8-(methylamino)-1-(phosphonooxymethyl)-4-[(3aS,6aS)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylic acid;
6-[5-Cyano-6-fluoro-8-(methylamino)-1-[(5-methyl-2-oxo-1,3-dioxol-4-yl)methyl]-4-[(3aS,6aS)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylic acid;
6-[5-Cyano-6-fluoro-8-(methylamino)-4-[(3aS,6aS)-5-methyl-5-[(5-methyl-2-oxo-1,3-dioxol-4-yl)methyl]-2,3,3a,4,6,6a-hexahydropyrrolo[2,3-c]pyrrol-5-ium-1-yl]-9H-pyrido[2,3-b]indol-3-yl]-1-methyl-4-oxo-1,8-naphthyridine-3-carboxylic acid;
6-[5-Cyano-6-fluoro-8-(methylamino)-4-[(3aS,6aS)-5-methyl-5-(phosphonooxymethyl)-2,3,3a,4,6,6a-hexahydropyrrolo[2,3-c]pyrrol-5-ium-1-yl]-9H-pyrido[2,3-b]indol-3-yl]-1-methyl-4-oxo-1,8-naphthyridine-3-carboxylic acid;
2-[(2S)-2-Amino-3-methyl-butanoyl]oxyethyl 6-[5-cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate;
2-[[2-[[6-[5-Cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carbonyl]amino]acetyl]amino]acetic acid;
6-[5-Cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-N-methylsulfonyl-4-oxo-1,8-naphthyridine-3-carboxamide;
1-Acetoxyethyl 6-[5-cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate;
1-Isopropoxycarbonyloxyethyl 6-[6-fluoro-5-methyl-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate;
(5-Methyl-2-oxo-1,3-dioxol-4-yl)methyl 6-[5-cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate;
6-[8-[(2-Aminoacetyl)oxymethoxycarbonyl-methyl-amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
6-[8-[1-Acetoxyethoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
6-[8-[Diethoxyphosphoryl(methyl)amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
6-[8-[[Benzyloxy(hydroxy)phosphoryl]-methyl-amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
6-[5,6-Difluoro-8-[methyl(phosphonooxymethoxycarbonyl)amino]-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
6-[8-[[tert-Butoxy(hydroxy)phosphoryl]oxymethoxycarbonyl-methyl-amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
(E)-4-[[[3-(6-Benzyloxycarbonyl-8-methyl-5-oxo-1,8-naphthyridin-3-yl)-5,6-difluoro-4-[(3aR,6aR)-5-methyl-2,3,3a,4,6,6a-hexahydropyrrolo[2,3-c]pyrrol-1-yl]-9H-pyrido[2,3-b]indol-8-yl]-methyl-carbamoyl]oxymethoxy]-4-oxo-but-2-enoic acid;
6-[5,6-Difluoro-8-[methyl-[[(E)-3-carboxyprop-2-enoyl]oxymethoxycarbonyl]amino]-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
6-[1-[[tert-Butoxy(hydroxy)phosphoryl]oxymethyl]-5,6-difluoro-8-(methylamino)-4-[(3aR,6aR)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylic acid;
6-[5,6-Difluoro-8-(methylamino)-1-(phosphonooxymethyl)-4-[(3aR,6aR)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylic acid;
6-[5,6-Difluoro-8-(methylamino)-4-[(3aR,6aR)-5-[[tert-butoxy(hydroxy)phosphoryl]oxymethyl]-5-methyl-2,3,3a,4,6,6a-hexahydropyrrolo[2,3-c]pyrrol-5-ium-1-yl]-9H-pyrido[2,3-b]indol-3-yl]-1-methyl-4-oxo-1,8-naphthyridine-3-carboxylic acid;
6-[5,6-Difluoro-8-(methylamino)-4-[(3aR,6aR)-5-methyl-5-(phosphonooxymethyl)-2,3,3a,4,6,6a-hexahydropyrrolo[2,3-c]pyrrol-5-ium-1-yl]-9H-pyrido[2,3-b]indol-3-yl]-1-methyl-4-oxo-1,8-naphthyridine-3-carboxylic acid;
6-[5,6-Difluoro-8-[formyl(methyl)amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
6-[5,6-Difluoro-8-[methyl(sulfo)amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
6-[5,6-Difluoro-8-[methyl-[[(E)-3-carboxyprop-2-enoyl]oxymethoxycarbonyl]amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
6-[5,6-Difluoro-8-[methyl(sulfo)amino]-4-[cis-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
6-[8-[[2-[(2-Aminoacetyl)amino]acetyl]-methyl-amino]-5,6-difluoro-4-[cis-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
Ethyl 6-[5,6-difluoro-8-(methylamino)-4-[cis-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate;
7-[5,6-Difluoro-8-[methyl(phosphonooxymethoxycarbonyl)amino]-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid;
7-[5,6-Difluoro-8-[methyl-[[(E)-3-carboxyprop-2-enoyl]oxymethoxycarbonyl]amino]-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid;
7-[8-[[2-[[2-[(2-Aminoacetyl)amino]acetyl]amino]acetyl]-methyl-amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid;
7-[8-[[2-[(2-Aminoacetyl)amino]acetyl]-methyl-amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid;
7-[5,6-Difluoro-8-(methylamino)-1-(phosphonooxymethyl)-4-[(3aS,6aS)-5-methyl-2,3,3a,4,6,6a-hexahydropyrrolo[2,3-c]pyrrol-1-yl]pyrido[2,3-b]indol-3-yl]-4-oxo-quinolizine-3-carboxylic acid;
2-[(2S)-2-Amino-3-methyl-butanoyl]oxyethyl 7-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate;
2-[[2-[[7-[5,6-Difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carbonyl]amino]acetyl]amino]acetic acid;
1-Isopropoxycarbonyloxyethyl 7-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate;
1-Acetoxyethyl 7-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate;
7-[5,6-Difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-N-methylsulfonyl-4-oxo-quinolizine-3-carboxamide;
6-[5,6-Difluoro-8-(methylamino)-4-[c/3′-5-methyl-2,3,3a,4,6,6a-hexahydropyrrolo[2,3-c]pyrrol-1-yl]-9H-pyrido[2,3-b]indol-3-yl]-1-methyl-N-methylsulfonyl-4-oxo-1,8-naphthyridine-3-carboxamide;
6-[5,6-Difluoro-8-[methyl(phosphonooxymethoxycarbonyl)amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
6-[4-(Dimethylamino)-5,6-difluoro-8-(methylamino)-1-(phosphonooxymethyl)pyrido[2,3-b]indol-3-yl]-1-methyl-4-oxo-1,8-naphthyridine-3-carboxylic acid; and
6-[5-Cyano-6-fluoro-8-[methyl-[1-[(2S)-2-amino-3-methyl-butanoyl]oxyethoxycarbonyl]amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
   or a pharmaceutically acceptable salt.

### Synthesis

The compounds of the present invention can be prepared by any conventional means. Suitable processes for synthesizing these compounds as well as their starting materials are provided in the schemes below and in the examples. All substituents, in particular, R¹ to R⁷ are as defined above unless otherwise indicated. Furthermore, and unless explicitly otherwise stated, all reactions, reaction conditions, abbreviations and symbols have the meanings well known to a person of ordinary skill in organic chemistry.

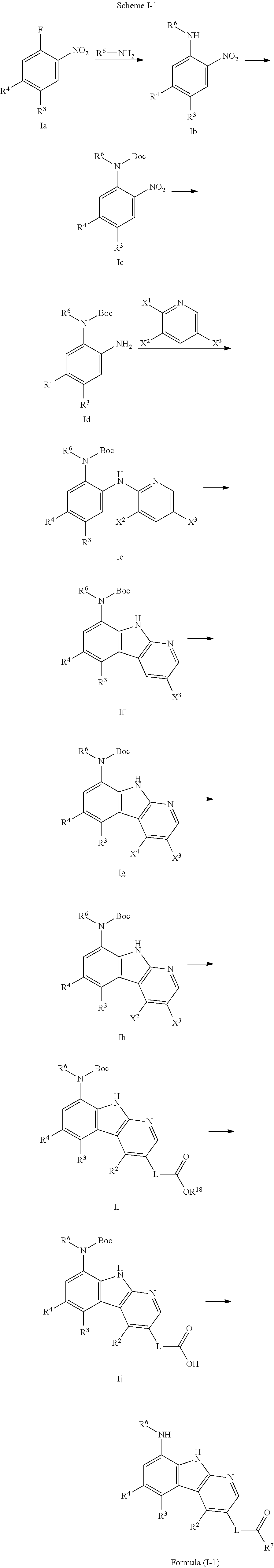

wherein X¹, X², X³ and X⁴ are independently halogen, boronic ester, or boronic acid; L is unsubstituted or substituted 4-oxo-1,8-naphthyridinyl, or 4-oxoquinolizinyl; R¹⁸ is C₁₋₆alkyl or Bn.

In one embodiment, the compound of formula (I-1) can be prepared according to Scheme 1-1. Nucleophilic substitution of ortho-fluoro nitrobenzene (la) with amine R⁶—NH₂ affords aniline (Ib). The aniline (Ib) can be protected with di-tert-butyl carbonate to give the protected aniline (Ic). The nitro group in aniline (Ic) can be reduced by a reducing agent, such as H₂ with palladium catalysts, to give the compound of formula (Id). Coupling of the compound of formula (Id) with tri-halogenated pyridine can be achieved using palladium catalysts and phosphine ligands to give the compound of formula (Ie). Cyclization of the compound of formula (Ie) using palladium catalysts and phosphine ligands gives the compound of formula (If). The compound of formula (Ig) can be obtained from the compound of formula (If) through oxidation of the pyridine followed by halogenation, such as treatment of POCl₃ or POBr₃. Alternatively, the compound of formula (Ig) can also be obtained by additional halogenation of the compound of formula (Ig) (when R³ is H) with halogenating reagent, such as NCS, followed by di-tert-butyl carbonate re-protection.

Coupling of the compound of formula (Ig) to introduce R² can be achieved through either nucleophilic substitution with an amine, in the presence of a base for certain C—N bond formation (with R₂ bearing a nucleophilic N), or a Buchwald-Hartwig cross coupling reaction for certain C—N bond formation (with R² bearing a basic N). Further coupling of the compound of formula (Ih) to introduce R¹ can be achieved using a palladium catalyzed Suzuki coupling to give the compound of formula (Ii). Chiral separation can be carried out for the compound of formula (Ih) or the compound of formula (Ii). Alternatively, the compound of formula (Ii) can also be obtained by converting the halide X³ of compound of formula (Ih) into a boronic ester or boronic acid, then coupling with the R¹ halide. Converting the ester of the compound of formula (Ii) into a carboxylic acid in the presence of a base, such as NaOH or LiOH, affords the compound of formula (Ij).

The compound of formula (I-1) can be prepared by the ester or the amide bond formation via coupling the compound of formula (Ij) carboxylic acid with an alcohol or amine, in the presence of a coupling reagent, such as HATU, followed by the deprotection of Boc group with an acid, such as TFA/DCM. Alternatively, the compound of formula (I) can also be prepared by the amide bond formation via first deprotection of Boc group, followed by the activation of the carboxylic acid into an acid chloride and reaction with amide, such as methanesulfonamide. Alternatively, the compound of formula (I-1) can also be prepared by the ester bond formation via reacting the carboxylic acid with an electrophile halide in the presence of a base such as K₂CO₃, in a solvent such as DMF.

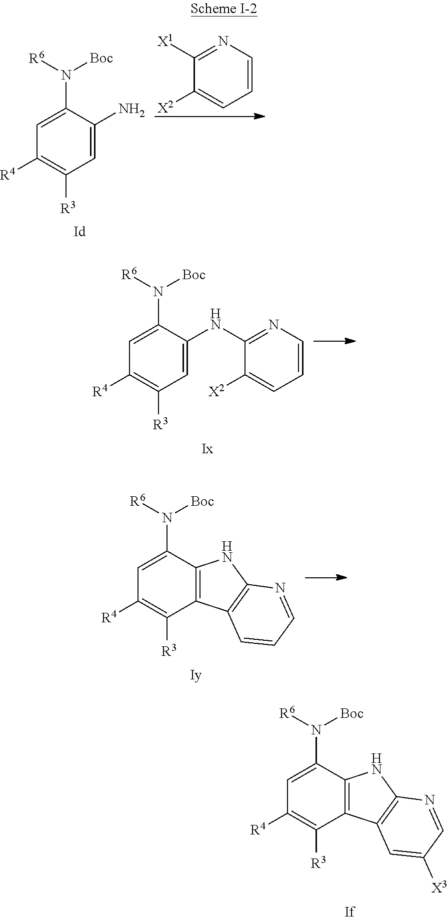

wherein X¹, X², X³ and X⁴ are independently halogen, boronic ester, or boronic acid.

In another embodiment, the compound of formula (If) can be prepared according to Scheme 1-2. Coupling of the compound of formula (Id) with di-halogenated pyridine can be achieved using palladium catalysts and phosphine ligands to give the compound of formula (Ix). Cyclization of the compound of formula (Ix) using palladium catalysts and phosphine ligands gives the compound of formula (Iy). The compound of formula (Iy) can be subject to halogenation using halogenating reagent, such as NCS, NBS, or NIS, to give the compound of formula (If).

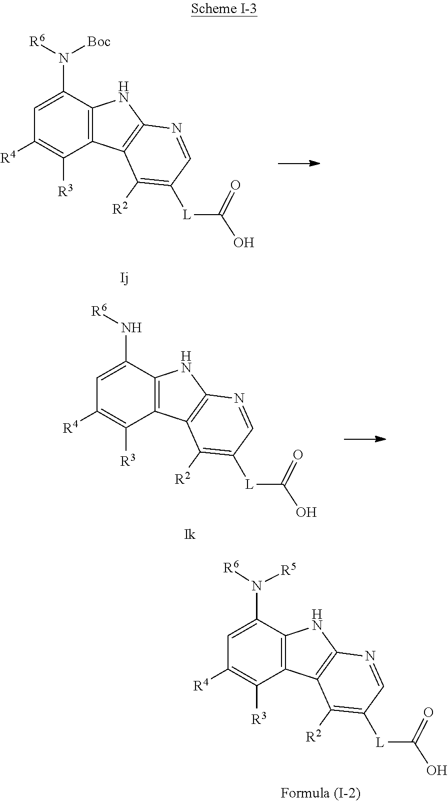

wherein L is unsubstituted or substituted 4-oxo-1,8-naphthyridinyl, or 4-oxoquinolizinyl.

In another embodiment, the compound of formula (I-2) can be prepared according to Scheme 1-3. The compound of formula (Ij) is deprotected to give the of compound of formula (Ik). The compound of formula (Ik) can then be derivatized into the compound of formula (I) by reaction with acylating or sulfonylating reagents, such as formic acid or sulfur trioxide.

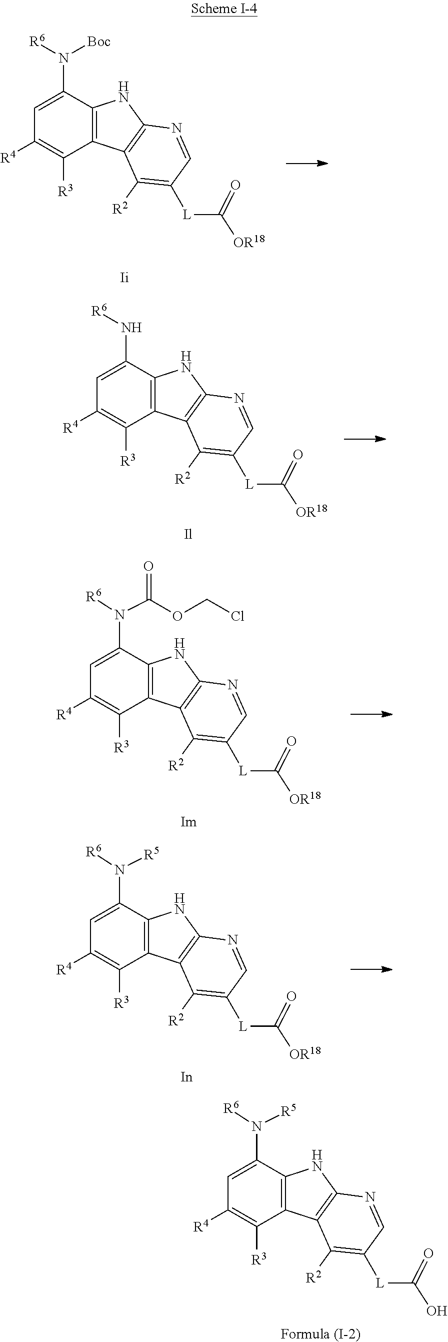

wherein L is unsubstituted or substituted 4-oxo-1,8-naphthyridinyl, or 4-oxoquinolizinyl; R¹⁸ is C₁₋₆alkyl or Bn.

In another embodiment, the compound of formula (I) can be prepared according to Scheme 1-4. Deprotection the compound of formula (Ii) affords the compound of formula (II). A carbamate linker formation by reaction of the compound of formula (II) with a chlorformate such as chloromethyl chloroformate gives the compound of formula (Im). SN2 nucleophilic displacement of halide of the compound of formula (Im) with agents such as potassium di-tert-butyl phosphate or sodium hydrogen fumarate gives the compound of formula (In). Compound of formula (In) can be converted to compound of formula (I-2) via hydrogenation (when R¹⁸ is Bn) in the presence of Pd/C or hydrolysis in the presence of a base, such as NaOH or LiOH (when R¹⁸ is C₁₋₆alkyl).

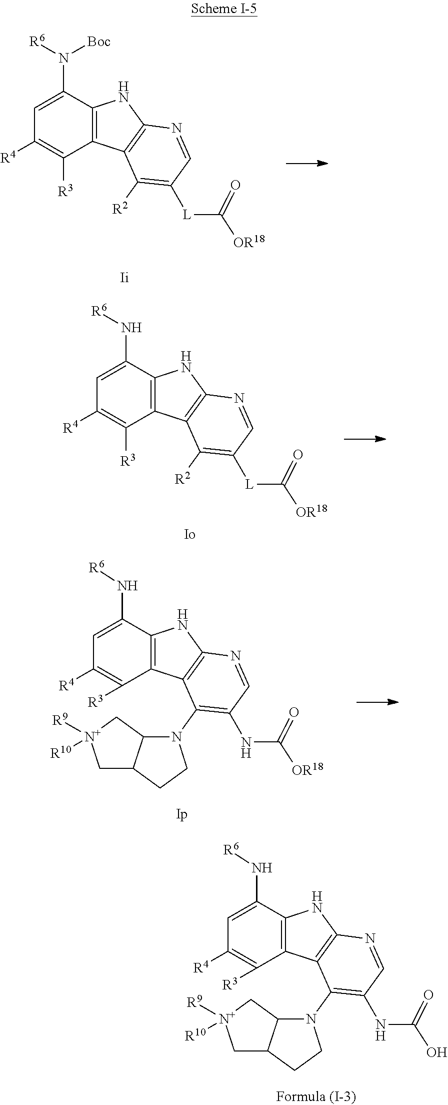

wherein R² is

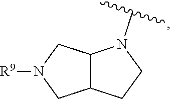

R¹⁸ is C₁₋₆alkyl or Bn.

In another embodiment, compound of formula (I-3) can be prepared according to Scheme 1-5. Deprotection of the compound of formula (Ii) affords the compound of formula (Io). Reaction of the compound of formula (Io) with halide such as di-tert-butyl chloromethyl phosphate or 4-(bromomethyl)-5-methyl-1,3-dioxol-2-one, in the presence of a base such as KOH, a phase transfer catalyst such as tetraethylammonium iodide gives the compound of formula (Ip) of a quaternary ammonium salt. The compound of formula (I) can then be obtained by converting the ester of compound of formula (Ip) into a carboxylic acid via hydrogenation (when R¹⁸ is Bn) in the presence of Pd/C or hydrolysis in the presence of a base, such as NaOH or LiOH (when R¹⁸ is C₁₋₆alkyl).

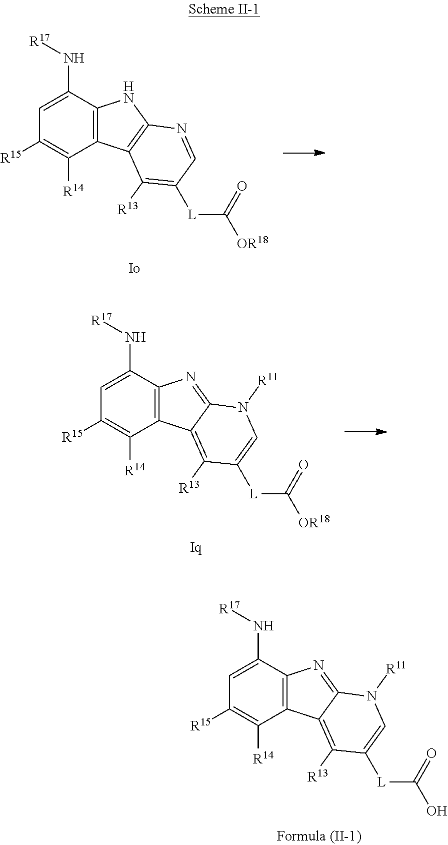

wherein L is unsubstituted or substituted 4-oxo-1,8-naphthyridinyl, or 4-oxoquinolizinyl; R¹⁸ is C₁₋₆alkyl or Bn.

In another embodiment, the compound of formula (II-1) can be prepared according to Scheme II-1. The compound of formula (Io) reacts with a halide such as di-tert-butyl chloromethyl phosphate, in the presence of a base such as Cs₂CO₃, in a solvent such as NMP to give the compound of formula (Iq). The compound of formula (II-1) can then be obtained by converting the ester of compound of formula (Iq) into a carboxylic acid via hydrogenation (when R¹⁸ is Bn) in the presence of Pd/C or hydrolysis in the presence of a base, such as NaOH or LiOH (when R¹⁸ is C₁₋₆alkyl).

This invention also relates to a process for the preparation of a compound of formula (I) or (II) comprising any of the following steps:

a) the coupling reaction of compound of formula (Ij),

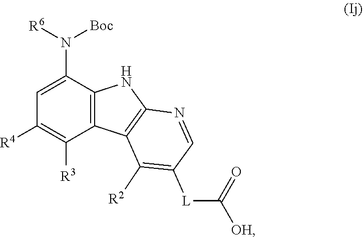

with an alcohol or amine in the presence of a coupling reagent followed by the deprotection of Boc group with an acid;

b) reaction of compound of formula (Ik),

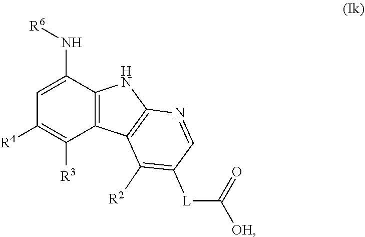

with acylating or sulfonylating reagents;

c) hydrolysis or hydrogentation of compound of formula (In),

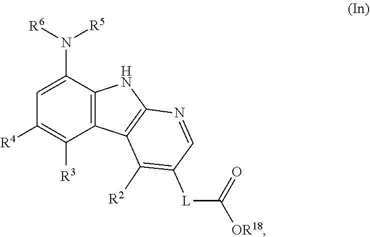

in the presence of a base or a catalyst;

d) hydrolysis or hydrogentation of compound of formula (Ip),

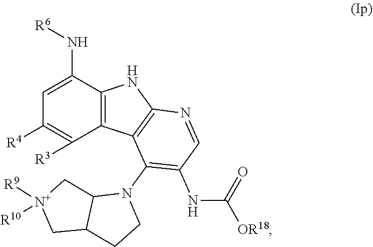

in the presence of a base or a catalyst;

e) hydrolysis or hydrogentation of compound of formula (Iq),

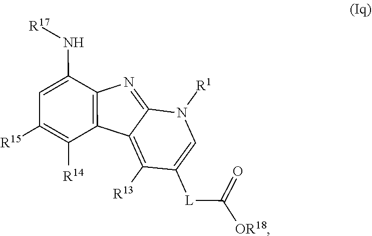

in the presence of a base or a catalyst;

wherein R¹ to R¹⁷ are defined above; L is unsubstituted or substituted 4-oxo-1,8-naphthyridinyl, or 4-oxoquinolizinyl; R¹⁸ is C₁₋₆alkyl or Bn; in step a), the coupling reagent can be, for example, HATU; the acid can be, for example, TFA;

in step b), the acylating reagent can be, for example, formic acid; the sulfonylating reagent can be, for example, sulfur trioxide;

in step c), d) and e), the base can be, for example, NaOH and LiOH; the catalyst can be, for example, Pd/C.

A compound of formula (I) or (II) when manufactured according to the above process is also an object of the invention.

### Pharmaceutical Compositions and Administration

Another embodiment provides pharmaceutical compositions or medicaments containing the compounds of the invention and a therapeutically inert carrier, diluent or excipient, as well as methods of using the compounds of the invention to prepare such compositions and medicaments. In one example, compounds of formula (I) or (II) may be formulated by mixing at ambient temperature at the appropriate pH, and at the desired 20 degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) or (II) is formulated in an acetate buffer, at pH 5. In another embodiment, the compounds of formula (I) or (II) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to reduced bacterial load or improve host survival through the inhibition of bacterial DNA gyrase and/or Topoisomerase IV. For example, such amount may be below the amount that is toxic to normal cells, or the mammal as a whole.

In one example, the pharmaceutically effective amount of the compound of the invention administered parenterally per dose will be in the range of about 0.1 to 1000 mg/kg, alternatively about 1 to 100 mg/kg of patient body weight per day. In another embodiment, oral unit dosage forms, such as tablets and capsules, preferably contain from about 5 to about 5000 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 10 to 500 mg of the compound of the invention compounded with about 40 to 400 mg anhydrous lactose, about 5 to 50 mg sodium croscarmellose, about 5 to 50 mg polyvinylpyrrolidone (PVP) K30, and about 1 to 10 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5 to 1000 mg) of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of formula (I) or (II), or a stereoisomer or pharmaceutically acceptable salt thereof. In a further embodiment includes a pharmaceutical composition comprising a compound of formula (I) or (II), or a stereoisomer or pharmaceutically acceptable salt thereof, together with a pharmaceutically acceptable carrier or excipient.

Another embodiment includes a pharmaceutical composition comprising a compound of formula (I) or (II) for use in the treatment and/or prophylaxis of bacterial infections.

In some embodiments, the compounds of this invention may be administered, as part of a single or multiple dosage regimen, orally, parenterally, by inhalation spray, topically, rectally, nasally, buccally, vaginally, or via an implanted reservoir. The term parenteral as used includes subcutaneous, intracutaneous, intravenous, intramuscular, intra-articular, intrasynovial, intrastemal, intrathecal, intralesional and intracranial injection or infusion techniques. Typically, the pharmaceutical compositions of the invention will be administered from about 1 to 5 times per day or alternatively, as a continuous infusion upon improvement of a patient's condition.

### Indications and Methods of Treatment

The compounds of the invention are useful for treatment and/or prophylaxis of bacterial infection in humans or other animals by administering to the subject in need of a therapeutically effective amount of compound of formula (I) or (II), or a pharmaceutically acceptable salt, or enantiomer or diastereomer thereof. The compounds and methods of the invention are particularly well suited for human patients infected by pathogens that include Staphylococcus aureus, Escherichia coli, Klebsiella pneumoniae, Acinetobacter baumannii and Pseudomonas aeruginosa. Examples of bacterial organisms that may also be controlled by the compounds of the invention include, but not limited to, the following Gram-Positive and Gram-Negative organisms: Streptococcus pneumoniae, Streptococcus pyogenes, Enterococcus faecalis, Enterococcus faecium, Enterobacter spp. species, Proteus spp. species, Serratia marcescens. Staphylococcus aureus, Coag. Neg. Staphylococci, Haemophilus influenzae, Bacillus anthraces, Mycoplasma pneumoniae, Moraxella catarrhalis, Chlamydophila pneumoniae, Chlamydia trachomatis, Legionella pneumophila, Mycobacterium tuberculosis, Helicobacter pylori, Staphylococcus saprophyticus, Staphylococcus epidermidis, Francisella tularensis, Yersinia pestis, Clostridium difficile, Bacteroides spp. species Neisseria gonorrhoeae, Neisseria meningitidis, Burkholderia pseudomallei, Burkholderia mallei, Borrelia burgdorferi, Mycobacterium avium complex, Mycobacterium abscessus, Mycobacterium kansasii, E. coli and Mycobacterium ulcer am.

Examples of bacterial infections may include, but not limited to, upper respiratory infections, lower respiratory infections, ear infections, pleuropulmonary and bronchial infections, complicated urinary tract infections, uncomplicated urinary tract infections, intra-abdominal infections, cardiovascular infections, a blood stream infection, sepsis, bacteremia, CNS infections, skin and soft tissue infections, GI infections, bone and joint infections, genital infections, eye infections, or granulomatous infections. Examples of specific bacterial infections include, but not limited to, uncomplicated skin and skin structure infections (uSSSI), complicated skin and skin structure infections (cSSSI), catheter infections, pharyngitis, sinusitis, otitis externa, otitis media, bronchitis, empyema, pneumonia, community-acquired bacterial pneumoniae (CABP), hospital-acquired pneumonia (HAP), hospital-acquired bacterial pneumonia, ventilator-associated pneumonia (VAP), diabetic foot infections, vancomycin resistant enterococci infections, cystitis and pyelonephritis, renal calculi, prostatitis, peritonitis, complicated intra-abdominal infections (cIAI) and other inter-abdominal infections, dialysis-associated peritonitis, visceral abscesses, endocarditis, myocarditis, pericarditis, transfusion-associated sepsis, meningitis, encephalitis, brain abscess, osteomyelitis, arthritis, genital ulcers, urethritis, vaginitis, cervicitis, gingivitis, conjunctivitis, keratitis, endophthalmitisa, an infection in cystic fibrosis patients or an infection of febrile neutropenic patients.

Furthermore, the invention relates to the use of a compound of formula (I) or (II) for the treatment and/or prophylaxis of bacterial infection. The invention relates to the use of a compound of formula (I) or (II) for the preparation of a medicament for the treatment and/or prophylaxis of bacterial infection. Another embodiment includes a method for the treatment or prophylaxis of bacterial infection which method comprises administering an effective amount of a compound of formula (I) or (II), or pharmaceutically acceptable salt thereof.

### EXAMPLES

The invention will be more fully understood by reference to the following examples. They should not, however, be construed as limiting the scope of the invention.

### Abbreviations

Abbreviations used herein are as follows:
ACN or MeCN acetonitrile
AcOK potassium acetate
[α]D₂₀ optical rotation at 20 degrees Celsius
B₂Pin₂ bis(pinacolato)diboron
BINAP 2,2′-bis(di phenylphosphino)-1,1′-binaphthalene
Boc₂O di-tert-butyl dicarbonate
CC₅₀ concentration results in the death of 50 percent of the cells (To remove?)
DBU 1,8-diazabicyclo[5.4.0]undec-7-ene
DIC N,N′-diisopropylcarbodumide
DIPA diisopropylamine
DIPEA N,N-diisopropylethylamine
DMA dimethylacetamide
DMAP 4-dimethylaminopyridine
EDCI 1-ethyl-3-(3-dimethylaminopropyl)carbodiimide
EtOAc ethyl acetate
Ex example
h hour
HATU: 1-[bis(dimethyl amino)methy 1 ene]-1H-1,2,3-triazolo[4,5-b]pyridinium 3-oxid hexafluorophosphate
HPLC: high performance liquid chromatography
HPLC-UV: high performance liquid chromatography with ultraviolet detector
IPA isopropyl alcohol
IV intravenous
LAH lithium aluminum hydride
LC-MS liquid chromatography-mass spectrometry
m-CPBA 3-chloroperoxybenzoic acid
NBS N-bromosuccinimide
NMP N-methyl-2-pyrrolidone
OD optical density
Pd-Ad₂nBuP biphenyl chloro[(di(l-adamantyl)-N-butylphosphine)-2-(2-aminobiphenyl)]palladium(II)
Pd₂(dba)₃ tris(dibenzylideneacetone)dipalladium(0)
PPA polyphosphoric Acid
prep-HPLC preparative high performance liquid chromatography
r.t. room temperature
Rt retention time
SFC supercritical fluid chromatography
TBME methyl tert-butyl ether
TEA tri ethyl amine
TFA trifluoroacetic acid
TsOH p-toluenesulfonic acid
TLC Thin Layer Chromatography
UV ultraviolet detector
x-phos 2-dicyclohexylphosphino-2′,4′,6′-triisopropylbiphenyl
wt weight

### GENERAL EXPERIMENTAL CONDITIONS

Intermediates and final compounds were purified by flash chromatography using one of the following instruments: i) Biotage SP1 system and the Quad 12/25 Cartridge module, ii) ISCO combi-flash chromatography instrument. Silica gel brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using X Bridge™ Perp C₁₈ (5 μm, OBD™ 30×100 mm) column or SunFire™ Perp C₁₈ (5 μm, OBD™ 30×100 mm) column.

Chiral Separation was conducted on Thar 350 preparative SFC using ChiralPak AD-10μ (200×50 mm I.D.) with mobile phase A for CO₂ and B for ethanol. LC/MS spectra were obtained using a Waters UPLC-SQD Mass. Standard LC/MS conditions were as follows (running time: 3 minutes):

Acidic condition: A: 0.1% formic acid (or 0.1% TFA) and 1% acetonitrile in H₂O; B: 0.1% formic acid (or 0.1% TFA) in acetonitrile;

Basic condition: A: 0.05% NH₃.H₂O in H₂O; B: acetonitrile;

Neutral condition: A: 0.1% NH₄HCO₃ in H₂O; B: acetonitrile.

Mass spectra (MS): generally only ions which indicate the parent mass are reported, and unless otherwise stated the mass ion quoted is the positive mass ion (M+H)⁺.

NMR Spectra were obtained using Bruker Avance 400 MHz.

All reactions involving air-sensitive reagents were performed under an nitrogen/or argon atmosphere. Reagents were used as received from commercial suppliers without further purification unless otherwise noted.

### PREPARATIVE EXAMPLES

### Intermediate A1

### tert-Butyl N-(3,4-dichloro-5,6-difluoro-9H-pyrido[2,3-b]indol-8-yl)-N-methyl-carbamate

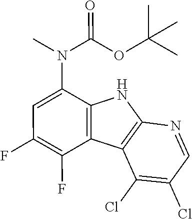 [SMILES: CN(C(=O)OC(C)(C)C)c1cc(F)c(F)c2c1[nH]c1ncc(Cl)c(Cl)c12]

The title compound was synthesized according to the following scheme:

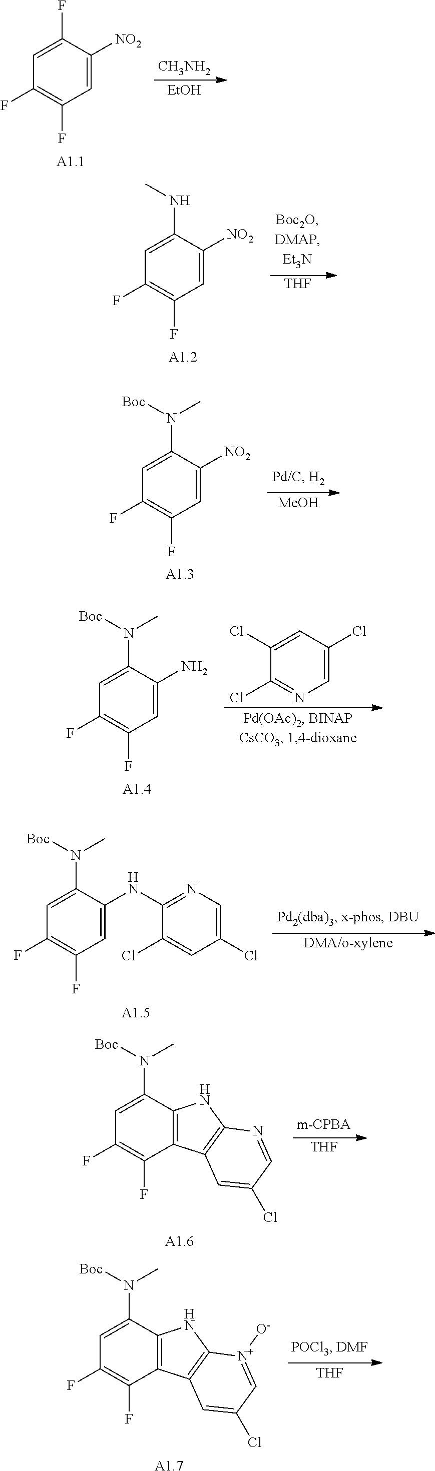

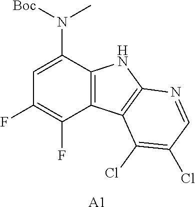

### Step (a) Preparation of 4,5-difluoro-N-methyl-2-nitro-aniline (Compound A1.2)

To a solution of 1,2,4-trifluoro-5-nitro-benzene (1.0 kg, 5.65 mol) in EtOH (2.0 L) was added methylamine solution (1.06 kg, 11.29 mmol, 33% in EtOH) dropwise at 0° C. over 3 h. After completion of the addition, the reaction mixture was stirred at 0° C. for additional 1 h before poured into ice-water (15.0 L). The resulting precipitates were collected by filtration, and re-suspended in petrolieum ether/EtOAc (10.0 L, v/v=4:1). The by-products were removed by filtration and the filtrate was dried over anhy. Na₂SO₄, filtered, and concentrated in vacuo to give a crude product. It was then purified by silica gel flash chromatography (petrolieum ether:EtOAc=100:1˜20:1) to give 4,5-difluoro-N-methyl-2-nitro-aniline_(250.0 g, 23.5% yield) as a yellow solid. MS (ESI): 189.1 ([M+H]⁺).

### Step (b) Preparation of tert-butyl N-(4,5-difluoro-2-nitro-phenyl)-N-methyl-carbamate (Compound A1.3)

To a mixture solution of 4,5-difluoro-N-methyl-2-nitro-aniline (250.0 g, 1.33 mol), Boc₂O (580.0 g, 2.66 mol), and Et₃N (403.0 g, 3.99 mol) in THF (1.0 L) was added DMAP (32.5 g, 0.266 mmol) and the resulting reaction mixture was stirred at 60° C. for 3 h. After TLC (petrolieum ether:EtOAc=10:1) showed the reaction was completed, MeOH (100.0 mL) was added. The mixture solution was concentrated in vacuo to give a crude product, which was purified by silica gel flash chromatography (petrolieum ether:EtOAc=100:1˜10:1) to give tert-butyl N-(4,5-difluoro-2-nitro-phenyl)-N-methyl-carbamate (305.0 g, 79.6% yield) as a white solid. MS (ESI): 189.1 ([M−Boc+H]⁺), 233.1 ([M−^{t}Bu+H]⁺), 311.1 ([M+Na]⁺).

### Step (c) Preparation of tert-butyl N-(2-amino-4,5-difluoro-phenyl)-N-methyl-carbamate (Compound A1.4)

To a solution of tert-butyl N-(4,5-difluoro-2-nitro-phenyl)-N-methyl-carbamate (104.0 g, 360.8 mmol) in MeOH (1.2 L) was added Pd/C (10.0 g, 50% H₂O) under N₂. After the suspension was degassed under vacuum and purged with H₂ several times, the reaction mixture was stirred at 30° C. for 18 h under H₂ (50 psi). The reaction mixture was filtered through Celite and the filterate was concentrated in vacuo to give a crude product of tert-butyl N-(2-amino-4,5-difluoro-phenyl)-N-methyl-carbamate (92.0 g, 98.7% yield) as a white solid. MS (ESI): 159.1 ([M−Boc+H]⁺), 203.1 ([M−^{t}Bu+H]⁺), 281.1 ([M+Na]⁺). It was used directly in the next step without further purification.

### Step (d) Preparation of tert-butyl N-[2-[(3,5-dichloro-2-pyridyl)amino]-4,5-difluoro-phenyl]-N-methyl-carbamate (Compound A1.5)

To a solution of tert-butyl N-(2-amino-4,5-difluoro-phenyl)-N-methyl-carbamate (90.0 g, 348.5 mmol) and 2,3,5-trichloropyridine (64.2 g, 352.0 mmol) in 1,4-dioxane (1.5 L) was added Cs₂CO₃ (227.0 g, 697.0 mmol). The mixture was degassed with nitrogen for 2 min before Pd(OAc)₂ (7.82 g, 34.85 mmol) and BINAP (32.55 g, 52.28 mmol, CAS: 98327-87-8) were added under nitrogen. The resulting suspension was heated to 110° C. and stirred for 3 h. After TLC (petrolieum ether:EtOAc=10:1) showed the reaction was completed, the mixture was cooled back to room temperature and diluted with EtOAc (1 L) and filtered. The filtrate was concentrated in vacuo to give a crude product, which was purified by silica gel flash chromatography (petrolieum ether:EtOAc=500:1˜20:1) to give tert-butyl N-[2-[(3,5-dichloro-2-pyridyl)amino]-4,5-difluoro-phenyl]-N-methyl-carbamate (124.0 g, 88.0% yield) as a white solid. MS (ESI): 404.0 ([{³⁵Cl}M+H]⁺), 406.0 ([{³⁷Cl}M+H]⁺), 348.0 ([{³⁵Cl}[M−^{t}Bu+H]⁺), 350.0 ([{³⁷Cl}M4Bu+H]⁺).

### Step (e) Preparation of tert-butyl N-(3-chloro-5,6-difluoro-9H-pyrido[2,3-b]indol-8-yl)-N-methyl-carbamate (Compound A1.6)

A mixture of tert-butyl N-[2-[(3,5-dichloro-2-pyridyl)amino]-4,5-difluoro-phenyl]-N-methyl-carbamate (123.0 g, 304.3 mmol), Pd₂(dba)₃ (55.73 g, 60.86 mmol, CAS: 51364-51-3), x-phos (58.03 g, 121.72 mmol, CAS: 564483-18-7) and DBU (92.7 g, 608.6 mmol, CAS: 6674-22-2) were dissolved in o-xylene/DMA (v/v=1:1, 220.0 mL). After being degassed with nitrogen for 5 min, the reaction mixture was heated at 130° C. for 1 h. After TLC (petrolieum ether:EtOAc=5:1) showed the reaction was completed, the reaction mixture was cooled back to room temperature and diluted with EtOAc (800 mL). The mixture was filtered and the filtrate was poured into water (500 mL) and extracted with EtOAc (1.2 L) three times. Combined organics were washed with aq. CaCl₂ solution (1N, 400 mL) three times, brine (400 mL) twice, dried over anhy. Na₂SO₄ and concentrated in vacuo to give a crude product, which was recrystallized in MeOH (800 mL) to give tert-butyl N-(3-chloro-5,6-difluoro-9H-pyrido[2,3-b]indol-8-yl)-N-methyl-carbamate (68.0 g, 60.8% yield) as a white solid. MS (ESI): 368.1 ([{³⁵Cl}M+H]⁺), 370.1 ([{³⁷Cl}M+H]⁺).

### Step (f) Preparation of tert-butyl N-(3-chloro-5,6-difluoro-1-oxido-9H-pyrido[2,3-b]indol-lium-8-yl)-N-methyl-carbamate (Compound A1.7)

To a solution of tert-butyl N-(3-chloro-5,6-difluoro-9H-pyrido[2,3-b]indol-8-yl)-N-methyl-carbamate (32.0 g, 87.0 mmol) in THF (400.0 mL) was added m-CPBA (26.5 g, 130.5 mmol), and the resulting reaction mixture was stirred at 50° C. for 2 h. After TLC (DCM:EtOH=40:1) showed the reaction was completed, the mixture was cooled back to room temperature, poured into aq. Na₂SO₃ solution (3.0 L, 10%) and stirred at room temperature for 1 h. The resulting precipitate was then collected by filtration, washed with aq. Na₂SO₃ solution (1.0 L, 5%), and dried under vacuum to give tert-butyl N-(3-chloro-5,6-difluoro-1-oxido-9H-pyrido[2,3-b]indol-lium-8-yl)-N-methyl-carbamate (32.0 g, 95.8% yield) as a yellow solid. MS (ESI): 384.1 ([{³⁵Cl}M+H]⁺), 386.0 ([{³⁷Cl}M+H]⁺).

### Step (g) Preparation of tert-Butyl N-(3,4-dichloro-5,6-difluoro-9H-pyrido[2,3-b]indol-8-yl)-N-methyl-carbamate (Intermediate A1)

To a stirred solution of tert-butyl N-(3-chloro-5,6-difluoro-1-oxido-9H-pyrido[2,3-b]indol-lium-8-yl)-N-methyl-carbamate (60.0 g, 0.156 mol) in DMF/THF (2.0 L, v/v=1:1) was added POCl₃ (267.42 g, 1.744 mol) dropwise under ice-salt bath. The resulting mixture was stirred at −5° C. 0° C. for 3 h before poured into satd. aq. solution of Na₂CO₃ (3.0 L) at 0° C. and extracted with EtOAc (1.0 L) four times. Combined organics was then washed with satd. aq. Na₂CO₃ solution (500 mL) twice, aq. CaCl₂ solution (1N, 500 mL) four times and brine (300 mL) twice. The separated organic layer was dried over anhy. Na₂SO₄ and concentrated in vacuo to give a crude product, which was recrystallized in MeOH (600 mL) to give tert-Butyl N-(3,4-dichloro-5,6-difluoro-9H-pyrido[2,3-b]indol-8-yl)-N-methyl-carbamate (57.3 g, 91.3% yield) as a white solid. ¹H NMR (400 MHz, DMSO-d6) δ: 12.624 (br. s., 1H), 8.652 (s, 1H), 7.572˜7.629 (dd, J=11.6, 6.8 Hz, 1H), 3.247 (s, 3H), 1.343 (s, 9H). MS (ESI): 402.1 ([{³⁵Cl}M+H]⁺), 404.1 ([{³⁷Cl}M+H]⁺).

### Intermediate A2

### tert-Butyl N-(3-bromo-4-chloro-5,6-difluoro-9H-pyrido[2,3-b]indol-8-yl)-N-methyl-carbamate

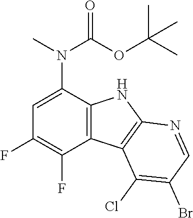 [SMILES: CN(C(=O)OC(C)(C)C)c1cc(F)c(F)c2c1[nH]c1ncc(Br)c(Cl)c12]

The title compound was synthesized according to the following scheme:

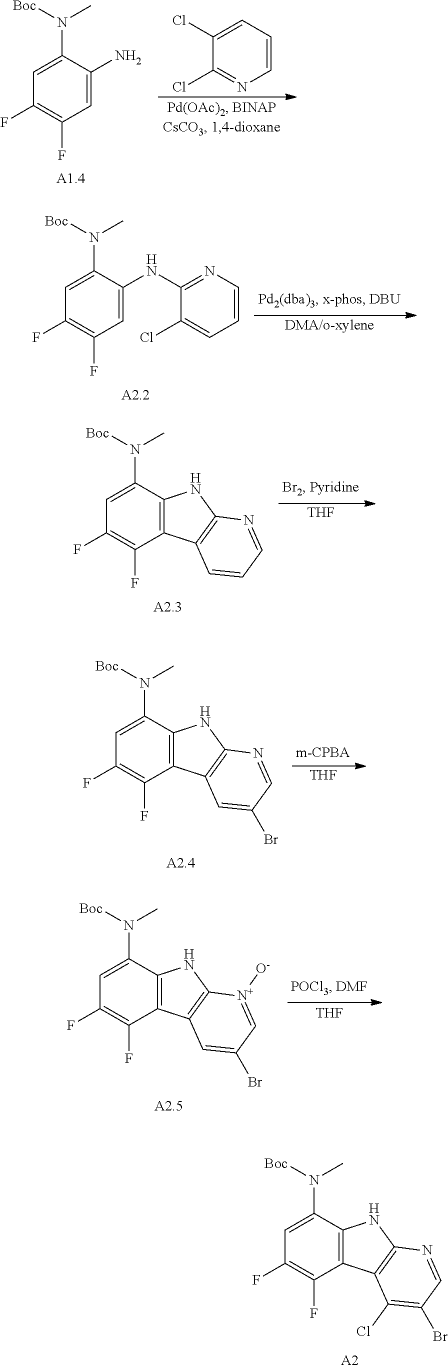

### Step (a) Preparation of tert-butyl N-[2-[(3-chloro-2-pyridyl)amino]-4,5-difluoro-phenyl]-N-methyl-carbamate (Compound A2.2)

To a stirred solution of tert-butyl N-(2-amino-4,5-difluoro-phenyl)-N-methyl-carbamate (142.0 g, 0.55 mol, compound A1.4) and 2,3-dichloropyridine (81.4 g, 0.55 mol) in dioxane (1.5 L) was added cesium carbonate (358.6 g, 1.10 mol). The mixture was degassed with nitrogen for 2 min before palladium(II) acetate (12.3 g, 0.055 mol) and BINAP (68.5 g, 0.110 mmol, CAS: 98327-87-8) were added under nitrogen. The resulting suspension was then heated to 110° C. and stirred for 3 h. The reaction mixture was then cooled back to r.t., diluted with EtOAc (1.0 L) and filtered. The filtrate was concentrated in vacuo to give a crude product, which was purified by silica gel flash chromatography (petrolieum ether:EtOAc=500:1˜20:1) to give tert-butyl N-[2-[(3-chloro-2-pyridyl)amino]-4,5-difluoro-phenyl]-N-methyl-carbamate (175.0 g, 86.1% yield) as a white solid. MS (ESI): 370.1 ([{³⁵Cl}M+H]⁺), 372.1 ([{³⁷Cl}M+H]⁺).

### Step (b) Preparation of tert-butyl N-(5,6-difluoro-9H-pyrido[2,3-b]indol-8-yl)-N-methyl-carbamate (Compound A2.3)

tert-Butyl N-[2-[(3-chloro-2-pyridyl)amino]-4,5-difluoro-phenyl]-N-methyl-carbamate (175.0 g, 473.24 mmol), Pd₂(dba)₃ (65.0 g, 71.0 mmol, CAS: 51364-51-3), x-phos (67.7 g, 142.0 mmol, CAS: 564483-18-7), and DBU (144 g, 946.5 mmol, CAS: 6674-22-2) were dissolved in a mixture solution of o-xylene/DMA (v/v=1:1, 260.0 mL). After the mixture solution was degassed with nitrogen for 5 min, it was heated at 130° C. for 3 h before cooled back to r.t. and diluted with EtOAc (500 mL). The mixture was filtered to remove part of catalyst and ligand, and the filtrate was poured into water (1.0 L) and extracted with EtOAc (2.0 L) three times. Combined organics was then washed with aq. Ca₂Cl₂ solution (1N, 500 mL) four times, brine (500 mL) twice, dried over anhy. Na₂SO₄, filtered, and concentrated in vacuo to give a crude product. It was then recrystallized in MeOH (800 mL) to give tert-butyl N-(5,6-difluoro-9H-pyrido[2,3-b]indol-8-yl)-N-methyl-carbamate (110 g, 69.7% yield) as a white solid. The mother liquid was purified by silica gel flash chromatography (petrolieum ether:EtOAc=50:1˜1:1) to give additional tert-butyl N-(5,6-difluoro-9H-pyrido[2,3-b]indol-8-yl)-N-methyl-carbamate (35.0 g, 22.2% yield) as a white solid. In total, 145 g of compound A2.3 (91.9% yield) was obtained. MS (ESI): 334.1 ([M+H]⁺).

### Step (c) Preparation of tert-butyl N-(3-bromo-5,6-difluoro-9H-pyrido[2,3-b]indol-8-yl)-N-methyl-carbamate (Compound A2.4)

To a stirred solution of tert-butyl N-(5,6-difluoro-9H-pyrido[2,3-b]indol-8-yl)-N-methyl-carbamate (140.0 g, 0.420 mol) and pyridine (166.1 g, 2.1 mol) in THF (2.0 L) was added bromine (335.6 g, 2.1 mol) at −60° C. After the addition, the mixture was warmed up and stirred at 15° C. for 2 h. After LC-MS showed the reaction was completed, the mixture was added dropwise into aq. sodium sulfite solution (5.0 L, 10%) and adjusted to pH=8.0 with aq. sodium carbonate solution at 0° C. and stirred at 25° C. for additional 1 h. The mixture was then extracted by EtOAc (2.0 L) three times. Combined organics was then washed with aq. sodium carbonate solution (1.0 L) twice and brine (1.0 L) three times. Separated organic layer was dried over anhy. Na₂SO₄, filtered, and concentrated in vacuo to give a crude product, which was recrystallized in MeOH (1.0 L) to give tert-butyl N-(3-bromo-5,6-difluoro-9H-pyrido[2,3-b]indol-8-yl)-N-methyl-carbamate (150.0 g, 86.6% yield) as a yellow solid. MS (ESI): 412.1 ([{⁷⁹Br}M+H]⁺), 414.1 ([{⁸¹Br}M+H]⁺).

### Step (d) Preparation of tert-butyl N-(3-bromo-5,6-difluoro-1-oxido-9H-pyrido[2,3-b]indol-1-ium-8-yl)-N-methyl-carbamate (Compound A2.5)

To a stirred solution of tert-butyl N-(3-bromo-5,6-difluoro-9H-pyrido[2,3-b]indol-8-yl)-N-methyl-carbamate (150.0 g, 363.9 mmol) in THF (1.2 L) was added 3-chlorobenzoperoxoic acid (148.0 g, 727.8 mmol, 85% wt). The mixture was degassed with nitrogen for 2 min before stirred at 50° C. for 2 h. After TLC (petrolieum ether:EtOAc=2:1) showed the starting material was consumed completely, the reaction was cooled back to r.t. and the mixture was poured into aq. sodium sulfite solution (6.0 L, 10%) and stirred for 1 h. The resulting precipitate was collected by filtration and washed by aq. sodium sulfite solution (1.0 L, 5%). The filter cake was evaporated to dryness under the reduced pressure to give tert-butyl N-(3-bromo-5,6-difluoro-1-oxido-9H-pyrido[2,3-b]indol-1-ium-8-yl)-N-methyl-carbamate (150.0 g, 96.3% yield) as a yellow solid. MS (ESI): 428.1 ([{⁷⁹Br}M+H]⁺), 430.1 ([{⁸¹Br}M+H]⁺).

### Step (e) Preparation of tert-Butyl N-(3-bromo-4-chloro-5,6-difluoro-9H-pyrido[2,3-b]indol-8-yl)-N-methyl-carbamate (Intermediate A2)

To a stirred solution of tert-butyl N-(3-bromo-5,6-difluoro-1-oxido-9H-pyrido[2,3-b]indol-1-ium-8-yl)-N-methyl-carbamate (150 g, 0.35 mol) in DMF/THF (2.5 L, v/v=1:1) was added phosphorus(V) oxychloride (421.3 g, 2.75 mol) dropwise under ice-salt bath. After the addition, the mixture was stirred at −5° C. 0° C. for 4 h until TLC (petrolieum ether:EtOAc=2:1) showed the starting material was consumed completely. The mixture was then poured into satd. aq. solution of sodium carbonate (5.0 L) at 0° C. and extracted with EtOAc (3.0 L) twice. Combined organics was then washed with satd. aq. solution of Na₂CO₃ (1.0 L) twice, aq. CaCl₂ solution (1N, 1.5 L) four times, and brine (1.0 L) twice. The organic layer was dried over anhy. Na₂SO₄, filtered, and concentrated in vacuo to give a crude product, which was recrystallized in MeOH (1.2 L) to give tert-Butyl N-(3-bromo-4-chloro-5,6-difluoro-9H-pyrido[2,3-b]indol-8-yl)-N-methyl-carbamate (142.0 g, 90.8% yield) as a white solid. ¹H NMR (400 MHz, DMSO-d6) δ: 12.85 (br. s., 1H), 8.75 (s, 1H), 7.69˜7.74 (dd, 0.7=11.6, 6.8 Hz, 1H), 3.22 (s, 3H), 1.22˜1.50 (m, 9H). MS (ESI): 446.0 ([{⁷⁹Br}M+H]⁺), 448.0 ([{⁸¹Br}M+H]⁺).

### Intermediate A3

### tert-Butyl N-(3,4-dichloro-5-cyano-6-fluoro-9H-pyrido[2,3-b]indol-8-yl)-N-methyl-carbamate

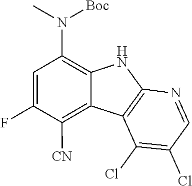

The titled compound was synthesized according to the following scheme:

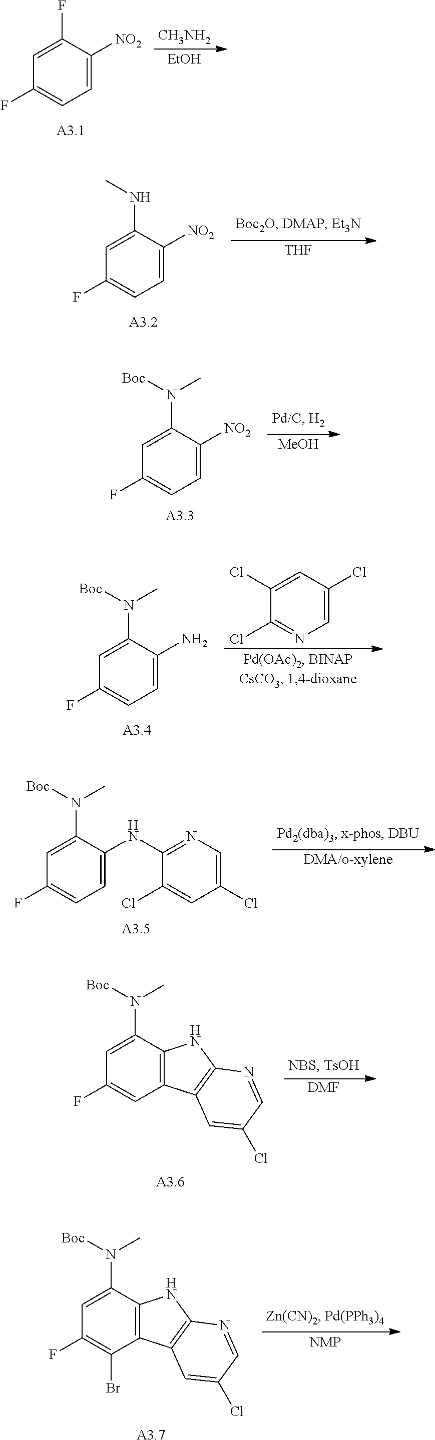

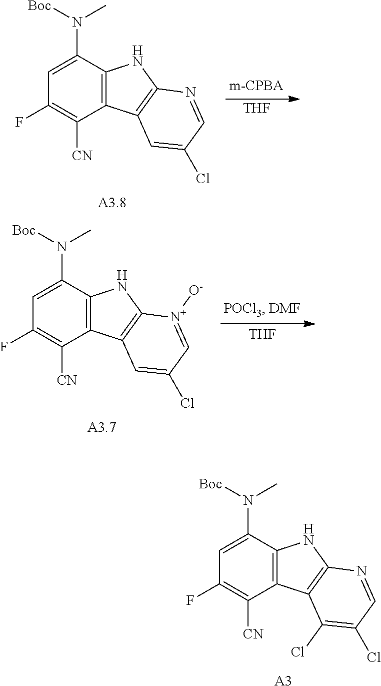

### Step (a) Preparation of 5-fluoro-N-methyl-2-nitro-aniline (Compound A3.2)

To a stirred solution of 2,4-difluoro-1-nitro-benzene (130.0 g, 0.817 mol) in EtOH (500.0 mL) was added methyl amine (230.7 g, 2.45 mmol, 33% in EtOH) dropwise under ice-water bath. After completion of the addition, the reaction mixture was stirred at 0° C. for 1.5 h and then poured into ice-water (4.0 L). The resulting crystal precipitate was collected by filtration and dried under vacuum to give 5-fluoro-N-methyl-2-nitro-aniline (132.0 g, 94.9% yield) as a yellow solid. MS (ESI): 171.1 (M+H]⁺).

### Step (b) Preparation of tert-butyl N-(5-fluoro-2-nitro-phenyl)-N-methyl-carbamate (Compound A3.3)

To a mixture solution of 5-fluoro-N-methyl-2-nitro-aniline (132.0 g, 0.776 mol), Boc₂O (338.0 g, 1.551 mol) and DIPEA (200.5 g, 1.551 mol) in THF (1.0 L) was added DMAP (19.0 g, 0.155 mmol) and the resulting reaction mixture was stirred at 70° C. for 12 h. After TLC (petrolieum ether:EtOAc=20:1) showed the starting material was consumed, the reaction mixture was concentrated in vacuo to give a crude product, which was purified by silica gel flash chromatography (petrolieum ether:EtOAc=100:1˜10:1) to give tert-butyl N-(5-fluoro-2-nitro-phenyl)-N-methyl-carbamate (190.0 g, 90.6% yield) as a white solid. MS (ESI): 171.1 ([M−Boc+H]⁺).

### Step (c) Preparation of tert-butyl N-(2-amino-5-fluoro-phenyl)-N-methyl-carbamate (Compound A3.4)

To a solution of tert-butyl N-(5-fluoro-2-nitro-phenyl)-N-methyl-carbamate (190.0 g, 0.703 mol) in MeOH (2 L) was added Pd/C (18.0 g, 50% H₂O) under N₂. After the suspension was degassed under vacuum and purged with H₂ several times, the reaction mixture was stirred at 40° C. for 5 h under H₂ atmosphere (50 psi). After TLC (petrolieum ether:EtOAc=5:1) showed the reaction was completed, the reaction mixture was cooled back to r.t. and filtered through Celite to remove catalyst. The filtrate was then concentrated under in vacuo to give a crude product of tert-butyl N-(2-amino-5-fluoro-phenyl)-N-methyl-carbamate (162.0 g, 95.9% yield) as a white solid. MS (ESI): 141.2 ([M−Boc+H]⁺), 185.1 ([M−^{t}Bu+H]⁺). It was used directly in the next step without further purification.

### Step (d) Preparation of tert-butyl N-[2-[(3,5-dichloro-2-pyridyl)amino]-5-fluoro-phenyl]-N-methyl-carbamate (Compound A3.5)

To a solution of tert-butyl N-(2-amino-5-fluoro-phenyl)-N-methyl-carbamate (142.0 g, 0.591 mol) and 2,3,5-trichloropyridine (108.0 g, 0.591 mol) in dioxane (2.0 L) was added Cs₂CO₃ (385.0 g, 1.18 mol). The mixture was degassed with nitrogen for 2 min before Pd(OAc)₂ (13.3 g, 0.059 mol) and BINAP (73.5 g, 0.118 mol) were added under nitrogen. After the charging was completed, the suspension was heated to 110° C. and stirred for 3 h. After TLC (petrolieum ether:EtOAc=20:1) showed the reaction was completed, the reaction mixture was cooled back to r.t., diluted with EtOAc (3.0 L) and filtered. The filtrate was concentrated in vacuo to give a crude product, which was purified by silica gel flash chromatography (petrolieum ether:EtOAc=500:1˜20:1) to give tert-butyl N-[2-[(3,5-dichloro-2-pyridyl)amino]-5-fluoro-phenyl]-N-methyl-carbamate (190.0 g, 83.2% yield) as a white solid. MS (ESI): 385.9 ([M{³⁵Cl}+H]⁺), 329.9 ([M{³⁵Cl}−^{t}Bu+H]⁺), 331.9 ([M{³⁷Cl}−^{t}Bu+H]⁺).

### Step (e) Preparation of tert-butyl N-(3-chloro-6-fluoro-9H-pyrido[2,3-b]indol-8-yl)-N-methyl-carbamate (Compound A3.6)

A mixture of tert-butyl N-[2-[(3,5-dichloro-2-pyridyl)amino]-5-fluoro-phenyl]-N-methyl-carbamate (184.0 g, 476.4 mmol), Pd₂(dba)₃ (87.3 g, 95.3 mmol), x-phos (90.8 g, 190.6 mmol) and DBU (145.0 g, 953.0 mmol) were dissolved in a mixture of o-xylene/DMA (v/v=1:1, 300.0 mL). After the mixture was degassed with nitrogen for 5 min, it was heated at 130° C. for 2 h. The mixture was then cooled back to r.t., diluted with EtOAc (2.0 L), and filtered. The filtrate was poured into water (1.0 L) and extracted with EtOAc (1.0 L) three times. Combined organics was washed with aq. CaCl₂ solution (1N, 500 mL) three times, and brine (500 mL) twice. The separated organic layer was dried over anhy. Na₂SO₄, filtered, and concentrated in vacuo to give a crude product, which was recrystallized in MeOH (1.5 L) to give tot-butyl N-(3-chloro-6-fluoro-9H-pyrido[2,3-b]indol-8-yl)-N-methyl-carbamate (93.0, 55.8% yield) as a yellow solid. MS (ESI): 350.2 ([M{³⁵Cl}+H]⁺).

### Step (0 Preparation of tert-butyl N-(5-bromo-3-chloro-6-fluoro-9H-pyrido[2,3-b]indol-8-yl)-N-methyl-carbamate (Compound A3.7)

A suspension of tert-butyl N-(3-chloro-6-fluoro-9H-pyrido[2,3-b]indol-8-yl)-N-methyl-carbamate (88.0 g, 251.6 mmol) in DMF (700.0 mL) was heated to 100° C. for full dissolvation. Afterwards, the mixture was cooled back to r.t. and added NBS (53.8 g, 302.0 mmol) and TsOH.H₂O (7.2 g, 37.7 mmol). The resulting mixture solution was then re-heated to 45° C. and stirred for 2 h. After LC-MS indicated the starting material was completely consumed, the mixture was cooled back to r.t., poured into aq. Na₂SO₃ solution (4.0 L, 10%) and stirred at r.t. for another 0.5 h. The resulting crystal precipitate was then collected by filtration and dried under vacuum to give a crude product of tot-butyl N-(5-bromo-3-chloro-6-fluoro-9H-pyrido[2,3-b]indol-8-yl)-N-methyl-carbamate (96.0 g, 89.0% yield) as a yellow solid. MS (ESI): 430.1 ([M{⁷⁹Br}+H]⁺). It was used directly in the next step without further purification.

### Step (g) Preparation of tot-butyl N-(3-chloro-5-cyano-6-fluoro-9H-pyrido[2,3-b]indol-8-yl)-N-methyl-carbamate (Compound A3.8)

To a stirred solution of tot-butyl N-(5-bromo-3-chloro-6-fluoro-9H-pyrido[2,3-b]indol-8-yl)-N-methyl-carbamate (80.0 g, 186.6 mmol) and Zn(CN)₂ (131.0 g, 1.12 mol) in NMP (300.0 mL) was added Pd(PPh₃)₄ (21.6 g, 18.7 mmol) in glovebox under argon. The resulting reaction mixture was stirred at 120° C. for 3 h under argon. After LC-MS showed the starting material was completely consumed, the reaction mixture was cooled back to r.t., diluted with EtOAc (2.5 L) and filtered. The filtrate was washed with aq. CaCl₂ solution (400 mL) four times, and brine (400 mL) three times. The organic layer was then dried over anhy. Na₂SO₄, filtered, and concentrated in vacuo to give a crude product, which was recrystallized in MeOH (250 mL) to give tert-butyl N-(3-chloro-5-cyano-6-fluoro-9H-pyrido[2,3-b]indol-8-yl)-N-methyl-carbamate (74.0 g, 86.3% yield) as a yellow solid. MS (ESI): 375.2 ([M{³⁵Cl}+H]⁺), 377.2 ([M{³⁷Cl}+H]⁺).

### Step (h) Preparation of tert-butyl N-(3-chloro-5-cyano-6-fluoro-1-oxido-9H-pyrido[2,3-b]indol-1-ium-8-yl)-N-methyl-carbamate (Compound A3.9)

To a solution of tert-butyl N-(3-chloro-5-cyano-6-fluoro-9H-pyrido[2,3-b]indol-8-yl)-N-methyl-carbamate (27.0 g, 72.04 mmol) in THF (150.0 mL) was added m-CPBA (29.3 g, 114.08 mmol, 85% wt). The reaction mixture was degassed with nitrogen for 2 min and stirred at 45° C. for 2 h. After TLC (petrolieum ether:EtOAc=2:1) showed the starting material was consumed completely, the mixture was cooled back to r.t., poured into 10% aq. Na₂SO₃ solution (2.0 L, 10%) and stirred for 1 h. The resulting precipitate was collected by filtration and washed by aq. Na₂SO₃ solution (100 mL). The filter cake was evaporated to dryness under the reduced pressure to give tert-butyl N-(3-chloro-5-cyano-6-fluoro-1-oxido-9H-pyrido[2,3-b]indol-1-ium-8-yl)-N-methyl-carbamate (27.0 g, 95.9% yield) as a yellow solid. MS (ESI): 391.2 ([M{³⁵Cl}+H]⁺), 335.1 ([M{³⁵Cl}-^{t}Bu+H]⁺).

### Step (i) Preparation of tert-butyl N-(3,4-dichloro-5-cyano-6-fluoro-9H-pyrido[2,3-b]indol-8-yl)-N-methyl-carbamate (Intermediate A3)

To a solution of tert-butyl N-(3-chloro-5-cyano-6-fluoro-1-oxido-9H-pyrido[2,3-b]indol-1-ium-8-yl)-N-methyl-carbamate (27.0 g, 69.09 mmol) in DMF/THF (500.0 mL, v/v=1:1) was added POCl₃ (15.9 g, 103.6 mmol) dropwise under ice-salt bath. The resulting reaction mixture was stirred at −40° C.-30° C. for 5 h. After TLC (petrolieum ether:EtOAc=2:1) showed the starting material was consumed completely, the mixture was poured into satd. aq. NaHCO₃ solution (1.0 L) at 0° C. and subsequently extracted by EtOAc (1.0 L) twice. Combined organics was washed with aq. NaHCO₃ solution (300.0 mL) twice, aq. CaCl₂ solution (300.0 mL) four times, and brine (300.0 mL) twice. The organic layer was then dried over Na₂SO₄, filtered, and concentrated in vacuo to give a crude product, which was recrystallized in THF/MeOH (150.0 mL, v/v=1:10) to give tert-butyl N-(3,4-dichloro-5-cyano-6-fluoro-9H-pyrido[2,3-b]indol-8-yl)-N-methyl-carbamate (18.0 g, 63.7% yield) as a yellow solid. MS (ESI): 409.2 ([{³⁵Cl}M+H]⁺), 411.2 ([{³⁷Cl}M+H]⁺). ¹H NMR (400 MHz, DMSO-d₆) δ ppm 13.15 (s, 1H), 8.75 (s, 1H), 7.85 (d, 0.7=10.8 Hz, 1H), 3.28 (s, 3H), 1.25 (br s, 9H).

### Intermediate B1

### Ethyl 1-methyl-4-oxo-6-(4,4,5,5-tetramethyl-1,3,2-dioxaborolan-2-yl)-1,8-naphthyridine-3-carboxylate

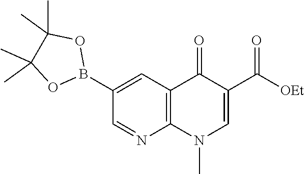 [SMILES: CCOC(=O)c1cn(C)c2ncc(B3OC(C)(C)C(C)(C)O3)cc2c1=O]

The titled compound was synthesized according to the following scheme:

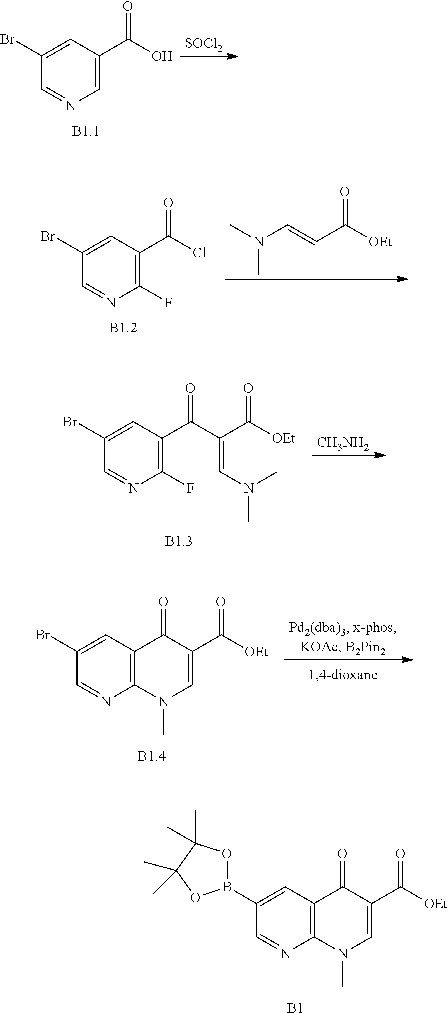

### Step (a) Preparation of 5-bromo-2-fluoro-pyridine-3-carbonyl chloride (Compound B1.2)

A solution of 5-bromo-2-fluoro-pyridine-3-carboxylic acid (40.0 g, 181.82 mmol) in SOCl₂ (200.0 mL) was stirred 80° C. for 2 h. Afterwards, the mixture solution was cooled back to r.t. and concentrated under the reduced pressure to give a crude product of 5-bromo-2-fluoro-pyridine-3-carbonyl chloride (42.0 g, 96.9% yield). It was used directly in the next step without further purification.

### Step (b) Preparation of ethyl (Z)-2-(5-bromo-2-fluoro-pyridine-3-carbonyl)-3-(dimethylamino)prop-2-enoate (Compound B13)

To a solution of 5-bromo-2-fluoro-pyridine-3-carbonyl chloride (42.0 g, 176.14 mmol) and Et₃N (35.6 g, 352.3 mmol) in THF (150.0 mL) was added ethyl (E)-3-(dimethylamino)prop-2-enoate (30.3 g, 211.37 mmol), and the resulting reaction mixture was stirred at 60° C. for 2 h. After LC-MS showed the starting material was fully consumed, the mixture was cooled back to r.t., diluted with EtOAc (400 mL) and filtered. The filtrate was concentrated in vacuo to give a crude product of ethyl (Z)-2-(5-bromo-2-fluoro-pyridine-3-carbonyl)-3-(dimethylamino)prop-2-enoate (58.0 g, 95.4% yield). MS (ESI): 345.0 ([{⁷⁹Br}M+H]⁺⁾, 347.1 ([{⁸¹Br}M+H]⁺). It was used directly in the next step without further purification.

### Step (c) Preparation of ethyl 6-bromo-1-methyl-4-oxo-1,8-naphthyridine-3-carboxylate (Compound B1.4)

To a solution of ethyl (Z)-2-(5-bromo-2-fluoro-pyridine-3-carbonyl)-3-(dimethylamino)prop-2-enoate (58.0 g, 0.168 mol) in THF (170 mL) was added methyl amine (800.0 mL, 1.6 mol, 2M in THF), and the resulting reaction mixture was stirred at 60° C. for 0.5 h. After cooling down to r.t., the mixture solution was evaporated to dryness under the reduced pressure. The residue was re-dissolved in EtOAc (300 mL) and washed with brine (300 mL). The organic layer was dried over anhy. Na₂SO₄, filtered, and concentrated in vacuo to give a crude product, which was recrystallized in MeOH (300 mL) to give ethyl 6-bromo-1-methyl-4-oxo-1,8-naphthyridine-3-carboxylate (49.5 g, 94.7% yield) as a white solid. MS (ESI): 311.0 ([{⁷⁹Br}M+H]⁺), 313.0 ([{⁸¹Br}M+H]⁺).

### Step (d) Preparation of ethyl 1-methyl-4-oxo-6-(4,4,5,5-tetramethyl-1,3,2-dioxaborolan-2-yl)-1,8-naphthyridine-3-carboxylate (Intermediate B1)

A mixture of ethyl 6-bromo-1-methyl-4-oxo-1,8-naphthyridine-3-carboxylate (24.0 g, 77.14 mmol), B₂Pin₂ (39.2 g, 154.28 mmol), Pd₂(dba)₃ (7.0 g, 7.71 mmol), x-phos (7.34 g, 15.4 mmol), and KOAc (22.7 g, 231.4 mmol) were dissolved in dioxane (250.0 mL). The solution was degassed with argon for 5 min before stirred at 100° C. for 2 h. The reaction mixture was cooled back to room temperature and precipitate was removed by filtration. The filtrate was concentrated under the reduced pressure to give a crude product, which was then recrystallized in petrolieum ether/MTBE (500 mL) to give ethyl 1-methyl-4-oxo-6-(4,4,5,5-tetramethyl-1,3,2-dioxaborolan-2-yl)-1,8-naphthyridine-3-carboxylate (27.0 g, yield: 97.7% yield) as a yellow solid. MS (ESI): 277.0 ([M-82+H]⁺). ¹H NMR (400 MHz, DMSO-d6) δ: 8.890-8.894 (d, J=1.6 Hz, 1H), 8.820 (s, 1H), 8.691˜8.694 (d, 0.7=1.2 Hz, 1H), 4.211˜4.265 (q, J=1.2 Hz, 2H), 3.922 (s, 3H), 1.232˜1.311 (m, 15H).

### Intermediate B2

### Ethyl 1-[tert-butoxycarbonyl(methyl)amino]-4-oxo-6-(4,4,5,5-tetramethyl-1,3,2-dioxaborolan-2-yl)-1,8-naphthyridine-3-carboxylate

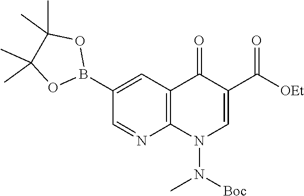 [SMILES: CCOC(=O)c1cn(N(C)C(=O)OC(C)(C)C)c2ncc(B3OC(C)(C)C(C)(C)O3)cc2c1=O]

The titled compound was synthesized according to the following scheme:

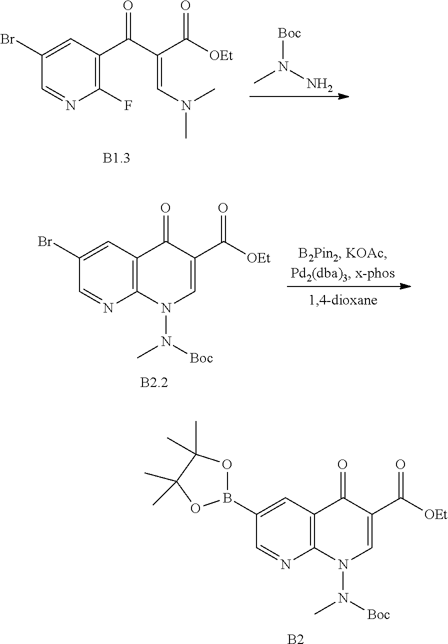

### Step (a) Preparation of ethyl 6-bromo-1-[tert-butoxycarbonyl(methyl)amino]-4-oxo-1,8-naphthyridine-3-carboxylate (Compound B2.2)

To a mixture of ethyl (Z)-2-(5-bromo-2-fluoro-pyridine-3-carbonyl)-3-(dimethylamino)prop-2-enoate (42.3 g, 123.0 mmol, compound B1.3) in THF (400.0 mL) was added tert-butyl N-amino-N-methyl-carbamate (32.3 g, 221.3 mmol), and the resulting reaction mixture was stirred at 60° C. for 2 h. After being cooled back to r.t., the mixture solution was evaporated to dryness under the reduced pressure to give a crude product, which was recrystallized in MeOH (300 mL) to give ethyl 6-bromo-1-[tert-butoxycarbonyl(methyl)amino]-4-oxo-1,8-naphthyridine-3-carboxylate (50.9 g, 97.3% yield) as a white solid. MS (ESI): 428.0 ([{⁸¹Br}M+H]⁺), 426.0 ([{⁷⁹Br}M+H]⁺). ¹H NMR (400 MHz, CDCl₃) δ ppm: 8.77˜8.76 (m, 1H), 8.67˜8.62 (m, 1H), 8.57˜8.56 (m, 1H), 4.36˜4.29 (m, 2H), 3.38˜3.36 (m, 3H), 1.48 (s, 3H), 1.37˜1.31 (m, 3H), 1.17 (s, 6H).

### Step (b) Preparation of ethyl 1-[tert-butoxycarbonyl(methyl)amino]-4-oxo-6-(4,4,5,5-tetramethyl-1,3,2-dioxaborolan-2-yl)-1,8-naphthyridine-3-carboxylate (Intermediate B2)

A mixture solution of ethyl 6-bromo-1-[tert-butoxycarbonyl(methyl)amino]-4-oxo-1,8-naphthyridine-3-carboxylate (35.0 g, 82.3 mmol2), B₂Pin₂ (41.9 g, 164.7 mmol), KOAc (20.3 g, 246.9 mmol), x-phos (3.9 g, 8.2 mmol) and Pd₂(dba)₃ (7.5 g, 8.2 mmol) in dioxane (400.0 mL) was stirred at 100° C. for 2 h under N₂. After being cooled back to r.t., the mixture was diluted with DCM (500 mL) and filtered. The liberate was concentrated in vacuo to give a crude product, which was recrystallized in petrolieum ether (400 mL) to give ethyl 1-[tert-butoxycarbonyl(methyl)amino]-4-oxo-6-(4,4,5,5-tetramethyl-1,3,2-dioxaborolan-2-yl)-1,8-naphthyridine-3-carboxylate (34.2 g, 87.7% yield) as yellow solid. MS (ESI): 392.1 ([M-82+H]⁺). ¹H NMR (400 MHz, DMSO-d₆) δ ppm: 8.99˜8.91 (m, 1H), 8.79 (m, 1H), 8.72-8.66 (m, 1H), 4.32˜4.26 (m, 2H), 3.40 (S, 3H), 1.49˜1.12 (m, 24H).

### Intermediate B3

### Benzyl 1-methyl-4-oxo-6-(4,4,5,5-tetramethyl-1,3,2-dioxaborolan-2-yl)-1,8-naphthyridine-3-carboxylate

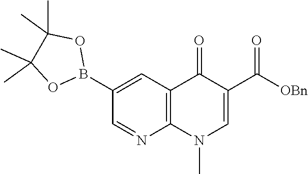 [SMILES: Cn1cc(C(=O)OCc2ccccc2)c(=O)c2cc(B3OC(C)(C)C(C)(C)O3)cnc21]

The titled compound was synthesized according to the following scheme:

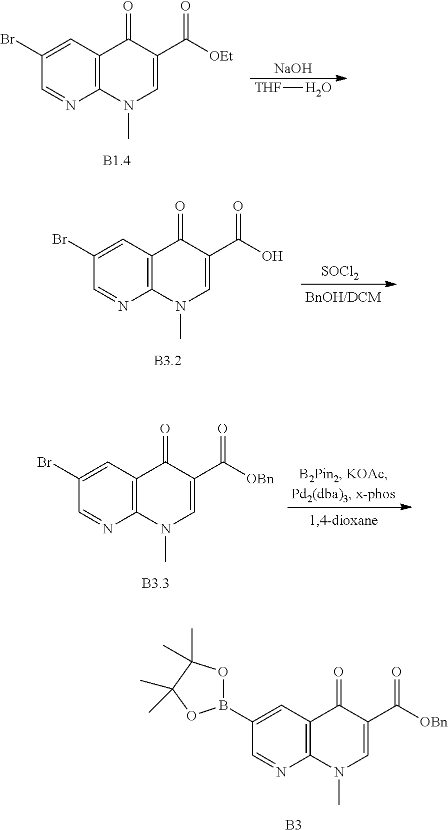

### Step (a) Preparation of 6-bromo-1-methyl-4-oxo-1,8-naphthyridine-3-carboxylic acid (Compound B3.2)

To a suspension of ethyl 6-bromo-1-methyl-4-oxo-1,8-naphthyridine-3-carboxylate (5.0 g, 16.07 mmol, compound B1.4) in THF (100 mL) and water (30 mL) was added NaOH (6.43 g, 160.7 mmol). The reaction mixture was stirred at 15° C. for 1 h until LC-MS showed the starting material was consumed completely. The mixture was acidified to pH 3˜4 by aq. HCl solution (2M) and the precipitate formed was collected by filtration. The filter cake was washed with water (50 mL) and dried under vacuum to give 6-bromo-10˜1-methyl-4-oxo-1,8-naphthyridine-3-carboxylic acid (4.4 g, 96.7% yield) as an off-white solid. MS (ESI): 283.0 ([{⁷⁹Br}M+H]⁺), 285.0 ([{⁸¹Br}+H]⁺). It was used directly in the next step without further purification.

### Step (b) Preparation of benzyl 6-bromo-1-methyl-4-oxo-1,8-naphthyridine-3-carboxylate (Compound B3.3)

A suspension of 6-bromo-1-methyl-4-oxo-1,8-naphthyridine-3-carboxylic acid (4.3 g, 15.19 mmol) in SOCl₂ (30.0 mL) was stirred at 80° C. for 0.5 h under N₂. Afterwards, the mixture was cooled back to r.t. and concentrated in vacuo. The residue was re-dissolved in DCM (15 mL) under N₂, followed by the addition of benzyl alcohol (4.93 g, 45.57 mmol), Et₃N (6.35 mL, 45.57 mmol) at 0° C. The mixture solution was allowed to stirred at 15° C. for 1 h until LC-MS showed the intermediate was consumed completely. The mixture was then washed with aq. HCl solution (50 mL, 0.5 M), and the separated organic layer was concentrated in vacuo to give a crude product, which was purified by silica gel flash chromatography (EtOAc, 100%) to give benzyl 6-bromo-1-methyl-4-oxo-1,8-naphthyridine-3-carboxylate (5.5 g, 95.1% yield) as an off-white solid. MS (ESI): 373.1 ([{⁷⁹Br}M+H]⁺), 375.1 ([{⁸¹Br}+H]⁺).

### Step (c) Preparation of benzyl 1-methyl-4-oxo-6-(4,4,5,5-tetramethyl-1,3,2-dioxaborolan-2-yl)-1,8-naphthyridine-3-carboxylate (Intermediate B3)

A mixture of benzyl 6-bromo-1-methyl-4-oxo-1,8-naphthyridine-3-carboxylate (5.0 g, 13.4 mmol3), B₂Pin₂ (10.21 g, 40.19 mmol), tris(dibenzylideneacetone)dipalladium (0) (368.05 mg, 0.400 mmol), x-phos (383.22 mg, 0.800 mmol), and AcOK (3.94 g, 40.19 mmol) were dissolved in 1,4-dioxane (100 mL) under Ar. The resulting reaction mixture was stirred at 100° C. for 2 h until LC-MS showed the starting material was consumed completely. The mixture was cooled back to r.t, diluted with DCM (100 mL) and filtered. The filtrate was concentrated in vacuo, and the residue was treated with TBME (960 mL). The precipitate was collected by filtration and the filter cake was dried under vacuum to give benzyl 1-methyl-4-oxo-6-(4,4,5,5-tetramethyl-1,3,2-dioxaborolan-2-yl)-1,8-naphthyridine-3-carboxylate (5.6 g, 96.5% yield) as a yellow solid. MS (ESI): 339.2 ([M-82+H]⁺). ¹H NMR (400 MHz, CHCl₃-d) δ ppm 9.17 (d, J=1.71 Hz, 1H), 9.02 (d, J=1.59 Hz, 1H), 8.62 (s, 1H), 7.52 (d, J=7.34 Hz, 2H), 7.36-7.42 (m, 2H), 7.33 (br d, J=7.21 Hz, 1H), 5.40 (s, 2H), 3.96 (s, 3H), 1.37 (s, 12H).

### Intermediate B4

### Benzyl 4-oxo-7-(4,4,5,5-tetramethyl-1,3,2-dioxaborolan-2-yl)quinolizine-3-carboxylate

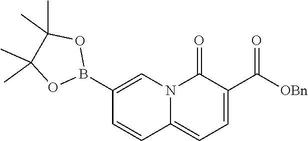 [SMILES: CC1(C)OB(c2ccc3ccc(C(=O)OCc4ccccc4)c(=O)n3c2)OC1(C)C]

The titled compound was synthesized according to the following scheme:

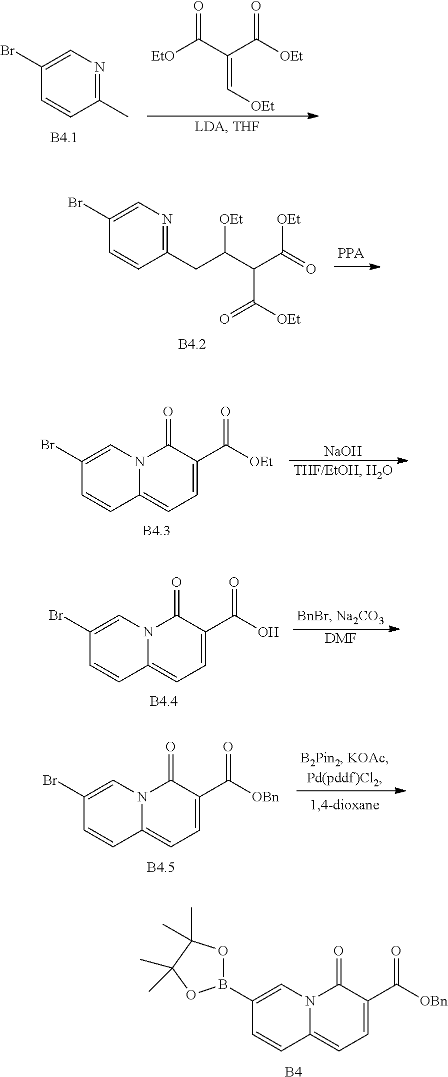

### Step (a) Preparation of diethyl 2-[2-(5-bromo-2-pyridyl)-1-ethoxy-ethyl]propanedioate (Compound B4.2)

To a solution of DIPA (52.90 g, 523.19 mmol) in THF (600 mL) was added n-BuLi (0.39 mL, 0.970 mmol) dropwise at −78° C. After the mixture solution was stirred at −30° C. for 1 h, 5-bromo-2-methylpyridine (90.0 g, 523.19 mmol) in THF (600 mL) was added dropwise at −78° C. and stirred for another 1 h. Afterwards, diethyl ethoxymethylenemalonate (113.13 g, 523.19 mmol) was added and the resulting reaction mixture was stirred at −78° C. for 2 h until LC-MS showed the starting material was consumed completely. Aqueous NH₄Cl solution was introduced to quench the reaction and the solution was extracted with EtOAc (500 mL) three times. Combined organics was then washed with brine, dried over anhy. Na₂SO₄, filtered, and concentrated in vacuo to give a crude product, which was purified by silica gel flash chromatography (petrolieum ether:EtOAc=100:1 to 5:1) to give diethyl 2-[2-(5-bromo-2-pyridyl)-1-ethoxy-ethyl]propanedioate (20 g, 9.9% yield) as yellow oil. MS (ESI): 388.0 ([{⁷⁹Br}M+H]⁺), 390.1 ([{⁸¹Br}M+H]⁺).

### Step (b) Preparation of ethyl 7-bromo-4-oxo-quinolizine-3-carboxylate (Compound B4.3)

A solution of diethyl 2-[2-(5-bromo-2-pyridyl)-1-ethoxy-ethyl]propanedioate_(42.0 g, 108.18 mmol) in PPA (40.0 mL, 0.200 mmol) was stirred at 130° C. for 2 h. After LC-MS showed the starting material was consumed, the reaction mixture was cooled back to r.t. and basified with aq. Na₂CO₃ solution to pH 9 and extracted with DCM (30 mL) three times. Combined organics was washed with brine, dried over anhy. Na₂SO₄, filtered and concentrated in vacuo to give a crude product of ethyl 7-bromo-4-oxo-quinolizine-3-carboxylate (4.5 g, 14.1% yield) as a yellow solid. MS (ESI): 296.0 ([{⁷⁹Br}M+H]⁺), 298.0 ([{⁸¹Br}M+H]⁺). It was used directly in the next step without further purification.

### Step (c) Preparation of 7-bromo-4-oxo-quinolizine-3-carboxylic acid (Compound B4.3)

To a solution of ethyl 7-bromo-4-oxo-quinolizine-3-carboxylate (8.5 g, 28.7 mmol) in EtOH (66.94 mL) and water (13.39 mL) was added NaOH (0.89 mL, 1.78 mmol), and the resulting reaction mixture was stirred at 15° C. for 2 h. After LC-MS showed the starting material was consumed, the mixture solution was concentrated under the reduced pressure and acidified with aq. HCl solution (1N) to pH 6 and extracted with DCM (30 mL) three times. Combined organics was then washed with brine, dried over anhy. Na₂SO₄, filtered and concentrated in vacuo to give a crude product of 7-bromo-4-oxo-quinolizine-3-carboxylic acid (8.0 g, 100% yield) as a yellow solid. MS (ESI): 290.0 ([{⁷⁹Br}M+H]⁺), 291.9 ([{⁸¹Br}M+Na]⁺). It was used directly in the next step without further purification.

### Step (d) Preparation of benzyl 7-bromo-4-oxo-quinolizine-3-carboxylate (Compound B4.4)

To a solution of 7-bromo-4-oxo-quinolizine-3-carboxylic acid (8.0 g, 29.84 mmol) in DMF (80 mL) was added BnBr (14.92 mL, 29.84 mmol) and Na₂CO₃ (1.0 mL, 29.84 mmol), and the resulting reaction mixture was stirred at 30° C. for 18 h. After LC-MS showed the starting material was consumed, the reaction mixture was concentrated in vacuo to give a crude product, which was purified by prep-HPLC (TFA as additive) to give benzyl 7-bromo-4-oxo-quinolizine-3-carboxylate (10 g, 93.6% yield) as a yellow solid. MS (ESI): 358.0 ([{⁷⁹Br}M+H]⁺), 360.0 ([{⁸¹Br}M+H]⁺).

### Step (e) Preparation of benzyl 4-oxo-7-(4,4,5,5-tetramethyl-1,3,2-dioxaborolan-2-yl)quinolizine-3-carboxylate (Intermediate B4)

To a solution of benzyl 7-bromo-4-oxo-quinolizine-3-carboxylate (1.0 g, 2.79 mmol) in THF (10 mL) was added Pd(dppf)Cl₂ (166.64 mg, 0.280 mmol, 0.100 eq), KOAc (547.2 mg, 5.58 mmol, 2 eq) and B₂Pin₂ (2.161 g, 8.38 mmol). After the mixture solution was degassed and purged with argon several times, the reaction mixture was stirred at 90° C. for 16 h. After LC-MS showed the starting material was consumed completely, the reaction mixture was cooled back to r.t and concentrated in vacuo to give a crude product, which was purified by prep-HPLC (TFA as additive) to give benzyl 4-oxo-7-(4,4,5,5-tetramethyl-1,3,2-dioxaborolan-2-yl)quinolizine-3-carboxylate (1.0 g, 88.4% yield) as a yellow solid. MS (ESI): 324.0 ([M+H]⁺).

### Intermediate C1

### cis-5-Methyl-2,3,3a,4,6,6a-hexahydro-1H-pyrrolo[2,3-c]pyrrole

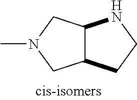

The titled compound was synthesized according to the following scheme:

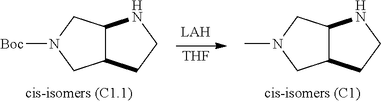

### Preparation of cis-5-Methyl-2,3,3a,4,6,6a-hexahydro-1H-pyrrolo[2,3-c]pyrrole (Intermediate C1)

To a stirred solution of LAH (4.02 g, 105.99 mmol) in THF (50 mL) was added cis-tot-butyl 2,3,3a,4,6,6a-hexahydro-1H-pyrrolo[2,3-c]pyrrole-5-carboxylate (4.5 g, 21.2 mmol, CAS: 132414-81-4) at 0° C. After the completion of addition, the resulting reaction mixture was allowed to warm up and stirred at 70° C. for 2 h under N₂. After TLC (DCM:MeOH=10:1) showed the starting material was consumed completely, the reaction mixture was cooled back to 0° C. and quenched by the addition of H₂O (5 mL), and followed by the addition of aq. NaOH solution (5 mL, 15%), H₂O (15 mL), and MgSO₄ (10 g). After the mixture solution was stirred at 25° C. for 1 h, it was filtered and the filtrate was concentrated in vacuo to give a crude product of cis-5-methyloctahydropyrrolo[2,3-c]pyrrole (2.6 g, 20.6 mmol, 97.2% yield) as yellow oil. It was used directly in the next step without further purification. ¹H NMR (400 MHz, CHCl₃-d) δ ppm 3.66-3.79 (m, 1H), 2.89-3.02 (m, 1H), 2.75 (dt, J=11.19, 6.50 Hz, 1H), 2.65 (dtd, J=12.40, 8.35, 8.35, 4.39 Hz, 1H), 2.46-2.54 (m, 2H), 2.38-2.45 (m, 1H), 2.34 (dd, J=9.28, 4.14 Hz, 1H), 2.27 (s, 3H), 1.85 (ddt, J=12.42, 8.78, 6.33, 6.33 Hz, 1H), 1.45-1.56 (m, 1H), 1.43 (s, 1H), 1.21-1.31 (m, 1H).

### Example 1.01

### 6-[5-Cyano-6-fluoro-8-[methyl(phosphonooxymethoxycarbonyl)amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid

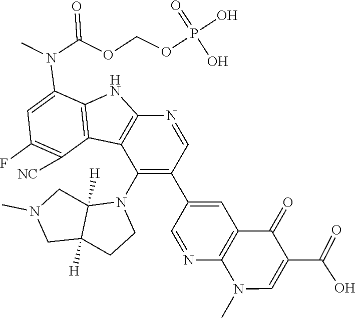 [SMILES: CN1C[C@@H]2CCN(c3c(-c4cnc5c(c4)c(=O)c(C(=O)O)cn5C)cnc4[nH]c5c(N(C)C(=O)OCOP(=O)(O)O)cc(F)c(C#N)c5c34)[C@@H]2C1]

The titled compound was synthesized according to the following scheme:

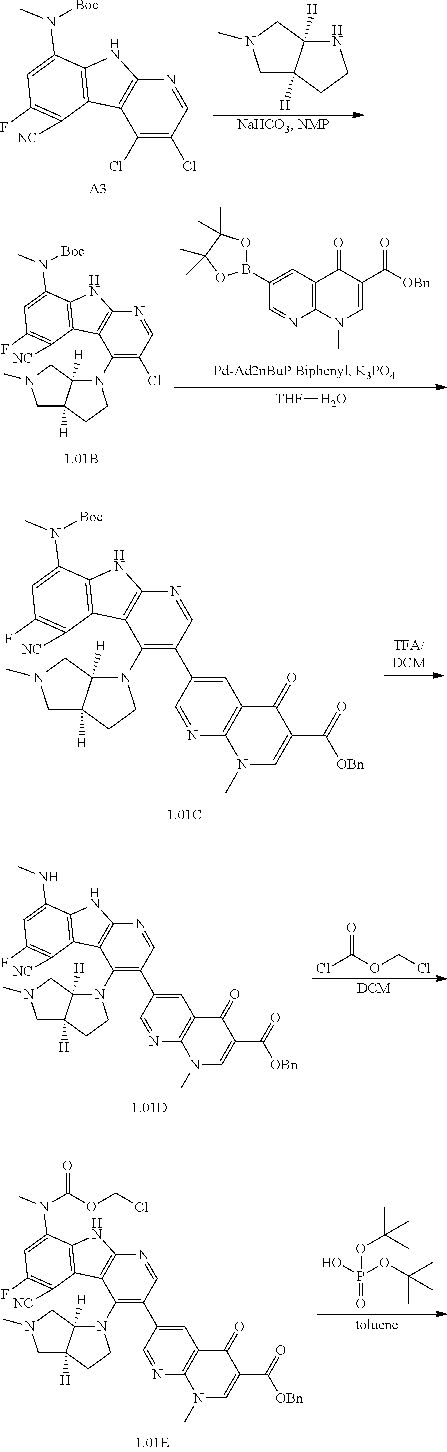

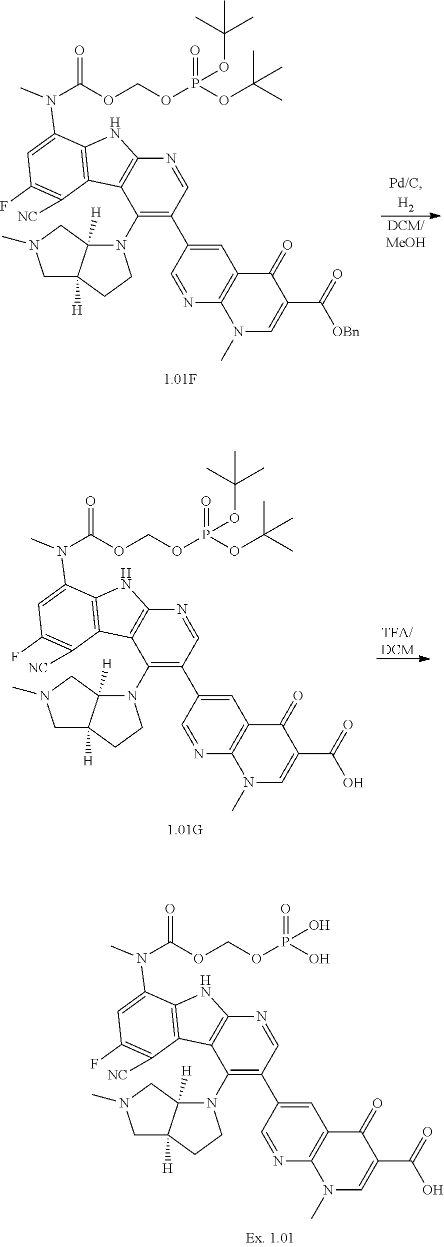 [SMILES: CN1C[C@@H]2CCN(c3c(-c4cnc5c(c4)c(=O)c(C(=O)OCc4ccccc4)cn5C)cnc4[nH]c5c(N(C)C(=O)OCOP(=O)(OC(C)(C)C)OC(C)(C)C)cc(F)c(C#N)c5c34)[C@@H]2C1 ; "DCM/MeOH" ; "Pd/C,H2" ; CN1C[C@@H]2CCN(c3c(-c4cnc5c(c4)c(=O)c(C(=O)O)cn5C)cnc4[nH]c5c(N(C)C(=O)OCOP(=O)(OC(C)(C)C)OC(C)(C)C)cc(F)c(C#N)c5c34)[C@@H]2C1 ; "TFA/DCM" ; CN1C[C@@H]2CCN(c3c(-c4cnc5c(c4)c(=O)c(C(=O)O)cn5C)cnc4[nH]c5c(N(C)C(=O)OCOP(=O)(O)O)cc(F)c(C#N)c5c34)[C@@H]2C1]

### Step (a) Preparation of tert-butyl N-[3-chloro-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-8-yl]-N-methyl-carbamate (Compound 1.01B)

A mixture solution of tert-butyl N-(3,4-dichloro-5-cyano-6-fluoro-9H-pyrido[2,3-b]indol-8-yl)-N-methyl-carbamate (3.3 g, 8.06 mmol, Intermediate A3), (3aS,6aS)-5-methyl-2,3,3a,4,6,6a-hexahydro-1H-pyrrolo[2,3-c]pyrrole (1.53 g, 12.1 mmol, CAS: 1176846-84-6) and NaHCO₃ (3.39 g, 40.32 mmol) in NMP (12 mL)) was stirred at 120° C. for 48 h. After TLC (petrolieum ether:EtOAc=2:1, 2% of TEA as additive) showed the starting material was consumed completely, the mixture was cooled back to r.t. and poured into EtOAc (500 mL). It was washed with aq. CaCl₂ solution (100 mL) three times and brine (100 mL) twice. The separated organic layer was dried over anhy. Na₂SO₄, filtered and concentrated in vacuo to give a crude product, which was purified by silica gel flash chromatography (petrolieum ether:EtOAc=10:1˜1:1, 2% of TEA as additive) to give tot-butyl N-[3-chloro-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-8-yl]-N-methyl-carbamate (add optical rotation value here, 3 g, 72.8% yield) as a light yellow solid. MS (ESI): 499.3 ([{³⁵Cl}M+H]⁺).

### Step (b) Preparation of benzyl 6-[8-[tert-butoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (Compound 1.01C)

A mixture of benzyl 1-methyl-4-oxo-6-(4,4,5,5-tetramethyl-1,3,2-dioxaborolan-2-yl)-1,8-naphthyridine-3-carboxylate (2.95 g, 7.01 mmol, Intermediate B3), tot-butyl N-[4-[(3aS,6aS)-5-methyl-2,3,3a,4,6,6a-hexahydropyrrolo[2,3-c]pyrrol-1-yl]-3-chloro-5-cyano-6-fluoro-9H-pyrido[2,3-b]indol-8-yl]-N-methyl-carbamate (2.0 g, 4.01 mmol), potassium phosphate (3.4 g, 16.03 mmol), and Pd-Ad2nBuP Biphenyl (536.0 mg, 0.800 mmol, cataCXium A-Pd-G2, CAS: 1375477-29-4) were dissolved in 1,4-dioxane (150 mL) and water (18 mL) under argon. The resulting reaction mixture was stirred at 70° C. for 12 h until LC-MS showed the starting material was consumed completely. The mixture was cooled back to r.t., and filtered. The filtrate was diluted with EtOAc (500 mL) and washed with brine (100 mL) three times. The separated organic layer was then dried over anhy. Na₂SO₄, filtered, and concentrated in vacuo to give a crude product, which was purified by prep-HPLC to give benzyl 6-[8-[tot-butoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (1.85 g, 60.5% yield)) as a light yellow solid. MS (ESI): 757.4 ([M+H]⁺).

### Step (c) Preparation of benzyl 6-[5-cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (Compound 1.01D)

To a solution of benzyl 6-[8-[tot-butoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (1.85 g, 2.44 mmol) in DCM (20 mL) was added TFA (8.0 mL, 103.84 mmol) and the resulting mixture was stirred at 25° C. for 1 h. After LC-MS showed the starting material was consumed completely, the mixture was concentrated in vacuo to give a crude product, which was purified by prep-HPLC to give benzyl 6-[5-cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (1.5 g, 1.95 mmol, 79.6% yield) as a yellow solid. MS (ESI): 657.3 ([M+H]⁺).

### Step (d) Preparation of benzyl 6-[8-[chloromethoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (Compound 1.01E)

To a solution of benzyl 6-[5-cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (TO g, 1.52 mmol) in DCM (30 mL) was added chloromethyl chloroformate (60.0 g, 465.33 mmol) and the resulting mixture was stirred at r.t. for 18 h. After LC-MS showed the starting material was consumed completely, the mixture solution was evaporated under the reduced pressure to dryness. The residue was re-dissolved in DCM and basified with aq. NH₄HCO₃ solution to pH=7. The solution was filtered and the filtrate was concentrated in vacuo to give a crude product, which was purified by prep-HPLC (formic acid as additive). The collected eluents were then basified with aq. NH₄HCO₃ solution to pH=7 and extracted with DCM (300 mL) twice. Combined organics was then washed with brine (200 mL) twice, dried by anhy. Na₂SO₄, filtered and concentrated in vacuo to give benzyl 6-[8-[chloromethoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (580 mg, 46.6% yield) as a yellow solid. MS (ESI): 749.3 ([{³⁵Cl}M+H]⁺).

### Step (e) Preparation of benzyl 6-[5-cyano-8-[di-tert-butoxyphosphoryloxymethoxycarbonyl(methyl)amino]-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (Compound 1.01F)

To a solution of benzyl 6-[8-[chloromethoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (330.0 mg, 0.440 mmol) in toluene (7 mL) was added potassium di-tert-butylphosphate (437.13 mg, 1.76 mmol). The resulting reaction mixture was stirred at 90° C. for 2 h until LC-MS showed the starting material was consumed completely. The mixture was then cooled back to r.t., poured into water (50 mL) and extracted with DCM (50 mL) three times. The organic layer was dried over anhy. Na₂SO₄, filtered, and concentrated in vacuo to give a crude product, which was purified by prep-HPLC (formic acids as additive). The collected eluents were then basified with aq. NH₄HCO₃ solution to pH 7˜8 and extracted with DCM (200 mL) twice. Combined organics was then dried over anhy. Na₂SO₄, filtered, and concentrated in vacuo to give benzyl 6-[5-cyano-8-[di-tert-butoxyphosphoryloxymethoxycarbonyl(methyl)amino]-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (250 mg, 57.9% yield) as a light yellow solid. MS (ESI): 923.4 ([M+H]⁺).

### Step (g) Preparation of 6-[5-cyano-8-[di-tert-butoxyphosphoryloxymethoxycarbonyl(methyl)amino]-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (Compound 1.01G)

To a solution of benzyl 6-[5-cyano-8-[di-tert-butoxyphosphoryloxymethoxycarbonyl(methyl)amino]-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (240.0 mg, 0.260 mmol) in DCM (4 mL) and MeOH (8 mL) was added palladium on carbon (100 mg). After the suspension was degassed under vacuum and purged with H₂ several times, the reaction mixture was stirred at 25° C. for 3 h under H₂ atmosphere (15 psi). After LC-MS showed the starting material was consumed completely, the reaction mixture was filtered through Celite. The filtrate was then concentrated in vacuo to give a crude product of 6-[5-cyano-8-[di-tert-butoxyphosphoryloxymethoxycarbonyl(methyl)amino]-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (190 mg, 0.230 mmol, 87.8% yield) as a light yellow solid. MS (ESI): 833.3 ([M+H]⁺). It was used directly in the next step without further purification.

### Step (h) Preparation of 6-[5-cyano-6-fluoro-8-[methyl(phosphonooxymethoxycarbonyl)amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (Example 1.01)

A mixture solution of 6-[4-[(3aS,6aS)-5-methyl-2,3,3a,4,6,6a-hexahydropyrrolo[2,3-c]pyrrol-1-yl]-5-cyano-8-[di-tert-butoxyphosphoryloxymethoxycarbonyl(methyl)amino]-6-fluoro-9H-pyrido[2,3-b]indol-3-yl]-1-methyl-4-oxo-1,8-naphthyridine-3-carboxylic acid (161.36 mg, 0.190 mmol) and TFA in DCM (5.0 mL) (v/v, DCM:TFA=20:1) was stirred at 25° C. for 2 h under nitrogen. After LC-MS showed the starting material was consumed completely, the solution was adjust to pH >7 by aq. NH₄HCO₃ solution. The mixture was concentrated in vacuo to give a crude product, which was purified by prep-HPLC (NH₄HCO₃ as additive) to give 6-[5-cyano-6-fluoro-8-[methyl(phosphonooxymethoxycarbonyl)amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (80.7 mg, 0.110 mmol, 49.8% yield) as a light yellow solid.

MS (ESI): 721.3 ([M+H]⁺). ¹H NMR (400 MHz, DMSO-d₆) δ ppm 12.53-14.43 (m, 1H), 9.10-9.36 (m, 2H), 8.74 (br s, 1H), 7.93-8.15 (m, 1H), 7.11-7.77 (m, 2H), 4.51-5.70 (m, 3H), 4.11-4.27 (m, 3H), 3.31 (br s, 4H), 2.93-3.09 (m, 3H), 2.54-2.64 (m, 1H), 2.12-2.46 (m, 6H), 1.48 (br s, 1H).

### Example 1.02

### 6-[5-Cyano-6-fluoro-8-[methyl-[[(E)-3-carboxyprop-2-enoyl]oxymethoxycarbonyl]amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid

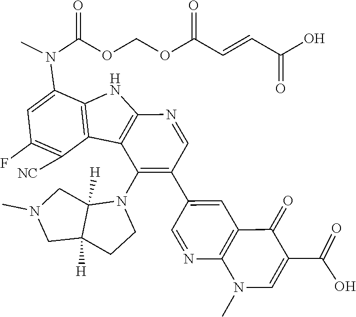 [SMILES: CN1C[C@@H]2CCN(c3c(-c4cnc5c(c4)c(=O)c(C(=O)O)cn5C)cnc4[nH]c5c(N(C)C(=O)OCOC(=O)/C=C/C(=O)O)cc(F)c(C#N)c5c34)[C@@H]2C1]

The titled compound was synthesized according to the following scheme:

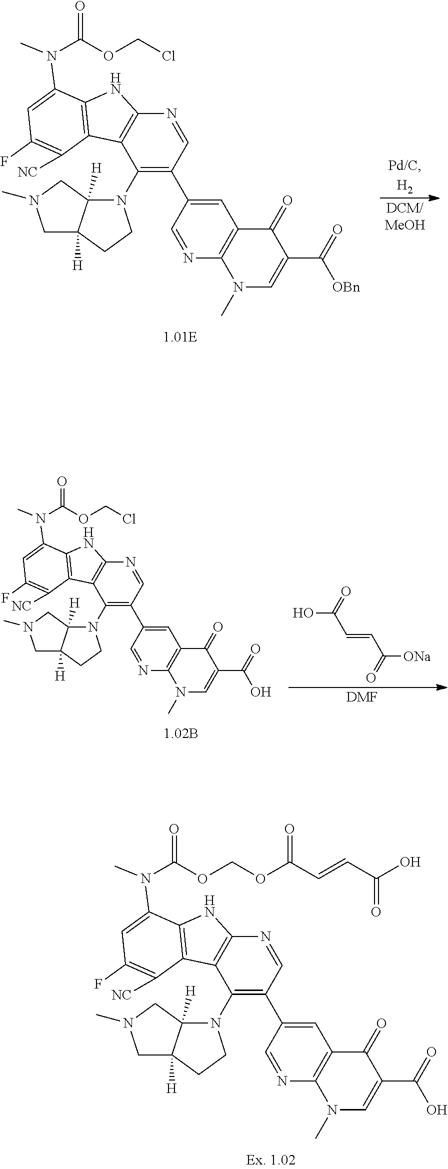

### Step (a) Preparation of 6-[8-[chloromethoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (Compound 1.02B)

To a solution of benzyl 6-[8-[chloromethoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (230 mg, 0.31 mmol, compound 1.01E) in DCM (2 mL) and MeOH (2 mL) was added palladium on carbon (115 mg). After the suspension was degassed under vacuum and purged with H₂ several times, the reaction mixture was stirred at 25° C. for 3 h under H₂ atmosphere (15 psi). After LC-MS showed the starting material was consumed completely, the reaction mixture was filtered through Celite. The filtrate was concentrated in vacuo to give a crude product of 6-[8-[chloromethoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (180 mg, 73.2% yield) as a light yellow solid. MS (ESI): 659.3 (([{³⁵Cl}M+H]⁺). It was used directly in the next step without further purification.

### Step (b) Preparation of 6-[5-cyano-6-fluoro-8-[methyl-[[(E)-3-carboxyprop-2-enoyl]oxymethoxycarbonyl]amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (Example 1.02)

To a solution of 6-[4-[(3aS,6aS)-5-methyl-2,3,3a,4,6,6a-hexahydropyrrolo[2,3-c]pyrrol-1-yl]-8-[chloromethoxycarbonyl(methyl)amino]-5-cyano-6-fluoro-9H-pyrido[2,3-b]indol-3-yl]-1-methyl-4-oxo-1,8-naphthyridine-3-carboxylic acid (180.0 mg, 0.270 mmol) in DMF (4 mL) was added sodium hydrogen fumarate (150.81 mg, 1.09 mmol, CAS: 5873-57-4). The resulting mixture was stirred at 90° C. for 2 h until LC-MS showed the starting material was consumed completely. The reaction mixture was cooled back to r.t. and filtered. The residue was subject to prep-HPLC purification (NH₄HCO₃ as additive) to give 6-[5-cyano-6-fluoro-8-[methyl-[[(E)-3-carboxyprop-2-enoyl]oxymethoxycarbonyl]amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (24.5 mg, 11.8% yield) as a light yellow solid. MS (ESI): 739.3 ([M+H]⁺). ¹H NMR (400 MHz, DMSO-d6) δ ppm 14.81 (br s, 1H), 12.24-12.96 (m, 1H), 9.27 (s, 1H), 9.17 (br s, 1H), 8.81 (br s, 1H), 8.00-8.19 (m, 1H), 7.74 (br d, J=10.3 Hz, 1H), 6.21-6.96 (m, 2H), 5.47-6.01 (m, 2H), 4.59 (br s, 1H), 4.18 (s, 3H), 3.07-3.20 (m, 3H), 2.95-3.02 (m, 2H), 2.79-2.85 (m, 1H), 2.83 (br d, J=7.3 Hz, 1H), 2.39-2.47 (m, 1H), 1.97-2.29 (m, 7H), 1.75 (br s, 1H), 1.49 (br s, 1H).

### Example 1.03

### 6-[5-Cyano-6-fluoro-8-[formyl(methyl)amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid

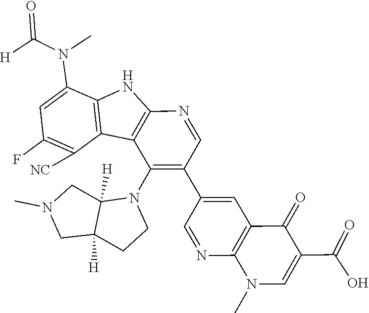 [SMILES: CN1C[C@@H]2CCN(c3c(-c4cnc5c(c4)c(=O)c(C(=O)O)cn5C)cnc4[nH]c5c(N(C)C=O)cc(F)c(C#N)c5c34)[C@@H]2C1]

The titled compound was synthesized according to the following scheme:

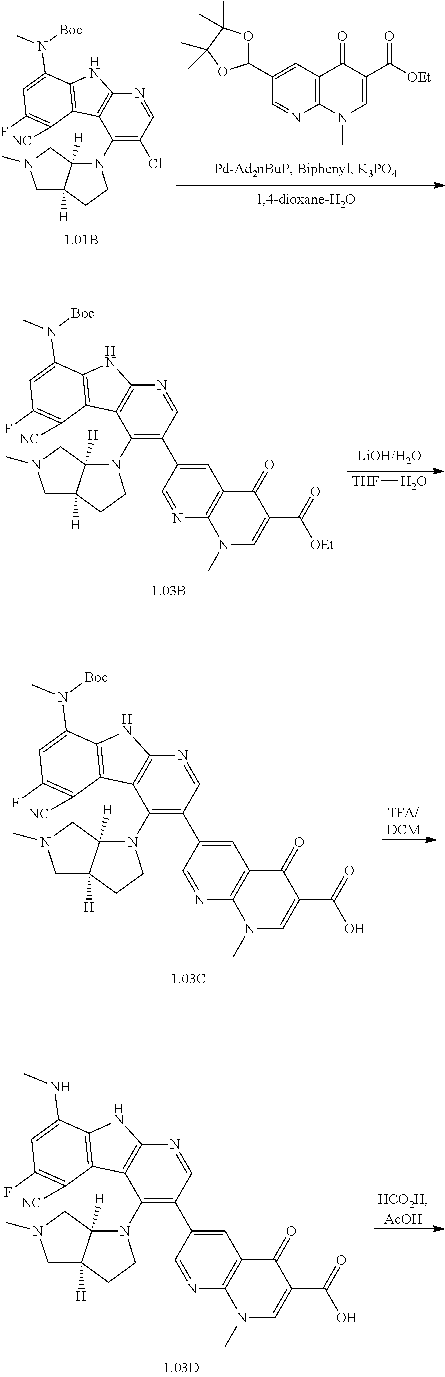

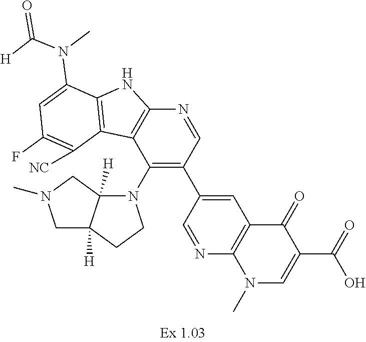 [SMILES: CN1C[C@@H]2CCN(c3c(-c4cnc5c(c4)c(=O)c(C(=O)O)cn5C)cnc4[nH]c5c(N(C)C=O)cc(F)c(C#N)c5c34)[C@@H]2C1]

### Step (a) Preparation of ethyl 6-[8-[tert-butoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (Compound 1.03B)

To a mixture solution of tert-butyl N-[3-chloro-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-8-yl]-N-methyl-carbamate (20.0 g, 40.1 mmol, compound 1.01B), ethyl 1-methyl-4-oxo-6-(4,4,5,5-tetramethyl-1,3,2-dioxaborolan-2-yl)-1,8-naphthyridine-3-carboxylate (21.5 g, 60.1 mmol, Intermediate B1), and K₃PO₄ (34.1 g, 160.4 mmol) in dioxane/H₂O (500.0 mL, v/v=5:l) was added Pd-Ad₂nBuP Biphenyl (4.02 g, 6.01 mmol) in a glove box under argon. The resulting reaction mixture was stirred at 90° C. for 12 h until TLC (petrolieum ether:EtOAc=1:2, 3% TEA as additive) showed the starting material was consumed completely. The mixture was cooled back to r.t. and filtered. The filtrate was diluted with EtOAc (1.2 L) and washed by brine (300 mL) twice. The organic layer was dried with anhy. Na₂SO₄, filtered, and concentrated in vacuo to give a crude product, which was purified by silica gel flash chromatography (petrolieum ether:EtOAc=100:1˜1:5, 3% TEA as additive) to give ethyl 6-[8-[tert-butoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (21.2 g, 76.3% yield) as a yellow solid. MS (ESI): 695.4 ([M+H]⁺).

### Step (b) Preparation of 6-[8-[tert-butoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (Compound 1.03C)

To a stirred solution of ethyl 6-[8-[tert-butoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (20.8 g, 29.94 mmol) in THF/EtOH, 120.0 mL, v/v=3:1) was added aq. LiOH solution (44.91 mL, 89.92 mmol, 2.0 N). The resulting reaction mixture was stirred at 15° C. for 3 h until LC-MS showed the starting material was consumed completely. After dilution with EtOAc (300 mL) and H₂O (50 mL), the mixture was adjusted to pH=6 with 1 N HCl aqueous solution and extracted with EtOAc (300 mL) twice. Combined organics was washed with brine (100.0 mL) twice, dried over anhy. Na₂SO₄, filtered, and concentrated in vacuo to give 6-[8-[tert-butoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (19.0 g, 95.2% yield) as a yellow solid. MS (ESI): 667.4 ([M+H]⁺). It was used directly in the next step without further purification.

### Step (c) Preparation of 6-[5-cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (Compound 1.03D)

To a stirred solution of 6-[8-[tert-butoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (18.9 g, 28.35 mmol) in DCM (50.0 mL) was added TFA (20.0 mL). The reaction mixture was stirred at 15° C. for 2 h until LC-MS showed the starting material was consumed completely. The mixture was evaporated to dryness under the reduced pressure. The residue was re-dissolved in MeOH (150 mL) and adjusted to pH=6 with NaHCO₃ solid. The mixture was filtered and the filtrate was concentrated in vacuo to give a crude product, which was purified by prep-HPLC (NH₃.H₂O as additive) to give 6-[5-cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (11.6 g, 72.2% yield) as a yellow solid. MS (ESI): 567.4 ([M+H]⁺), 284.3 ([M/2+H]⁺). ¹H NMR (400 MHz, DMSO-d6) δ ppm 14.82 (br s, 1H), 11.89 (br s, 1H), 9.25 (s, 1H), 9.16 (d, J=2.3 Hz, 1H), 8.76 (d, J=2.3 Hz, 1H), 8.03 (s, 1H), 6.83 (br d, J=3.3 Hz, 1H), 6.65 (d, 0.7=13.1 Hz, 1H), 4.60˜4.74 (m, 1H), 4.17 (s, 3H), 2.89˜3.05 (m, 5H), 2.71˜2.81 (m, 1H), 2.46 (br d, J=9.0 Hz, 1H), 2.07˜2.31 (m, 6H), 1.66 (dd, 0.7=9.7, 5.1 Hz, 1H), 1.42 (br s, 1H).

### Step (d) Preparation of 6-[5-cyano-6-fluoro-8-[formyl(methyl)amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (Example 1.031

A solution of 6-[5-cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (30.0 mg, 0.050 mmol) in acetic acid (2.19 mL) and HCOOH (2.0 mL, 0.530 mmol) was stirred at 15° C. for 2 h. After LC-MS showed the starting material was consumed completely, the mixture was concentrated in vacuo to give a crude product, which was purified by prep-HPLC (TFA as additive) to give 6-[5-cyano-6-fluoro-8-[formyl(methyl)amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (8 mg, 23.9% yield) as a yellow solid. MS (ESI): 595.3 ([M+H]⁺). ¹H NMR (400 MHz, DMSO-d₆) δ ppm: ¹H NMR (400 MHz, DMSO-d₆) δ=12.91 (br s, 1H), 12.54 (br s, 1H), 9.34-9.19 (m, 2H), 8.76 (d, J=2.0 Hz, 1H), 8.50 (s, 1H), 8.53-8.36 (m, 1H), 8.39 (s, 1H), 8.19 (br s, 1H), 7.84-7.73 (m, 1H), 4.73 (br s, 1H), 4.19 (s, 3H), 3.59-3.56 (m, 2H), 3.35 (s, 3H), 3.23 (br s, 3H), 2.88-2.61 (m, 5H), 2.30-2.07 (m, 1H), 1.65 (br s, 1H).

### Example 1.04

### 6-[5-Cyano-6-fluoro-8-(methylamino)-1-(phosphonooxymethyl)-4-[(3aS,6aS)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylic acid

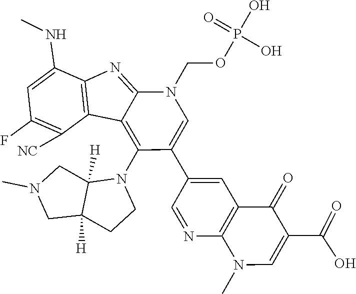 [SMILES: CNc1cc(F)c(C#N)c2c3c(N4CC[C@H]5CN(C)C[C@H]54)c(-c4cnc5c(c4)c(=O)c(C(=O)O)cn5C)cn(COP(=O)(O)O)c-3nc12]

The titled compound was synthesized according to the following scheme:

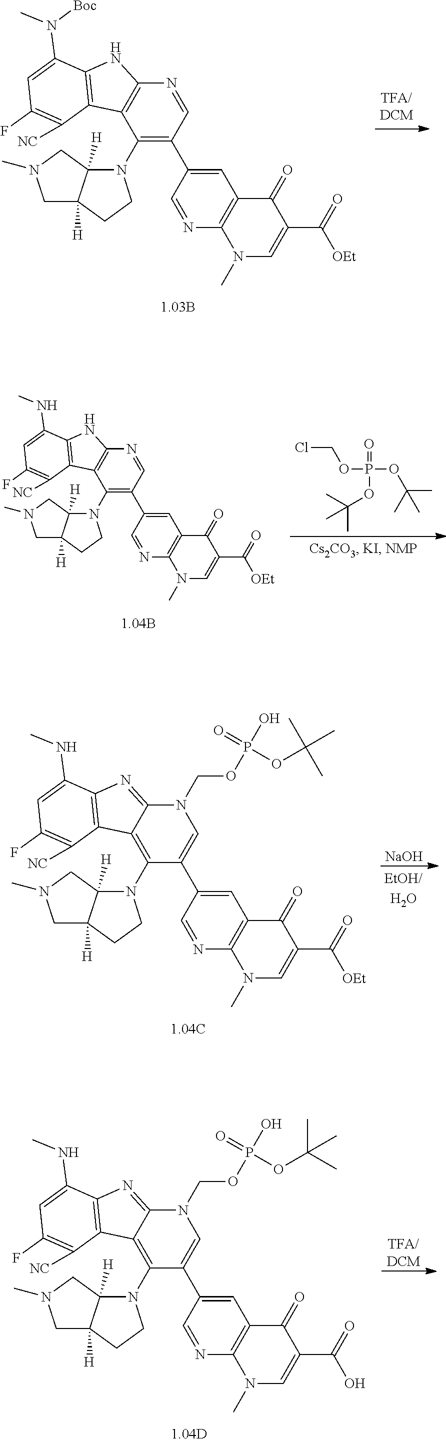

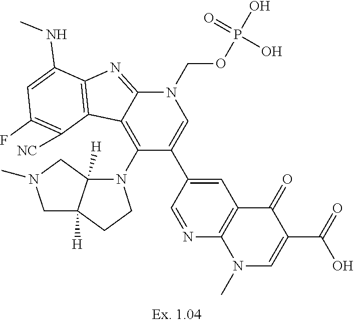 [SMILES: CNc1cc(F)c(C#N)c2c3c(N4CC[C@H]5CN(C)C[C@H]54)c(-c4cnc5c(c4)c(=O)c(C(=O)O)cn5C)cn(COP(=O)(O)O)c-3nc12]

### Step (a) Preparation of ethyl 6-[5-cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (Compound 1.04B)

To a solution of ethyl 6-[8-[tert-butoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (510 mg, 0.73 mmol, compound 1.03B) in DCM (5 mL) was added trifluoroacetic acid (2 mL, 25.9 mmol), and the reaction mixture was stirred at 20° C. for 1 h. After LC-MS showed the starting material was consumed, the mixture was diluted with DCM (50 mL), poured into water (50 mL), adjusted to pH=8 by aq. NaOH solution (2 N), and extracted with DCM (100 mL). The organic layer was washed with brine (100 mL), dried over anhy. Na₂SO₄, filtered, and concentrated in vacuo to give a crude product of ethyl 6-[5-cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (410 mg, 93.9% yield) as a yellow solid. MS (ESI): 595.3 ([M+H]⁺). It was used directly in the next step without further purification.

### Step (b) Preparation of ethyl 6-[1-[[tert-butoxy(hydroxy)phosphoryl]oxymethyl]-5-cyano-6-fluoro-8-(methylamino)-4-[(3aS,6aS)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylate (Compound 1.04C)

To a mixture solution of ethyl 6-[5-cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (350 mg, 0.59 mmol), cesium carbonate (553.3 mg, 1.7 mmol), and potassium iodide (0.09 mL, 1.67 mmol) in NMP (5.0 mL) was added di-tert-butyl chloromethyl phosphate (345.8 mg, 1.34 mmol), and the reaction mixture was stirred at 20° C. for 4 h. After LC-MS showed the starting material was consumed, the mixture was diluted with DCM (100 mL), poured into water (100 mL), and extracted with DCM (100 mL) twice. Combined organics was then washed with brine (100 mL), dried over anhy. Na₂SO₄, filtered, concentrated in vacuo to give a crude product, which was purified by prep-HPLC (NH₄HCO₃ as additive) to give ethyl 6-[1-[[tert-butoxy(hydroxy)phosphoryl]oxymethyl]-5-cyano-6-fluoro-8-(methylamino)-4-[(3aS,6aS)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylate (65.0 mg, 14.5% yield) as a yellow solid. MS (ESI): 761.4 ([M+H]⁺).

### Step (c) Preparation of 6-[1-[[tert-butoxy(hydroxy (phosphoryl]oxymethyl]-5-cyano-6-fluoro-8-(methylamino)-4-[(3aS,6aS)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylic acid (Compound 1.04D)

To a solution of ethyl 6-[1-[[tert-butoxy(hydroxy)phosphoryl]oxymethyl]-5-cyano-6-fluoro-8-(methylamino)-4-[(3aS,6aS)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylate (60 mg, 0.08 mmol) in EtOH (3.0 mL) and water (0.5 mL) was added lithium hydroxide (0.21 mL, 1.67 mmol), and the reaction mixture was stirred at 20° C. for 1 h. After LC-MS showed the starting material was consumed, the mixture was adjusted to pH=6 with aq. HCl solution (1M), and purified by prep-HPLC to give 6-[1-[[tert-butoxy(hydroxy)phosphoryl]oxymethyl]-5-cyano-6-fluoro-8-(methylamino)-4-[(3aS,6aS)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylic acid (55.0 mg, 95.2% yield) as a yellow solid. MS (ESI): 733.3 ([M+H]⁺).

### Step (d) Preparation of 6-[5-cyano-6-fluoro-8-(methylamino)-1-(phosphonooxymethyl)-4-[(3aS,6aS)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylic acid (Example 1.04)

To a solution of 6-[1-[[tert-butoxy(hydroxy)phosphoryl]oxymethyl]-5-cyano-6-fluoro-8-(methylamino)-4-[(3aS,6aS)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylic acid (50.0 mg, 0.07 mmol) in DCM (5.0 mL) was added TFA/DCM (3.0 mL, 0.07 mmol, v/v=1:3), and the reaction mixture was stirred at 20° C. for 1 h. After LC-MS showed the starting material was consumed, the mixture was concentrated in vacuo to give a crude product, which was purified by prep-HPLC (TFA as additive) to give 6-[5-cyano-6-fluoro-8-(methylamino)-1-(phosphonooxymethyl)-4-[(3aS,6aS)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylic acid (14.5 mg, 26.4% yield) as a yellow solid. MS (ESI): 677.3 ([M+H]⁺). ¹H NMR (400 MHz, DMSO-d6) δ ppm 9.30 (br d, J=10.88 Hz, 2H), 8.98 (br s, 1H), 8.49 (br s, 1H), 7.23˜7.25 (m, 1H), 6.66˜6.73 (m, 1H), 6.65˜6.66 (m, 1H), 6.07˜6.17 (m, 2H), 4.17 (br s, 4H), 3.01˜3.39 (m, 3H), 2.94 (br s, 3H), 2.68 (br d, J=1.83 Hz, 4H), 2.30-2.37 (m, 1H), 2.11 (br s, 1H), 1.73 (br s, 2H).

### Example 1.05

### 6-[5-Cyano-6-fluoro-8-(methylamino)-1-[(5-methyl-2-oxo-1,3-dioxol-4-yl)methyl]-4-[(3aS,6aS)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylic acid

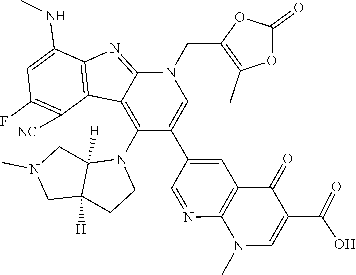 [SMILES: CNc1cc(F)c(C#N)c2c3c(N4CC[C@H]5CN(C)C[C@H]54)c(-c4cnc5c(c4)c(=O)c(C(=O)O)cn5C)cn(Cc4oc(=O)oc4C)c-3nc12]

The titled compound was synthesized according to the following scheme:

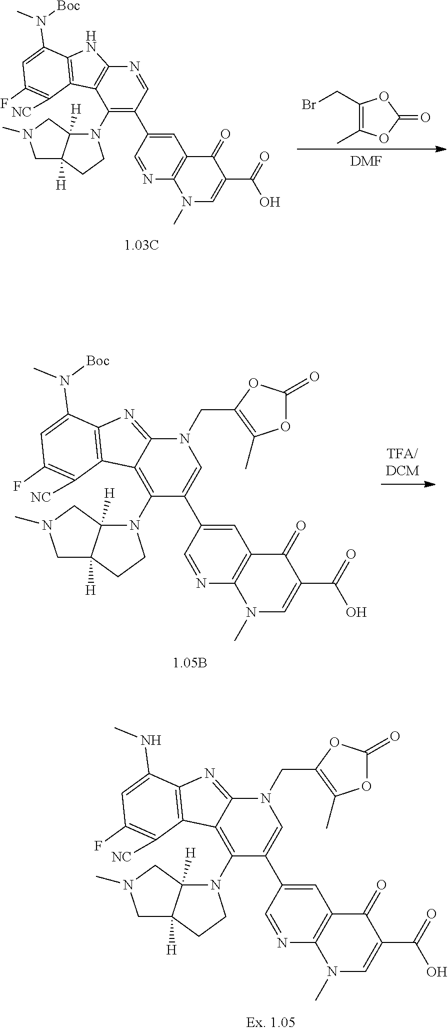

### Step (a) Preparation of 6-[8-[tert-butoxycarbonyl(methyl)amino]-5-cyano-6-fluoro-1-[(5-methyl-2-oxo-1,3-dioxol-4-yl)methyl]-4-[(3aS,6aS)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylic acid (Compound 1.05B)

To a solution of 6-[8-[tert-butoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (190 mg, 0.28 mmol, compound 1.03C) in DMF (3 mL) was added 4-(bromomethyl)-5-methyl-1,3-dioxol-2-one (95.0 mg, 0.490 mmol) at 0° C., and the resulting reaction mixture was stirred at 20° C. for 12 h. After LC-MS showed the starting material was consumed, the reaction mixture was then concentrated in vacuo to give a crude product, which was further purified by prep-HPLC (TFA as additive) to give 6-[8-[tert-butoxycarbonyl(methyl)amino]-5-cyano-6-fluoro-1-[(5-methyl-2-oxo-1,3-dioxol-4-yl)methyl]-4-[(3aS,6aS)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylic acid (45 mg, 20.3% yield) as a yellow solid. MS (ESI): 779.2 ([M+H]⁺). Analytical HPLC Rt=2.97 min (faster eluted compared to compound 1.06B).

### Step (b) Preparation of 6-[5-cyano-6-fluoro-8-(methylamino)-1-[(5-methyl-2-oxo-1,3-dioxol-4-yl)methyl]-4-[(3aS,6aS)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylic acid

### Example 1.05

To a solution of 6-[8-[tert-butoxycarbonyl(methyl)amino]-5-cyano-6-fluoro-1-[(5-methyl-2-oxo-1,3-dioxol-4-yl)methyl]-4-[(3aS,6aS)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylic acid (40.0 mg, 0.05 mmol) in DCM (3.0 mL) was added TFA (1.0 mL, 13 mmol), and the resulting reaction mixture was stirred at 20° C. for 1 h. After LC-MS showed the starting material was consumed, the mixture solution was concentrated in vacuo to give a crude product, which was purified by prep-HPLC (TFA as additive) to give 6-[5-cyano-6-fluoro-8-(methylamino)-1-[(5-methyl-2-oxo-1,3-dioxol-4-yl)methyl]-4-[(3aS,6aS)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylic acid (22.3 mg, 47.1% yield) as a yellow solid. MS (ESI): 679.2 ([M+H]⁺). ¹H NMR (400 MHz, DMSO-d6) δ ppm 14.65 (br s, 1H), 12.09 (br s, 1H), 9.29˜9.37 (m, 1H), 9.16˜9.28 (m, 1H), 8.91 (br s, 1H), 8.73 (br s, 1H), 8.24 (br s, 1H), 8.11 (s, 1H), 6.61˜6.74 (m, 1H), 5.67 (br d, J=11.62 Hz, 2H), 4.86˜5.21 (m, 1H), 4.19 (s, 3H), 3.25 (br d, J=7.83 Hz, 2H), 3.03 (s, 5H), 2.59˜2.93 (m, 6H), 2.29 (s, 3H), 1.97˜2.15 (m, 1H), 1.63˜1.79 (m, 1H).

### Example 1.06

### 6-[5-Cyano-6-fluoro-8-(methylamino)-4-[(3aS,6aS)-5-methyl-5-[(5-methyl-2-oxo-1,3-dioxol-4-yl)methyl]-2,3,3a,4,6,6a-hexahydropyrrolo[2,3-c]pyrrol-5-ium-1-yl]-9H-pyrido[2,3-b]indol-3-yl]-1-methyl-4-oxo-1,8-naphthyridine-3-carboxylic acid

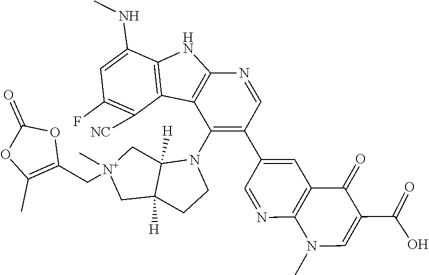 [SMILES: CNc1cc(F)c(C#N)c2c1[nH]c1ncc(-c3cnc4c(c3)c(=O)c(C(=O)O)cn4C)c(N3CC[C@H]4C[N+](C)(Cc5oc(=O)oc5C)C[C@H]43)c12]

The titled compound was synthesized according to the following scheme:

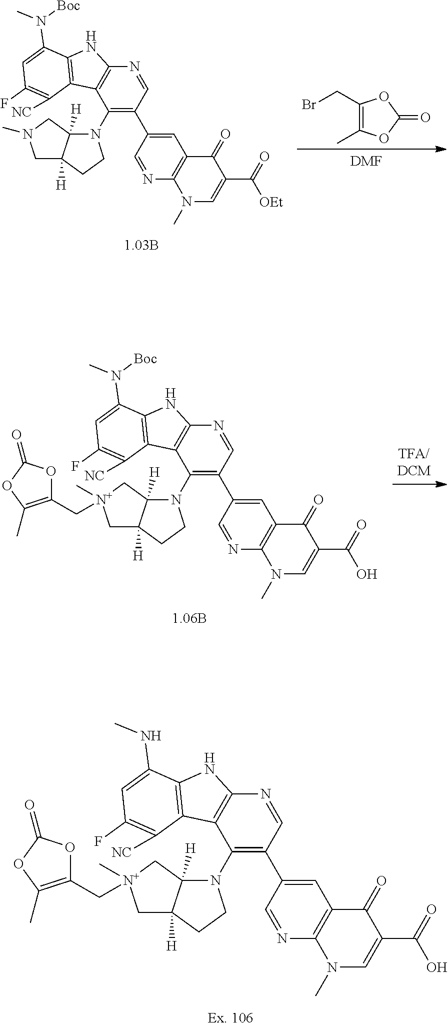

### Step (a) Preparation of 6-[8-[tert-butoxycarbonyl(methyl)amino]-5-cyano-6-fluoro-4-[(3aS,6aS)-5-methyl-5-[(5-methyl-2-oxo-1,3-dioxol-4-yl)methyl]-2,3,3a,4,6,6a-hexahydropyrrolo[2,3-c]pyrrol-5-ium-1-yl]-9H-pyrido[2,3-b]indol-3-yl]-1-methyl-4-oxo-1,8-naphthyridine-3-carboxylic acid (Compound 1.06B)

During the synthesis of Example 1.05B, 6-[8-[tert-butoxycarbonyl(methyl)amino]-5-cyano-6-fluoro-4-[(3aS,6aS)-5-methyl-5-[(5-methyl-2-oxo-1,3-dioxol-4-yl)methyl]-2,3,3a,4,6,6a-hexahydropyrrolo[2,3-c]pyrrol-5-ium-1-yl]-9H-pyrido[2,3-b]indol-3-yl]-1-methyl-4-oxo-1,8-naphthyridine-3-carboxylic acid was also obtained as another yellow solid (46 mg, 20.8% yield) after prep-HPLC purification. MS (ESI): 779.3 ([M+H]⁺). Analytical HPLC Rt=3.37 min (slower eluted compared to compound 1.05B).

### Step (b) Preparation of 6-[5-cyano-6-fluoro-8-(methylamino)-4-[(3aS,6aS)-5-methyl-5-[(5-methyl-2-oxo-1,3-dioxol-4-yl)methyl]-2,3,3a,4,6,6a-hexahydropyrrolo[2,3-c]pyrrol-5-ium-1-yl]-9H-pyrido[2,3-b]indol-3-yl]-1-methyl-4-oxo-1,8-naphthyridine-3-carboxylic acid (Example 1.06)

To a solution of 6-[8-[tert-butoxycarbonyl(methyl)amino]-5-cyano-6-fluoro-4-[(3aS,6aS)-5-methyl-5-[(5-methyl-2-oxo-1,3-dioxol-4-yl)methyl]-2,3,3a,4,6,6a-hexahydropyrrolo[2,3-c]pyrrol-5-ium-1-yl]-9H-pyrido[2,3-b]indol-3-yl]-1-methyl-4-oxo-1,8-naphthyridine-3-carboxylic acid (50 mg, 0.06 mmol) in DCM (3.0 mL) was added TFA (1.0 mL, 12.9 mmol), and the resulting reaction mixture was stirred at 20° C. for 1 h. After LC-MS showed the starting material was consumed, the mixture solution was concentrated in vacuo to give a crude product, which was purified by prep-HPLC (TFA additive) to give 6-[5-cyano-6-fluoro-8-(methylamino)-4-[(3aS,6aS)-5-methyl-5-[(5-methyl-2-oxo-1,3-dioxol-4-yl)methyl]-2,3,3a,4,6,6a-hexahydropyrrolo[2,3-c]pyrrol-5-ium-1-yl]-9H-pyrido[2,3-b]indol-3-yl]-1-methyl-4-oxo-1,8-naphthyridine-3-carboxylic acid (2.0 mg, 3.1% yield) as a yellow solid. MS (ESI): 679.2 ([M+H]⁺). ¹H NMR (400 MHz, DMSO-d6) δ ppm 14.56˜14.85 (m, 1H), 12.09 (br s, 1H), 9.16˜9.33 (m, 2H), 8.67˜8.74 (m, 1H), 8.04˜8.17 (m, 1H), 7.01˜7.05 (m, 1H), 6.86˜6.98 (m, 1H), 6.65˜6.73 (m, 1H), 4.84˜5.02 (m, 1H), 4.32˜4.43 (m, 1H), 4.10˜4.22 (m, 3H), 3.81˜4.06 (m, 2H), 3.48˜3.53 (m, 1H), 3.34˜3.45 (m, 2H), 3.21˜3.31 (m, 2H), 2.93˜3.20 (m, 6H), 2.67˜2.87 (m, 2H), 2.52˜2.56 (m, 1H), 2.43 (br s, 1H), 2.17˜2.40 (m, 2H), 2.07˜2.15 (m, 1H), 1.82˜1.95 (m, 1H), 1.50˜1.79 (m, 3H).

### Example 1.07

### 6-[5-Cyano-6-fluoro-8-(methylamino)-4-[(3aS,6aS)-5-methyl-5-(phosphonooxymethyl)-2,3,3a,4,6,6a-hexahydropyrrolo[2,3-c]pyrrol-5-ium-1-yl]-9H-pyrido[2,3-b]indol-3-yl]-1-methyl-4-oxo-1,8-naphthyridine-3-carboxylic acid

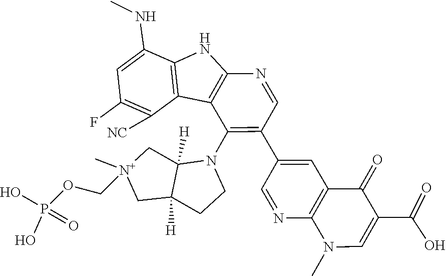 [SMILES: CNc1cc(F)c(C#N)c2c1[nH]c1ncc(-c3cnc4c(c3)c(=O)c(C(=O)O)cn4C)c(N3CC[C@H]4C[N+](C)(COP(=O)(O)O)C[C@H]43)c12]

The titled compound was synthesized according to the following scheme:

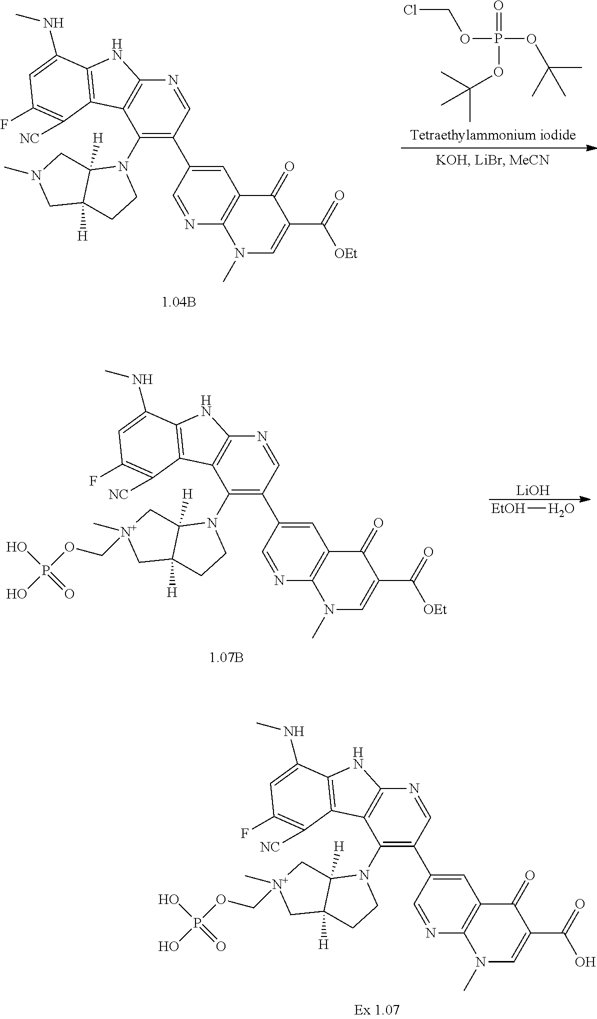

### Step (a) Preparation of ethyl 6-[5-cyano-6-fluoro-8-(methylamino)-4-[(3aS,6aS)-5-methyl-5-(phosphonooxymethyl)-2,3,3a,4,6,6a-hexahydropyrrolo[2,3-c]pyrrol-5-ium-1-yl]-9H-pyrido[2,3-b]indol-3-yl]-1-methyl-4-oxo-1,8-naphthyridine-3-carboxylate (Compound 1.07B)

A mixture solution of ethyl 6-[5-cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (120.0 mg, 0.2 mmol, compound 1.04B), di-tert-butyl chloromethyl phosphate (49.0 mg, 0.2 mmol), lithium bromide (17.5 mg, 0.2 mmol), potassium hydroxide (11.3 mg, 0.2 mmol), and tetraethylammonium iodide (52.0 mg, 0.2 mmol) in MeCN (6 mL) was stirred at 20° C. for 12 h. Afterwards, the mixture was diluted with DCM (100 mL), poured into water (100 mL), and extracted with DCM (100 mL). The organic layer was washed with brine (100 mL), dried over anhy. Na₂SO₄, filtered, and concentrated in vacuo to give a crude product, which was purified prep-HPLC (first with 0.1% NH₄HCO₃ in water as additive and then 0.1% TFA in water as additive) to give ethyl 6-[5-cyano-6-fluoro-8-(methylamino)-4-[(3aS,6aS)-5-methyl-5-(phosphonooxymethyl)-2,3,3a,4,6,6a-hexahydropyrrolo[2,3-c]pyrrol-5-ium-1-yl]-9H-pyrido[2,3-b]indol-3-yl]-1-methyl-4-oxo-1,8-naphthyridine-3-carboxylate (20 mg, 14.0% yield) as a yellow solid. MS (ESI): 705.3 ([M+H]⁺).

### Step (b) Preparation of 6-[5-cyano-6-fluoro-8-(methylamino)-4-[(3aS,6aS)-5-methyl-5-(phosphonooxymethyl)-2,3,3a,4,6,6a-hexahydropyrrolo[2,3-c]pyrrol-5-ium-1-yl]-9H-pyrido[2,3-b]indol-3-yl]-1-methyl-4-oxo-1,8-naphthyridine-3-carboxylic acid (Example 1.07)

To a solution of ethyl 6-[5-cyano-6-fluoro-8-(methylamino)-4-[(3aS,6aS)-5-methyl-5-(phosphonooxymethyl)-2,3,3a,4,6,6a-hexahydropyrrolo[2,3-c]pyrrol-5-ium-1-yl]-9H-pyrido[2,3-b]indol-3-yl]-1-methyl-4-oxo-1,8-naphthyridine-3-carboxylate (13.0 mg, 0.02 mmol) in EtOH (3.2 mL) and water (0.65 mL) was added lithium hydroxide monohydrate (6.5 mg, 0.15 mmol), and the reaction mixture was stirred at 20° C. for 1 h. After LC-MS showed the starting material was consumed, the mixture was purified by prep-HPLC (TFA as additive) to give 6-[5-cyano-6-fluoro-8-(methylamino)-4-[(3aS,6aS)-5-methyl-5-(phosphonooxymethyl)-2,3,3a,4,6,6a-hexahydropyrrolo[2,3-c]pyrrol-5-ium-1-yl]-9H-pyrido[2,3-b]indol-3-yl]-1-methyl-4-oxo-1,8-naphthyridine-3-carboxylic acid (3.8 mg, 14.5% yield) as a yellow solid. MS (ESI): 677.3 ([M+H]⁺). ¹H NMR (400 MHz, Solvent) ppm 9.05 (br s, 1H), 8.96 (s, 1H), 8.69 (br s, 1H), 7.98 (br s, 1H), 6.44 (br d, J=13.08 Hz, 1H), 4.05 (m, 3H), 3.83 (br s, 2H), 3.26 (br s, 6H), 2.95 (br s, 3H), 2.90 (s, 3H), 1.81˜1.91 (m, 1H), 1.10˜1.12 (m, 1H).

### Example 1.08

### 2-[(2S)-2-Amino-3-methyl-butanoyl]oxyethyl 6-[5-cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate

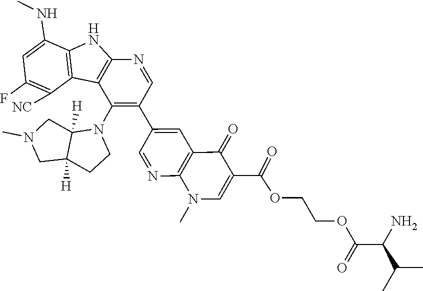 [SMILES: CNc1cc(F)c(C#N)c2c1[nH]c1ncc(-c3cnc4c(c3)c(=O)c(C(=O)OCCOC(=O)[C@@H](N)C(C)C)cn4C)c(N3CC[C@H]4CN(C)C[C@H]43)c12]

The titled compound was synthesized according to the following scheme:

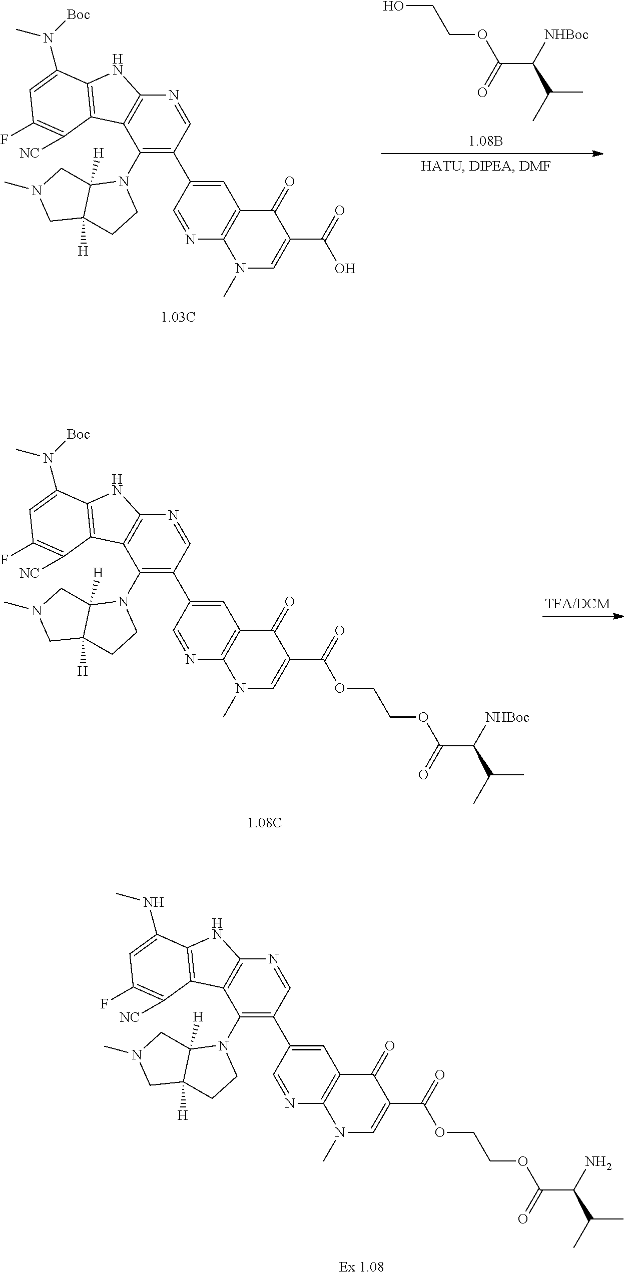

### Step (a) Preparation of (S)-2-hydroxy ethyl 2-(tert-butoxycarbonylamino)-3-methyl-butanoate (Compound 1.08B)

To a solution of (2S)-2-(tert-butoxycarbonylamino)-3-methyl-butanoic acid (500.0 mg, 2.3 mmol) and ethylene glycol (0.13 mL, 2.3 mmol) in DCM (4.72 mL) was added DMAP (281.16 mg, 2.3 mmol) and DIC (1.15 mL, 2.3 mmol), and the resulting reaction mixture was stirred at 25° C. for 1 h. After LC-MS showed the starting material was consumed, the mixture was concentrated in vacuo to give a crude product, which was purified by prep-HPLC (TFA as additive) to give (S)-2-hydroxyethyl 2-(tert-butoxycarbonylamino)-3-methyl-butanoate (370 mg, 61.5% yield) as colorless oil. MS (ESI): 284.1 ([M+Na]⁺).

### Step (b) Preparation of 2-[(2S)-2-(tert-butoxycarbonylamino)-3-methyl-butanoyl]oxyethyl 6-[8-[tert-butoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (Compound 1.08C)

To a solution of 6-[8-[tert-butoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (100.0 mg, 0.150 mmol, compound 1.03C) and (S)-2-hydroxyethyl 2-(tert-butoxycarbonylamino)-3-methyl-butanoate (58.8 mg, 0.22 mmol) in DMF (5.11 mL) was added HATU (68.4 mg, 0.18 mmol) and DIPEA (0.1 mL, 0.6 mmol). The resulting reaction mixture was stirred at 25° C. for 12 h before it was poured into water (100 mL) and extracted with EtOAc (100 mL) three times. Combined organics was then washed with aq. CaCl₂ solution (100 mL) three times, brine (100 mL), dried with anhy. Na₂SO₄, filtered, and concentrated in vacuo. The residue was purified first by prep-TLC (DCM:MeOH=20:1) and then prep-HPLC (TFA as additive) to give 2-[(2S)-2-(tert-butoxycarbonylamino)-3-methyl-butanoyl]oxyethyl 6-[8-[tert-butoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (50 mg, 36.6% yield) as a yellow solid. MS (ESI): 910.5 ([M+H]⁺).

### Step (c) Preparation of 2-[(2S)-2-Amino-3-methyl-butanoyl]oxyethyl 6-[5-cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate

### Example 1.08

To a solution of 2-[(2S)-2-(tert-butoxycarbonylamino)-3-methyl-butanoyl]oxyethyl 6-[8-[tert-butoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (30.0 mg, 0.030 mmol) in DCM (3 mL) was added TFA (0.01 mL, 0.160 mmol), and the resulting mixture solution was stirred at 25° C. for 1 h. After LC-MS showed the starting material was consumed, the mixture solution was adjust to pH=6˜8 by aq. NH₄HCO₃ solution and evaporated to dryness under the reduced pressure. The residue was then purified by prep-HPLC (NH₄HCO₃ as additive) to give 2-(((S)-2-amino-3-methylbutanoyl)oxy)ethyl 6-(5-cyano-6-fluoro-8-(methylamino)-4-((3aS,6aS)-5-methylhexahydropyrrolo[2,3-c]pyrrol-1(2H)-yl)-9H-pyrido[2,3-b]indol-3-yl)-1-methyl-4-oxo-1,4-dihydro-1,8-naphthyridine-3-carboxylate (10.1 mg, 41.7% yield) as a yellow solid. MS (ESI): 710.2 ([M+H]⁺). ¹H NMR (400 MHz, DMSO-d₆) δ ppm: 11.89 (br s, 1H), 8.97 (d, J=2.4 Hz, 1H), 8.88 (s, 1H), 8.54 (d, J=2.4 Hz, 1H), 7.98 (s, 1H), 6.85 (br s, 1H), 6.64 (d, J=13.1 Hz, 1H), 4.64 (br d, J=6.2 Hz, 1H), 4.40-4.47 (m, 4H), 4.35-4.39 (m, 1H), 4.00 (s, 3H), 2.99 (d, J=4.6 Hz, 3H), 2.88-2.95 (m, 2H), 2.74 (br d, J=6.5 Hz, 2H), 2.38 (br d, J=8.4 Hz, 1H), 2.21 (br d, J=8.9 Hz, 3H), 2.08 (s, 3H), 1.88-1.94 (m, 1H), 1.69 (br dd, J=9.4, 5.0 Hz, 1H), 1.42 (br s, 1H), 0.86 (dd, 0.7=12.1, 6.8 Hz, 6H).

### Example 1.09

### 2-[[2-[[6-[5-Cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carbonyl]amino]acetyl]amino]acetic acid

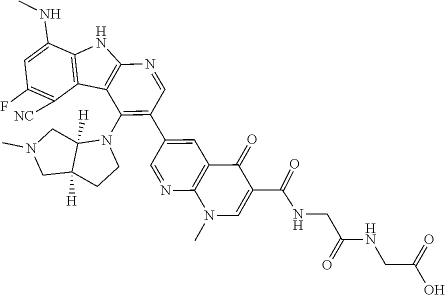 [SMILES: CNc1cc(F)c(C#N)c2c1[nH]c1ncc(-c3cnc4c(c3)c(=O)c(C(=O)NCC(=O)NCC(=O)O)cn4C)c(N3CC[C@H]4CN(C)C[C@H]43)c12]

The titled compound was synthesized according to the following scheme:

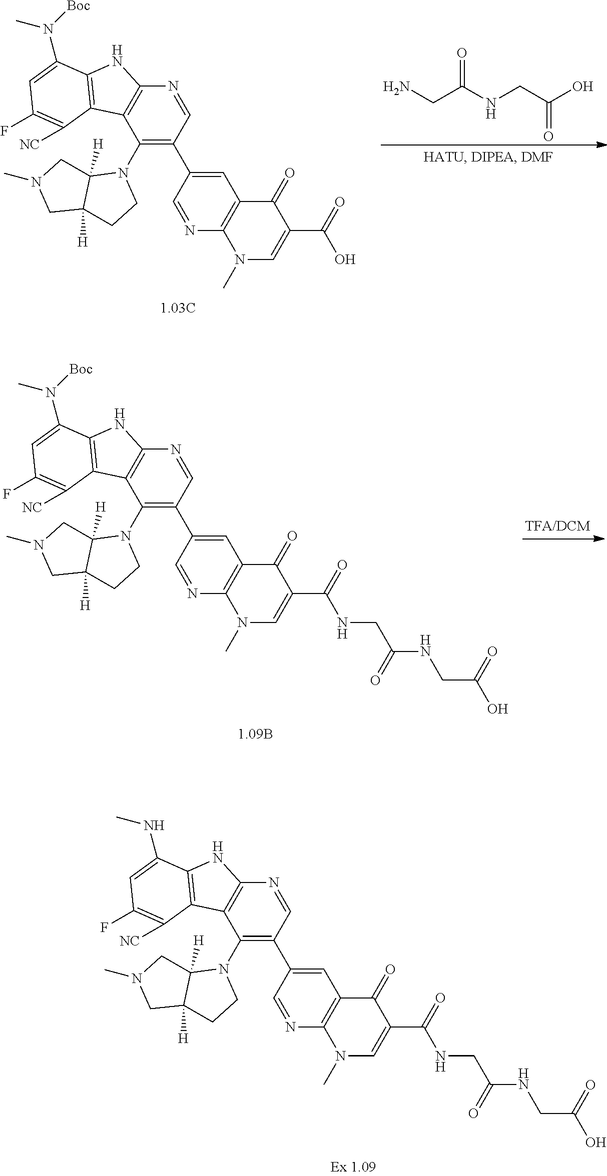 [SMILES: CN1C[C@@H]2CCN(c3c(-c4cnc5c(c4)c(=O)c(C(=O)O)cn5C)cnc4[nH]c5c(N(C)C(=O)OC(C)(C)C)cc(F)c(C#N)c5c34)[C@@H]2C1 ; NCC(=O)NCC(=O)O ; CN1C[C@@H]2CCN(c3c(-c4cnc5c(c4)c(=O)c(C(=O)NCC(=O)NCC(=O)O)cn5C)cnc4[nH]c5c(N(C)C(=O)OC(C)(C)C)cc(F)c(C#N)c5c34)[C@@H]2C1 ; CNc1cc(F)c(C#N)c2c1[nH]c1ncc(-c3cnc4c(c3)c(=O)c(C(=O)NCC(=O)NCC(=O)O)cn4C)c(N3CC[C@H]4CN(C)C[C@H]43)c12]

### Step (a) Preparation of 2-[[2-[[6-[8-[tert-butoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carbonyl]amino]acetyl]amino]acetic acid (Compound 1.09B)

To a solution of 6-[8-[tert-butoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (30.0 mg, 0.04 mmol, compound 1.03C) in DMF (1.5 mL) was added HATU (20.5 mg, 0.050 mmol) and DIPEA (0.02 mL, 0.130 mmol) under N₂. The mixture was stirred at 25° C. for 0.5 h before glycylglycine (11.9 mg, 0.09 mmol) was added and the resulting reaction mixture was stirred at 25° C. for another 2 h. After LC-MS showed the starting material was consumed completely, the mixture was subject to prep-HPLC purification (TFA as additive) to give 2-[[2-[[6-[8-[tert-butoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carbonyl]amino]acetyl]amino]acetic acid (16 mg, 44.2% yield) as a yellow solid. MS (ESI): 781.4 ([M+H]⁺).

### Step (b) Preparation of 2-[[2-[[6-[8-[tert-butoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carbonyl]amino]acetyl]amino]acetic acid (Example 1.09)

To a solution of 2-[[2-[[6-[8-[tert-butoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carbonyl]amino]acetyl]amino]acetic acid (14.0 mg, 0.020 mmol) in DCM (5 mL) was added TFA (0.04 mL, 0.540 mmol), and the resulting mixture was stirred at 25° C. for 1 h. After LC-MS showed the starting material was consumed completely, the mixture solution was concentrated in vacuo to give a crude product, which was purified by prep-HPLC (TFA as additive) to give 2-[[2-[[6-[5-cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carbonyl]amino]acetyl]amino]acetic acid (13.6 mg, 94.6% yield) as a yellow solid. MS (ESI): 681.2 ([M+H]⁺). ¹H NMR (400 MHz, DMSO-d6) δ ppm 11.99-12.16 (m, 1H), 9.99-10.16 (m, 1H), 9.34 (s, 1H), 9.07-9.19 (m, 2H), 8.67 (d, J=2.32 Hz, 1H), 8.41 (q, J=5.87 Hz, 1H), 8.06-8.25 (m, 1H), 6.94 (s, 1H), 6.66-6.79 (m, 1H), 4.64 (s, 1H), 4.06-4.17 (m, 5H), 3.81 (d, J=5.87 Hz, 2H), 3.02 (d, J=2.45 Hz, 4H), 2.66-2.89 (m, 5H), 2.55 (s, 2H), 2.06-2.28 (m, 1H).

### Example 1.10

### 6-[5-Cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-N-methylsulfonyl-4-oxo-1,8-naphthyridine-3-carboxamide

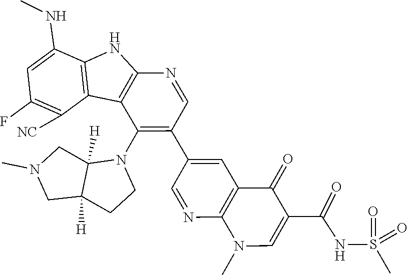 [SMILES: CNc1cc(F)c(C#N)c2c1[nH]c1ncc(-c3cnc4c(c3)c(=O)c(C(=O)NS(C)(=O)=O)cn4C)c(N3CC[C@H]4CN(C)C[C@H]43)c12]

To a solution of 6-[5-cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (80.0 mg, 0.14 mmol, compound 1.03D) in DCM (5 mL) was added thionyl chloride (33.6 mg, 0.280 mmol) at 0° C. Afterwards, the reaction mixture was allowed to warm up to r.t. and stirred at 25° C. for 2 h before it was added into a solution of methanesulfonamide (67.2 mg, 0.71 mmol) in DCM (5 mL). The resulting mixture solution was stirred at 25° C. for addition 16 h and then concentrated under the reduced pressure. The residue was subject to prep-HPLC (NH₃H₂O as additive) purification to give 6-[5-cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-N-methylsulfonyl-4-oxo-1,8-naphthyridine-3-carboxamid (8.1 mg, 9.0% yield) as a yellow solid. MS (ESI): 644.1 ([M+H]⁺). ¹H NMR (400 MHz, DMSO-d₆) δ ppm: 11.87 (br s, 1H), 9.18 (s, 1H), 9.10 (d, 0.7=2.2 Hz, 1H), 8.72 (d, 0.7=2.3 Hz, 1H), 8.00 (s, 1H), 6.82 (br d, 0.7=4.2 Hz, 1H), 6.65 (d, 0.7=13.0 Hz, 1H), 4.14 (s, 3H), 3.39 (br s, 3H), 2.99 (br d, 0.7=4.5 Hz, 3H), 2.93 (br d, 0.7=10.8 Hz, 2H), 2.73-2.79 (m, 2H), 2.41 (br d, 0.7=5.0 Hz, 2H), 2.19-2.23 (m, 2H), 2.11 (s, 3H), 1.69 (br dd, 0.7=9.8, 5.2 Hz, 1H), 1.42 (br s, 1H).

### Example 1.11

### 1-Acetoxyethyl 6-[5-cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate

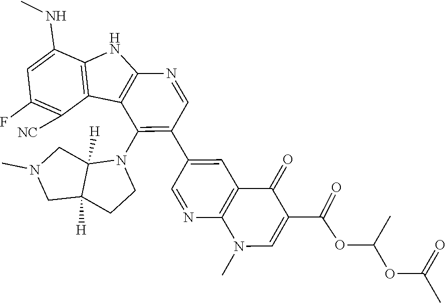 [SMILES: CNc1cc(F)c(C#N)c2c1[nH]c1ncc(-c3cnc4c(c3)c(=O)c(C(=O)OC(C)OC(C)=O)cn4C)c(N3CC[C@H]4CN(C)C[C@H]43)c12]

To a solution of 6-[5-cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (100 mg, 0.18 mmol, compound 1.03D) in DMF (1 mL) was added potassium carbonate (48.1 mg, 0.35 mmol), and the mixture was stirred at 20° C. for 0.5 h before 1-bromoethyl acetate (65.5 mg, 0.39 mmol) was added. The resulting mixture was stirred at 20° C. for another 16 h before concentrated under reduced pressure. The residue was subject to Prep-HPLC (TFA as additive) purification to give 1-acetoxyethyl 6-[5-cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (4.5 mg, 4.0% yield) as a yellow solid. MS (ESI): 653.3 ([M+H]⁺). ¹H NMR (400 MHz, DMSO-d₆) δ ppm: 12.08 (br s, 1H), 9.60 (br s, 1H), 9.07-8.95 (m, 2H), 8.53 (br s, 1H), 8.20-8.02 (m, 1H), 6.95 (q, J=5.3 Hz, 2H), 6.69 (br d, 0.7=13.1 Hz, 1H), 4.86-4.41 (m, 1H), 4.04 (s, 3H), 3.29-3.05 (m, 4H), 3.01 (s, 4H), 2.74-2.65 (m, 4H), 2.26-2.13 (m, 1H), 2.07 (s, 4H), 1.66 (br s, 1H), 1.53 (d, J=5.4 Hz, 3H).

### Example 1.12

### 1-Isopropoxycarbonyloxyethyl 6-[6-fluoro-5-methyl-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate

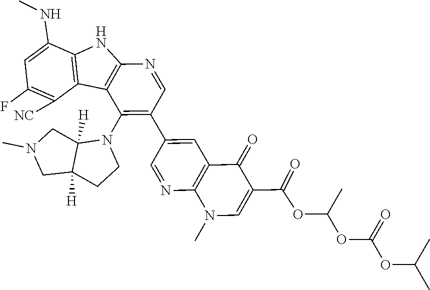 [SMILES: CNc1cc(F)c(C#N)c2c1[nH]c1ncc(-c3cnc4c(c3)c(=O)c(C(=O)OC(C)OC(=O)OC(C)C)cn4C)c(N3CC[C@H]4CN(C)C[C@H]43)c12]

To a solution of 6-[5-cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (100 mg, 0.18 mmol, compound 1.03D) in DMSO (2 mL) was added potassium carbonate (48.1 mg, 0.35 mmol), and the reaction mixture was stirred at 20° C. for 0.5 h before 1-chloroethyl isopropyl carbonate (67.3 mg, 0.40 mmol) was added. The resulting mixture was stirred at 20° C. for another 16 h and then concentrated under the reduced pressure. The residue was subject to prep-HPLC (TFA as additive) purification to give 1-isopropoxycarbonyloxyethyl 6-[6-fluoro-5-methyl-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (11.5 mg, 9.0% yield) as a yellow solid. MS (ESI): 697.4 ([M+H]⁺). ¹H NMR (400 MHz, DMSO-d₆) δ ppm: 12.14 (br s, 1H), 9.74 (br s, 1H), 9.08-8.89 (m, 2H), 8.53 (s, 1H), 8.20-8.00 (m, 1H), 7.21-7.03 (m, 1H), 6.90-6.79 (m, 1H), 6.68 (br d, J=12.8 Hz, 1H), 4.87-4.74 (m, 1H), 4.05 (s, 3H), 3.00 (s, 4H), 2.74-2.67 (m, 4H), 1.56 (d, J=5.4 Hz, 3H), 1.24 (d, J=6.2 Hz, 6H).

### Example 1.13

### (5-Methyl-2-oxo-1,3-dioxol-4-yl)methyl 6-[5-cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate

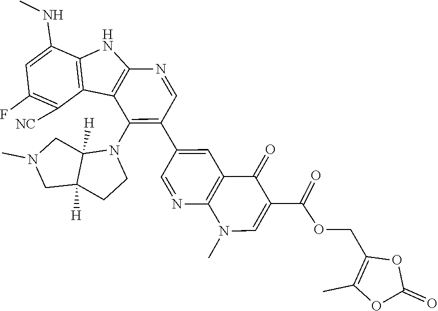 [SMILES: CNc1cc(F)c(C#N)c2c1[nH]c1ncc(-c3cnc4c(c3)c(=O)c(C(=O)OCc3oc(=O)oc3C)cn4C)c(N3CC[C@H]4CN(C)C[C@H]43)c12]

The titled compound was synthesized according to the following scheme:

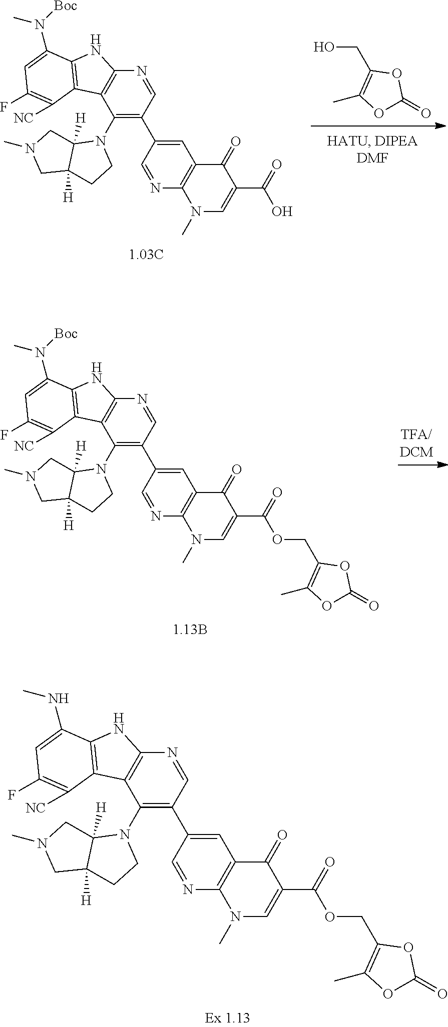

### Step (a) Preparation of (5-methyl-2-oxo-1,3-dioxol-4-yl)methyl 6-[8-[tert-butoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (Compound 1.13B)

A mixture of 6-[8-[tert-butoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (100 mg, 0.15 mmol, compound 1.03C), 4-(hydroxymethyl)-5-methyl-1,3-dioxol-2-one (100 mg, 0.77 mmol), DCC (100 mg, 0.48 mmol), and DMAP (30 mg, 0.25 mmol) in DMF (3.0 mL) was stirred at 20° C. for 12 h. After LC-MS showed the starting material was consumed, the mixture solution was concentrated in vacuo to give a crude product, which was purified by prep-HPLC (TFA as additive) to give (5-methyl-2-oxo-1,3-dioxol-4-yl)methyl 6-[8-[tert-butoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (45.0 mg, 38.5% yield) as an off-white solid. MS (ESI): 779.3 ([M+H]⁺).

### Step (b) Preparation of (5-methyl-2-oxo-1,3-dioxol-4-yl)methyl 6-[5-cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (Example 1.13)

To a solution of (5-methyl-2-oxo-1,3-dioxol-4-yl)methyl 6-[8-[tert-butoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (45.0 mg, 0.06 mmol) in DCM (3.0 mL) was added TFA (1.0 mL, 12.9 mmol), and the resulting reaction mixture was stirred at 20° C. for 1 h. After LC-MS showed the starting material was consumed, the mixture solution was concentrated in vacuo to give a crude product, which was purified by prep-HPLC (TFA as additive) to give (5-methyl-2-oxo-1,3-dioxol-4-yl)methyl 6-[5-cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (11.1 mg, 18.7% yield) as a yellow solid. MS (ESI): 679.3 ([M+H]⁺). ¹H NMR (400 MHz, DMSO-d6) δ ppm 12.03˜12.14 (m, 1H), 9.53 (br s, 1H), 8.95˜9.10 (m, 2H), 8.54 (d, J=2.32 Hz, 1H), 8.07˜8.20 (m, 1H), 8.07˜8.20 (m, 1H), 6.97 (br s, 1H), 6.67˜6.78 (m, 1H), 5.16 (s, 2H), 4.68 (br s, 1H), 4.05 (s, 3H), 3.67 (br s, 3H), 3.42 (br s, 1H), 3.23 (br s, 2H), 3.07˜3.15 (m, 1H), 3.02 (s, 3H), 2.65˜2.87 (m, 4H), 2.31˜2.36 (m, 1H), 2.24 (s, 3H), 2.04˜2.15 (m, 1H), 1.68 (br s, 1H).

### Example 1.14

### 6-[8-[(2-Aminoacetyl)oxymethoxycarbonyl-methyl-amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid

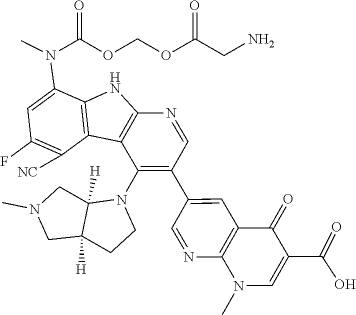 [SMILES: CN1C[C@@H]2CCN(c3c(-c4cnc5c(c4)c(=O)c(C(=O)O)cn5C)cnc4[nH]c5c(N(C)C(=O)OCOC(=O)CN)cc(F)c(C#N)c5c34)[C@@H]2C1]

The titled compound was synthesized according to the following scheme:

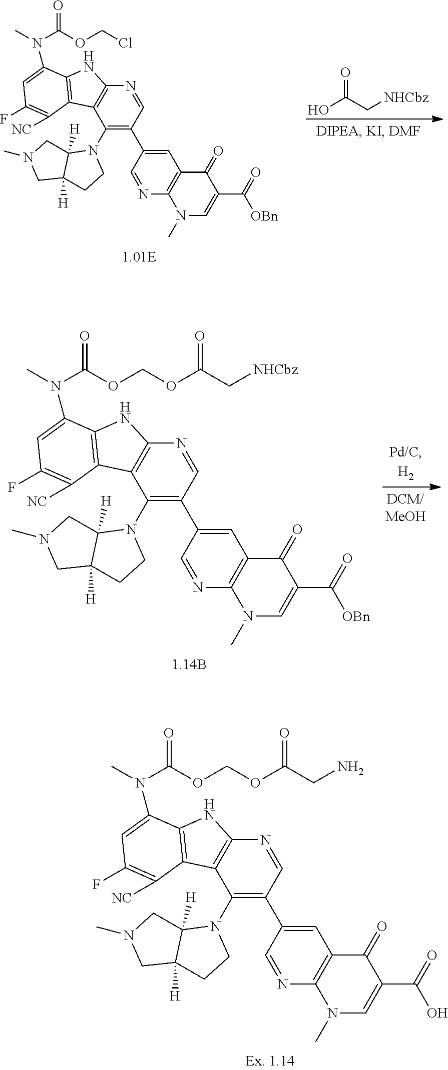 [SMILES: CN1C[C@@H]2CCN(c3c(-c4cnc5c(c4)c(=O)c(C(=O)OCc4ccccc4)cn5C)cnc4[nH]c5c(N(C)C(=O)OCCl)cc(F)c(C#N)c5c34)[C@@H]2C1 ; O=C(O)CNC(=O)OCc1ccccc1 ; CN1C[C@@H]2CCN(c3c(-c4cnc5c(c4)c(=O)c(C(=O)OCc4ccccc4)cn5C)cnc4[nH]c5c(N(C)C(=O)OCOC(=O)CNC(=O)OCc6ccccc6)cc(F)c(C#N)c5c34)[C@@H]2C1 ; CN1C[C@@H]2CCN(c3c(-c4cnc5c(c4)c(=O)c(C(=O)O)cn5C)cnc4[nH]c5c(N(C)C(=O)OCOC(=O)CN)cc(F)c(C#N)c5c34)[C@@H]2C1]

### Step (a) Preparation of benzyl 6-[8-[[2-(benzyloxycarbonylamino)acetyl]oxymethoxycarbonyl-methyl-amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (Compound 1.14B)

To a solution of benzyl 6-[8-[chloromethoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (200.0 mg, 0.270 mmol, compound 1.01E), DIPEA (0.23 mL, 1.33 mmol), and potassium iodide (0.07 mL, 1.33 mmol) in DMF (12 mL) was added N-benzyloxycarbonylglycine (279.23 mg, 1.33 mmol, CAS: 1138-80-3). The resulting reaction mixture was stirred at 50° C. for 2 h until LC-MS showed the starting material was consumed completely. The mixture solution was diluted with EtOAc (300.0 mL) and washed with water (100.0 mL) three times, and brine (100 mL) three times. The organic layer was dried over anhy. Na₂SO₄, filtered, and concentrated in vacuo to give a crude product, which was purified by prep-HPLC (formic acid as additive) purification. The collected eluents was basified to pH >7 with NH₄HCO₃ solid and extracted with EtOAc (150.0 mL) three times. Combined organics was dried over anhy. Na₂SO₄, filtered, and evaporated to dryness to give benzyl 6-[8-[[2-(benzyloxycarbonylamino)acetyl]oxymethoxycarbonyl-methyl-amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (150 mg, 55.6% yield) as a yellow solid. MS (ESI): 922.4 (([{³⁵Cl}M+H]⁺).

### Step (b) Preparation of 6-[8-[(2-aminoacetyl)oxymethoxycarbonyl-methyl-amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (Example 1.14)

A solution of benzyl 6-[8-[[2-(benzyloxycarbonylamino)acetyl]oxymethoxycarbonyl-methyl-amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (130.0 mg, 0.140 mmol) in DCM (0.919 mL) and MeOH (0.919 mL) was added palladium on carbon (130 mg). After the suspension was degassed under vacuum and purged with H₂ several times, the mixture solution was stirred at 25° C. for 3 h under H₂ atmosphere (15 psi). After LC-MS showed the starting material was consumed completely, the reaction mixture was filtered through Celite. The filtrate was then concentrated in vacuo to give a crude product of 6-[8-[(2-aminoacetyl)oxymethoxycarbonyl-methyl-amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (50 mg, 20.6% yield) as a yellow solid. MS (ESI): 698.3 ([M+H]⁺).

### Example 1.15

### 6-[8-[1-Acetoxyethoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid

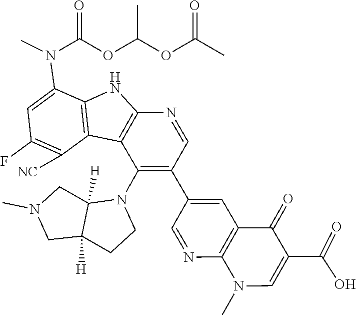 [SMILES: CC(=O)OC(C)OC(=O)N(C)c1cc(F)c(C#N)c2c1[nH]c1ncc(-c3cnc4c(c3)c(=O)c(C(=O)O)cn4C)c(N3CC[C@H]4CN(C)C[C@H]43)c12]

The titled compound was synthesized according to the following scheme:

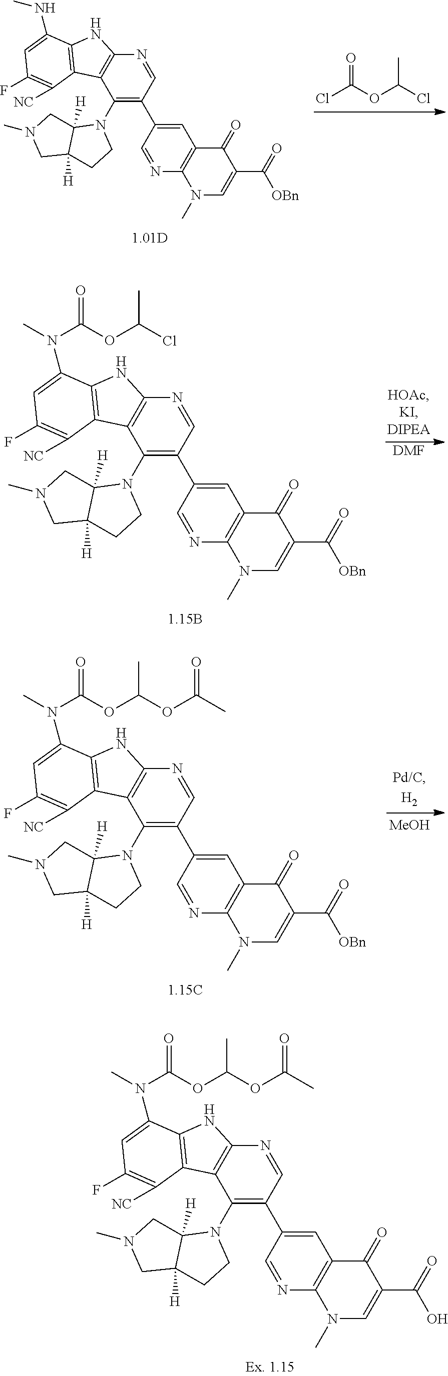

### Step (a) Preparation of benzyl 6-[8-[1-chloroethoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (Compound 1.15)

A mixture solution of benzyl 6-[5-cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (200 mg, 0.30 mmol, compound 1.01D) and 1-chloroethyl chloroformate (40.0 g, 279.8 mmol) was stirred at 25° C. for 12 h. After LC-MS showed the starting material was consumed completely, the mixture was concentrated in vacuo to give a crude product of benzyl 6-[8-[1-chloroethoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (180 mg, 77.4% yield) as a light yellow solid. MS (ESI): 763.2 ([M+H]⁺). It was used directly in the next step without further purification.

### Step (b) Preparation of benzyl 6-[8-[1-acetoxyethoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (Compound 1.15C)

To a solution of benzyl 6-[8-[1-chloroethoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (150 mg, 0.20 mmol), DIPEA (0.17 mL, 0.98 mmol), and potassium iodide (0.05 mL, 0.980 mmol) in DMF (15 mL) was added acetic acid (59.0 mg, 0.98 mmol). The resulting reaction mixture was stirred at 50° C. for 2 h until LC-MS showed the starting material was consumed. The mixture solution was diluted with EtOAc (400.0 mL) and washed with water (100.0 mL) three times and brine (100 mL) three times. Combined organics was dried over anhy. Na₂SO₄, filtered, and concentrated in vacuo to give a crude product, which was purified by prep-TLC (EtOAc:MeCN=1:1) to give benzyl 6-[8-[1-acetoxy ethoxy carbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (90 mg, 46.0% yield) as a yellow solid. MS (ESI): 787.3 ([M+H]⁺).

### Step (c) Preparation of 6-[8-[1-acetoxyethoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (Example 1.15)

To a solution of benzyl 6-[8-[1-acetoxyethoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (80.0 mg, 0.10 mmol) in THF (4 mL) was added palladium on carbon (80.0 mg). After the suspension was degassed under vacuum and purged with H₂ several times, the mixture solution was stirred at 25° C. for 1 h under H₂ atmosphere (15 psi). After LC-MS showed the starting material was consumed completely, the reaction mixture was filtered through Celite. The filtrate was then concentrated in vacuo to give a crude product, which was purified by prep-HPLC (NH₄HCO₃ as additive) to give 6-[8-[1-acetoxyethoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (30.3 mg, 42.4% yield) as a light yellow solid. MS (ESI): 697.2 ([M+H]⁺). ¹H NMR (400 MHz, DMSO-d₆) δ ppm 12.65 (br s, 1H), 9.08-9.39 (m, 2H), 8.75 (br s, 1H), 8.12 (br s, 1H), 7.72 (br d, J=10.8 Hz, 1H), 6.77 (br s, 1H), 4.58 (br s, 1H), 4.16 (br s, 3H), 3.44-3.60 (m, 2H), 2.79-3.02 (m, 3H), 2.53 (d, J=2.0 Hz, 2H), 2.45 (br s, 1H), 1.97-2.27 (m, 9H), 1.43-1.72 (m, 3H), 1.18 (br s, 2H).

### Example 1.16

### 6-[8-[Diethoxyphosphoryl(methyl)amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid

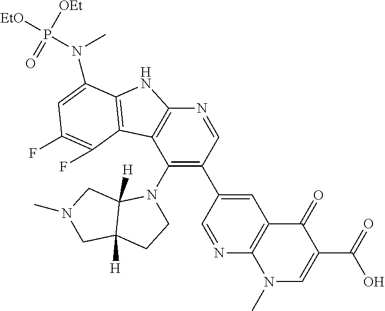 [SMILES: CCOP(=O)(OCC)N(C)c1cc(F)c(F)c2c1[nH]c1ncc(-c3cnc4c(c3)c(=O)c(C(=O)O)cn4C)c(N3CC[C@@H]4CN(C)C[C@@H]43)c12]

The titled compound was synthesized according to the following scheme:

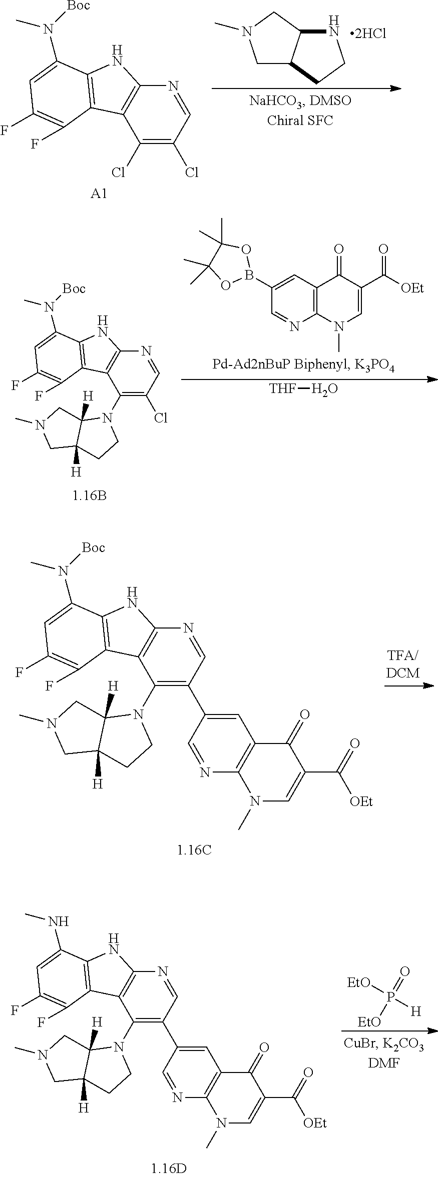

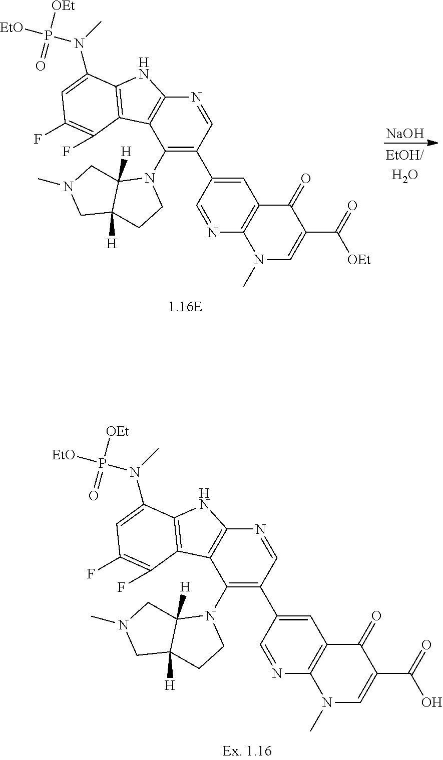 [SMILES: CCOC(=O)c1cn(C)c2ncc(-c3cnc4[nH]c5c(N(C)P(=O)(OCC)OCC)cc(F)c(F)c5c4c3N3CC[C@@H]4CN(C)C[C@@H]43)cc2c1=O ; "NaOH" ; CCOP(=O)(OCC)N(C)c1cc(F)c(F)c2c1[nH]c1ncc(-c3cnc4c(c3)c(=O)c(C(=O)O)cn4C)c(N3CC[C@@H]4CN(C)C[C@@H]43)c12]

### Step (a) Preparation of tert-butyl N-[3-chloro-5,6-difluoro-4-[(3aR,6aR)-5-methyl-2,3,3a,4,6,6a-hexahydropyrrolo[2,3-c]pyrrol-1-yl]-9H-pyrido[2,3-b]indol-8-yl]-N-methyl-carbamate (Compound 1.16B)

A mixture solution of tert-butyl N-(3,4-dichloro-5,6-difluoro-9H-pyrido[2,3-b]indol-8-yl)-N-methyl-carbamate (24.0 g, 59.67 mmol, Intermediate A1), 5-methyl-1H-hexahydropyrrolo[2,3-c]pyrrole dihydrochloride (14.9 g, 74.59 mmol, CAS: 1197193-15-9) and NaHCO₃ (25.1 g, 298.4 mmol) in DMSO (40.0 mL) was stirred at 45° C. for 1 h until evolution of CO₂ had ceased. The reaction mixture was then allowed to heated at 120° C. for another 47 h until LC-MS showed the starting material was consumed completely. The mixture was cooled back to r.t., poured into water (500.0 mL), and the resulting precipitate was collected by filtration. The filter cake was then recrystallized in MeOH/EtOAc (v/v=5:1, 400 mL) to give a racemic mixture of compound 1.16B as a white solid. It was subject to chiral SFC separation (column: Chiralcel OD, 50×4.6 mm I.D., particle size 3 μm (Daicel); mobile phase: A: 95% CO₂, B: 5% IPA (0.05% Et₂NH); flow rate: 3 mL/min)) to give tert-butyl N-[3-chloro-5,6-difluoro-4-[(3aR,6aR)-5-methyl-2,3,3a,4,6,6a-hexahydropyrrolo[2,3-c]pyrrol-1-yl]-9H-pyrido[2,3-b]indol-8-yl]-N-methyl-carbamate (9.9 g, 41.1% yield, >98% ee, optical rotation [α]_{D}=+26.6°, HPLC Rf=6.29 min) as a white solid. MS (ESI): 492.2 ([{³⁵Cl}M+H]⁺), 494.2 ([{³⁷Cl}M+H]⁺). Another enantiomeric product obtained matched with compound 1.26 B, tert-butyl N-[3-chloro-5,6-difluoro-4-[(3aR,6aR)-5-methyl-2,3,3a,4,6,6a-hexahydropyrrolo[2,3-c]pyrrol-1-yl]-9H-pyrido[2,3-b]indol-8-yl]-N-methyl-carbamate (9.4 g, 39% yield, >98% ee, optical rotation [α]_{D}=+27.7°, HPLC Rf=5.45 min).

### Step (b) Preparation of ethyl 6-[8-[tert-butoxycarbonyl(methyl)amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (Compound 1.160

To a solution of tert-butyl N-[3-chloro-5,6-difluoro-4-[(3aR,6aR)-5-methyl-2,3,3a,4,6,6a-hexahydropyrrolo[2,3-c]pyrrol-1-yl]-9H-pyrido[2,3-b]indol-8-yl]-N-methyl-carbamate (100.0 mg, 0.203 mmol), benzyl 1-methyl-4-oxo-6-(4,4,5,5-tetramethyl-1,3,2-dioxaborolan-2-yl)-1,8-naphthyridine-3-carboxylate (109 mg, 0.31 mmol, Intermediate B3), and K₃PO₄ (172 mg, 0.812 mmol) in THF/H₂O (4.0 mL, v/v=5:l) was added Pd-Ad2nBuP Biphenyl (27.1 mg, 0.04 mmol, cataCXium A-Pd-G2, CAS: 1375477-29-4) in glovebox under argon. The mixture solution was stirred at 70° C. for 12 h until LC-MS showed the starting material was consumed completely. The mixture was then cooled back to r.t., poured into water (50 mL) and extracted with EtOAc (100 mL) twice. Combined organics was washed with brine (50 mL) twice, dried with anhy. Na₂SO₄, filtered, and concentrated in vacuo to give a crude product, which was purified by prep-HPLC (TFA as additive) to give ethyl 6-[8-[tert-butoxycarbonyl(methyl)amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (115.0 mg, 82.3% yield) as a yellow solid. MS (ESI): 688.4 ([M+H]⁺).

### Step (c) Preparation of ethyl 6-[5,6-difluoro-8-(methylamino)-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (Compound 1.16D)

To a solution of ethyl 6-[8-[tert-butoxycarbonyl(methyl)amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (300 mg, 0.44 mmol) in DCM (5 mL) was added TFA (18.95 mL, 75.8 mmol), and the resulting mixture solution was stirred at 20° C. for 2 h. After LC-MS showed the starting material was consumed, the mixture was concentrated in vacuo to give a crude product, which was purified by prep-HPLC (TFA as additive) to give ethyl 6-[5,6-difluoro-8-(methylamino)-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (200 mg, 78.0% yield) as a yellow solid. MS (ESI): 588.3 ([M+H]⁺).

### Step (d) Preparation of ethyl 6-[8-[diethoxyphosphoryl(methyl)amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (Compound 1.16E)

To a solution of ethyl 6-[5,6-difluoro-8-(methylamino)-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (50.0 mg, 0.09 mmol) in DMF (2 mL) was added diethyl phosphite (23.5 mg, 0.17 mmol) and CuBr (15.9 mg, 0.11 mmol) under nitrogen. The mixture was stirred at 60° C. for 15 h until LC-MS showed the starting material was consumed completely. The mixture was cooled back to r.t. and concentrated in vacuo to give a crude product, which was purified by prep-HPLC (TFA as additive) to give ethyl 6-[8-[diethoxyphosphoryl(methyl)amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (15 mg, 24.4% yield) as a black solid. MS (ESI): 724.3 ([M+H]⁺).

### Step (e) Preparation of 6-[8-[diethoxyphosphoryl(methyl)amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (Example 1.16)

To a solution of ethyl 6-[8-[diethoxyphosphoryl(methyl)amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (74.0 mg, 0.10 mmol) in EtOH (2 mL) was added NaOH (41.9 mg, 1.02 mmol), and the resulting mixture was stirred at 25° C. for 2 h. After LC-MS showed the starting material was consumed completely, the solution was acidified with aq. HCl solution (1N) to pH=6, and extracted with DCM (50 mL) three times. Combined organics was washed with brine, dried over anhy. Na₂SO₄, filtered and concentrated in vacuo to give a crude product, which was purified by prep-HPLC (TFA as additive). The combined eluents were evaporated to dryness under the reduced pressure, and the residue was re-dissolved in MeOH and basified with aq. NaHCO₃ solution to pH=7. The solution was subject to another Prep-HPLC (NH₄HCO₃ as additive) purification to give 6-[8-[diethoxyphosphoryl(methyl)amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (21.6 mg, 29.2% yield) as a white solid. MS (ESI): 696.4 ([M+H]⁺). ¹H NMR (400 MHz, DMSO-d6) δ12.51 (br s, 1H), 9.29 (s, 1H), 9.12 (br s, 1H), 8.69 (d, J=2.1 Hz, 1H), 8.31 (br d, J=15.5 Hz, 1H), 7.54-7.42 (m, 1H), 7.35-7.04 (m, 1H), 4.19 (s, 3H), 4.17-4.06 (m, 4H), 3.66 (br s, 1H), 3.57-3.32 (m, 3H), 3.12 (br d, J=9.3 Hz, 2H), 3.19-2.99 (m, 1H), 3.01 (br s, 1H), 2.94-2.78 (m, 2H), 2.74-2.63 (m, 4H), 2.21-1.99 (m, 1H), 1.96-1.75 (m, 1H), 1.35 (s, 2H), 1.29-1.16 (m, 7H).

### Example 1.17

### 6-[8-[[Benzyloxy(hydroxy)phosphoryl]-methyl-amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid

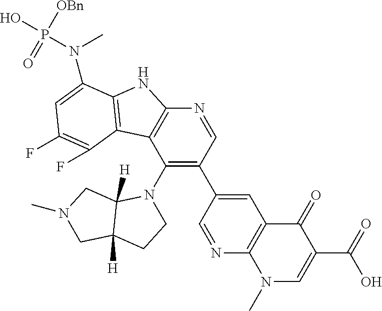 [SMILES: CN1C[C@H]2CCN(c3c(-c4cnc5c(c4)c(=O)c(C(=O)O)cn5C)cnc4[nH]c5c(N(C)P(=O)(O)OCc6ccccc6)cc(F)c(F)c5c34)[C@H]2C1]

The titled compound was synthesized according to the following scheme:

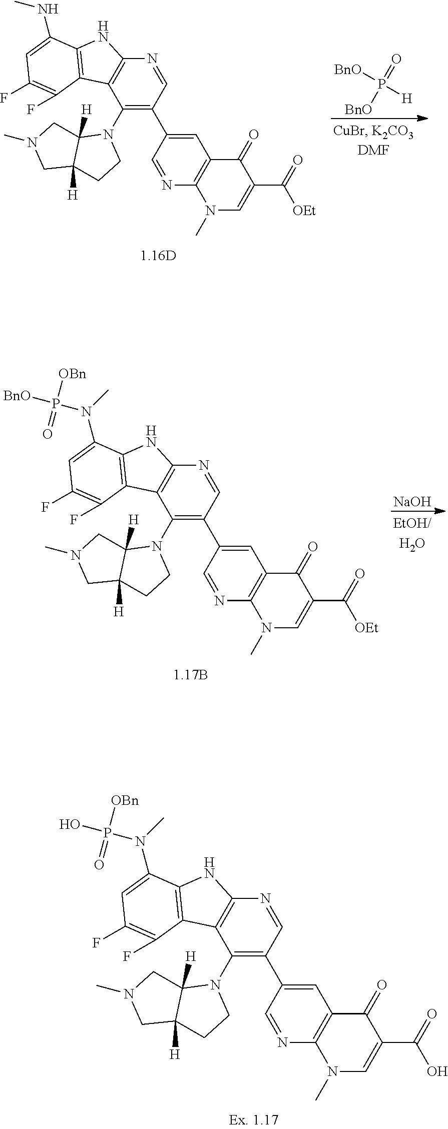 [SMILES: CCOC(=O)c1cn(C)c2ncc(-c3cnc4[nH]c5c(NC)cc(F)c(F)c5c4c3N3CC[C@@H]4CN(C)C[C@@H]43)cc2c1=O ; O=[PH](OCc1ccccc1)OCc1ccccc1 ; CCOC(=O)c1cn(C)c2ncc(-c3cnc4[nH]c5c(N(C)P(=O)(OCc6ccccc6)OCc6ccccc6)cc(F)c(F)c5c4c3N3CC[C@@H]4CN(C)C[C@@H]43)cc2c1=O ; "NaOH" ; CN1C[C@H]2CCN(c3c(-c4cnc5c(c4)c(=O)c(C(=O)O)cn5C)cnc4[nH]c5c(N(C)P(=O)(O)OCc6ccccc6)cc(F)c(F)c5c34)[C@H]2C1]

### Step (a) Preparation of ethyl 6-[8-[dibenzyloxyphosphoryl(methyl)amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (Compound 1.17B)

To a solution of ethyl 6-[5,6-difluoro-8-(methylamino)-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (150.0 mg, 0.260 mmol, compound 1.16D) in DMF (2 mL) was added Et₃N (129.15 mg, 1.28 mmol), dibenzyl phosphite (133.88 mg, 0.510 mmol) and CuBr (47.74 mg, 0.330 mmol). The mixture solution was stirred at 50° C. for 15 h until LC-MS showed the starting material was consumed completely. The mixture solution was cooled back to r.t., concentrated in vacuo to give a crude product, which was purified by prep-HPLC (TFA as additive) to give ethyl 6-[8-[dibenzyloxyphosphoryl(methyl)amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (90 mg, 44.9% yield) as a yellow solid. MS (ESI): 848.4 ([M+H]⁺).

### Step (b) Preparation of 6-[8-[[benzyloxy(hydroxy)phosphoryl]-methyl-amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (Example 1.17)

To a solution of ethyl 6-[8-[dibenzyloxyphosphoryl(methyl)amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (90.0 mg, 0.110 mmol) in EtOH (2 mL) was added aq. NaOH solution (2.0 mL, 4 mmol), and the resulting solution was stirred at 65° C. for 15 h. After LC-MS showed the starting material was consumed completely, the mixture was acidified with aq. HCl solution (1N) to pH=7. The solution was subject to prep-HPLC (NH₃.H₂O as additive) purification to give benzyloxy(hydroxy)phosphoryl]-methyl-amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (5.1 mg, 6.5% yield) as a yellow solid. MS (ESI): 730.3 ([M+H]⁺). ¹H NMR (400 MHz, DMSO-d₆) δ 14.81 (br s, 1H), 14.18 (br s, 1H), 9.27 (s, 1H), 9.14 (br s, 1H), 8.71 (br s, 1H), 8.16 (br s, 1H), 7.25 (br s, 1H), 7.23-7.05 (m, 4H), 7.00 (br s, 1H), 7.05-6.93 (m, 1H), 4.78-4.27 (m, 3H), 4.18 (s, 3H), 3.22 (br d, J=7.0 Hz, 7H), 2.89 (br s, 1H), 2.97-2.76 (m, 1H), 2.46-2.44 (m, 1H), 2.06 (br d, J=18.3 Hz, 1H), 1.70 (br s, 2H).

### Example 1.18

### 6-[5,6-Difluoro-8-[methyl(phosphonooxymethoxycarbonyl)amino]-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid

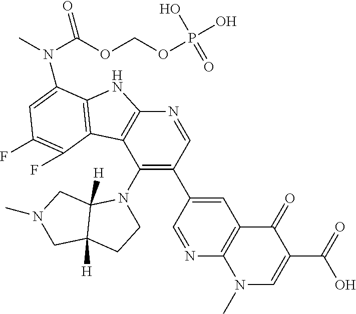 [SMILES: CN1C[C@H]2CCN(c3c(-c4cnc5c(c4)c(=O)c(C(=O)O)cn5C)cnc4[nH]c5c(N(C)C(=O)OCOP(=O)(O)O)cc(F)c(F)c5c34)[C@H]2C1]

The titled compound was synthesized according to the following scheme:

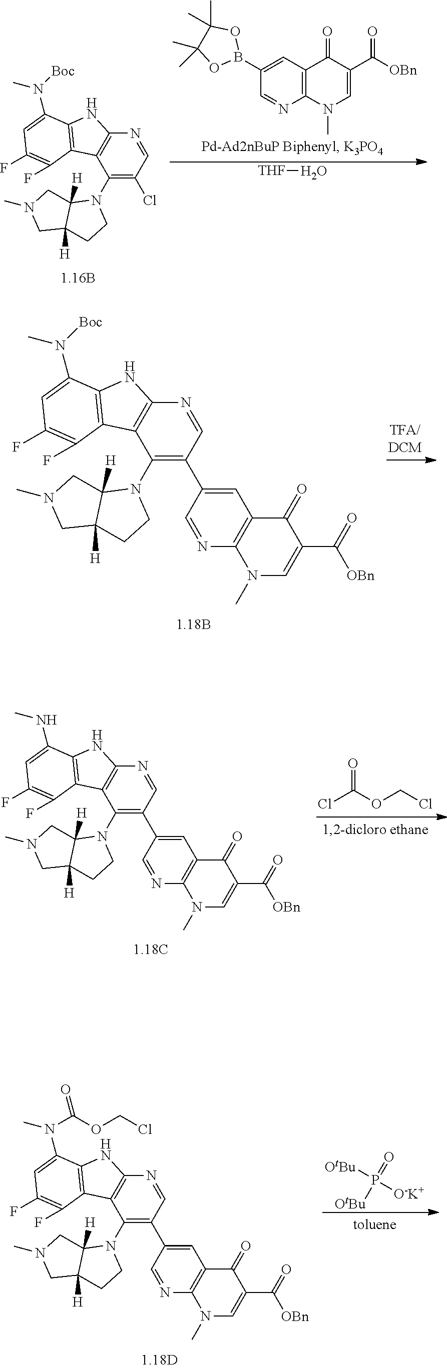

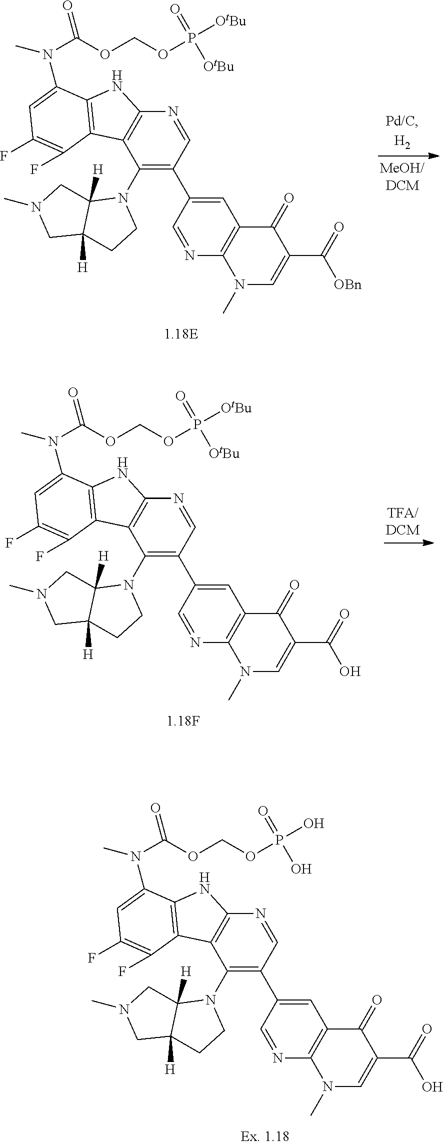 [SMILES: CN1C[C@H]2CCN(c3c(-c4cnc5c(c4)c(=O)c(C(=O)OCc4ccccc4)cn5C)cnc4[nH]c5c(N(C)C(=O)OCOP(=O)(OC(C)(C)C)OC(C)(C)C)cc(F)c(F)c5c34)[C@H]2C1 ; CN1C[C@H]2CCN(c3c(-c4cnc5c(c4)c(=O)c(C(=O)O)cn5C)cnc4[nH]c5c(N(C)C(=O)OCOP(=O)(OC(C)(C)C)OC(C)(C)C)cc(F)c(F)c5c34)[C@H]2C1 ; CN1C[C@H]2CCN(c3c(-c4cnc5c(c4)c(=O)c(C(=O)O)cn5C)cnc4[nH]c5c(N(C)C(=O)OCOP(=O)(O)O)cc(F)c(F)c5c34)[C@H]2C1]

### Step (a) Preparation of benzyl 6-[8-[tert-butoxycarbonyl(methyl)amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (Compound 1.18B)

To a solution of tert-butyl N-[3-chloro-5,6-difluoro-4-[(3aR,6aR)-5-methyl-2,3,3a,4,6,6a-hexahydropyrrolo[2,3-c]pyrrol-1-yl]-9H-pyrido[2,3-b]indol-8-yl]-N-methyl-carbamate (500 mg, 1.02 mmol, compound 1.16B) in THF (10 mL) and water (1 mL) was added benzyl 1-methyl-4-oxo-6-(4,4,5,5-tetramethyl-1,3,2-dioxaborolan-2-yl)-1,8-naphthyridine-3-carboxylate (640 mg, 1.52 mmol, Intermediate B3), Pd-Ad2nBuP Biphenyl (34 mg, 0.05 mmol, cataCXium A-Pd-G2, CAS: 1375477-29-4), and K₃PO₄ (647.21 mg, 3.05 mmol) under nitrogen. The resulting reaction mixture was stirred at 70° C. for 12 h until LC-MS showed the start material was consumed completely. The reaction mixture was then cooled back to r.t., poured into water (300 mL), and extracted with EtOAc (100 mL) three times. Combined organics was washed with brine, dried over anhy. Na₂SO₄, filtered and concentrated in vacuo to give a crude product, which was purified by prep-HPLC (TFA as additive) to give benzyl 6-[8-[tert-butoxycarbonyl(methyl)amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (350 mg, 36.5% yield) as a yellow solid. MS (ESI): 750.3 (M+H)⁺.

### Step (b) Preparation of benzyl 6-[5,6-difluoro-8-(methylamino)-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (Compound 1.18C)

To a solution of benzyl 6-[8-[tert-butoxy carbonyl(methyl)amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (2.5 g, 3.33 mmol) in DCM (30 mL) was added TFA (1.03 mL, 13.3 mmol), and the resulting reaction mixture was stirred at 20° C. for 2 h. After LC-MS showed the starting material was consumed completely, the mixture was concentrated in vacuo to give a crude product, which was purified by prep-HPLC (TFA as additive) to give benzyl 6-[5,6-difluoro-8-(methylamino)-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (1.8 g, 83.1% yield) as a yellow solid. MS (ESI): 650.1 (M+H)⁺.

### Step (c) Preparation of benzyl 6-[8-[chloromethoxycarbonyl(methyl)amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (Compound 1.18D)

To a solution of benzyl 6-[5,6-difluoro-8-(methylamino)-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (1.7 g, 2.62 mmol) in 1,2-dichloro ethane (340 mL) was added chloromethyl chloroformate (1.33 g, 10.47 mmol), and the reaction mixture was stirred at 20° C. for 2 h. After LC-MS showed the starting material was consumed completely, the reaction mixture was concentrated in vacuo to give a crude product, which was then purified by prep-HPLC (TFA as additive). The collected eluents were adjust to pH >7 with aq. NH₄HCO₃ solution to give benzyl 6-[8-[chloromethoxycarbonyl(methyl)amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (1.1 g, 56.6% yield) as a yellow solid. MS (ESI): 742.3 (M+H)⁺.

### Step (d) Preparation of benzyl 6-[8-[di-tert-butoxyphosphoryloxymethoxycarbonyl(methyl)amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (Compound 1.18E)

To a solution of benzyl 6-[8-[chloromethoxycarbonyl(methyl)amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (200 mg, 0.27 mmol) in toluene (8 mL) was added potassium di-tert-butyl phosphate (201 mg, 0.81 mmol). The resulting reaction mixture was stirred at 90° C. for 2 h until LC-MS showed the starting material was consumed completely. The mixture solution was cooled back to r.t., poured into water (50 mL), and extracted with DCM (50 mL) three times. Combined organics was dried over anhy. Na₂SO₄, filtered, and concentrated in vacuo to give a crude product, which was then purified by prep-HPLC (TFA as additive). The collected eluents were adjust to pH >7 by aq. NH₄HCO₃ solution to give benzyl 6-[8-[di-tert-butoxyphosphoryloxymethoxycarbonyl(methyl)amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (50 mg, 20.3% yield) as a yellow solid. MS (ESI): 916.5 (M+H)⁺.

### Step (e) Preparation of 6-[8-[di-tert-butoxyphosphoryloxymethoxycarbonyl(methyl)amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (Compound 1.18F)

To a solution of benzyl 6-[8-[di-tert-butoxyphosphoryloxymethoxycarbonyl(methyl)amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (50 mg, 0.05 mmol) in DCM (1 mL) and MeOH (1 mL) was added palladium (0.58 mg, 0.01 mmol). After degassed under vacuum and flushed with H₂ several times, the reaction mixture was stirred at 10° C. for 1 h under a H₂ balloon. After LC-MS showed the starting material was consumed, mixture was filtered through Celite. The filtrate was concentrated in vacuo to give a crude product of 6-[8-[di-tert-butoxyphosphoryloxymethoxycarbonyl(methyl)amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (30 mg, 66.6% yield) as a yellow solid. MS (ESI): 826.5 (M+H)⁺. It was used directly in the next step without further purification.

### Step (f) Preparation of 6-[5,6-difluoro-8-[methyl(phosphonooxymethoxycarbonyl)amino]-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (Example 1.18)

To a solution of 6-[8-[di-tert-butoxyphosphorylmethoxycarbonyl(methyl)amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (29.0 mg, 0.040 mmol) in DCM (1 mL) was added TFA (0.05 mL), and the resulting mixture was stirred at 10° C. for 0.5 h. After LC-MS showed the starting material was consumed, the reaction mixture was adjust to pH >7 with aq. NH₄HCO₃ solution. It was subject to prep-HPLC (NH₄HCO₃ as additive) purification to give 6-[5,6-difluoro-8-[methyl(phosphonooxymethoxycarbonyl)amino]-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (19.5 mg, 74.9% yield) as a white solid. MS (ESI): 714.3 ([M+H]⁺). ¹H NMR (400 MHz, DMSO-d₆) δ ppm 9.26 (s, 1H) 9.14 (br s, 1H) 8.72 (br d, J=6 Hz, 1H) 8.15 (s, 1H) 7.53-7.69 (m, 1H) 5.35-5.66 (m, 2H) 4.43 (br s, 1H) 4.17 (s, 3H) 3.16-3.25 (m, 2H) 2.98 (br s, 2H) 2.83 (br s, 1H) 2.53 (d, J=2 Hz, 1H) 2.21 (br s, 1H) 2.06-2.16 (m, 3H) 1.99 (br s, 1H) 1.89 (br s, 1H) 1.65 (br s, 1H).

### Example 1.19

### 6-[8-[[tert-Butoxy(hydroxy)phosphoryl]oxymethoxycarbonyl-methyl-amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid

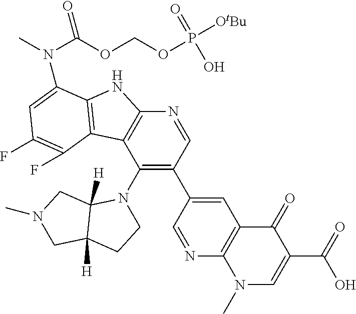 [SMILES: CN1C[C@H]2CCN(c3c(-c4cnc5c(c4)c(=O)c(C(=O)O)cn5C)cnc4[nH]c5c(N(C)C(=O)OCOP(=O)(O)OC(C)(C)C)cc(F)c(F)c5c34)[C@H]2C1]

The titled compound was synthesized according to the following scheme:

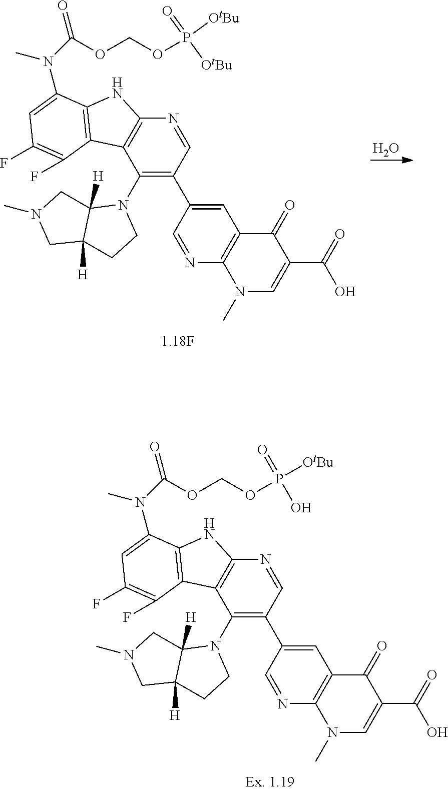 [SMILES: CN1C[C@H]2CCN(c3c(-c4cnc5c(c4)c(=O)c(C(=O)O)cn5C)cnc4[nH]c5c(N(C)C(=O)OCOP(=O)(OC(C)(C)C)OC(C)(C)C)cc(F)c(F)c5c34)[C@H]2C1 ; O ; CN1C[C@H]2CCN(c3c(-c4cnc5c(c4)c(=O)c(C(=O)O)cn5C)cnc4[nH]c5c(N(C)C(=O)OCOP(=O)(O)OC(C)(C)C)cc(F)c(F)c5c34)[C@H]2C1]

A solution of 6-[8-[di-tert-butoxyphosphoryloxymethoxycarbonyl(methyl)amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (30.0 mg, 0.040 mmol, compound 1.18F) in water (1 mL) was stirred at 60° C. for 0.5 h. After LC-MS showed the starting material was consumed, the reaction mixture was subject to prep-HPLC (NH₄HCO₃ as additive) purification to give 6-[8-[[tert-butoxy(hydroxy)phosphoryl]oxymethoxycarbonyl-methyl-amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (11 mg, 37.8% yield) as a yellow solid. MS (ESI): 770.5 ([M+H]⁺). ¹H NMR (400 MHz, DMSO-d₆) δ ppm 14.57-14.90 (m, 1H) 13.32 (br s, 1H) 9.27 (br s, 1H) 9.11 (br s, 1H) 8.66 (br s, 1H) 8.08-8.33 (m, 1H) 8.08-8.33 (m, 1H) 7.62 (br s, 1H) 5.46 (br s, 2H) 4.37 (br s, 1H) 4.18 (br s, 2H) 3.41-3.57 (m, 2H) 3.25-3.31 (m, 3H) 2.92 (br s, 2H) 2.52-2.57 (m, 3H) 2.36-2.45 (m, 2H) 1.88-2.10 (m, 2H) 1.74 (br d, J=15 Hz, 1H) 0.95-1.46 (m, 9H).

### Example 1.20

### (E)-4-[[[3-(6-Benzyloxycarbonyl-8-methyl-5-oxo-1,8-naphthyridin-3-yl)-5,6-difluoro-4-[(3aR,6aR)-5-methyl-2,3,3a,4,6,6a-hexahydropyrrolo[2,3-c]pyrrol-1-yl]-9H-pyrido[2,3-b]indol-8-yl]-methyl-carbamoyl]oxymethoxy]-4-oxo-but-2-enoic acid

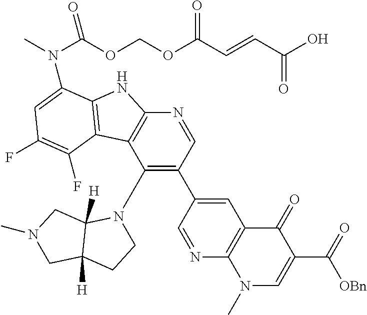 [SMILES: CN1C[C@H]2CCN(c3c(-c4cnc5c(c4)c(=O)c(C(=O)OCc4ccccc4)cn5C)cnc4[nH]c5c(N(C)C(=O)OCOC(=O)/C=C/C(=O)O)cc(F)c(F)c5c34)[C@H]2C1]

The titled compound was synthesized according to the following scheme:

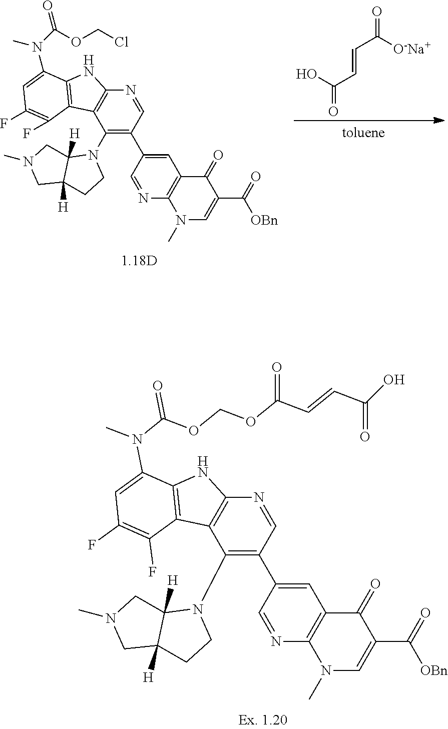

To a solution of benzyl 6-[8-[chloromethoxycarbonyl(methyl)amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (100 mg, 0.13 mmol, compound 1.18D) in DMF (3 mL) was added sodium hydrogen fumarate (55.8 mg, 0.40 mmol). The resulting reaction mixture was stirred at 95° C. for 2 h until LC-MS showed the starting material was consumed completely. The mixture was cooled back to r.t. and subject to prep-HPLC (NH₄HCO₃ as additive) purification to give (E)-4-[[[3-(6-Benzyloxycarbonyl-8-methyl-5-oxo-1,8-naphthyridin-3-yl)-5,6-difluoro-4-[(3aR,6aR)-5-methyl-2,3,3a,4,6,6a-hexahydropyrrolo[2,3-c]pyrrol-1-yl]-9H-pyrido[2,3-b]indol-8-yl]-methyl-carbamoyl]oxymethoxy]-4-oxo-but-2-enoic acid (25 mg, 22.2% yield) as a white solid. MS (ESI): 822.5 ([M+H]⁺). ¹H NMR (400 MHz, DMSO-d₆) δ ppm 12.42 (br s, 1H) 8.86-9.04 (m, 2H) 8.55 (br s, 1H) 8.14 (br d, J=17 Hz, 1H) 7.65 (br s, 1H) 7.53 (d, J=7 Hz, 2H) 7.28-7.47 (m, 3H) 6.53-6.89 (m, 2H) 6.46 (br d, J=15 Hz, 1H) 5.93 (br s, 1H) 5.71 (br s, 1H) 5.34 (s, 2H) 4.15-4.44 (m, 2H) 4.03 (s, 3H) 3.25-3.28 (m, 3H) 2.99 (br s, 1H) 2.81 (br s, 1H) 2.46 (br s, 1H) 2.20-2.40 (m, 2H) 2.07-2.17 (m, 3H) 1.96 (br s, 2H) 1.65 (br s, 1H).

### Example 1.21

### 6-[5,6-Difluoro-8-[methyl-[[(E)-3-carboxyprop-2-enoyl]oxymethoxycarbonyl]amino]-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid

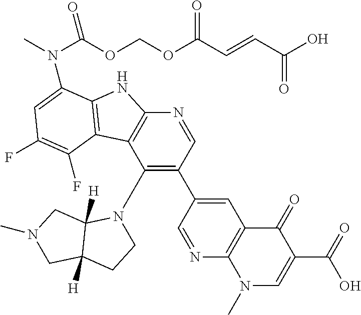 [SMILES: CN1C[C@H]2CCN(c3c(-c4cnc5c(c4)c(=O)c(C(=O)O)cn5C)cnc4[nH]c5c(N(C)C(=O)OCOC(=O)/C=C/C(=O)O)cc(F)c(F)c5c34)[C@H]2C1]

The titled compound was synthesized according to the following scheme:

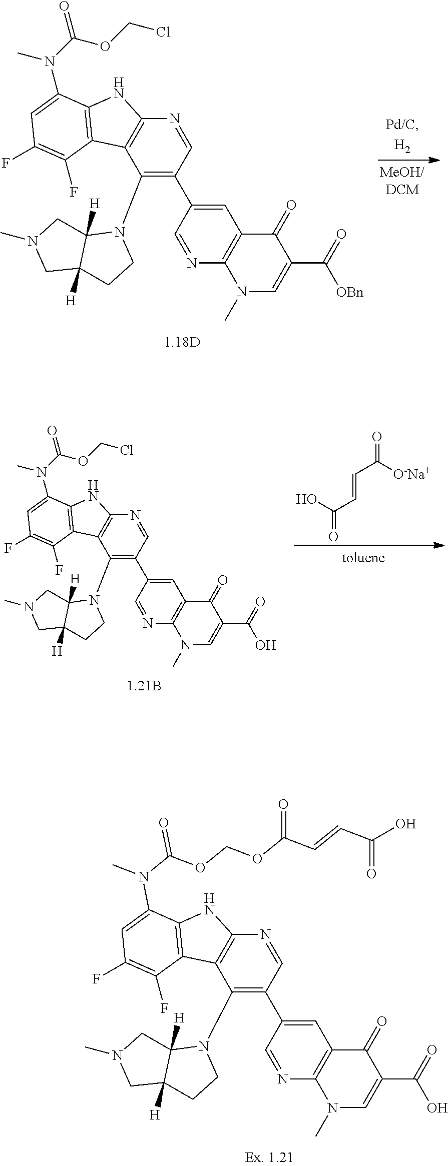

### Step (a) Preparation of 6-[8-[chloromethoxycarbonyl(methyl)amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (Compound 1.21B)

To a solution of benzyl 6-[8-[chloromethoxycarbonyl(methyl)amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (250 mg, 0.34 mmol, compound 1.18D) in MeOH (3 mL) was added palladium on carbon (0.03 mL, 0.34 mmol). After degassed under vacuum and flushed with H₂ several times, the reaction mixture was stirred at 20° C. for 2 h. After LC-MS showed the starting material was consumed, the reaction mixture was filtered through Celite. The filtrated was concentrated in vacuo to give a crude product of benzyl 6-[8-[chloromethoxycarbonyl(methyl)amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (168 mg, 51.3% yield) as a yellow solid. MS (ESI): 652.0 (M+H)⁺. It was used directly in the next step without further purification.

### Step (b) Preparation of 6-[5,6-difluoro-8-[methyl-[[(E)-3-carboxyprop-2-enoyl]oxymethoxycarbonyl]amino]-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (Example 1.21)

To a solution of benzyl 6-[8-[chloromethoxycarbonyl(methyl)amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (168 mg, 0.20 mmol) in DMF (1 mL) was added sodium hydrogen fumarate (84.7 mg, 0.61 mmol). The resulting reaction mixture was stirred at 90° C. for 2 h until LC-MS showed the starting material was consumed completely. The reaction mixture was cooled back to r.t. and filtered. The filtrate was subject to prep-HPLC (NH₄HCO₃ as additive) purification to give 6-[5,6-difluoro-8-[methyl-[[(E)-3-carboxyprop-2-enoyl]oxymethoxycarbonyl]amino]-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (10.3 mg, 6.7% yield) as a white solid. MS (ESI): 732.3 ([M+H]⁺). ¹H NMR (400 MHz, DMSO-d₆) δ ppm 12.32-12.63 (m, 1H) 9.27 (s, 1H) 9.09-9.18 (m, 1H) 8.73 (br s, 1H) 8.12-8.23 (m, 1H) 7.58-7.73 (m, 1H) 6.85 (br d, J=15 Hz, 1H) 6.40-6.59 (m, 1H) 5.92 (br d, J=3 Hz, 1H) 5.71 (br s, 1H) 4.41 (br s, 1H) 4.18 (s, 3H) 3.31-3.34 (m, 5H) 2.99 (br s, 2H) 2.84 (br s, 1H) 2.52-2.56 (m, 1H) 2.24 (br d, J=1 Hz, 1H) 2.10-2.18 (m, 3H) 1.87-2.04 (m, 2H) 1.58-1.72 (m, 1H) 1.66 (br s, 1H).

### Example 1.22

### 6-[1-[[tert-Butoxy(hydroxy)phosphoryl]oxymethyl]-5,6-difluoro-8-(methylamino)-4-[(3aR,6aR)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylic acid

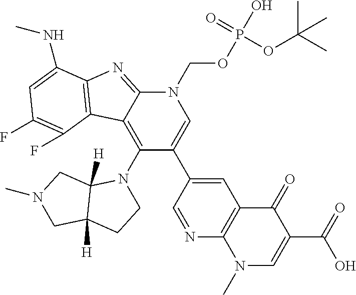 [SMILES: CNc1cc(F)c(F)c2c3c(N4CC[C@@H]5CN(C)C[C@@H]54)c(-c4cnc5c(c4)c(=O)c(C(=O)O)cn5C)cn(COP(=O)(O)OC(C)(C)C)c-3nc12]

The titled compound was synthesized according to the following scheme:

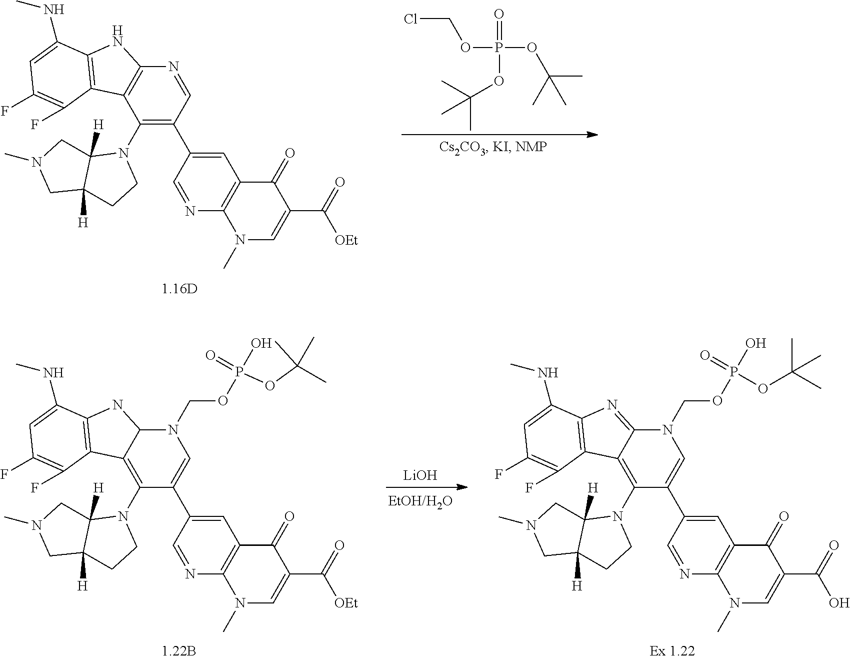 [SMILES: CCOC(=O)c1cn(C)c2ncc(-c3cnc4[nH]c5c(NC)cc(F)c(F)c5c4c3N3CC[C@@H]4CN(C)C[C@@H]43)cc2c1=O ; CC(C)(C)OP(=O)(OCCl)OC(C)(C)C ; CCOC(=O)c1cn(C)c2ncc(C3=CN(COP(=O)(O)OC(C)(C)C)C4Nc5c(NC)cc(F)c(F)c5C4=C3N3CC[C@@H]4CN(C)C[C@@H]43)cc2c1=O ; "LiOH" ; CNc1cc(F)c(F)c2c3c(N4CC[C@@H]5CN(C)C[C@@H]54)c(-c4cnc5c(c4)c(=O)c(C(=O)O)cn5C)cn(COP(=O)(O)OC(C)(C)C)c-3nc12]

### Step (a) Preparation of ethyl 6-[1-[[tert-butoxy(hydroxy)phosphoryl]oxymethyl]-5,6-difluoro-8-(methyl amino)-4-[(3aR,6aR)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylate (Compound 1.22B)

To a mixture solution of ethyl 6-[5,6-difluoro-8-(methylamino)-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (460 mg, 0.78 mmol, compound 1.16D), cesium carbonate (736 mg, 2.26 mmol), and potassium iodide (368 mg, 2.22 mmol) in anhydrous NMP (5.0 mL) was added di-tert-butyl chloromethyl phosphate (460 mg, 1.78 mmol). The resulting reaction mixture was stirred at 20° C. for 12 h until LC-MS showed the starting material was consumed completely. The mixture was then diluted with DCM (100 mL), poured into water (100 mL), and extracted with DCM (100 mL) twice. Combined organics was washed with brine (100 mL), dried over anhy. Na₂SO₄, filtered, and concentrated in vacuo to give a crude product, which was purified by prep-HPLC (NH₃.H₂O as additive) to give ethyl 6-[1-[[tert-butoxy(hydroxy)phosphoryl]oxymethyl]-5,6-difluoro-8-(methylamino)-4-[(3aR,6aR)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylate (150 mg, 25.4% yield) as an orange solid. MS (ESI): 754.4 ([M+H]⁺). ¹H NMR (400 MHz, METHANOL-d4) δ ppm 8.91˜9.02 (m, 2H), 8.46˜8.83 (m, 1H), 7.87˜8.26 (m, 1H), 6.07˜6.53 (m, 3H), 4.76 (br s, 1H), 4.25˜4.42 (m, 2H), 4.12 (br s, 3H), 2.96 (s, 7H), 2.54˜2.66 (m, 2H), 2.42 (br s, 3H), 1.91˜2.09 (m, 1H), 1.68 (br s, 1H), 1.39 (br d, J=12.59 Hz, 12H).

### Step (b) Preparation of 6-[1-[[tert-Butoxy(hydroxy)phosphoryl]oxymethyl]-5,6-difluoro-8-(methylamino)-4-[(3aR,6aR)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylic acid (Example 1.22)

To a solution of ethyl 6-[1-[[tert-butoxy(hydroxy)phosphoryl]oxymethyl]-5,6-difluoro-8-(methylamino)-4-[(3aR,6aR)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylate (150 mg, 0.2 mmol) in EtOH (2.5 mL) was added lithium hydroxide (0.5 mL, 4 mmol), and the reaction mixture was stirred at 20° C. for 1 h. After LC-MS showed the starting material was consumed, the mixture was adjusted to pH=6 with aq. HCl solution (1N). It was then subject to prep-HPLC (NH₃.H₂O as additive) purification to give 6-[1-[[tert-butoxy(hydroxy)phosphoryl]oxymethyl]-5,6-difluoro-8-(methylamino)-4-[(3aR,6aR)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylic acid (35.0 mg, 23.5% yield) as a red solid. MS (ESI): 726.4 ([M+H]⁺). ¹H NMR (400 MHz, DMSO-d6) δ ppm 14.42˜14.94 (m, 1H), 9.27 (s, 1H), 9.11˜9.18 (m, 1H), 9.11˜9.18 (m, 1H), 8.83 (s, 1H), 8.31˜8.40 (m, 1H), 8.31˜8.40 (m, 1H), 6.80˜7.60 (m, 1H), 6.34˜6.71 (m, 1H), 5.93˜6.19 (m, 2H), 5.12˜5.52 (m, 1H), 4.78 (br s, 1H), 4.17 (s, 3H), 2.97˜3.15 (m, 3H), 2.90 (br s, 5H), 2.59˜2.66 (m, 1H), 2.22˜2.30 (m, 1H), 2.13˜2.21 (m, 3H), 1.87˜2.04 (m, 2H), 1.61˜1.70 (m, 1H), 1.27 (br s, 9H).

### Example 1.23

### 6-[5,6-Difluoro-8-(methylamino)-1-(phosphonooxymethyl)-4-[(3aR,6aR)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylic acid

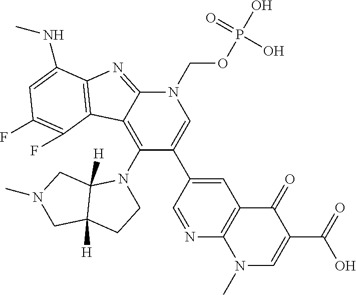 [SMILES: CNc1cc(F)c(F)c2c3c(N4CC[C@@H]5CN(C)C[C@@H]54)c(-c4cnc5c(c4)c(=O)c(C(=O)O)cn5C)cn(COP(=O)(O)O)c-3nc12]

The titled compound was synthesized according to the following scheme:

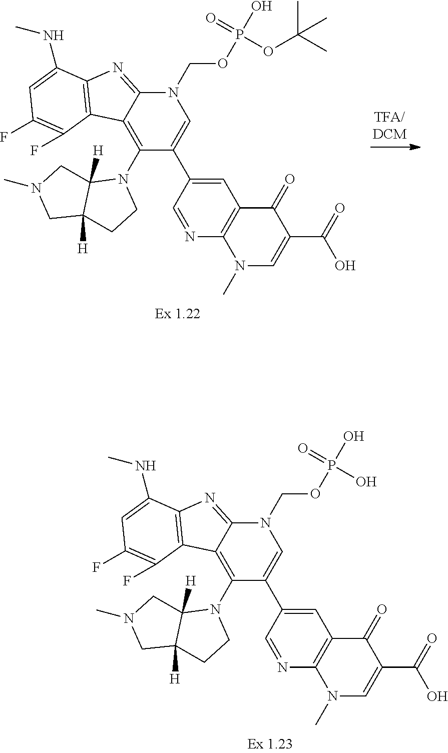 [SMILES: CNc1cc(F)c(F)c2c3c(N4CC[C@@H]5CN(C)C[C@@H]54)c(-c4cnc5c(c4)c(=O)c(C(=O)O)cn5C)cn(COP(=O)(O)OC(C)(C)C)c-3nc12 ; CNc1cc(F)c(F)c2c3c(N4CC[C@@H]5CN(C)C[C@@H]54)c(-c4cnc5c(c4)c(=O)c(C(=O)O)cn5C)cn(COP(=O)(O)O)c-3nc12]

To a solution of 6-[1-[[tert-butoxy(hydroxy)phosphoryl]oxymethyl]-5,6-difluoro-8-(methylamino)-4-[(3aR,6aR)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylic acid (23.0 mg, 0.03 mmol) in DCM (2.0 mL) was added TFA (0.2 mL, 0.03 mmol), and the reaction mixture was stirred at 20° C. for 2 h. After LC-MS showed the starting material was consumed, the mixture was subject to prep-HPLC (NH₃.H₂O as additive) purification to give 6-[5,6-difluoro-8-(methylamino)-1-(phosphonooxymethyl)-4-[(3aR,6aR)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylic acid (12.2 mg, 56.9% yield) as a yellow solid. MS (ESI): 670.3 ([M+H]⁺). ¹H NMR (400 MHz, DMSO-d6) δ ppm 8.92˜9.07 (m, 2H), 8.66 (br s, 1H), 8.03 (s, 1H), 7.25˜7.25 (m, 1H), 6.34˜6.47 (m, 1H), 5.92˜6.03 (m, 2H), 4.59˜4.70 (m, 1H), 4.11˜4.13 (m, 3H), 2.86˜3.01 (m, 2H), 2.80˜2.85 (m, 3H), 2.64˜2.71 (m, 1H), 2.34 (br s, 2H), 2.13 (br s, 4H), 1.82˜1.96 (m, 1H), 1.50˜1.64 (m, 1H).

### Example 1.24

### 6-[5,6-Difluoro-8-(methylamino)-4-[(3aR,6aR)-5-[[tert-butoxy(hydroxy)phosphoryl]oxymethyl]-5-methyl-2,3,3a,4,6,6a-hexahydropyrrolo[2,3-c]pyrrol-5-ium-1-yl]-9H-pyrido[2,3-b]indol-3-yl]-1-methyl-4-oxo-1,8-naphthyridine-3-carboxylic acid

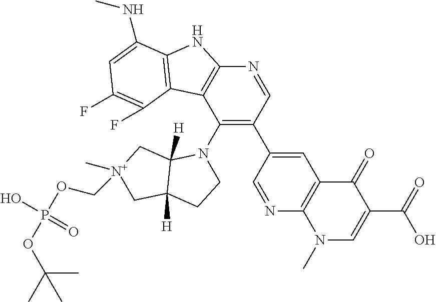 [SMILES: CNc1cc(F)c(F)c2c1[nH]c1ncc(-c3cnc4c(c3)c(=O)c(C(=O)O)cn4C)c(N3CC[C@@H]4C[N+](C)(COP(=O)(O)OC(C)(C)C)C[C@@H]43)c12]

The titled compound was synthesized according to the following scheme:

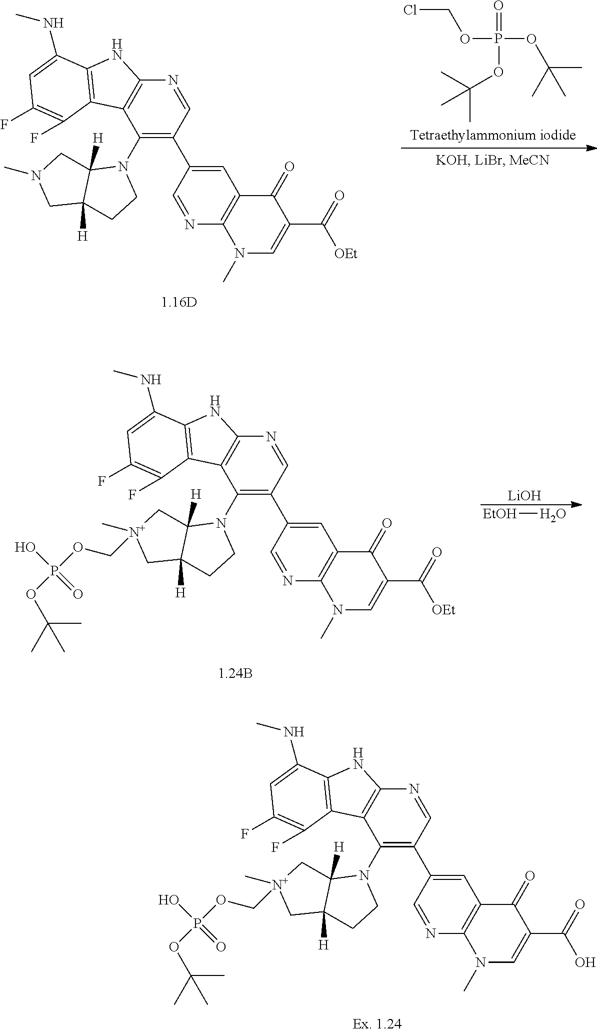

### Step (a) Preparation of ethyl 6-[5,6-difluoro-8-(methylamino)-4-[(3aR,6aR)-5-[[tert-butoxy(hydroxy)phosphoryl]oxymethyl]-5-methyl-2,3,3a,4,6,6a-hexahydropyrrolo[2,3-c]pyrrol-5-ium-1-yl]-9H-pyrido[2,3-b]indol-3-yl]-1-methyl-4-oxo-1,8-naphthyridine-3-carboxylate (Compound 1.24B)

To a solution of ethyl 6-[5,6-difluoro-8-(methylamino)-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (105.0 mg, 0.180 mmol, compound 1.16D), lithium bromide (46.55 mg, 0.540 mmol), potassium hydroxide (30.08 mg, 0.540 mmol), and tetraethylammonium iodide (137.85 mg, 0.540 mmol) in anhydrous ACN (2.0 mL) was added di-tert-butyl chloromethyl phosphate (138.67 mg, 0.540 mmol). The resulting reaction mixture was stirred at 20° C. for 12 h. After LC-MS showed the starting material was consumed completely, the mixture was poured into water (20 mL) and lyophilized to give a crude product. It was re-dissolved in MeOH and subject to prep-HPLC (NH₄HCO₃ as additive) purification to give ethyl 6-[5,6-difluoro-8-(methylamino)-4-[(3aR,6aR)-5-[[tert-butoxy(hydroxy)phosphoryl]oxymethyl]-5-methyl-2,3,3a,4,6,6a-hexahydropyrrolo[2,3-c]pyrrol-5-ium-1-yl]-9H-pyrido[2,3-b]indol-3-yl]-1-methyl-4-oxo-1,8-naphthyridine-3-carboxylate (35.0 mg, 24.2% yield) as a grey solid. MS (ESI): 754.4 ([M+H]⁺). ¹H NMR (400 MHz, DMSO-d6) δ ppm 12.13˜12.34 (m, 1H), 8.88˜8.91 (m, 1H), 8.83˜8.85 (m, 1H), 8.37˜8.40 (m, 1H), 8.22˜8.24 (m, 1H), 6.46˜6.60 (m, 1H), 5.94˜6.07 (m, 1H), 5.94˜6.07 (m, 1H), 4.58˜4.67 (m, 1H), 4.49˜4.57 (m, 1H), 4.35˜4.48 (m, 1H), 4.26 (br d, J=7.09 Hz, 2H), 4.00 (s, 4H), 3.66˜3.75 (m, 1H), 3.09˜3.31 (m, 4H), 3.00 (s, 3H), 2.84 (br s, 4H), 1.98˜2.12 (m, 1H), 1.69˜1.80 (m, 1H), 1.28˜1.35 (m, 3H), 1.15˜1.22 (m, 9H).

### Step (b) Preparation of 6-[5,6-difluoro-8-(methylamino)-4-[(3aR,6aR)-5-[tert-butoxy(hydroxy)phosphoryl)oxymethyl]-5-methyl-2,3,3a,4,6,6a-hexahydropyrrolo[2,3-c]pyrrol-5-ium-1-yl]-9H-pyrido[2,3-b]indol-3-yl]-1-methyl-4-oxo-1,8-naphthyridine-3-carboxylic acid (Example 1.24)

A mixture solution of ethyl 6-[5,6-difluoro-8-(methylamino)-4-[(3aR,6aR)-5-[[tert-butoxy(hydroxy)phosphoryl]oxymethyl]-5-methyl-2,3,3a,4,6,6a-hexahydropyrrolo[2,3-c]pyrrol-5-ium-1-yl]-9H-pyrido[2,3-b]indol-3-yl]-1-methyl-4-oxo-1,8-naphthyridine-3-carboxylate (45.0 mg, 0.060 mmol) and lithium hydroxide (44.9 mg, 1.88 mmol) in EtOH (2.0 mL) and water (0.5 mL) was stirred at 20° C. for 2 h. After LC-MS showed the starting material was consumed, the mixture was diluted with MeOH (2.0 mL), and subject to prep-HPLC (NH₄HCO₃ as additive) purification to give 6-[5,6-difluoro-8-(methylamino)-4-[(3aR,6aR)-5-[[tert-butoxy(hydroxy)phosphoryl]oxymethyl]-5-methyl-2,3,3a,4,6,6a-hexahydropyrrolo[2,3-c]pyrrol-5-ium-1-yl]-9H-pyrido[2,3-b]indol-3-yl]-1-methyl-4-oxo-1,8-naphthyridine-3-carboxylic acid (40.0 mg, 92.3% yield) as a grey solid. MS (ESI): 726.4 ([M+H]⁺). ¹H NMR (400 MHz, MeOH-d4) δ ppm 9.37˜9.61 (m, 1H), 8.86˜9.21 (m, 1H), 8.57 (s, 1H), 7.83˜8.29 (m, 1H), 6.26˜6.70 (m, 1H), 6.17 (br s, 1H), 4.40˜4.68 (m, 2H), 4.08 (br s, 4H), 3.90˜4.03 (m, 3H), 3.47˜3.73 (m, 1H), 3.37 (s, 1H), 2.83˜3.09 (m, 3H), 2.61 (br s, 3H), 2.06˜2.42 (m, 2H), 1.85˜1.99 (m, 1H), 1.85˜1.99 (m, 1H), 1.56˜1.70 (m, 1H), 1.23˜1.40 (m, 9H).

### Example 1.25

### 6-[5,6-Difluoro-8-(methylamino)-4-[(3aR,6aR)-5-methyl-5-(phosphonooxymethyl)-2,3,3a,4,6,6a-hexahydropyrrolo[2,3-c]pyrrol-5-ium-1-yl]-9H-pyrido[2,3-b]indol-3-yl]-1-methyl-4-oxo-1,8-naphthyridine-3-carboxylic acid

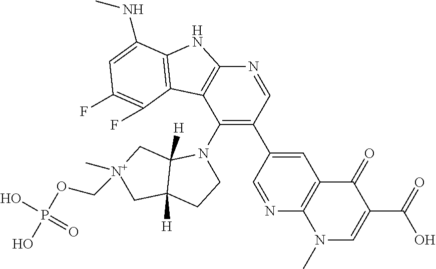 [SMILES: CNc1cc(F)c(F)c2c1[nH]c1ncc(-c3cnc4c(c3)c(=O)c(C(=O)O)cn4C)c(N3CC[C@@H]4C[N+](C)(COP(=O)(O)O)C[C@@H]43)c12]

To a solution 6-[5,6-difluoro-8-(methylamino)-4-[(3aR,6aR)-5-[[tert-butoxy(hydroxy)phosphoryl]oxymethyl]-5-methyl-2,3,3a,4,6,6a-hexahydropyrrolo[2,3-c]pyrrol-5-ium-1-yl]-9H-pyrido[2,3-b]indol-3-yl]-1-methyl-4-oxo-1,8-naphthyridine-3-carboxylic acid (29.7 mg, 0.04 mmol, Example 1.24) in DCM (1.0 mL) was added TFA/DCM (0.4 mL, 0.01 mmol, v/v=1:5), and the reaction mixture was stirred at 20° C. for 1 h. After LC-MS showed the starting material was consumed, the mixture was concentrated in vacuo to give a crude product, which was purified by prep-HPLC (NH₄HCO₃ as additive) to give 6-[5,6-difluoro-8-(methylamino)-4-[(3aR,6aR)-5-methyl-5-(phosphonooxymethyl)-2,3,3a,4,6,6a-hexahydropyrrolo[2,3-c]pyrrol-5-ium-1-yl]-9H-pyrido[2,3-b]indol-3-yl]-1-methyl-4-oxo-1,8-naphthyridine-3-carboxylic acid (7 mg, 21.5% yield) as a yellow solid. MS (ESI): 670.3 ([M+H]⁺). ¹H NMR (400 MHz, DMSO-d6) δ ppm 9.23˜9.29 (m, 1H), 9.05˜9.12 (m, 1H), 8.61˜8.68 (m, 1H), 8.22˜8.29 (m, 1H), 6.54˜6.64 (m, 1H), 5.59˜5.96 (m, 1H), 4.43˜4.61 (m, 2H), 4.28˜4.39 (m, 1H), 4.18 (s, 4H), 3.92˜4.02 (m, 2H), 3.56˜3.69 (m, 1H), 3.07˜3.17 (m, 1H), 2.88 (br s, 6H) 2.64˜2.71 (m, 2H), 2.30˜2.36 (m, 2H), 2.04˜2.11 (m, 1H), 1.76 (s, 1H).

### Example 1.26

### 6-[5,6-Difluoro-8-[formyl(methyl)amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid

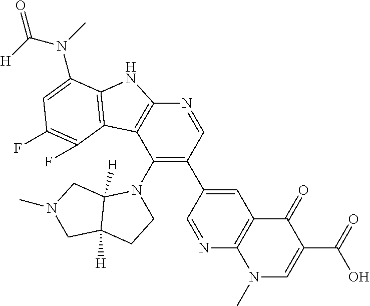 [SMILES: CN1C[C@@H]2CCN(c3c(-c4cnc5c(c4)c(=O)c(C(=O)O)cn5C)cnc4[nH]c5c(N(C)C=O)cc(F)c(F)c5c34)[C@@H]2C1]

The titled compound was synthesized according to the following scheme:

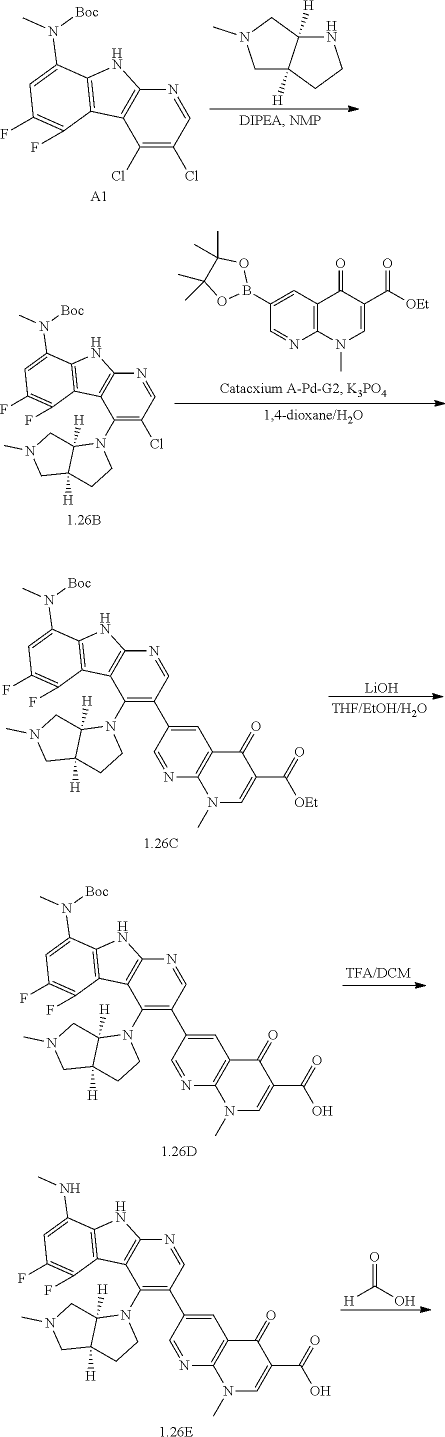

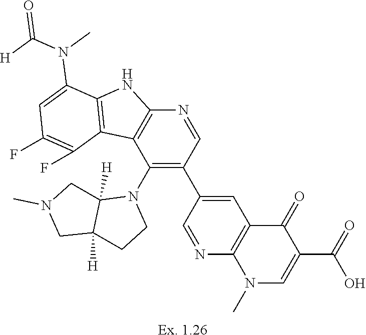 [SMILES: CN1C[C@@H]2CCN(c3c(-c4cnc5c(c4)c(=O)c(C(=O)O)cn5C)cnc4[nH]c5c(N(C)C=O)cc(F)c(F)c5c34)[C@@H]2C1 ; "Ex. 1.26"]

### Step (a) Preparation of tert-butyl N-[3-chloro-5,6-difluoro-4-[(3aS,6aS)-5-methyl-2,3,3a,4,6,6a-hexahydropyrrolo[2,3-c]pyrrol-1-yl]-9H-pyrido[2,3-b]indol-8-yl]-N-methyl-carbamate (Compound 1.26B)

A solution of tert-butyl N-(3,4-dichloro-5,6-difluoro-9H-pyrido[2,3-b]indol-8-yl)-N-methyl-carbamate (10.8 g, 26.87 mmol, Intermediate A1), (3aS,6aS)-5-methyl-2,3,3a,4,6,6a-hexahydro-1H-pyrrolo[2,3-c]pyrrole (6.1 g, 48.36 mmol, CAS: 1176846-84-6), and DIPEA (14.04 mL, 80.6 mmol) in NMP (15 mL) was stirred at 118° C. for 48 h. After TLC (petrolieum ether:EtOAc=2:1, 2% of Et₃N as additive) showed the starting material was consumed, the mixture was poured into EtOAc (500 mL). The separated organic layer was then washed with aq. CaCl₂ solution (100 mL) three times, brine (100 mL) twice, dried over anhy. Na₂SO₄, filtered, and concentrated in vacuo to give a crude product, which was purified by silica gel flash chromatography (petrolieum ether:EtOAc=10:1˜1, 2% of Et₃N as additive) to give tert-butyl N-[3-chloro-5,6-difluoro-4-[(3aS,6aS)-5-methyl-2,3,3a,4,6,6a-hexahydropyrrolo[2,3-c]pyrrol-1-yl]-9H-pyrido[2,3-b]indol-8-yl]-N-methyl-carbamate (12.0 g, 88.8% yield, >98% ee, optical rotation [α]_{D}=+27.7°) as a white solid. MS (ESI): 492.3 ([{³⁵Cl}M+H]⁺).

### Step (b) Preparation of ethyl 6-[8-[tert-butoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (Compound 1.26C)

To a solution of tert-butyl N-[3-chloro-5,6-difluoro-4-[(3aS,6aS)-5-methyl-2,3,3a,4,6,6a-hexahydropyrrolo[2,3-c]pyrrol-1-yl]-9H-pyrido[2,3-b]indol-8-yl]-N-methyl-carbamate (10.0 g, 20.33 mmol), ethyl 1-methyl-4-oxo-6-(4,4,5,5-tetramethyl-1,3,2-dioxaborolan-2-yl)-1,8-naphthyridine-3-carboxylate (14.6 g, 40.65 mmol, Intermediate B1), and potassium phosphate (17.3 g, 81.31 mmol) in 1,4-dioxane (300 mL) and water (40 mL) was added Pd-Ad2nBuP Biphenyl (2.71 g, 4 mmol, cataCXium A-Pd-G2, CAS: 1375477-29-4) in a glove box under argon. The reaction mixture was stirred at 90° C. for 12 h until LC-MS showed the starting material was consumed completely. The reaction mixture was cooled back to r.t., filtered and the filtrate was diluted with EtOAc (800 mL). The organic phase was washed with brine (200 mL) three times, dried over anhy. Na₂SO₄, filtered, and concentrated in vacuo to give a crude product. It was purified twice by silica gel flash chromatography (first with DCM:MeOH=100:1˜10:1 and then with petrolieum ether:EtOAc=100:1˜1:3, 2% Et₃N as additive) to give ethyl 6-[8-[tert-butoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (10.5 g, 65.6% yield)) as a white solid. MS (ESI): 688.5 ([M+H]⁺).

### Step (c) Preparation of 6-[8-[tert-butoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (Compound 1.26D)

To a solution of ethyl 6-[8-[tert-butoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (10.0 g, 14.54 mmol) in THF (60 mL) and EtOH (15 mL) was added aq. LiOH solution (21.8 mL, 43.6 mmol). The resulting mixture was stirred at 15° C. for 4 h until LC-MS showed the starting material was consumed completely. The mixture was diluted with EtOAc (200 mL) and H₂O (50 mL), adjusted pH to 6 with aq. HCl solution (1N), and extracted with DCM/MeOH (10:1, 400 mL) twice. Combined organics was then washed with brine (200.0 mL) twice, dried over anhy. Na₂SO₄, filtered, and concentrated in vacuo to give a crude product of 6-[8-[tert-butoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (9.5 g, 88.5% yield) as a white solid. MS (ESI): 660.3 ([M+H]⁺). It was used directly in the next step without further purification.

### Step (d) Preparation of 6-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (Compound 1.26E)

To a solution of 6-[4-[(3aS,6aS)-5-methyl-2,3,3a,4,6,6a-hexahydropyrrolo[2,3-c]pyrrol-1-yl]-8-[tert-butoxycarbonyl(methyl)amino]-5,6-difluoro-9H-pyrido[2,3-b]indol-3-yl]-1-methyl-4-oxo-1,8-naphthyridine-3-carboxylic acid (9.45 g, 14.33 mmol) in DCM (80 mL) was added TFA (20.0 mL, 259.6 mmol), and the resulting reaction mixture was stirred at 15° C. for 1 h. After LC-MS showed the starting material was consumed completely, the mixture solution was evaporated to dryness under the reduced pressure. The residue was re-dissolved in MeOH (100 mL) and adjusted with aq. NaHCO₃ solution to pH=6. The resulting suspension was then filtered and the filtrate was concentrated in vacuo to give a crude product, which was further purified by prep-HPLC (NH₃.H₂O as additive) to give 6-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (4.87 g, 59.5% yield) as a yellow solid. MS (ESI): 560.3 ([M+H]⁺), 280.8 ([M/2+H]⁺). ¹H NMR (400 MHz, DMSO-d6) δ ppm 14.76 (br s, 1H), 12.09 (br s, 1H), 9.19 (s, 1H), 9.08 (d, J=2.2 Hz, 1H), 8.68 (d, J=2.4 Hz, 1H), 8.10 (s, 1H), 6.54 (dd, J=13.6, 6.2 Hz, 1H), 5.91 (br d, J=3.7 Hz, 1H), 4.44 (br s, 1H), 4.14 (s, 3H), 2.77-3.01 (m, 6H), 2.44 (br d, J=9.3 Hz, 2H), 2.15 (br t, J=8.2 Hz, 1H), 2.08 (s, 3H), 1.88-2.02 (m, 1H), 1.79 (br dd, J=9.7, 5.3 Hz, 1H), 1.52-1.65 (m, 1H).

### Step (e) Preparation of 6-[5,6-difluoro-8-[formyl(methyl)amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (Example 1.26)

A solution of 6-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid_(30 mg, 0.054 mmol) in formic acid (0.5 mL) was stirred at 100° C. for 10 min under microwave. Afterwards, the reaction mixture was cooled back to r.t. and the residue formic acid was removed under the reduced pressure to give a crude product. It was then subject to prep-HPLC (formic acid as additive) purification to give 6-[5,6-difluoro-8-[formyl(methyl)amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (7.1 mg, 21.4% yield) as a white solid. MS (ESI): 588.2 ([M+H]⁺). ¹H NMR (400 MHz, MeOD-d4) δ 9.06 (s, 1H), 8.98 (s, 1H), 8.73 (s, 1H), 8.27-8.33 (m, 1H), 8.15 (m, 1H), 7.54-7.42 (m, 1H), 7.39-7.47 (m, 1H), 4.27 (m, 1H), 4.13 (s, 3H), 3.39 (m, 2H), 3.35 (s, 3H), 3.04 (m, 2H), 2.60-2.90 (m, 3H), 2.47-2.51 (m, 3H), 2.10 (m, 1H), 1.75 (m, 1H).

### Example 1.27

### 6-[5,6-Difluoro-8-[methyl(sulfo)amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid

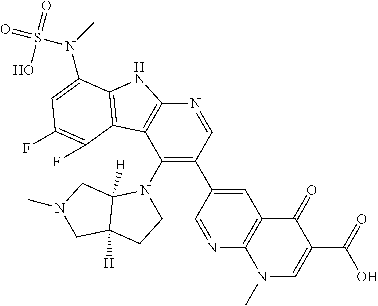 [SMILES: CN1C[C@@H]2CCN(c3c(-c4cnc5c(c4)c(=O)c(C(=O)O)cn5C)cnc4[nH]c5c(N(C)S(=O)(=O)O)cc(F)c(F)c5c34)[C@@H]2C1]

The titled compound was synthesized according to the following scheme:

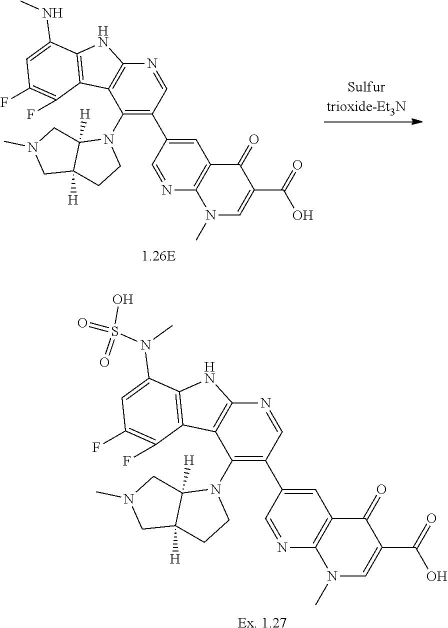 [SMILES: CNc1cc(F)c(F)c2c1[nH]c1ncc(-c3cnc4c(c3)c(=O)c(C(=O)O)cn4C)c(N3CC[C@H]4CN(C)C[C@H]43)c12 ; "1.26E" ; CN1C[C@@H]2CCN(c3c(-c4cnc5c(c4)c(=O)c(C(=O)O)cn5C)cnc4[nH]c5c(N(C)S(=O)(=O)O)cc(F)c(F)c5c34)[C@@H]2C1 ; "Ex. 1.27"]

A mixture solution of 6-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (30 mg, 0.054 mmol, compound 1.26E), sulfur trioxide triethylamine complex (38 mg, 0.21 mmol), and Et₃N (32.5 mg, 0.32 mmol) in DMF (2.0 mL) was stirred at 100° C. for 10 min under microwave. Afterwards, the mixture solution was cooled back to r.t., and subject to prep-HPLC (p formic acid as additive) purification to give 6-[5,6-difluoro-8-[methyl(sulfo)amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (7.9 mg, 22% yield) as a white solid. MS (ESI): 640.2 ([M+H]⁺). ¹H NMR (400 MHz, MeOD-d4) δ9.19 (s, 1H), 9.09 (s, 1H), 8.90 (d, J=4 Hz, 1H), 8.32 (s, 1H), 7.67 (m, 1H), 4.26 (s, 3H), 3.69 (m, 1H), 3.45 (m, 2H), 3.25 (s, 3H), 3.01-3.15 (m, 3H), 2.91 (s, 2H), 2.76 (s, 3H), 2.30 (m, 1H), 1.94 (m, 1H).

### Example 1.28

### 6-[5,6-Difluoro-8-[methyl-[[(E)-3-carboxyprop-2-enoyl]oxymethoxycarbonyl]amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid

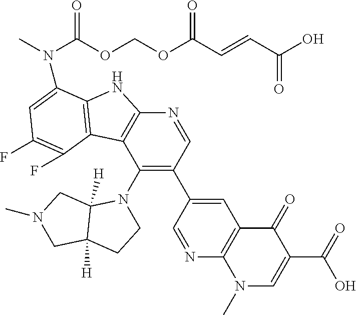 [SMILES: CN1C[C@@H]2CCN(c3c(-c4cnc5c(c4)c(=O)c(C(=O)O)cn5C)cnc4[nH]c5c(N(C)C(=O)OCOC(=O)/C=C/C(=O)O)cc(F)c(F)c5c34)[C@@H]2C1]

The titled compound was synthesized according to the following scheme:

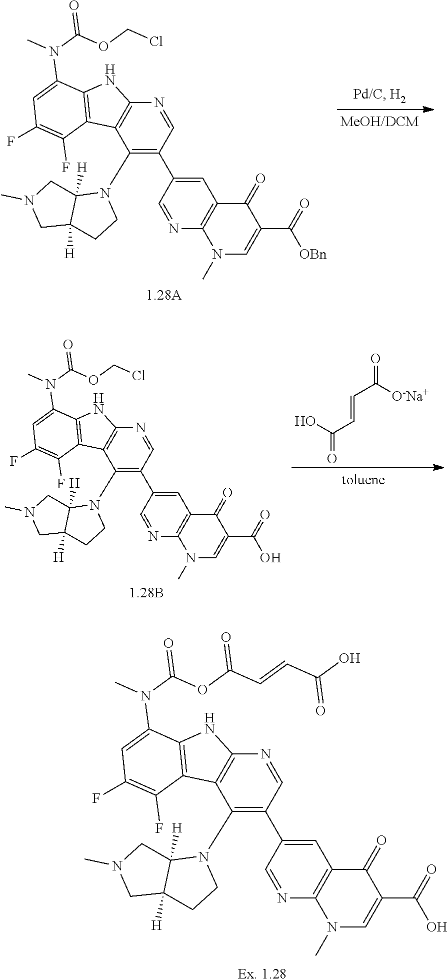

### Step (a) Preparation of benzyl 6-[8-[chloromethoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (Compound 1.28A)

In analogy to the synthesis of compound 1.18D (steps a, b, and c), tert-butyl N-[3-chloro-5,6-difluoro-4-[(3aS,6aS)-5-methyl-2,3,3a,4,6,6a-hexahydropyrrolo[2,3-c]pyrrol-1-yl]-9H-pyrido[2,3-b]indol-8-yl]-N-methyl-carbamate was used instead of tert-butyl N-[3-chloro-5,6-difluoro-4-[(3aR,6aR)-5-methyl-2,3,3a,4,6,6a-hexahydropyrrolo[2,3-c]pyrrol-1-yl]-9H-pyrido[2,3-b]indol-8-yl]-N-methyl-carbamate (compound 1.16B in step a) to give benzyl 6-[8-[chloromethoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate as a yellow solid. MS (ESI): 742.3 ([M+H]⁺).

### Step (b) Preparation of 6-[8-[chloromethoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (Compound 1.28B)

To a solution of benzyl 6-[8-[chloromethoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (300 mg, 0.40 mmol) in DCM (1.8 mL) and MeOH (1.8 mL) was added palladium on carbon (60.0 mg, 0.06 mmol). After the suspension was degassed under vacuum and purged with H₂ several times, the mixture solution was stirred at 20° C. for 2 h under H₂ atmosphere (15 psi). After LC-MS showed the starting material was consumed completely, the reaction mixture was filtered through Celite. The filtrate was then concentrated in vacuo to give a crude product of 6-[8-[chloromethoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (100 mg, 37.9% yield) as a yellow solid. MS (ESI): 652.3 ([M+H]⁺). It was used directly in the next step without further purification.

### Step (c) Preparation of 6-[5,6-difluoro-8-[methyl-[[(E)-3-carboxyprop-2-enoyl]oxymethoxycarbonyl]amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (Example 1.28)

To a solution of 6-[8-[chloromethoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (100 mg, 0.15 mmol) in DMF (2.7 mL) was added sodium hydrogen fumarate (63.5 mg, 0.46 mmol). The resulting reaction mixture was stirred at 90° C. for 1 h until LC-MS showed the starting material was consumed. The mixture was filtered and the filtrate was concentrated in vacuo to give a crude product, which was purified by prep-HPLC (NH₄HCO₃ as additive) to give 6-[5,6-Difluoro-8-[methyl-[[(E)-3-carboxyprop-2-enoyl]oxymethoxycarbonyl]amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (11 mg, 3.1% yield) as a white solid. MS (ESI): 732.1 ([M+H]⁺). ¹H NMR (400 MHz, DMSO-d₆) δ ppm: 14.82 (br s, 1H), 12.47 (br d, 0.7=13.4 Hz, 1H), 9.27 (s, 1H), 9.11-9.15 (m, 1H), 8.71-8.75 (m, 1H), 8.10-8.24 (m, 1H), 7.59-7.72 (m, 1H), 6.43-6.91 (m, 2H), 5.65-5.96 (m, 2H), 4.41 (br s, 1H), 4.18 (s, 3H), 3.31 (br s, 3H), 2.99 (br s, 2H), 2.83 (br s, 1H), 2.53 (d, J=2.0 Hz, 2H), 2.23-2.30 (m, 1H), 2.11-2.18 (m, 3H), 1.90-2.06 (m, 2H), 1.61-1.69 (m, 1H).

### Example 1.29

### 6-[5,6-Difluoro-8-[methyl(sulfo)amino]-4-[cis-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid

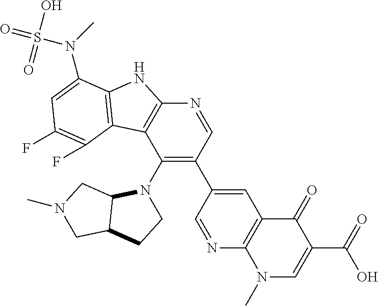

The titled compound was synthesized according to the following scheme:

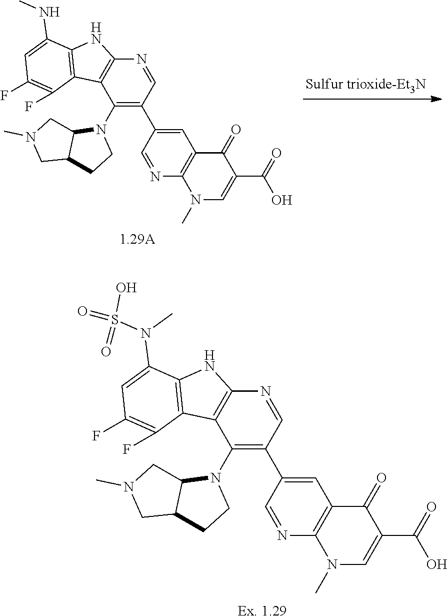

### Step (a) Preparation of 6-[5,6-difluoro-8-(methylamino)-4-[cis-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (Compound 1.29A)

In analogy to the synthesis of compound 1.26E, c/s-5-methyl-2,3,3a,4,6,6a-hexahydro-1H-pyrrolo[2,3-c]pyrrole (Intermediate Cl) was used in the step (a) instead of (3aS,6aS)-5-methyl-2,3,3a,4,6,6a-hexahydro-1H-pyrrolo[2,3-c]pyrrole to give 6-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid after steps b, c, and d as a white solid. MS (ESI): 492.3 ([{³⁵Cl}M+H]⁺).

### Step (b) Preparation of 6-[5,6-difluoro-8-[methyl(sulfo)amino]-4-[cis-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (Example 1.29)

In analogy to the synthesis of Example 1.27, 6-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (30 mg, 0.054 mmol) was used instead of 6-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (compound 1.26E) to give 6-[5,6-difluoro-8-[methyl(sulfo)amino]-4-[cis-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (7.9 mg, 22% yield) as a white solid after prep-HPLC purification, (formic acid as additive. MS (ESI): 640.2 ([M+H]⁺). ¹H NMR (400 MHz, MeOD-d4) δ9.19 (s, 1H), 9.09 (s, 1H), 8.90 (d, J=4 Hz, 1H), 8.32 (s, 1H), 7.67 (m, 1H), 4.26 (s, 3H), 3.69 (m, 1H), 3.45 (m, 2H), 3.25 (s, 3H), 3.01-3.15 (m, 3H), 2.91 (s, 2H), 2.76 (s, 3H), 2.30 (m, 1H), 1.94 (m, 1H).

### Example 1.30

### 6-[8-[[2-[(2-Aminoacetyl)amino]acetyl]-methyl-amino]-5,6-difluoro-4-[cis-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid

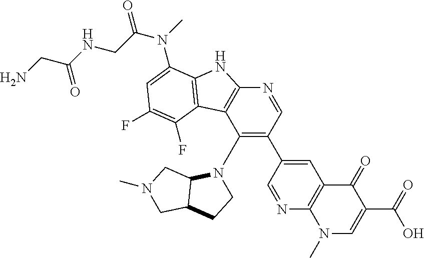

The titled compound was synthesized according to the following scheme:

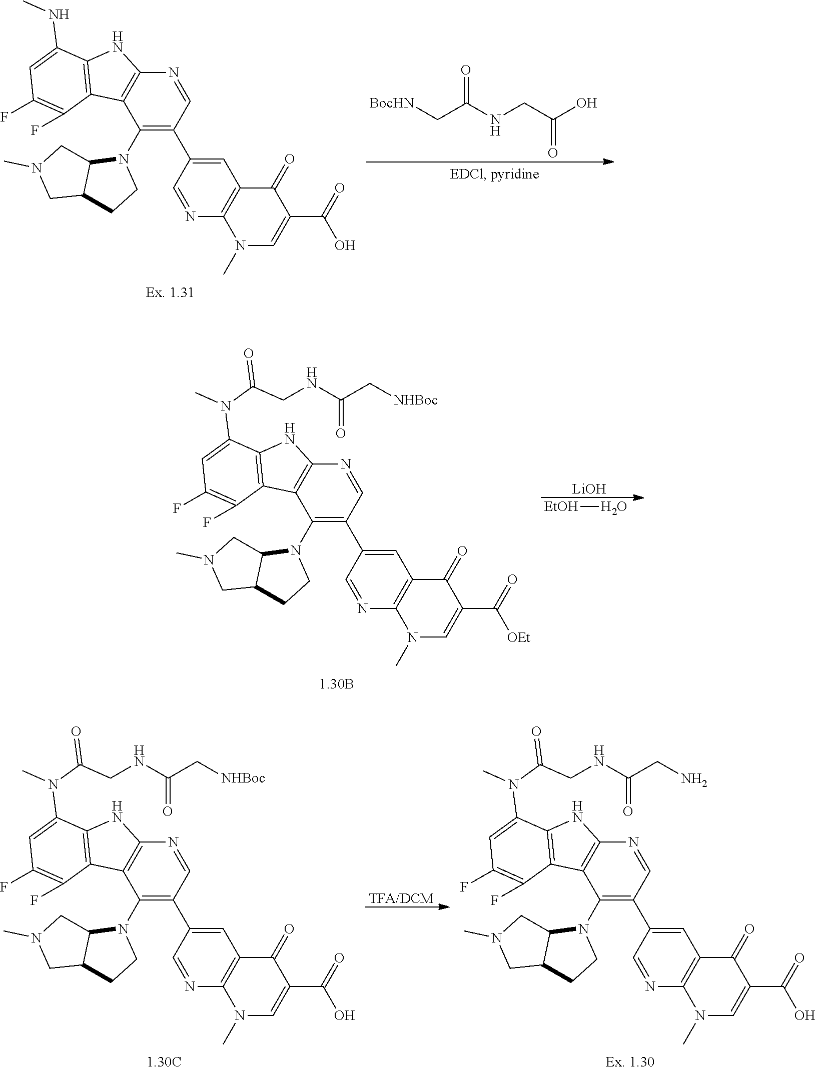

### Step (a) Preparation of ethyl 6-[8-[[2-[[2-(tert-butoxycarbonylamino)acetyl]amino]acetyl]-methyl-amino]-5,6-difluoro-4-[cis-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (Compound 1.30B1

A mixture solution of ethyl 6-[5,6-difluoro-8-(methylamino)-4-[c/3′-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (200 mg, 0.34 mmol, Example 1.31), 2-[[2-(tert-butoxycarbonylamino)acetyl]amino]acetic acid (200 mg, 0.86 mmol), and EDCI (200 mg, 1.04 mmol) in pyridine (5 mL) was stirred at 80° C. for 90 h. After LC-MS showed the starting material was consumed completely, the reaction mixture was cooled back to r.t. and concentrated in vacuo. The crude product was then subject to prep-HPLC (TFA as additive) purification to give ethyl 6-[8-[[2-[[2-(tert-butoxycarbonylamino)acetyl]amino]acetyl]-methyl-amino]-5,6-difluoro-4-[cis-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (55 mg, 20.2% yield) as a yellow solid. MS (ESI): 802.6 ([M+H]⁺).

### Step (b) Preparation of 6-[8-[[2-[[2-(tert-butoxycarbonylamino)acetyl]amino]acetyl]-methyl-amino]-5,6-difluoro-4-[cis-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (Compound 1.30C)

To a solution of ethyl 6-[8-[[2-[[2-(tert-butoxycarbonylamino)acetyl]amino]acetyl]-methyl-amino]-5,6-difluoro-4-[cis-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (50.0 mg, 0.06 mmol) in EtOH (2 mL) and water (2 mL) was added lithium hydroxide (10.0 mg, 0.420 mmol), and the resulting reaction mixture was stirred at 20° C. for 1 h. After LC-MS showed the starting material was consumed, the solution was adjusted to pH=5˜6 with aq. HCl solution (1N) and concentrated in vacuo to give a crude product of 6-[8-[[2-[[2-(tert-butoxycarbonylamino)acetyl]amino]acetyl]-methyl-amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (40 mg, 82.9% yield) as a yellow solid. MS (ESI): 774.2 ([M+H]⁺). It was used directly in the next step without further purification.

### Step (c) Preparation of 6-[8-[[2-[(2-aminoacetyl)amino]acetyl]-methyl-amino]-5,6-difluoro-4-[cis-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (Example 1.30)

To a solution of 6-[8-[[2-[[2-(tert-butoxycarbonylamino)acetyl]amino]acetyl]-methyl-amino]-5,6-difluoro-4-[cis-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (40.0 mg, 0.05 mmol) in DCM (1 mL) was added TFA (0.2 mL, 2.6 mmol), and the resulting reaction mixture was stirred at 10° C. for 1 h until LC-MS indicated the starting material was consumed. The mixture solution was concentrated in vacuo to give a crude product, which was purified by prep-HPLC (NH₃.H₂O as additive) to give 6-[8-[[2-[(2-aminoacetyl)amino]acetyl]-methyl-amino]-5,6-difluoro-4-[cis-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (13.1 mg, 35.8% yield) as an off-white solid. MS (ESI): 674.1 ([M+H]⁺). ¹H NMR (400 MHz, DMSO-d₆) δ ppm: 9.26 (m, 1H), 9.13 (m, 1H), 8.73 (m, 1H), 8.22 (m, 1H), 8.15 (m, 1H), 7.81 (m, 1H), 7.58 (m, 1H), 4.47 (m, 1H), 4.35 (m, 1H), 4.19 (s, 3H), 4.05 (m, 1H), 3.80 (m, 1H), 3.61 (m, 1H), 3.11 (s, 3H), 3.00 (m, 2H), 2.85 (m, 1H), 2.62 (m, 2H), 2.49 (m, 2H), 2.15 (m, 4H), 2.03 (m, 1H), 1.90 (m, 1H), 1.69 (m, 1H).

### Example 1.31

### Ethyl 6-[5,6-difluoro-8-(methylamino)-4-[cis-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate

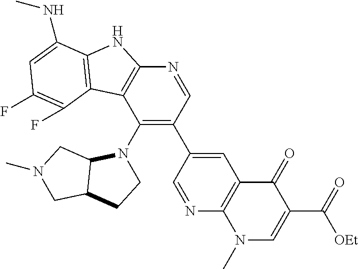

The titled compound was synthesized according to the following scheme:

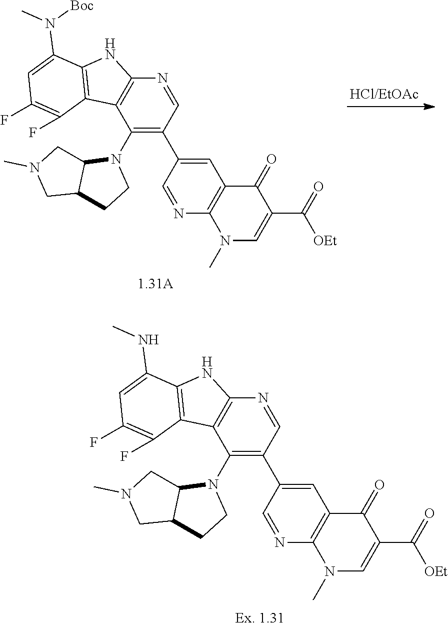

### Step (a) Preparation of ethyl 6-[8-[tert-butoxycarbonyl(methyl)amino]-5,6-difluoro-4-[cis-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (Compound 1.31A)

In analogy to the synthesis of compound 1.16C, cis-5-methyl-2,3,3a,4,6,6a-hexahydro-1H-pyrrolo[2,3-c]pyrrole (CAS: 876130-70-0) was used instead of 5-methyl-1H-hexahydropyrrolo[2,3-c]pyrrole dihydrochloride in step (a) to give ethyl 6-[8-[tert-butoxycarbonyl(methyl)amino]-5,6-difluoro-4-[cis-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (59.6% yield) as a yellow solid after step (b). MS (ESI): 688.2 ([M+H]⁺).

### Step (b) Preparation of ethyl 6-[5,6-difluoro-8-(methylamino)-4-[cis-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (Example 1.31)

To a solution of ethyl 6-[8-[tert-butoxycarbonyl(methyl)amino]-5,6-difluoro-4-[cis-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (250 mg, 0.36 mmol) in EtOAc (5 mL) was added HCl in EtOAc solution (1.0 mL, 4 N), and the resulting reaction mixture was stirred at 15° C. for 1 h. After LC-MS showed the staring material was consumed completely, the mixture solution was concentrated in vacuo to give a crude product of ethyl 6-[5,6-difluoro-8-(methylamino)-4-[cis-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (210 mg, 98.3% yield) as a yellow solid. MS (ESI): 588.2 ([M+H]⁺).

### Example 1.32

### 7-[5,6-Difluoro-8-[methyl(phosphonooxymethoxycarbonyl)amino]-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid

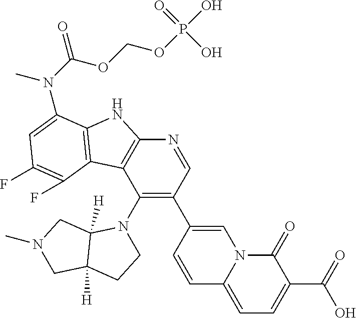 [SMILES: CN1C[C@@H]2CCN(c3c(-c4ccc5ccc(C(=O)O)c(=O)n5c4)cnc4[nH]c5c(N(C)C(=O)OCOP(=O)(O)O)cc(F)c(F)c5c34)[C@@H]2C1]

The titled compound was synthesized according to the following scheme:

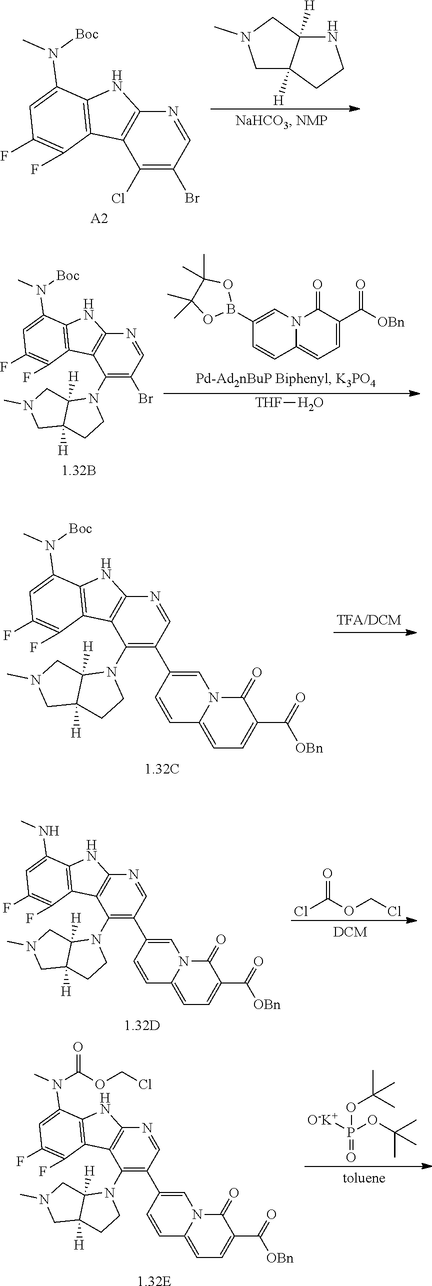

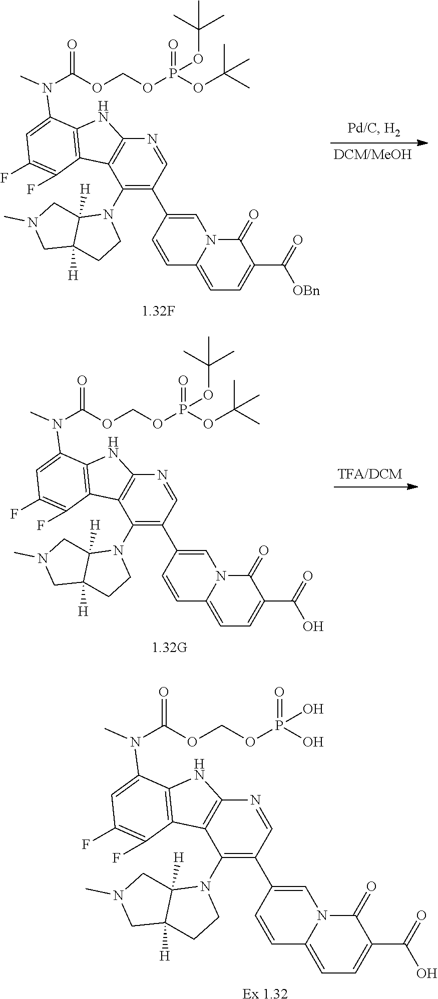 [SMILES: CN1C[C@@H]2CCN(c3c(-c4ccc5ccc(C(=O)OCc6ccccc6)c(=O)n5c4)cnc4[nH]c5c(N(C)C(=O)OCOP(=O)(OC(C)(C)C)OC(C)(C)C)cc(F)c(F)c5c34)[C@@H]2C1 ; "1.32F" ; CN1C[C@@H]2CCN(c3c(-c4ccc5ccc(C(=O)O)c(=O)n5c4)cnc4[nH]c5c(N(C)C(=O)OCOP(=O)(OC(C)(C)C)OC(C)(C)C)cc(F)c(F)c5c34)[C@@H]2C1 ; "1.32G" ; CN1C[C@@H]2CCN(c3c(-c4ccc5ccc(C(=O)O)c(=O)n5c4)cnc4[nH]c5c(N(C)C(=O)OCOP(=O)(O)O)cc(F)c(F)c5c34)[C@@H]2C1 ; "Ex 1.32"]

### Step (a) Preparation of tert-butyl N-[3-bromo-5,6-difluoro-4-[(3aS,6aS)-5-methyl-2,3,3a,4,6,6a-hexahydropyrrolo[2,3-c]pyrrol-1-yl]-9H-pyrido[2,3-b]indol-8-yl]-N-methyl-carbamate (Compound 1.32B)

In analogy to the synthesis of compound 1.01B, tert-butyl N-(3-bromo-4-chloro-5,6-difluoro-9H-pyrido[2,3-b]indol-8-yl)-N-methyl-carbamate (Intermediate A2) was used instead of tert-butyl N-(3,4-dichloro-5-cyano-6-fluoro-9H-pyrido[2,3-b]indol-8-yl)-N-methyl-carbamate (Intermediate A3) to give tert-butyl N-[3-bromo-5,6-difluoro-4-[(3aS,6aS)-5-methyl-2,3,3a,4,6,6a-hexahydropyrrolo[2,3-c]pyrrol-1-yl]-9H-pyrido[2,3-b]indol-8-yl]-N-methyl-carbamate as a light yellow solid (88.3% yield) after silica gel flash chromatograph purification (petrolieum ether:EtOAc=10:1˜1:1, 2% Et₃N as additive). MS (ESI): 536.2 ([{⁷⁹Br}M+H]⁺).

### Step (b) Preparation of benzyl 7-[8-[tert-butoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate (Compound 1.32C)

In analogy to the synthesis of compound 1.01C, tert-butyl N-[3-bromo-5,6-difluoro-4-[(3aS,6aS)-5-methyl-2,3,3a,4,6,6a-hexahydropyrrolo[2,3-c]pyrrol-1-yl]-9H-pyrido[2,3-b]indol-8-yl]-N-methyl-carbamate (compound 1.32B) and benzyl 1-methyl-4-oxo-6-(4,4,5,5-tetramethyl-1,3,2-dioxaborolan-2-yl)-1,8-naphthyridine-3-carboxylate (Intermediate B4) were used instead of tert-butyl N-[3-chloro-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-8-yl]-N-methyl-carbamate (compound 1.01B) and benzyl 1-methyl-4-oxo-6-(4,4,5,5-tetramethyl-1,3,2-dioxaborolan-2-yl)-1,8-naphthyridine-3-carboxylate (Intermediate B3) to give benzyl 7-[8-[tert-butoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate (77.0% yield) as a yellow solid after prep-HPLC purification (TFA as additive). MS (ESI): 735.3 ([M+H]⁺).

### Step (c) Preparation of benzyl 7-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate (Compound 1.32D)

In analogy to the synthesis of compound 1.01D, benzyl 7-[8-[tert-butoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate (compound 1.32C) was used instead of benzyl 6-[8-[tert-butoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (compound 1.01C) to give benzyl 7-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate (97.5%) as a yellow solid after prep-HPLC purification (TFA as additive). MS (ESI): 635.2 ([M+H]⁺).

### Step (d) Preparation of benzyl 7-[8-[chloromethoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate (Compound 1.32E)

In analogy to the synthesis of compound 1.01E, benzyl 7-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate (compound 1.32D) was used instead of benzyl 6-[5-cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (compound 1.01D) to give benzyl 7-[8-[chloromethoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate as a yellow solid (53.6% yield) after prep-HPLC purification (TFA as additive) and subsequent work up. MS (ESI): 727.2 ([{³⁵Cl}M+H]⁺).

### Step (e) Preparation of benzyl 7-[8-[ditert butoxyphosphoryloxymethoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate (Compound 1.32F)

In analogy to the synthesis of compound 1.01F, benzyl 7-[8-[chloromethoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate (compound 1.32E) was used instead of benzyl 6-[8-[chloromethoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (compound 1.01E) to give benzyl 7-[8-[ditert butoxyphosphoryloxymethoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate as a light yellow solid (57.9% yield) after prep-HPLC purification (TFA as additive) and subsequent work up. MS (ESI): 901.3 ([M+H]⁺).

### Step (f) Preparation of 7-[8-[ditert-butoxyphosphoryloxymethoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid (Compound 1.32G)

In analogy to the synthesis of compound 1.01G, benzyl 7-[8-[ditert butoxyphosphoryloxymethoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate (compound 1.32F) was used instead of benzyl 6-[5-cyano-8-[di-tert-butoxyphosphoryloxymethoxycarbonyl(methyl)amino]-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (compound 1.01F) to give 7-[8-[ditert-butoxyphosphoryloxymethoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid (87.8% yield) as a crude product of light yellow solid.

MS (ESI): 811.3 ([M+H]⁺).

### Step (g) Preparation of 7-[5,6-difluoro-8-[methyl(phosphonooxymethoxycarbonyl)amino]-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid (Example 1.32)

In analogy to the synthesis of Example 1.01, 7-[8-[ditert-butoxyphosphoryloxymethoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid (compound 1.32G) was used instead of 6-[5-cyano-8-[di-tert-butoxyphosphoryloxymethoxycarbonyl(methyl)amino]-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (compound 1.01G) to give 7-[5,6-difluoro-8-[methyl(phosphonooxymethoxycarbonyl)amino]-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid as a light yellow solid (49.8% yield) after prep-HPLC purification (NH₄HCO₃ as additive). MS (ESI): 699.1 ([M+H]⁺). ¹H NMR (400 MHz, DMSO-d6) δ ppm 12.88-13.65 (m, 1H), 9.18 (s, 1H), 8.38-8.50 (m, 1H), 8.07-8.30 (m, 3H), 7.58 (br dd, J=11.5, 6.6 Hz, 1H), 7.36 (br d, J=8.6 Hz, 1H), 5.49 (br s, 1H), 4.50 (br s, 1H), 3.26 (br s, 6H), 3.17 (br s, 2H), 2.97 (br s, 1H), 2.73 (br s, 1H), 2.63 (br s, 1H), 2.40 (br s, 2H), 2.01 (br s, 1H), 1.73 (br s, 1H).

### Example 1.33

### 7-[5,6-Difluoro-8-[methyl-[[(E)-3-carboxyprop-2-enoyl]oxymethoxycarbonyl]amino]-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid

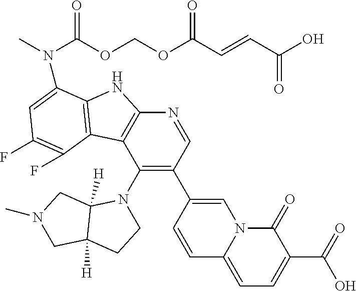 [SMILES: CN1C[C@@H]2CCN(c3c(-c4ccc5ccc(C(=O)O)c(=O)n5c4)cnc4[nH]c5c(N(C)C(=O)OCOC(=O)/C=C/C(=O)O)cc(F)c(F)c5c34)[C@@H]2C1]

The titled compound was synthesized according to the following scheme:

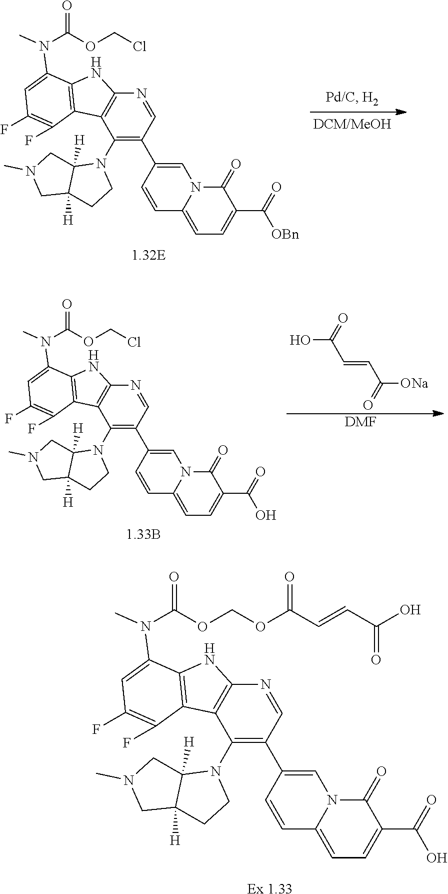

### Step (a) Preparation of 7-[8-[chloromethoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid (compound 1.33B)

In analogy to the synthesis of compound 1.02B, benzyl 7-[8-[chloromethoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate (compound 1.32E) was used instead of benzyl 6-[8-[chloromethoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (compound 1.01E) to give a crude product of 7-[8-[chloromethoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid (93.3% yield) as a light yellow solid. MS (ESI): 637.2 ([M+H]⁺).

### Step (b) Preparation of 7-[5,6-difluoro-8-[methyl-[[(E)-3-carboxyprop-2-enoyl]oxymethoxycarbonyl]amino]-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid (Example 1.33)

In analogy to the synthesis of Example 1.02, 7-[8-[chloromethoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid (compound 1.33B) was used instead of 6-[4-[(3aS,6aS)-5-methyl-2,3,3a,4,6,6a-hexahydropyrrolo[2,3-c]pyrrol-1-yl]-8-[chloromethoxycarbonyl(methyl)amino]-5-cyano-6-fluoro-9H-pyrido[2,3-b]indol-3-yl]-1-methyl-4-oxo-1,8-naphthyridine-3-carboxylic acid (compound 1.02B) to give 7-[5,6-difluoro-8-[methyl-[[(E)-3-carboxyprop-2-enoyl]oxymethoxycarbonyl]amino]-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid (42% yield) as a yellow solid after prep-HPLC purification (NH₄HCO₃ as additive). MS (ESI): 717.1 ([M+H]⁺). ¹H NMR (400 MHz, DMSO-d6) δ ppm 12.52 (br s, 1H), 9.22 (s, 1H), 8.45 (d, J=8.6 Hz, 1H), 8.16-8.34 (m, 3H), 7.61-7.76 (m, 1H), 7.36 (br d, J=8.6 Hz, 1H), 6.37-6.87 (m, 2H), 5.60-5.96 (m, 2H), 4.36-4.53 (m, 1H), 3.25-3.29 (m, 3H), 3.17-3.19 (m, 1H), 3.12 (br s, 1H), 2.90 (br s, 2H), 2.56-2.64 (m, 2H), 2.37 (br d, J=9.3 Hz, 1H), 2.18 (s, 3H), 1.88-2.05 (m, 2H), 1.71 (br d, J=4.4 Hz, 1H).

### Example 1.34

### 7-[8-[[2-[[2-[(2-Aminoacetyl)amino]acetyl]amino]acetyl]-methyl-amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid

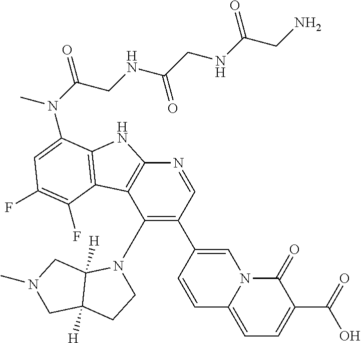 [SMILES: CN1C[C@@H]2CCN(c3c(-c4ccc5ccc(C(=O)O)c(=O)n5c4)cnc4[nH]c5c(N(C)C(=O)CNC(=O)CNC(=O)CN)cc(F)c(F)c5c34)[C@@H]2C1]

The titled compound was synthesized according to the following scheme:

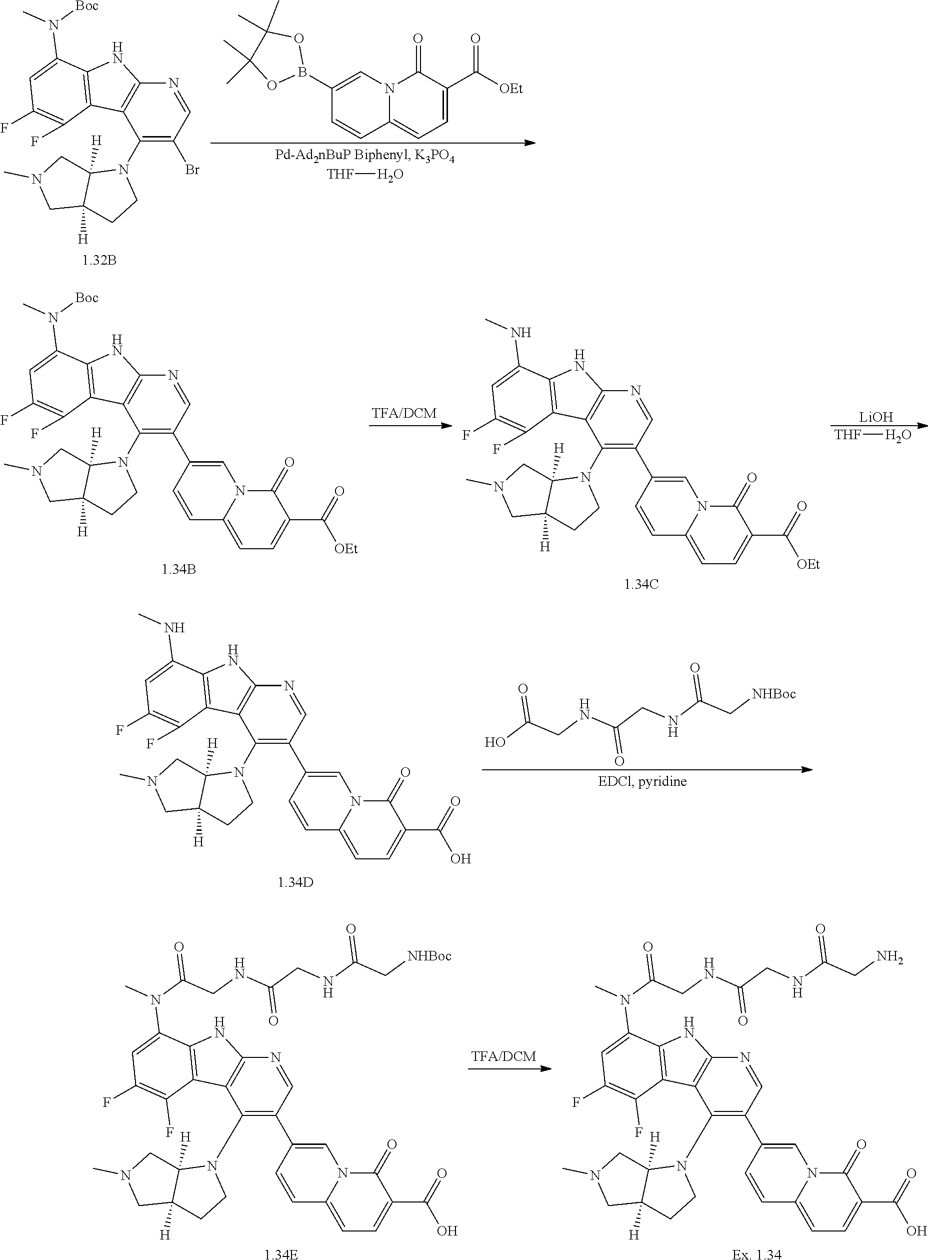

### Step (a) Preparation of ethyl 7-[8-[tert-butoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate (Compound 1.34B)

In analogy to the synthesis of compound 1.03B, tert-butyl N-[3-bromo-5,6-difluoro-4-[(3aS,6aS)-5-methyl-2,3,3a,4,6,6a-hexahydropyrrolo[2,3-c]pyrrol-1-yl]-9H-pyrido[2,3-b]indol-8-yl]-N-methyl-carbamate (compound 1.32B) was used instead of tert-butyl N-[3-chloro-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-8-yl]-N-methyl-carbamate (20.0 g, 40.1 mmol, compound 1.01B) to give ethyl 7-[8-[tert-butoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate as a yellow solid (73.0% yield) after prep-HPLC purification (TFA as additive). MS (ESI): 673.3 ([M+H]⁺).

### Step (b) Preparation of ethyl 7-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate (Compound 1.34C)

In analogy to the synthesis of compound 1.03D, ethyl 7-[8-[tert-butoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate (compound 1.34B) was used instead of 6-[8-[tert-butoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (compound 1.03C) to give a crude product of ethyl 7-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate (93.2% yield) after prep-HPLC purification (NH₃.H₂O as additive) as a yellow solid. MS (ESI): 573.3 ([M+H]⁺).

### Step (c) Preparation of 7-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid (Compound 1.34D)

In analogy to the synthesis of compound 1.03C, ethyl 7-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate (compound 1.34C) was used instead of ethyl 6-[8-[tert-butoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (compound 1.03B) to give a crude product of 7-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid (96% yield) as a yellow solid. MS (ESI): 545.1.

### Step (d) Preparation of 7-[8-[[2-[[2-[[2-(tert-butoxycarbonylamino)acetyl]amino]acetyl]amino]acetyl]-methyl-amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid (Compound 1.34E)

A mixture solution of 7-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid (200 mg, 0.37 mmol), 2-[[2-[[2-(tert-butoxycarbonylamino)acetyl]amino]acetyl]amino]acetic acid (200 mg, 0.69 mmol), and EDCI (200 mg, 1.04 mmol) in pyridine (2 mL) was stirred at 80° C. for 16 h until LC-MS showed the starting material was consumed. The mixture solution was cooled back to r.t., concentrated in vacuo to give a crude product, which was purified by prep-HPLC (NH₃.H₂O as additive) to give 7-[8-[[2-[[2-[[2-(tert-butoxy carbonylamino)acetyl]amino]acetyl]amino]acetyl]-methyl-amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid (80 mg, 26.7% yield) as a yellow solid. MS (ESI): 816.6 ([M+H]⁺).

### Step (e) Preparation of 7-[8-[[2-[[2-[(2-aminoacetyl)amino]acetyl]amino]acetyl]-methyl-amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid (Example 1.34)

To a solution of 7-[8-[[2-[[2-[[2-(tot-butoxy carbonylamino)acetyl]amino]acetyl]amino]acetyl]-methyl-amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid (80 mg, 0.10 mmol) in DCM (5 mL) was added TFA (1.0 mL, 12.98 mmol), and the resulting reaction mixture was stirred at 20° C. for 1 h. The reaction mixture was then concentrated in vacuo to give a crude product, which was purified by prep-HPLC (NH₃.H₂O as additive) to give 7-[8-[[2-[[2-[(2-aminoacetyl)amino]acetyl]amino]acetyl]-methyl-amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid (45 mg, 61.9% yield) as a yellow solid. MS (ESI): 716.3 ([M+H]⁺). ¹H NMR (400 MHz, DMSO-d₆, T=80) δ ppm: 9.24 (s, 1H), 8.47 (d, J=8.4 Hz, 1H), 8.31 (s, 1H), 8.15 (m, 2H), 7.70 (m, 1H), 7.63 (m, 1H), 7.32 (m, 1H), 4.44 (m, 1H), 3.76 (m, 2H), 3.31 (m, 4H), 3.26 (m, 2H), 3.16 (m, 3H), 3.00 (m, 1H), 2.45 (m, 2H), 2.25 (m, 1H), 2.10 (s, 3H), 2.05 (m, 1H), 1.90 (m, 1H), 1.74 (m, 1H).

### Example 1.35

### 7-[8-[[2-[(2-Aminoacetyl)amino]acetyl]-methyl-amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid

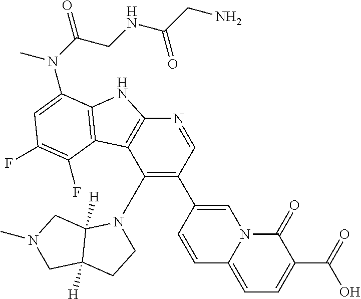 [SMILES: CN1C[C@@H]2CCN(c3c(-c4ccc5ccc(C(=O)O)c(=O)n5c4)cnc4[nH]c5c(N(C)C(=O)CNC(=O)CN)cc(F)c(F)c5c34)[C@@H]2C1]

The titled compound was synthesized according to the following scheme:

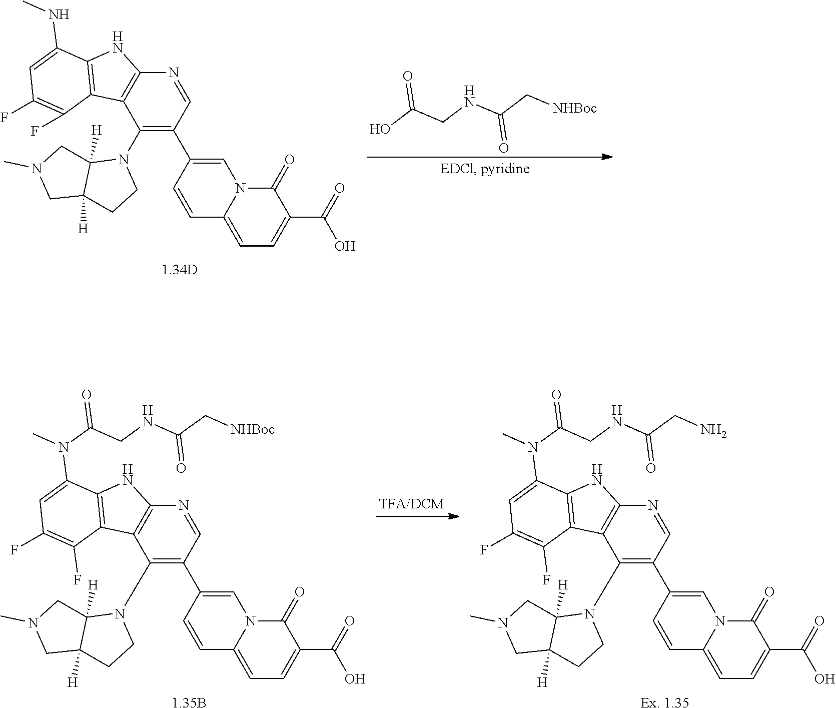

### Step (a) Preparation of 7-[8-[[2-[[2-(tert-butoxycarbonylamino)acetyl]amino]acetyl]-methyl-amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid (Compound 1.35B)

In analogy to the synthesis of compound 1.34E, 2-[[2-(tert-butoxycarbonylamino)acetyl]amino]acetic acid was used instead of 2-[[2-[[2-(tert-butoxycarbonylamino)acetyl]amino]acetyl]amino]acetic acid to give 7-[8-[[2-[[2-(tert-butoxycarbonylamino)acetyl]amino]acetyl]-methyl-amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid (32.3% yield) as a yellow solid after prep-HPLC purification (NH₃.H₂O as additive). MS (ESI): 759.6 ([M+H]⁺).

### Step (b) Preparation of 7-[8-[[2-[(2-aminoacetyl)amino]acetyl]-methyl-amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid (Example 1.35)

In analogy to the synthesis of Example 1.34, 7-[8-[[2-[[2-(tot-butoxycarbonylamino)acetyl]amino]acetyl]-methyl-amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid was used instead of 7-[8-[[2-[[2-[[2-(tert-butoxy carbonylamino)acetyl]amino]acetyl]amino]acetyl]-methyl-amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid (compound 1.34E) to give 7-[8-[[2-[(2-aminoacetyl)amino]acetyl]-methyl-amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid (59.3% yield) as a yellow solid after prep-HPLC purification (TFA as additive). MS (ESI): 659.2 ([M+H]⁺). ¹H NMR (400 MHz, DMSO-d₆, T=80) δ ppm: 12.65 (m, 1H), 9.24 (m, 1H), 8.52 (d, J=8.4 Hz, 1H), 8.37 (m, 2H), 8.22 (d, J=8.4 Hz, 1H), 8.05 (m, 1H), 7.94 (m, 1H), 7.75 (m, 1H), 7.39 (d, J=8.4 Hz, 1H), 4.32 (m, 1H), 3.85 (m 4H), 3.63 (m, 2H), 3.51 (m, 2H), 3.36 (m, 3H), 2.73 (s, 3H), 2.55 (m, 3H), 2.40 (m, 1H), 2.18 (m, 1H), 1.93 (m, 1H).

### Example 1.36

### 7-[5,6-Difluoro-8-(methylamino)-1-(phosphonooxymethyl)-4-[(3aS,6aS)-5-methyl-2,3,3a,4,6,6a-hexahydropyrrolo[2,3-c]pyrrol-1-yl]pyrido[2,3-b]indol-3-yl]-4-oxo-quinolizine-3-carboxylic acid

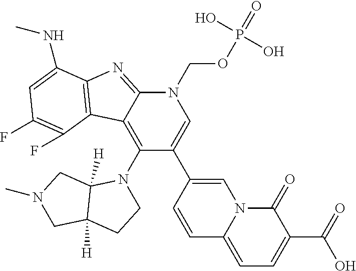 [SMILES: CNc1cc(F)c(F)c2c3c(N4CC[C@H]5CN(C)C[C@H]54)c(-c4ccc5ccc(C(=O)O)c(=O)n5c4)cn(COP(=O)(O)O)c-3nc12]

The titled compound was synthesized according to the following scheme:

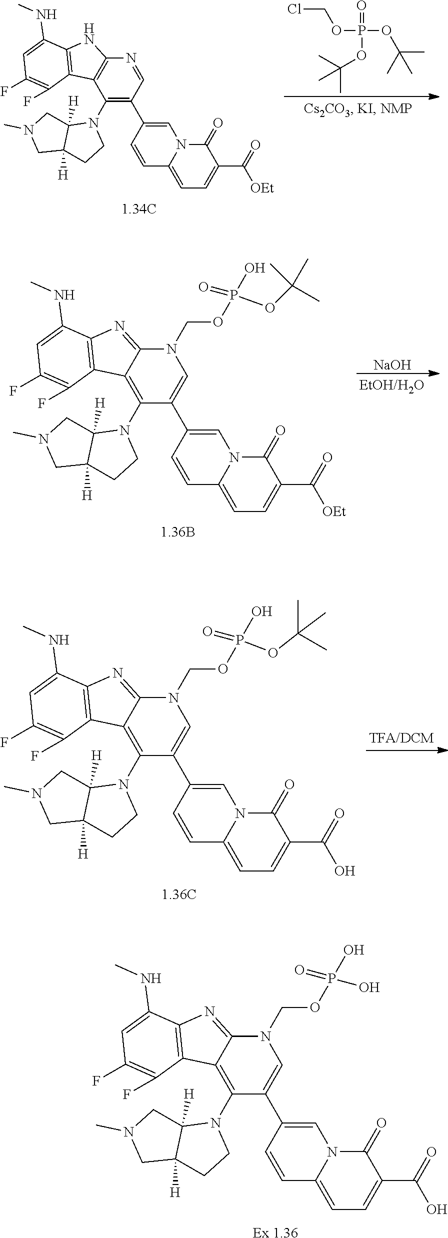

### Step (a) Preparation of ethyl 7-[1-[[tert-butoxy(hydroxy)phosphoryl]oxymethyl]-5,6-difluoro-8-(methylamino)-4-[(3aS,6aS)-5-methyl-2,3,3a,4,6,6a-hexahydropyrrolo[2,3-c]pyrrol-1-yl]pyrido[2,3-b]indol-3-yl]-4-oxo-quinolizine-3-carboxylate (Compound 1.36B)

In analogy to the synthesis of compound 1.04C, ethyl 7-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate (compound 1.34C) was used instead of ethyl 6-[5-cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (compound 1.04B) to give ethyl 7-[1-[[tert-butoxy(hydroxy)phosphoryl]oxymethyl]-5,6-difluoro-8-(methylamino)-4-[(3aS,6aS)-5-methyl-2,3,3a,4,6,6a-hexahydropyrrolo[2,3-c]pyrrol-1-yl]pyrido[2,3-b]indol-3-yl]-4-oxo-quinolizine-3-carboxylate (42.0% yield) as a yellow solid after prep-HPLC purification (NH₄HCO₃ as additive). MS (ESI): 739.3 ([M+H]⁺).

### Step (b) Preparation of 7-[1-[[tert-butoxy(hydroxy)phosphoryl]oxymethyl]-5,6-difluoro-8-(methylamino)-4-[(3aS,6aS)-5-methyl-2,3,3a,4,6,6a-hexahydropyrrolo[2,3-c]pyrrol-1-yl]pyrido[2,3-b]indol-3-yl]-4-oxo-quinolizine-3-carboxylic acid (Compound 1.36C)

In analogy to the synthesis of compound 1.04D, ethyl 1-[1-[[tert-butoxy(hydroxy)phosphoryl]oxymethyl]-5,6-difluoro-8-(methylamino)-4-[(3aS,6aS)-5-methyl-2,3,3a,4,6,6a-hexahydropyrrolo[2,3-c]pyrrol-1-yl]pyrido[2,3-b]indol-3-yl]-4-oxo-quinolizine-3-carboxylate (compound 1.36B) was used instead of ethyl 6-[1-[[tert-butoxy(hydroxy)phosphoryl]oxymethyl]-5-cyano-6-fluoro-8-(methylamino)-4-[(3aS,6aS)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylate (compound 1.04C) to give a crude product of 7-[1-[[tert-butoxy(hydroxy)phosphoryl]oxymethyl]-5,6-difluoro-8-(methylamino)-4-[(3aS,6aS)-5-methyl-2,3,3a,4,6,6a-hexahydropyrrolo[2,3-c]pyrrol-1-yl]pyrido[2,3-b]indol-3-yl]-4-oxo-quinolizine-3-carboxylic acid (95.3% yield) as a yellow solid. MS (ESI): 711.3 ([M+H]⁺).

### Step (c) Preparation of 7-[5,6-difluoro-8-(methylamino)-1-(phosphonooxymethyl)-4-[(3aS,6aS)-5-methyl-2,3,3a,4,6,6a-hexahydropyrrolo[2,3-c]pyrrol-1-yl]pyrido[2,3-b]indol-3-yl]-4-oxo-quinolizine-3-carboxylic acid (Example 1.36)

In analogy to the synthesis of Example 1.04, 1-[1-[[tert-butoxy(hydroxy)phosphoryl]oxymethyl]-5,6-difluoro-8-(methylamino)-4-[(3aS,6aS)-5-methyl-2,3,3a,4,6,6a-hexahydropyrrolo[2,3-c]pyrrol-1-yl]pyrido[2,3-b]indol-3-yl]-4-oxo-quinolizine-3-carboxylic acid (compound 1.36C) was used instead of ethyl 6-[1-[[tert-butoxy(hydroxy)phosphoryl]oxymethyl]-5-cyano-6-fluoro-8-(methylamino)-4-[(3aS,6aS)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylate (compound 1.04C) to give 7-[5,6-difluoro-8-(methylamino)-1-(phosphonooxymethyl)-4-[(3aS,6aS)-5-methyl-2,3,3a,4,6,6a-hexahydropyrrolo[2,3-c]pyrrol-1-yl]pyrido[2,3-b]indol-3-yl]-4-oxo-quinolizine-3-carboxylic acid (9.2% yield) as a yellow solid after prep-HPLC purification (TFA as additive). MS (ESI): 655.3 ([M+H]⁺). ¹H NMR (400 MHz, DMSO-d6) δ ppm 14.14 (s, 1H), 9.37 (br s, 1H), 8.54 (br s, 1H), 8.49 (br d, J=8.25 Hz, 1H), 8.24 (br d, J=12.51 Hz, 2H,) 7.40 (br d, J=8.25 Hz, 1H), 6.70 (br dd, J=12.51, 6.00 Hz, 1H), 6.54 (br s, 1H), 6.08 (br d, J=9.88 Hz, 2H), 4.99 (br s, 2H), 3.12 (br s, 5H), 2.92 (br s, 3H), 2.68 (br s, 2H), 1.90˜2.06 (m, 2H), 1.76 (s, 1H) 1.20-1.32 (m, 3H).

### Example 1.37

### 2-[(2S)-2-Amino-3-methyl-butanoyl]oxyethyl 7-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate

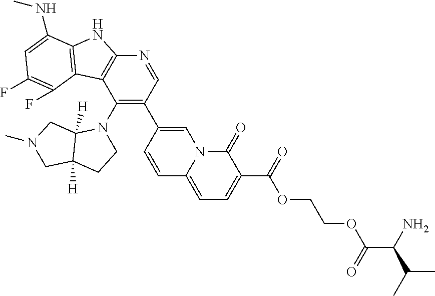 [SMILES: CNc1cc(F)c(F)c2c1[nH]c1ncc(-c3ccc4ccc(C(=O)OCCOC(=O)[C@@H](N)C(C)C)c(=O)n4c3)c(N3CC[C@H]4CN(C)C[C@H]43)c12]

The titled compound was synthesized according to the following scheme:

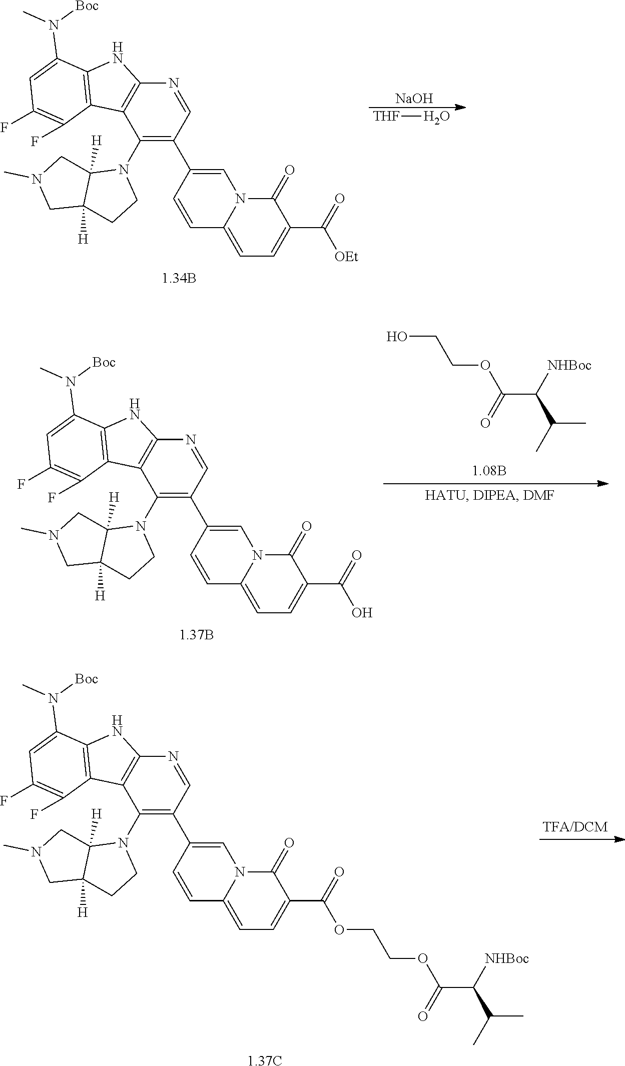

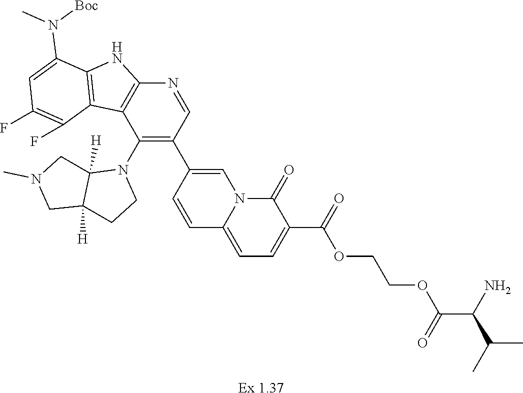 [SMILES: CC(C)[C@H](N)C(=O)OCCOC(=O)c1ccc2ccc(-c3cnc4[nH]c5c(N(C)C(=O)OC(C)(C)C)cc(F)c(F)c5c4c3N3CC[C@H]4CN(C)C[C@H]43)cn2c1=O ; "Ex 1.37"]

### Step (a) Preparation of 7-[8-[tert-butoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid (Compound 1.37B)

In analogy to the synthesis of compound 1.03C, ethyl 7-[8-[tert-butoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate (compound 1.34B) was used instead of ethyl 6-[8-[tert-butoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (compound 1.03B) to give a crude product of 7-[8-[tert-butoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid (78.3% yield) as a yellow solid. MS (ESI): 650.2 ([M+H]⁺).

### Step (b) Preparation of 2-[(2S)-2-(tert-butoxycarbonylamino)-3-methyl-butanoyl]oxyethyl 7-[8-[tert-butoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate (Compound 1.37C)

In analogy to the synthesis of compound 1.08C, 7-[8-[tert-butoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid (compound 1.37B) was used instead of 6-[8-[tert-butoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (compound 1.03C) to give 2-[(2S)-2-(tert-butoxycarbonylamino)-3-methyl-butanoyl]oxyethyl 7-[8-[tert-butoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate (25.4% yield) as a yellow solid after prep-HPLC purification (NH₄HCO₃ as additive). MS (ESI): 888.5 ([M+H]⁺).

### Step (c) Preparation of 2-[(2S)-2-amino-3-methyl-butanoyl]oxyethyl 7-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate (Example 1.37)

In analogy to the synthesis of Example 1.08, 2-[(2S)-2-(tert-butoxycarbonylamino)-3-methyl-butanoyl]oxyethyl 7-[8-[tert-butoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate (compound 1.37C) was used instead of 2-[(2S)-2-(tert-butoxycarbonylamino)-3-methyl-butanoyl]oxy ethyl 6-[8-[tert-butoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (compound 1.08C) to give 2-[(2S)-2-amino-3-methyl-butanoyl]oxyethyl 7-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate (49.5% yield) as a yellow solid after prep-HPLC purification (NH₄CO₃ as additive). MS (ESI): 688.4 ([M+H]⁺). ¹H NMR (400 MHz, DMSO-d₆) δ ppm: ¹H NMR (400 MHz, DMSO-d₆) δ=11.76 (br s, 1H), 9.14 (s, 1H), 8.27 (d, 0.7=8.6 Hz, 1H), 8.19 (s, 1H), 8.10-8.04 (m, 1H), 8.01-7.96 (m, 1H), 6.96 (d, 0.7=8.6 Hz, 1H), 6.58 (dd, J=6.2, 13.6 Hz, 1H), 5.71 (br d, 0.7=4.0 Hz, 1H), 4.52-4.33 (m, 5H), 3.17-3.03 (m, 3H), 2.89 (d, 0.7=4.9 Hz, 4H), 2.45 (br d, 0.7=10.5 Hz, 2H), 2.21 (br t, 0.7=8.3 Hz, 1H), 2.13-2.07 (m, 3H), 2.02-1.89 (m, 2H), 1.83 (br dd, 0.7=5.1, 9.6 Hz, 1H), 1.66 (br s, 1H), 0.86 (dd, 0.7=6.8, 10.3 Hz, 6H).

### Example 1.38

### 2-[[2-[[7-[5,6-Difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carbonyl]amino]acetyl]amino]acetic acid

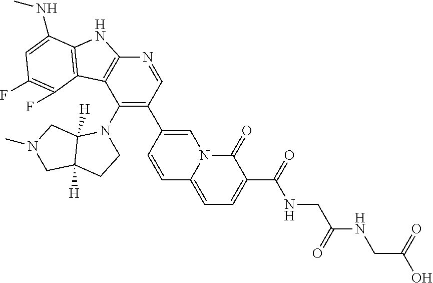 [SMILES: CNc1cc(F)c(F)c2c1[nH]c1ncc(-c3ccc4ccc(C(=O)NCC(=O)NCC(=O)O)c(=O)n4c3)c(N3CC[C@H]4CN(C)C[C@H]43)c12]

The titled compound was synthesized according to the following scheme:

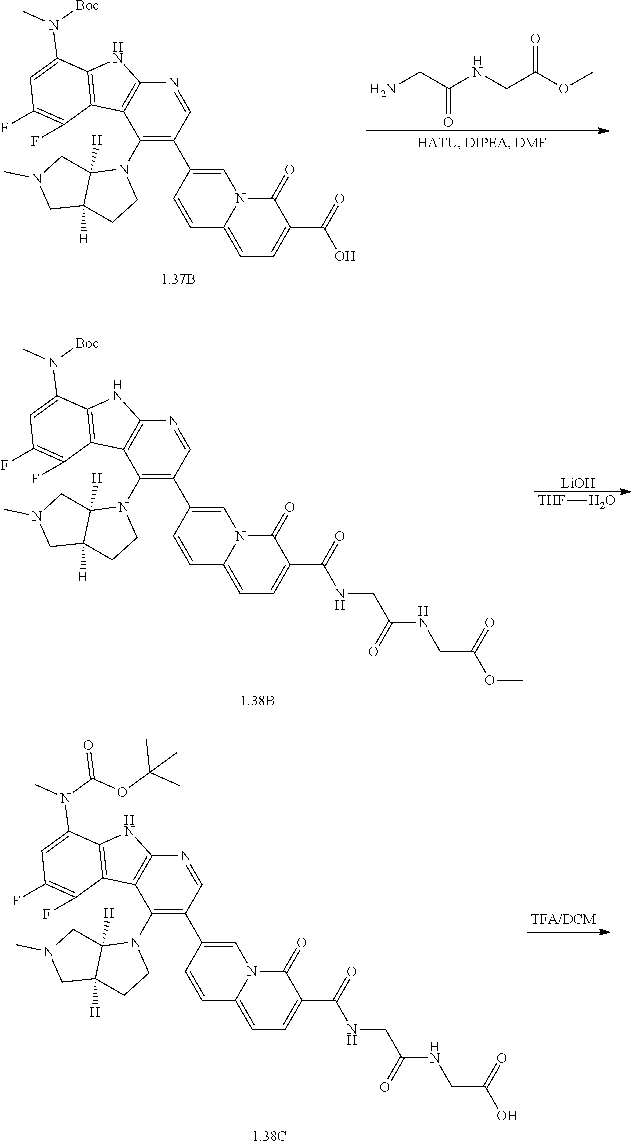

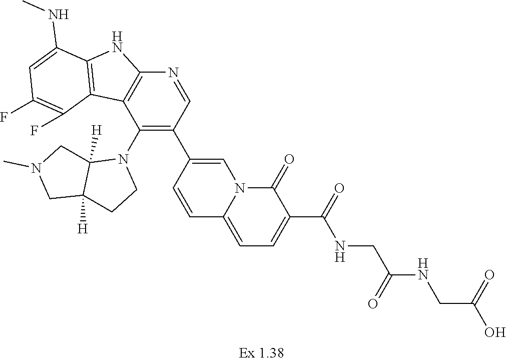 [SMILES: CNc1cc(F)c(F)c2c1[nH]c1ncc(-c3ccc4ccc(C(=O)NCC(=O)NCC(=O)O)c(=O)n4c3)c(N3CC[C@H]4CN(C)C[C@H]43)c12 ; "Ex 1.38"]

### Step (a) Preparation of methyl 2-[[2-[[7-[8-[tert-butoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carbonyl]amino]acetyl]amino]acetate (Compound 1.38B)

A mixture solution of 7-[8-[tert-butoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid (40.0 mg, 0.06 mmol, compound 1.37B), HATU (47.2 mg, 0.12 mmol) and DIPEA (0.03 mL, 0.190 mmol) in DMF (2 mL) was stirred at 20° C. for 0.5 h before methyl 2-[(2-aminoacetyl)amino]acetate (18.1 mg, 0.12 mmol) was added, and the resulting reaction mixture was stirred at 25° C. for another 16 h. After LC-MS showed the starting material was consumed, the mixture solution was concentrated in vacuo to give a crude product, which was further purified by prep-HPLC (TFA as additive) to give methyl 2-[[2-[[7-[8-[tert-butoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carbonyl]amino]acetyl]amino]acetate (40 mg, 83.4% yield) as a yellow solid. MS (ESI): 773.4 ([M+H]⁺).

### Step (b) Preparation of 2-[[2-[[7-[8-[tert-butoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carbonyl]amino]acetyl]amino]acetic acid (Compound 1.38C)

To a solution of methyl 2-[[2-[[7-[8-[tert-butoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carbonyl]amino]acetyl]amino]acetate (38.0 mg, 0.05 mmol) in THF (5 mL) was added aq. lithium hydroxide solution (1 N, 0.25 mL, 0.25 mmol), and the resulting reaction mixture was stirred at 40° C. for 2 h. After LC-MS showed the starting material was consumed, the mixture solution was concentrated in vacuo to give a cruder product of 2-[[2-[[7-[8-[tert-butoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carbonyl]amino]acetyl]amino]acetic acid (37 mg, 97.2% yield) as a yellow solid. MS (ESI): 759.4 ([M+H]⁺). It was used directly in the next step without further purification.

### Step (c) Preparation of 2-[[2-[[7-[5,6-Difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carbonyl]amino]acetyl]amino]acetic acid (Example 1.38)

To a solution of 2-[[2-[[7-[8-[tert-butoxycarbonyl(methyl)amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carbonyl]amino]acetyl]amino]acetic acid_(37.0 mg, 0.05 mmol) in DCM (5.09 mL) was added TFA (0.19 mL, 2.44 mmol), and the resulting reaction mixture was stirred at 25° C. for 1 h. After LC-MS showed the starting material was consumed, the mixture solution was concentrated in vacuo to give a crude product, which was further purified by prep-HPLC (TFA as additive) to give 2-[[2-[[7-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carbonyl]amino]acetyl]amino]acetic acid (22.6 mg, 51.5% yield) as a yellow solid. MS (ESI): 659.3 ([M+H]⁺). ¹H NMR (400 MHz, DMSO-d6) δ ppm 11.85-12.03 (m, 1H), 9.88-10.05 (m, 1H), 9.11-9.65 (m, 2H), 8.57 (d, J=8.38 Hz, 1H), 8.40 (t, J=5.25 Hz, 1H), 8.25-8.37 (m, 1H), 8.11 (d, J=8.88 Hz, 1H), 7.94 (d, J=8.88 Hz, 1H), 7.23 (dd, J=8.44, 3.44 Hz, 1H), 6.54-6.75 (m, 1H), 4.32 (s, 1H), 4.10 (d, J=4.88 Hz, 2H), 3.81 (d, J=5.75 Hz, 1H), 3.35 (dd, J=17.45, 8.82 Hz, 3H), 3.10-3.21 (m, 1H), 2.91 (s, 4H), 2.68-2.75 (m, 3H), 2.52-2.55 (m, 2H), 2.18 (s, 1H), 1.99-2.11 (m, 1H), 1.75-1.95 (m, 1H).

### Example 1.39

### 1-Isopropoxycarbonyloxyethyl 7-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate

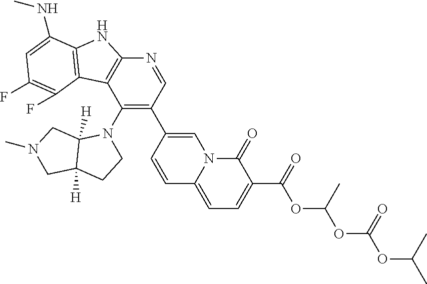 [SMILES: CNc1cc(F)c(F)c2c1[nH]c1ncc(-c3ccc4ccc(C(=O)OC(C)OC(=O)OC(C)C)c(=O)n4c3)c(N3CC[C@H]4CN(C)C[C@H]43)c12]

To a solution of 7-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid (100 mg, 0.180 mmol, compound 1.34D) in DMSO (5 mL) was added K₂CO₃ (50 mg, 0.360 mmol), and the reaction mixture was stirred at 20° C. for 0.5 h before 1-chloroethyl isopropyl carbonate (70.0 mg, 0.420 mmol) was added. The resulting reaction mixture was stirred at 20° C. for another 15.5 h before it was concentrated under the reduced pressure. The residue was subject to prep-HPLC (TFA as additive) purification to give 1-Isopropoxycarbonyloxyethyl 7-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate (132.2 mg, 90.4% yield) as a yellow solid. MS (ESI): 675.4 ([M+H]⁺). ¹H NMR (400 MHz, DMSO-d₆) δ ppm: 11.77 (r s, 1H), 9.23 (s, 1H), 8.30 (m, 2H), 8.03 (m, 1H), 7.95 (m, 1H), 6.98 (d, J=8.4 Hz, 1H), 6.87 (m, 1H), 6.61 (m, 1H), 4.82 (m, 1H), 4.33 (m, 1H), 3.64 (m, 1H), 3.12 (m, 2H), 2.94 (s, 3H), 2.71 (s, 3H), 2.54 (m, 1H), 2.51 (m, 3H), 2.16 (m, 1H), 1.89 (m, 1H), 1.58 (d, J=5.6 Hz, 3H), 1.26 (d, 0.7=6.4 Hz, 6H).

### Example 1.40

### 1-Acetoxyethyl 7-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate

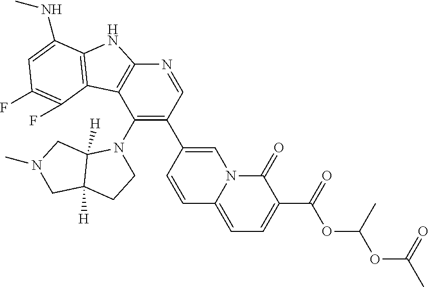 [SMILES: CNc1cc(F)c(F)c2c1[nH]c1ncc(-c3ccc4ccc(C(=O)OC(C)OC(C)=O)c(=O)n4c3)c(N3CC[C@H]4CN(C)C[C@H]43)c12]

To a solution of 7-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid (200 mg, 0.37 mmol, compound 1.34D) in DMF (5 mL) was added K₂CO₃ (100 mg, 0.72 mmol), and the mixture was stirred at 20° C. for 0.5 h before 1-bromoethyl acetate (100 mg, 0.60 mmol) was added. The resulting mixture was stirred at 20° C. for another 15.5 h before it was concentrated under reduce pressure. The residue was subject to prep-HPLC (TFA as additive) purification to give 1-acetoxyethyl 7-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate (15 mg, 4.9% yield) as a yellow solid. MS (ESI): 631.3 ([M+H]⁺). ¹H NMR (400 MHz, DMSO-d₆) δ ppm: 11.75 (ms, 1H), 9.23 (s, 1H), 8.31 (d, J=8.4 Hz, 2H), 8.031 (m, 1H), 7.94 (m, 1H), 7.00 (m, 2H), 6.62 (m, 1H), 4.33 (m, 1H), 3.64 (m, 2H), 3.41 (m, 1H), 2.94 (s, 3H), 2.72 (s, 3H), 2.54 (m, 2H), 2.13 (m, 2H), 2.07 (s, 3H), 1.90 (m, 2H), 1.56 (d, J=5.6 Hz, 3H).

### Example 1.41

### 7-[5,6-Difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-N-methylsulfonyl-4-oxo-quinolizine-3-carboxamide

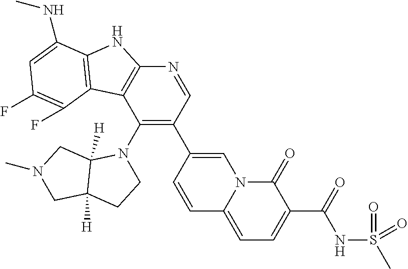 [SMILES: CNc1cc(F)c(F)c2c1[nH]c1ncc(-c3ccc4ccc(C(=O)NS(C)(=O)=O)c(=O)n4c3)c(N3CC[C@H]4CN(C)C[C@H]43)c12]

To a solution of 7-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid (100 mg, 0.18 mmol, compound 1.34D) in DCM (5 mL) was added thionyl chloride (43.7 mg, 0.37 mmol) at 0° C. After the addition, the mixture solution was allowed to warm-up and stirred at 25° C. for 2 h until LC-MS showed the starting material was consumed completely. The mixture solution was then added to a solution of methanesulfonamide (87.3 mg, 0.92 mmol) in DCM (5 mL) and the resulting solution was stirred at 25° C. for another 16 h. The reaction mixture was concentrated in vacuo to give a crude product, which was purified by prep-HPLC (NH₄HCO₃ as additive) to give 7-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-N-methylsulfonyl-4-oxo-quinolizine-3-carboxamide (7.5 mg, 6.2% yield) as a yellow solid. MS (ESI): 622.1 ([M+H]⁺). ¹H NMR (400 MHz, DMSO-d₆) δ ppm: 11.82 (br s, 1H), 9.26 (s, 1H), 8.48 (d, J=8.6 Hz, 1H), 8.20-8.23 (m, 1H), 8.13-8.18 (m, 1H), 7.30 (br d, J=8.6 Hz, 1H), 6.58 (br dd, 0.7=13.5, 6.2 Hz, 1H), 5.75 (br d, J=4.4 Hz, 1H), 4.45 (br s, 1H), 4.16 (br d, J=7.0 Hz, 1H), 3.17 (s, 3H), 2.89 (br d, J=4.6 Hz, 3H), 2.70 (s, 1H), 2.26 (br s, 1H), 2.15-2.21 (m, 2H), 2.13 (br s, 3H), 1.97-2.05 (m, 1H), 1.90 (dt, 0.7=15.1, 7.5 Hz, 2H), 1.68 (br s, 1H).

### Example 1.42

### 6-[5,6-Difluoro-8-(methylamino)-4-[cis-5-methyl-2,3,3a,4,6,6a-hexahydropyrrolo[2,3-c]pyrrol-1-yl]-9H-pyrido[2,3-b]indol-3-yl]-1-methyl-N-methylsulfonyl-4-oxo-1,8-naphthyridine-3-carboxamide

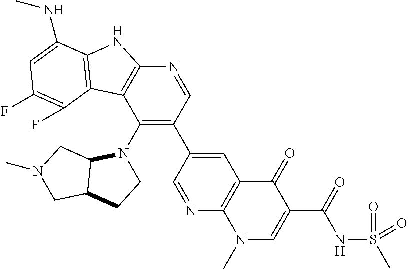

In analogy to the synthesis of Example 1.10, 6-[5,6-difluoro-8-(methylamino)-4-[cis-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (compound 1.29A) was used instead of 6-[5-cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (compound 1.03D) to give 6-[5,6-Difluoro-8-(methylamino)-4-[cis-5-methyl-2,3,3a,4,6,6a-hexahydropyrrolo[2,3-c]pyrrol-1-yl]-9H-pyrido[2,3-b]indol-3-yl]-1-methyl-N-methylsulfonyl-4-oxo-1,8-naphthyridine-3-carboxamide (8% yield) as a yellow solid after prep-HPLC purification (NH₃.H₂O as additive). MS (ESI): 637.1 ([M+H]⁺). ¹H NMR (400 MHz, DMSO-d₆) δ ppm: 12.97 (d, 1H), 11.93 (d, 1H), 9.47 (m, 1H), 9.30 (s, 1H), 9.19 (m, 1H), 9.10 (m, 1H), 8.68 (s, 1H), 8.26 (d, 1H), 6.64 (m, 1H), 4.18 (d, 3H), 4.12 and 3.98 (m, 1H), 3.66 (m, 1H), 3.44 (s, 3H), 3.10 (m, 1H), 3.02 (m, 1H), 2.91 (s, 3H), 2.82 (m, 2H), 2.65 (m, 5H), 2.17 (m, 1H), 2.06 (m, 1H), 1.82 (m, 1H).

### Example 1.43

### 6-[5,6-Difluoro-8-[methyl(phosphonooxymethoxycarbonyl)amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid

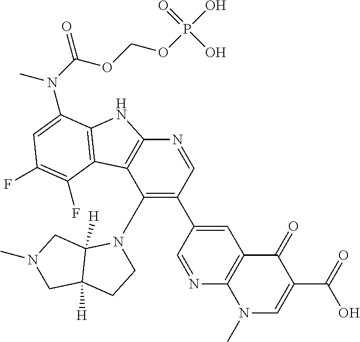 [SMILES: CN1C[C@@H]2CCN(c3c(-c4cnc5c(c4)c(=O)c(C(=O)O)cn5C)cnc4[nH]c5c(N(C)C(=O)OCOP(=O)(O)O)cc(F)c(F)c5c34)[C@@H]2C1]

In analogy to the synthesis of Example 1.18, tot-butyl N-[3-chloro-5,6-difluoro-4-[(3aS,6aS)-5-methyl-2,3,3a,4,6,6a-hexahydropyrrolo[2,3-c]pyrrol-1-yl]-9H-pyrido[2,3-b]indol-8-yl]-N-methyl-carbamate (compound 1.26B) was used instead of tot-butyl N-[3-chloro-5,6-difluoro-4-[(3aR,6aR)-5-methyl-2,3,3a,4,6,6a-hexahydropyrrolo[2,3-c]pyrrol-1-yl]-9H-pyrido[2,3-b]indol-8-yl]-N-methyl-carbamate (compound 1.16B) in step a to afford 6-[5,6-difluoro-8-[methyl(phosphonooxymethoxycarbonyl)amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid after steps b, c, d, e, and f as a yellow solid. MS (ESI): 714.0 ([M+H]⁺). ¹H NMR (400 MHz, DMSO-d₆) δ ppm: 12.87-13.85 (m, 1H), 9.25 (br s, 1H), 9.10 (br s, 1H), 8.67 (br s, 1H), 8.14 (br d, J=7.9 Hz, 1H), 7.59 (br d, J=6.1 Hz, 1H), 5.37-5.62 (m, 1H), 4.34 (br s, 1H), 4.16 (s, 3H), 3.30 (br s, 3H), 3.26 (br s, 3H), 3.07 (br s, 1H), 2.88 (br s, 2H), 2.56-2.64 (m, 2H), 2.18-2.38 (m, 2H), 1.97 (br s, 1H), 1.67 (br s, 1H).

### Example 44

### 6-[4-(Dimethylamino)-5,6-difluoro-8-(methylamino)-1-(phosphonooxymethyl)pyrido[2,3-b]indol-3-yl]-1-methyl-4-oxo-1,8-naphthyridine-3-carboxylic acid

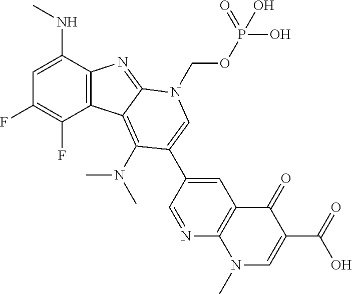 [SMILES: CNc1cc(F)c(F)c2c3c(N(C)C)c(-c4cnc5c(c4)c(=O)c(C(=O)O)cn5C)cn(COP(=O)(O)O)c-3nc12]

The titled compound was synthesized according to the following scheme:

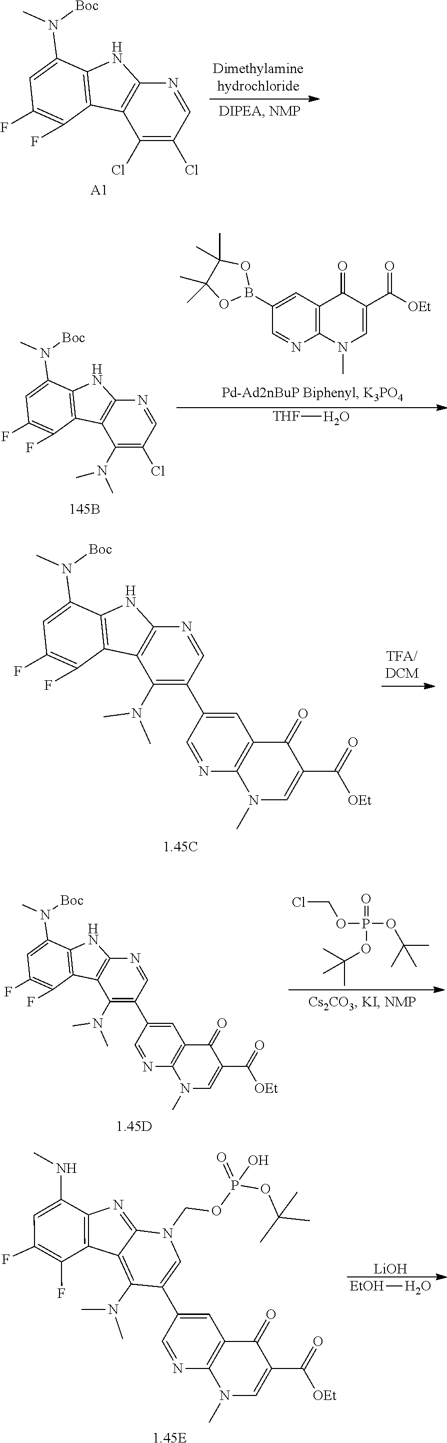

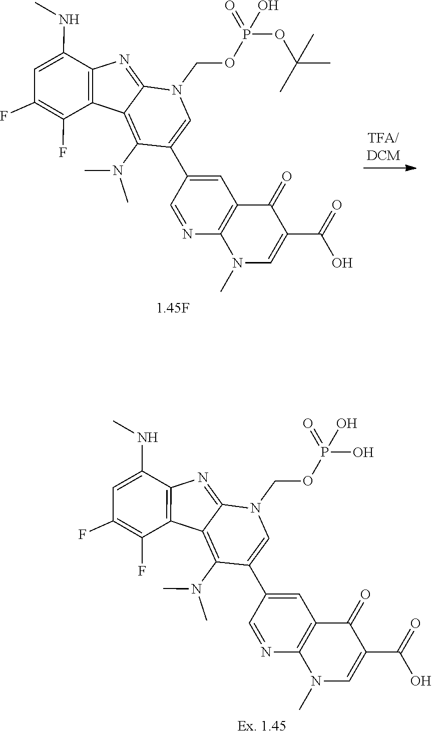 [SMILES: CNc1cc(F)c(F)c2c3c(N(C)C)c(-c4cnc5c(c4)c(=O)c(C(=O)O)cn5C)cn(COP(=O)(O)OC(C)(C)C)c-3nc12 ; "1.45F" ; CNc1cc(F)c(F)c2c3c(N(C)C)c(-c4cnc5c(c4)c(=O)c(C(=O)O)cn5C)cn(COP(=O)(O)O)c-3nc12 ; "Ex. 1.45"]

### Step (a) Preparation of tert-butyl N-[3-chloro-4-(dimethylamino)-5,6-difluoro-9H-pyrido[2,3-b]indol-8-yl]-N-methyl-carbamate (Compound 1.45B)

A mixture solution of tert-butyl N-(3,4-dichloro-5,6-difluoro-9H-pyrido[2,3-b]indol-8-yl)-N-methyl-carbamate (300.0 mg, 0.75 mmol, Intermediate A1), dimethylamine hydrochloride (150.0 mg, 1.8 mmol), and DIPEA (290.0 mg, 2.2 mmol) in anhydrous NMP (5 mL) was stirred at 100° C. for 12 h until LCMS showed the starting material was consumed. The reaction mixture was cooled back to r.t., diluted with EtOAc (100 mL), poured into water (100 mL), and extracted with EtOAc (100 mL). The organic layer was washed with brine (100 mL), dried over anhy. Na₂SO₄, filtered, and concentrated to give a crude product, which was purified by prep-TLC (petrolieum ether:EtOAc=2:1) to give tert-butyl N-[3-chloro-4-(dimethylamino)-5,6-difluoro-9H-pyrido[2,3-b]indol-8-yl]-N-methyl-carbamate (320 mg, 94.0% yield) as a yellow solid. MS (ESI): 411.2 ([M+H]⁺).

### Step (b) Preparation of ethyl 6-[8-[tert-butoxycarbonyl(methyl)amino]-4-(dimethylamino)-5,6-difluoro-9H-pyrido[2,3-b]indol-3-yl]-1-methyl-4-oxo-1,8-naphthyridine-3-carboxylate (Compound 1.45C)

To a mixture of tert-butyl N-[3-chloro-4-(dimethylamino)-5,6-difluoro-9H-pyrido[2,3-b]indol-8-yl]-N-methyl-carbamate (0.32 g, 0.78 mmol), ethyl 1-methyl-4-oxo-6-(4,4,5,5-tetramethyl-1,3,2-dioxaborolan-2-yl)-1,8-naphthyridine-3-carboxylate (0.44 g, 1.22 mmol, Intermediate B1), and K₃PO₄ (0.5 g, 2.34 mmol) in THF (10 mL) and water (2 mL) was added Pd-Ad2nBuP Biphenyl (29.1 mg, 0.04 mmol, cataCXium A-Pd-G2, CAS: 1375477-29-4) in glovebox under Ar. The reaction mixture was stirred at 70° C. for 2 h until LCMS showed the starting material was consumed. The mixture solution was cooled back to r.t., diluted with EtOAc (100 mL), poured into water (80 mL), and extracted with EtOAc (100 mL). The organic layer was washed with brine (100 mL), filtered, and concentrated in vacuo to give a crude product, which was purified by prep-HPLC (TFA as additive) to give ethyl 6-[8-[tert-butoxycarbonyl(methyl)amino]-4-(dimethylamino)-5,6-difluoro-9H-pyrido[2,3-b]indol-3-yl]-1-methyl-4-oxo-1,8-naphthyridine-3-carboxylate (400 mg, 84.7% yield) as a yellow solid. MS (ESI): 564.3 ([M+H]⁺).

### Step (c) Preparation of ethyl 6-[4-(dimethylamino)-5,6-difluoro-8-(methylamino)-9H-pyrido[2,3-b]indol-3-yl]-1-methyl-4-oxo-1,8-naphthyridine-3-carboxylate (Compound 1.45D)

To a solution of ethyl 6-[8-[tert-butoxycarbonyl(methyl)amino]-4-(dimethylamino)-5,6-difluoro-9H-pyrido[2,3-b]indol-3-yl]-1-methyl-4-oxo-1,8-naphthyridine-3-carboxylate (400.0 mg, 0.66 mmol) in DCM (3.0 mL) was added TFA (1.0 mL, 13.0 mmol), and the resulting reaction mixture was stirred at 20° C. for 1 h. After LC-MS showed the starting material was consumed, the mixture was then concentrated in vacuo to give a crude product, which was purified by prep-HPLC (TFA as additive) to give ethyl 6-[4-(dimethylamino)-5,6-difluoro-8-(methylamino)-9H-pyrido[2,3-b]indol-3-yl]-1-methyl-4-oxo-1,8-naphthyridine-3-carboxylate (330 mg, 98.8% yield) as a yellow solid. MS (ESI): 507.3 ([M+H]⁺).

### Step (d) Preparation of ethyl 6-[1-[[tert-butoxy(hydroxy)phosphoryl]oxymethyl]-4-(dimethylamino)-5,6-difluoro-8-(methylamino)pyrido[2,3-b]indol-3-yl]-1-methyl-4-oxo-1,8-naphthyridine-3-carboxylate (Compound 1.45E)

A mixture solution of ethyl 6-[4-(dimethylamino)-5,6-difluoro-8-(methylamino)-9H-pyrido[2,3-b]indol-3-yl]-1-methyl-4-oxo-1,8-naphthyridine-3-carboxylate (120.0 mg, 0.24 mmol), di-tert-butyl chloromethyl phosphate (224.0 mg, 0.87 mmol), cesium carbonate (232.0 mg, 0.71 mmol), and potassium iodide (120.0 mg, 0.72 mmol) in NMP (6.0 mL) was stirred at 20° C. for 12 h. After LC-MS showed the starting material was consumed, the mixture was poured into water (50.0 mL), and extracted with EtOAc (50.0 mL). The organic layer was dried over anhy. Na₂SO₄, filtered, and concentrated in vacuo to give a crude product, which was purified by prep-HPLC (NH₄HCO₃ as additive) to give ethyl 6-[1-[[tert-butoxy(hydroxy)phosphoryl]oxymethyl]-4-(dimethylamino)-5,6-difluoro-8-(methylamino)pyrido[2,3-b]indol-3-yl]-1-methyl-4-oxo-1,8-naphthyridine-3-carboxylate (20 mg, 75.3% yield) as colorless oil. MS (ESI): 673.3 ([M+H]⁺).

### Step (e) Preparation of 6-[1-[[tert-butoxy(hydroxy)phosphoryl]oxymethyl]-4-(dimethylamino)-5,6-difluoro-8-(methylamino)pyrido[2,3-b]indol-3-yl]-1-methyl-4-oxo-1,8-naphthyridine-3-carboxylic acid (Compound 1.45F)

To a solution of ethyl 6-[1-[[tert-butoxy(hydroxy)phosphoryl]oxymethyl]-4-(dimethylamino)-5,6-difluoro-8-(methylamino)pyrido[2,3-b]indol-3-yl]-1-methyl-4-oxo-1,8-naphthyridine-3-carboxylate_4 (20.0 mg, 0.03 mmol) in EtOH (5.0 mL) and water (1.0 mL) was added LiOH.H₂O (12.5 mg, 0.3 mmol), and the resulting reaction mixture was stirred at 20° C. for 2 h. After LC-MS showed the starting material was consumed, the mixture solution was adjusted to pH=5 with aq. HCl solution (1N). It was concentrated in vacuo to give a crude product, which was purified by prep-HPLC (TFA as additive) to give 6-[1-[[tert-butoxy(hydroxy)phosphoryl]oxymethyl]-4-(dimethylamino)-5,6-difluoro-8-(methylamino)pyrido[2,3-b]indol-3-yl]-1-methyl-4-oxo-1,8-naphthyridine-3-carboxylic acid (10 mg, 52.2% yield) as a yellow solid. MS (ESI): 645.2 ([M+H]⁺).

### Step (f) Preparation of 6-[4-(dimethylamino)-5,6-difluoro-8-(methylamino)-1-(phosphonooxymethyl)pyrido[2,3-b]indol-3-yl]-1-methyl-4-oxo-1,8-naphthyridine-3-carboxylic acid (Ex, 1.45)

To a solution of 6-[1-[[tert-butoxy(hydroxy)phosphoryl]oxymethyl]-4-(dimethylamino)-5,6-difluoro-8-(methylamino)pyrido[2,3-b]indol-3-yl]-1-methyl-4-oxo-1,8-naphthyridine-3-carboxylic acid (10.0 mg, 0.02 mmol) in DCM (0.6 mL) was added TFA/DCM (v:v=1:4, 0.5 mL, 0.02 mmol), and the resulting reaction mixture was stirred at 20° C. for 1 h. After LC-MS showed the starting material was consumed, the mixture was concentrated in vacuo to give a crude product of 6-[4-(dimethylamino)-5,6-difluoro-8-(methylamino)-1-(phosphonooxymethyl)pyrido[2,3-b]indol-3-yl]-1-methyl-4-oxo-1,8-naphthyridine-3-carboxylic acid. MS (ESI): 589.2 ([M+H]⁺).

### Example 45

### 6-[5-Cyano-6-fluoro-8-[methyl-[1-[(2S)-2-amino-3-methyl-butanoyl]oxyethoxycarbonyl]amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid

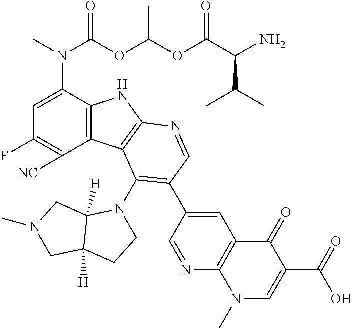 [SMILES: CC(OC(=O)[C@@H](N)C(C)C)OC(=O)N(C)c1cc(F)c(C#N)c2c1[nH]c1ncc(-c3cnc4c(c3)c(=O)c(C(=O)O)cn4C)c(N3CC[C@H]4CN(C)C[C@H]43)c12]

The titled compound was synthesized according to the following scheme:

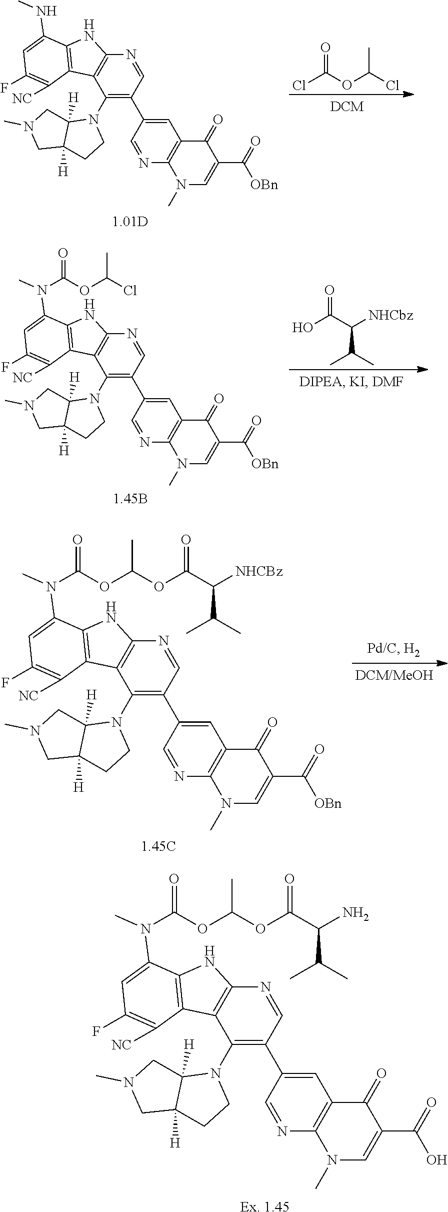

### Step (a) Preparation of benzyl 6-[8-[1-chloroethoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (Compound 1.45B)

In analogy to the synthesis of 1.01E, 1-chloroethyl chloroformate was used instead of chloromethyl chloroformate in step d to give a crude product of benzyl 6-[8-[1-chloroethoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (77.4% yield) as a light yellow solid. MS (ESI): 763.2 ([M+H]⁺).

### Step (b) Preparation of benzyl 6-[5-cyano-6-fluoro-8-[methyl-[1-[(2S)-2-(benzyloxycarbonylamino)-3-methyl-butanoyl]oxyethoxycarbonyl]amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (Compound 1.45C)

In analogy to the synthesis of 1.14B, (2S)-2-(benzyloxycarbonylamino)-3-methyl-butanoic acid was used instead of N-benzyloxy carbonyl glycine to give benzyl 6-[5-cyano-6-fluoro-8-[methyl-[1-[(2S)-2-(benzyloxycarbonylamino)-3-methyl-butanoyl]oxyethoxycarbonyl]amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (44.3% yield) as a light yellow solid after prep-HPLC purification (formic acid as additive). MS (ESI): 978.4 (([M+H]⁺).

### Step (c) Preparation of 6-[5-cyano-6-fluoro-8-[methyl-[1-[(2S)-2-amino-3-methyl-butanoyl]oxyethoxycarbonyl]amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (Ex, 1.45)

In analogy to the synthesis of Example 1.14, benzyl 6-[5-cyano-6-fluoro-8-[methyl-[1-[(2S)-2-(benzyloxycarbonylamino)-3-methyl-butanoyl]oxyethoxycarbonyl]amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (compound 1.45C) was used instead of benzyl 6-[8-[[2-(benzyloxycarbonylamino)acetyl]oxymethoxycarbonyl-methyl-amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate (compound 1.14B) to give 6-[5-cyano-6-fluoro-8-[methyl-[1-[(2S)-2-amino-3-methyl-butanoyl]oxyethoxycarbonyl]amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid (45.7% yield) as a yellow solid after prep-HPLC purification (NH₄HCO₃ as additive). MS (ESI): 754.4 ([M+H]⁺).

### Example 46

### Lyophilisation Solubility Assay (LYSA):

The solubility of compounds of present invention was assessed by LYSA assay. Samples were prepared in duplicate from 10 mM DMSO stock solutions (20 μL) diluted in 30 μL pure DMSO. After evaporation of DMSO with a centrifugal vacuum evaporator, the residue was dissolved in 0.05 M phosphate buffer (150 μL, pH 6.5), which was stirred for one hour and shook for two hours. After one night, the solution was filtered using a microtiter filter plate. Then the filtrate and its 1/10 dilution were analyzed by HPLC-UV. In addition, a four-point calibration curve was prepared from the 10 mM stock solutions and used for the solubility determination of the compounds. The results were in μg/mL and summarized in Table 1. Particular compounds of the present invention were found to have much improved solubility (LYSA @ pH 6.5) compared to the parent molecules (1.03D, 1.26E and 1.34D).

TABLE 1
Solubility data of Examples

| | | Example No. of | |
|---|---|---|---|
| Example No. of | | corresponding | |
| parent molecule | Lysa (μg/mL) | prodrugs | Lysa (μg/mL) |
| 1.03D | 194 | 1.01 | >595 |
| | | 1.02 | >895 |
| | | 1.03 | >730 |
| | | 1.04 | >950 |
| | | 1.07 | >1060 |
| | | 1.08 | 480 |
| 1.26E | 137 | 1.26 | >783 |
| | | 1.27 | >685 |
| | | 1.28 | >945 |
| | | 1.43 | 595 |
| 1.34D | 54 | 1.32 | >875 |
| | | 1.33 | 680 |
| | | 1.34 | >930 |
| | | 1.35 | >915 |
| | | 1.36 | >1000 |
| | | 1.37 | 315 |

### Example 47

### Plasma Stability Assay

The degradation of prodrug and formation of active drug of present invention were monitored by current plasma stability assay. The assay is conducted in human plasma (purchased from Bioreclamation, Lot No. BRH1380385) or mouse plasma (prepared in-house using CD-1 mouse purchased from Vital River). Plasma is spiked with the test prodrug (10 μM final concentration) dissolved in DMSO (or water) and incubated at 37° C. Aliquots are removed at 0, 5, 15 and 30 minutes of incubation time, quenched with appropriate volume of internal standard solution (I.S., 0.2 μM of tolbutamide, prepared in ACN and water (1:1, v/v)), according to the mass spectrometer intensity of both analytes. Samples are centrifuged, the supernatant are removed and analyzed by LC-MS/MS.

The degradation of prodrug was illustrated by calculation of percent remaining of prodrug at 30 min compared to that at 0 min (Table 2).

TABLE 2
Plasma stability data of Examples

| | Percent |
|---|---|
| | Remaining (%) |
| Example No. | H/M |
| 1.02 | 88.2/70.2 |
| 1.04 | 95.9/79.1 |
| 1.08 | 22.3/1.3 |
| 1.13 | 1.9/0.6 |
| 1.15 | 50.5/4.7 |
| 1.18 | 67.2/72.0 |
| 1.21 | 88.7/56.0 |
| 1.39 | 98.4/0.1 |

### Example 48

### 50% Growth Inhibitory Concentration (IC₅₀) Determination Assay:

The in vitro antimicrobial activity of the compounds against S. aureus (ATCC29213), K. pneumoniae (ATCC10031), and A. baumannii (ATCC17978), was determined according to the following procedure:

The assay used a 10-points Iso-Sensitest broth medium to measure quantitatively the in vitro activity of the compounds against S. aureus ATCC29213, K. pneumoniae ATCC 10031, and A. baumannii ATCC 17978.

Stock compounds in DMSO were serially two-fold diluted (range from 50 to 0.097 μM final concentration) in 384 wells microtiter plates and inoculated with 49 μL the bacterial suspension in Iso-Sensitest broth medium to have a final cell concentration of 5×10⁵ CFU/mL in a final volume/well of 50 μL/well. Microtiter plates were incubated at 35±2° C.

Bacterial cell growth was determined with the measurement of optical density at λ=600 nm each 20 minutes over a time course of 16 h.

Growth inhibition was calculated during the logarithmic growth of the bacterial cells with determination of the concentration inhibiting 50% (IC₅₀) of the growth.

The parent compounds of the present invention were tested for their concentration inhibiting 50% (IC₅₀). The data of IC₅₀ over S. aureus (ATCC29213), K. pneumoniae (ATCC10031), and A. baumannii (ATCC17978) are illustrated in Table 6. Particular compounds of the present invention were found to have IC₅₀≤1 μM.

TABLE 3
IC₅₀ values of the parent compounds of this invention against
S. aureus, K. pneumoniae and A. baumannii

| | IC₅₀ (μM) | | |
|---|---|---|---|
| Example | S. aureus | K. pneumoniae | A. baumannii |
| No. | ATCC29213 | ATCC10031 | ATCC17978 |
| 1.03D | <0.098* | <0.098* | 0.045 |
| 1.26E | <0.098* | <0.098* | 0.039 |
| 1.34D | <0.098* | <0.098* | <0.098* |

1. A compound of formula (I), 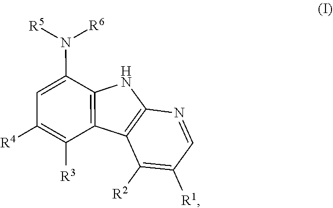
wherein
R¹ is 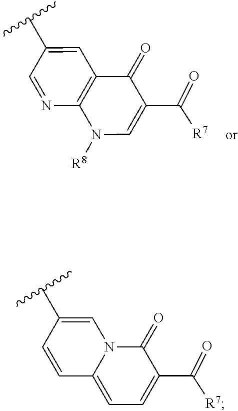
wherein R⁷ is selected from the group consisting of OH; (C₁₋₆alkyl-2-oxo-1,3-dioxol-4-yl)C₁₋₆alkoxy; aminoC₁₋₆alkylcarbonyloxyC₁₋₆alkoxy; C₁₋₆alkoxy; C₁₋₆alkoxycarbonyloxyC₁₋₆alkoxy; C₁₋₆alkylcarbonyloxyC₁₋₆alkoxy; C₁₋₆alkylsulfonylamino; carboxyC₁₋₆alkylaminocarbonylC₁₋₆alkylamino and phenylC₁₋₆alkoxy; and
R⁸ is C₁₋₆alkyl;
R² is selected from 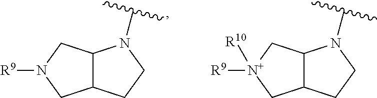
and —N(R⁹)₂;
wherein R⁹ is C₁₋₆alkyl; and
R¹⁰ is selected from the group consisting of C₁₋₆alkyl-2-oxo-1,3-dioxol-4-yl; C₁₋₆alkoxy(hydroxy)phosphoryloxyC₁₋₆alkyl and phosphonooxyC₁₋₆alkyl;
R³ is halogen or cyano;
R⁴ is halogen;
R⁵ is selected from the group consisting of H; (aminoC₁₋₆alkyl carbonyl)aminoC₁₋₆alkylcarbonyl; ((aminoC₁₋₆alkylcarbonyl)aminoC₁₋₆alkylcarbonyl)aminoC₁₋₆alkylcarbonyl; (C₁₋₆alkoxy(hydroxy)phosphoryl)C₁₋₆alkoxycarbonyl; (C₁₋₆alkoxy)₂phosphoryl; aminoC₁₋₆alkylcarbonyloxyC₁₋₆alkoxycarbonyl; C₁₋₆alkylcarbonyloxyC₁₋₆alkoxycarbonyl; carboxyC₂₋₆ alkenylcarbonyloxyC₁₋₆alkoxycarbonyl; formyl; phenylC₁₋₆alkoxy(hydroxy)phosphoryl; phosphonooxyC₁₋₆alkoxycarbonyl and sulfonyl; and
R⁶ is C₁₋₆alkyl;
with the proviso that when R⁵ is H and R⁷ is OH, R² is not 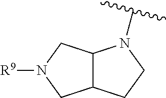
or —N(R⁹)₂;
or a pharmaceutically acceptable salt thereof.

2. A compound of formula (I) according to claim 1, wherein
R¹ is 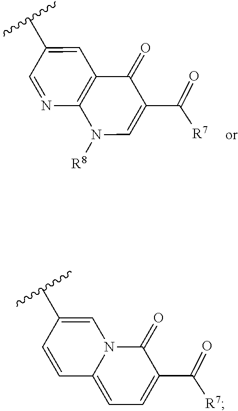
wherein R⁷ is selected from the group consisting of OH; (5-methyl-2-oxo-1,3-dioxol-4-yl)methoxy; acetoxyethoxy; amino(methyl)butanoyloxyethoxy; benzyloxy; carboxymethylaminocarbonylmethylamino; ethoxy; isopropoxycarbonyloxyethoxy and methylsulfonylamino; and
R⁸ is methyl;
R² is selected from 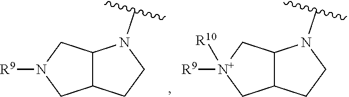
and —N(R⁹)₂;
wherein R⁹ is methyl;
R¹⁰ is selected from the group consisting of 5-methyl-2-oxo-1,3-dioxol-4-ylmethyl; tert-butoxy(hydroxy)phosphoryloxymethyl and phosphonooxymethyl;
R³ is fluoro or cyano;
R⁴ is fluoro;
R⁵ is selected from the group consisting of H; ((aminoacetyl)aminoacetyl)aminoacetyl; (aminoacetyl)aminoacetyl; (tert-butoxy(hydroxy)phosphoryloxy)methoxycarbonyl; acetoxyethoxycarbonyl; aminoacetyloxymethoxycarbonyl; amino(methyl)butanoyloxyethoxycarbony; benzyloxy(hydroxy)phosphoryl; carboxypropenoyloxymethoxycarbonyl; diethoxyphosphoryl; formyl; phosphonooxymethoxycarbonyl and sulfo; and
R⁶ is methyl;
with the proviso that when R⁵ is H and R⁷ is OH, R² is not 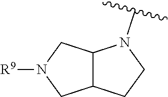
or —N(R⁹)₂;
or a pharmaceutically acceptable salt thereof.

3. A compound according to claim 1, wherein
R¹ is 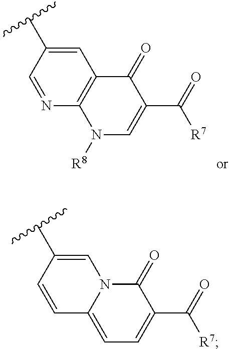
wherein R⁷ is selected from the group consisting of (C₁₋₆alkyl-2-oxo-1,3-dioxol-4-yl)C₁₋₆alkoxy; aminoC₁₋₆alkylcarbonyloxyC₁₋₆alkoxy; C₁₋₆alkoxy; C₁₋₆alkoxycarbonyloxyC₁₋₆alkoxy; C₁₋₆alkylcarbonylC₁₋₆alkoxy; C₁₋₆alkylsulfonylamino; carboxyC₁₋₆alkylaminocarbonylC₁₋₆alkylamino and phenylC₁₋₆alkoxy;
R⁸ is C₁₋₆alkyl;
R² is 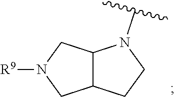
wherein R⁹ is C₁₋₆alkyl;
R³ is halogen or cyano;
R⁴ is halogen;
R⁵ is H;
R⁶ is C₁₋₆alkyl;
or a pharmaceutically acceptable salt thereof.

4. A compound according to claim 3, or a pharmaceutically acceptable salt thereof, wherein R⁷ is selected from the group consisting of (C₁₋₆alkyl-2-oxo-1,3-dioxol-4-yl)C₁₋₆alkoxy; aminoC₁₋₆alkyl carbonyloxyC₁₋₆alkoxy; C₁₋₆alkoxycarbonyloxyC₁₋₆alkoxy and C₁₋₆alkyl carbonyloxyC₁₋₆alkoxy.

5. A compound according to claim 4, or a pharmaceutically acceptable salt thereof, wherein R⁷ is selected from the group consisting of (5-methyl-2-oxo-1,3-dioxol-4-yl)methoxy; acetoxyethoxy; amino(methyl)butanoyloxyethoxy and isopropoxycarbonyloxyethoxy.

6. A compound according to claim 5, or a pharmaceutically acceptable salt thereof, wherein R³ is fluoro or cyano; R⁴ is fluoro; R⁶ is methyl; R⁸ is methyl; and R⁹ is methyl.

7. A compound according to claim 1, wherein
R¹ is 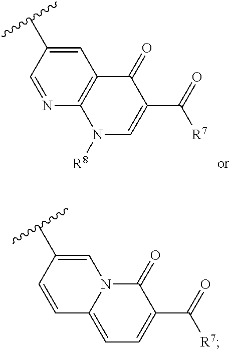
wherein R⁷ is OH; and
R⁸ is C₁₋₆alkyl;
R² is 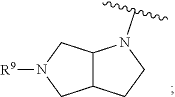
wherein R⁹ is C₁₋₆alkyl;
R³ is halogen or cyano;
R⁴ is halogen;
R⁵ is selected from the group consisting of ((aminoC₁₋₆alkylcarbonyl)aminoC₁₋₆alkylcarbonyl; ((aminoC₁₋₆alkylcarbonyl)aminoC₁₋₆alkylcarbonyl)aminoC₁₋₆alkylcarbonyl; (C₁₋₆alkoxy(hydroxy)phosphoryl)C₁₋₆alkoxycarbonyl; (C₁₋₆alkoxy)₂phosphoryl; aminoC₁₋₆alkylcarbonyloxyC₁₋₆alkoxycarbonyl; C₁₋₆alkylcarbonyloxyC₁₋₆alkoxycarbonyl; carboxyC₂₋₆alkenylcarbonyloxyC₁₋₆alkoxycarbonyl; formyl; phenylC₁₋₆alkoxy(hydroxy)phosphoryl; phosphonooxyC₁₋₆alkoxycarbonyl and sulfonyl; and
R⁶ is C₁₋₆alkyl;
or a pharmaceutically acceptable salt thereof.

8. A compound according to claim 7, or a pharmaceutically acceptable salt thereof, wherein R⁵ is selected from the group consisting of C₁₋₆alkylcarbonyloxyC₁₋₆alkoxycarbonyl; carboxyC₂₋₆alkenylcarbonyloxyC₁₋₆alkoxycarbonyl and phosphonooxyC₁₋₆alkoxycarbonyl.

9. A compound according to claim 8, or a pharmaceutically acceptable salt thereof, wherein R⁵ is selected from the group consisting of acetoxyethoxycarbonyl; carboxypropenoyloxymethoxycarbonyl and phosphonooxymethoxycarbonyl.

10. A compound according to claim 9, or a pharmaceutically acceptable salt thereof, wherein R³ is fluoro or cyano; R⁴ is fluoro; R⁶ is methyl; R⁸ is methyl; and R⁹ is methyl.

11. A compound of formula (II), 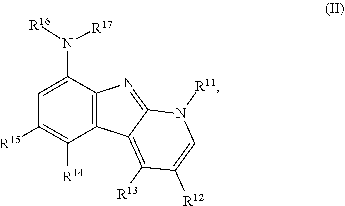
wherein
R¹¹ is selected from the group consisting of C₁₋₆alkyl-2-oxo-1,3-dioxol-4-yl; C₁₋₆alkoxy(hydroxy)phosphoryloxyC₁₋₆alkyl and phosphonooxyC₁₋₆alkyl;
R¹² is 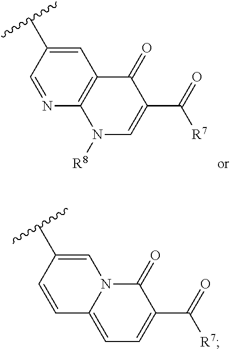
wherein R⁷ is OH;
R⁸ is C₁₋₆alkyl;
R¹³ is 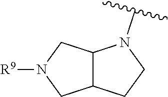
or —N(R⁹)₂; wherein R⁹ is C₁₋₆alkyl;
R¹⁴ is halogen or cyano;
R¹⁵ is halogen;
R¹⁶ is H; and
R¹⁷ is C₁₋₆alkyl;
or a pharmaceutically acceptable salt thereof.

12. A compound according to claim 11, wherein
R¹¹ is selected from the group consisting of 5-methyl-2-oxo-1,3-dioxol-4-ylmethyl; tert-butoxy(hydroxy)phosphoryloxymethyl and phosphonooxymethyl;
R¹² is 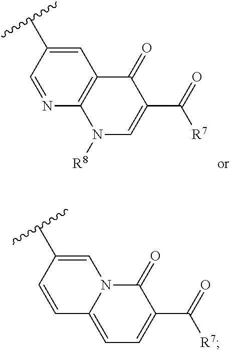
wherein R⁷ is OH; R⁸ is methyl;
R¹³ is 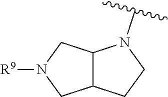
or —N(R⁹)₂; wherein R⁹ is methyl;
R¹⁴ is fluoro or cyano;
R¹⁵ is fluoro;
R¹⁶ is H; and
R¹⁷ is methyl;
or a pharmaceutically acceptable salt thereof.

13. A compound selected from:
6-[5-Cyano-6-fluoro-8-[methyl(phosphonooxymethoxycarbonyl)amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
6-[5-Cyano-6-fluoro-8-[methyl-[[(E)-3-carboxyprop-2-enoyl]oxymethoxycarbonyl]amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
6-[5-Cyano-6-fluoro-8-[formyl(methyl)amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
6-[5-Cyano-6-fluoro-8-(methylamino)-1-(phosphonooxymethyl)-4-[(3aS,6aS)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylic acid;
6-[5-Cyano-6-fluoro-8-(methylamino)-1-[(5-methyl-2-oxo-1,3-dioxol-4-yl)methyl]-4-[(3aS,6aS)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylic acid;
6-[5-Cyano-6-fluoro-8-(methylamino)-4-[(3aS,6aS)-5-methyl-5-[(5-methyl-2-oxo-1,3-dioxol-4-yl)methyl]-2,3,3a,4,6,6a-hexahydropyrrolo[2,3-c]pyrrol-5-ium-1-yl]-9H-pyrido[2,3-b]indol-3-yl]-1-methyl-4-oxo-1,8-naphthyridine-3-carboxylic acid;
6-[5-Cyano-6-fluoro-8-(methylamino)-4-[(3aS,6aS)-5-methyl-5-(phosphonooxymethyl)-2,3,3a,4,6,6a-hexahydropyrrolo[2,3-c]pyrrol-5-ium-1-yl]-9H-pyrido[2,3-b]indol-3-yl]-1-methyl-4-oxo-1,8-naphthyridine-3-carboxylic acid;
2-[(2S)-2-Amino-3-methyl-butanoyl]oxyethyl 6-[5-cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate;
2-[[2-[[6-[5-Cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carbonyl]amino]acetyl]amino]acetic acid;
6-[5-Cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-N-methylsulfonyl-4-oxo-1,8-naphthyridine-3-carboxamide;
1-Acetoxyethyl 6-[5-cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate;
1-Isopropoxycarbonyloxyethyl 6-[6-fluoro-5-methyl-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate;
(5-Methyl-2-oxo-1,3-dioxol-4-yl)methyl 6-[5-cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate;
6-[8-[(2-Aminoacetyl)oxymethoxycarbonyl-methyl-amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
6-[8-[1-Acetoxyethoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
6-[8-[Diethoxyphosphoryl(methyl)amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
6-[8-[[Benzyloxy(hydroxy)phosphoryl]-methyl-amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
6-[5,6-Difluoro-8-[methyl(phosphonooxymethoxycarbonyl)amino]-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
6-[8-[[/c/7-Butoxy(hydroxy)phosphoryl]oxymethoxycarbonyl-methyl-amino]-5,6-difluoro-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
(E)-4-[[[3-(6-Benzyloxycarbonyl-8-methyl-5-oxo-1,8-naphthyridin-3-yl)-5,6-difluoro-4-[(3aR,6aR)-5-methyl-2,3,3a,4,6,6a-hexahydropyrrolo[2,3-c]pyrrol-1-yl]-9H-pyrido[2,3-b]indol-8-yl]-methyl-carbamoyl]oxymethoxy]-4-oxo-but-2-enoic acid;
6-[5,6-Difluoro-8-[methyl-[[(E)-3-carboxyprop-2-enoyl]oxymethoxycarbonyl]amino]-4-[(3aR,6aR)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
6-[1-[[tert-Butoxy(hydroxy)phosphoryl]oxymethyl]-5,6-difluoro-8-(methylamino)-4-[(3aR,6aR)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylic acid;
6-[5,6-Difluoro-8-(methylamino)-1-(phosphonooxymethyl)-4-[(3aR,6aR)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylic acid;
6-[5,6-Difluoro-8-(methylamino)-4-[(3aR,6aR)-5-[[tert-butoxy(hydroxy)phosphoryl]oxymethyl]-5-methyl-2,3,3a,4,6,6a-hexahydropyrrolo[2,3-c]pyrrol-5-ium-1-yl]-9H-pyrido[2,3-b]indol-3-yl]-1-methyl-4-oxo-1,8-naphthyridine-3-carboxylic acid;
6-[5,6-Difluoro-8-(methylamino)-4-[(3aR,6aR)-5-methyl-5-(phosphonooxymethyl)-2,3,3a,4,6,6a-hexahydropyrrolo[2,3-c]pyrrol-5-ium-1-yl]-9H-pyrido[2,3-b]indol-3-yl]-1-methyl-4-oxo-1,8-naphthyridine-3-carboxylic acid;
6-[5,6-Difluoro-8-[formyl(methyl)amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
6-[5,6-Difluoro-8-[methyl(sulfo)amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
6-[5,6-Difluoro-8-[methyl-[[(E)-3-carboxyprop-2-enoyl]oxymethoxycarbonyl]amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
6-[5,6-Difluoro-8-[methyl(sulfo)amino]-4-[cis-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
6-[8-[[2-[(2-Aminoacetyl)amino]acetyl]-methyl-amino]-5,6-difluoro-4-[cis-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
Ethyl 6-[5,6-difluoro-8-(methylamino)-4-[cis-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate;
7-[5,6-Difluoro-8-[methyl(phosphonooxymethoxycarbonyl)amino]-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid;
7-[5,6-Difluoro-8-[methyl-[[(E)-3-carboxyprop-2-enoyl]oxymethoxycarbonyl]amino]-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid;
7-[8-[[2-[[2-[(2-Aminoacetyl)amino]acetyl]amino]acetyl]-methyl-amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid;
7-[8-[[2-[(2-Aminoacetyl)amino]acetyl]-methyl-amino]-5,6-difluoro-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid;
7-[5,6-Difluoro-8-(methylamino)-1-(phosphonooxymethyl)-4-[(3aS,6aS)-5-methyl-2,3,3a,4,6,6a-hexahydropyrrolo[2,3-c]pyrrol-1-yl]pyrido[2,3-b]indol-3-yl]-4-oxo-quinolizine-3-carboxylic acid;
2-[(2S)-2-Amino-3-methyl-butanoyl]oxyethyl 7-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate;
2-[[2-[[7-[5,6-Difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carbonyl]amino]acetyl]amino]acetic acid;
1-Isopropoxycarbonyloxyethyl 7-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate;
1-Acetoxyethyl 7-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate;
7-[5,6-Difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-N-methylsulfonyl-4-oxo-quinolizine-3-carboxamide;
6-[5,6-Difluoro-8-(methylamino)-4-[cis-5-methyl-2,3,3a,4,6,6a-hexahydropyrrolo[2,3-c]pyrrol-1-yl]-9H-pyrido[2,3-b]indol-3-yl]-1-methyl-N-methylsulfonyl-4-oxo-1,8-naphthyridine-3-carboxamide;
6-[5,6-Difluoro-8-[methyl(phosphonooxymethoxycarbonyl)amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
6-[4-(Dimethylamino)-5,6-difluoro-8-(methylamino)-1-(phosphonooxymethyl)pyrido[2,3-b]indol-3-yl]-1-methyl-4-oxo-1,8-naphthyridine-3-carboxylic acid; and
6-[5-Cyano-6-fluoro-8-[methyl-[1-[(2S)-2-amino-3-methyl-butanoyl]oxyethoxycarbonyl]amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
or a pharmaceutically acceptable salt.

14. A process for the preparation of a compound of claim 1 comprising any of the following reactions:
a) the coupling reaction of compound of formula (Ij), 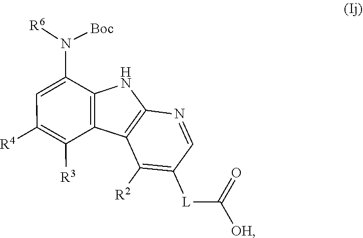
with an alcohol or amine in the presence of a coupling reagent followed by the deprotection of Boc group with an acid;
b) reaction of compound of formula (Ik), 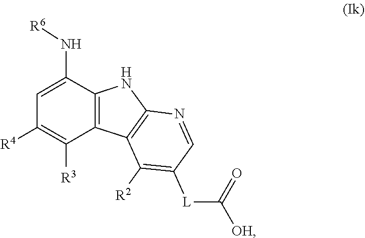
with acylating or sulfonylating reagents;
c) hydrolysis or hydrogentation of compound of formula (In), 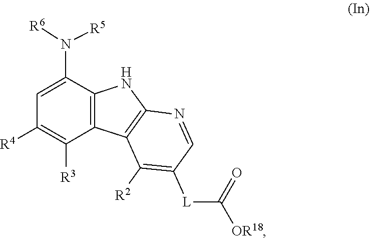
in the presence of a base or a catalyst;
d) hydrolysis or hydrogentation of compound of formula (Ip), 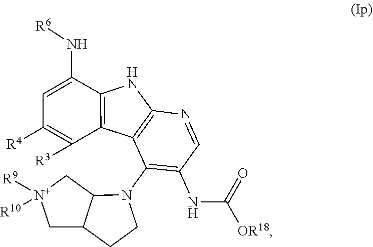
in the presence of a base or a catalyst;
e) hydrolysis or hydrogentation of compound of formula (Iq), 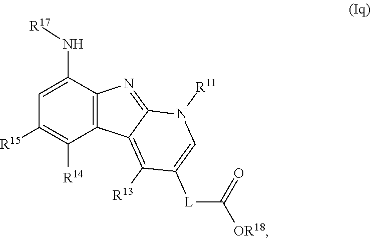
in the presence of a base or a catalyst;
wherein L is unsubstituted or substituted 4-oxo-1,8-naphthyridinyl, or 4-oxoquinolizinyl; R¹⁸ is C₁₋₆alkyl or Bn.

15. (canceled)

16. A pharmaceutical composition comprising a compound of claim 1 and a therapeutically inert carrier.

17-26. (canceled)

27. A method for the treatment or prophylaxis of bacterial infection, which method comprises administering an effective amount of a compound of claim 1.

28. The method according to claim 27, wherein the bacterial infection is caused by Streptococcus pneumoniae, Streptococcus pyogenes, Enterococcus faecalis, Enterococcus faecium, Enterobacter spp. species, Proteus spp. species, Serratia marcescens, Staphylococcus aureus, Coag. Neg. Staphylococci, Haemophilus influenzae, Bacillus anthraces, Mycoplasma pneumoniae, Moraxella catarrhalis, Chlamydophila pneumoniae, Chlamydia trachomatis, Legionella pneumophila, Mycobacterium tuberculosis, Helicobacter pylori, Staphylococcus saprophyticus, Staphylococcus epidermidis, Francisella tularensis, Yersinia pestis, Clostridium difficile, Bacteroides spp. species Neisseria gonorrhoeae, Neisseria meningitidis, Burkholderia pseudomallei, Burkholderia mallei, Borrelia burgdorferi, Mycobacterium avium complex, Mycobacterium abscessus, Mycobacterium kansasii, E. coli or Mycobacterium ulcerans.

29. The method according to claim 28, wherein the bacterial infection is caused by Acinetobacter baumannii, Klebsiella pneumoniae, Pseudomonas aeruginosa, Staphylococcus aureus or E. coli.

30. The compound of claim 13, wherein the compound is selected from
6-[5-Cyano-6-fluoro-8-[methyl(phosphonooxymethoxycarbonyl)amino]-4-[rac-(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
6-[5-Cyano-6-fluoro-8-[methyl-[[(E)-3-carboxyprop-2-enoyl]oxymethoxycarbonyl]amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
6-[5-Cyano-6-fluoro-8-(methylamino)-1-(phosphonooxymethyl)-4-[(3aS,6aS)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylic acid;
6-[5-Cyano-6-fluoro-8-(methylamino)-1-[(5-methyl-2-oxo-1,3-dioxol-4-yl)methyl]-4-[(3aS,6aS)-5-methyl-2,3,3a,4,6,6a-hexahydropyrrolo[2,3-c]pyrrol-1-yl]pyrido[2,3-b]indol-3-yl]-1-methyl-4-oxo-1,8-naphthyridine-3-carboxylic acid;
2-[(2S)-2-Amino-3-methyl-butanoyl]oxyethyl 6-[5-cyano-6-fluoro-8-(methylamino)-4-[(3aS,6aS)-5-methyl-2,3,3a,4,6,6a-hexahydropyrrolo[3,4-b]pyrrol-1-yl]-9H-pyrido[2,3-b]indol-3-yl]-1-methyl-4-oxo-1,8-naphthyridine-3-carboxylate;
1-Acetoxyethyl 6-[5-cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate;
1-Isopropoxycarbonyloxyethyl 6-[6-fluoro-5-methyl-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate;
(5-Methyl-2-oxo-1,3-dioxol-4-yl)methyl 6-[5-cyano-6-fluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylate;
6-[8-[1-Acetoxyethoxycarbonyl(methyl)amino]-5-cyano-6-fluoro-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
6-[5,6-Difluoro-8-[methyl-[[(E)-3-carboxyprop-2-enoyl]oxymethoxycarbonyl]amino]-4-[(3aS,6aS)-5-methyl-2,3,3a,4,6,6a-hexahydropyrrolo[2,3-c]pyrrol-1-yl]-9H-pyrido[2,3-b]indol-3-yl]-1-methyl-4-oxo-1,8-naphthyridine-3-carboxylic acid;
7-[5,6-Difluoro-8-[methyl(phosphonooxymethoxycarbonyl)amino]-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid;
7-[5,6-Difluoro-8-[methyl-[[(E)-3-carboxyprop-2-enoyl]oxymethoxycarbonyl]amino]-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylic acid
7-[5,6-Difluoro-8-(methylamino)-1-(phosphonooxymethyl)-4-[(3aS,6aS)-5-methyl-2,3,3a,4,6,6a-hexahydropyrrolo[3,4-b]pyrrol-1-yl]pyrido[2,3-b]indol-3-yl]-4-oxo-quinolizine-3-carboxylic acid;
2-[(2S)-2-Amino-3-methyl-butanoyl]oxyethyl 7-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate;
1-Isopropoxycarbonyloxyethyl 7-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate;
1-Acetoxyethyl 7-[5,6-difluoro-8-(methylamino)-4-[(3aS,6aS)-5-methyl-2,3,3a,4,6,6a-hexahydropyrrolo[2,3-c]pyrrol-1-yl]-9H-pyrido[2,3-b]indol-3-yl]-4-oxo-quinolizine-3-carboxylate; and
6-[5,6-Difluoro-8-[methyl(phosphonooxymethoxycarbonyl)amino]-4-[(3aS,6aS)-5-methyl-2,3,3a,4,6,6a-hexahydropyrrolo[3,4-b]pyrrol-1-yl]-9H-pyrido[2,3-b]indol-3-yl]-1-methyl-4-oxo-1,8-naphthyridine-3-carboxylic acid.

31. The process of claim 14, wherein, in step a), the coupling reagent is HATU, and the acid is TFA.

32. The process of claim 14, wherein, in step b), the acylating reagent is formic acid, and the sulfonylating reagent is sulfur trioxide.

33. The process of claim 14, wherein, in step c), d) and e), the base is NaOH or LiOH, and the catalyst is Pd/C.
